(19) United States
(12) Patent Application Publication
(10) Pub. No.: US 2017/0022171 A1
(43) Pub. Date: Jan. 26, 2017

(54) 2,5-DISUBSTITUTED CYCLOPENTANECARBOXYLIC ACIDS AND THEIR USE

(71) Applicant: Bayer Pharma Aktiengesellschaft, Berlin (DE)

(72) Inventors: Hartmut BECK, Wuppertal (DE); Volkhart Min-Jian LI, Velbert (DE); Yolanda CANCHO GRANDE, Leverkusen (DE); Andreas TIMMERMANN, Düsseldorf (DE); Dirk BROHM, Mettmann (DE); Hannah JÖRISSEN, Heiligenhaus (DE); Pamela BOGNER, München (DE); Michael GERISCH, Wuppertal (DE); Dieter LANG, Velbert (DE)

(73) Assignee: Bayer Pharma Aktiengesellschaft, Berlin (DE)

(21) Appl. No.: 15/301,392

(22) PCT Filed: Mar. 31, 2015
(86) PCT No.: PCT/EP2015/056984

§ 371 Date: Oct. 2, 2016

(30) Foreign Application Priority Data
Apr. 3, 2014 (EP) 14163307.3

(51) Int. Cl. C07D 253/08; A61K 31/53; C07D 413/12; A61K 45/06

(52) U.S. Cl. CPC C07D 253/08; A61K 45/06; A61K 31/53; C07D 413/12

(57) ABSTRACT

The present application relates to novel 2,5-disubstituted cyclopentanecarboxylic acid derivatives, to processes for preparation thereof, to the use thereof alone or in combinations for treatment and/or prevention of diseases and to the use thereof for production of medicaments for treatment and/or prevention of diseases, especially for treatment and/or prevention of respiratory, pulmonary and cardiovascular disorders.

The present application relates to novel 2,5-disubstituted cyclopentanecarboxylic acid derivatives, to processes for preparation thereof, to the use thereof alone or in combinations for treatment and/or prevention of diseases and to the use thereof for production of medicaments for treatment and/or prevention of diseases, especially for treatment and/or prevention of respiratory, pulmonary and cardiovascular disorders.

Human macrophage elastase (HME, EC 3.4.24.65) forms part of the family of the matrix metallopeptidases (MMPs) and is also called human matrix metallopeptidase 12 (hMMP-12). The protein is formed, activated and released to an increased degree, inter alia, by macrophages after contact with “stimulating” substances or particles. Such substances and particles may be present, for example, as extraneous substances in suspended particles as occur in cigarette smoke or industrial dusts, inter alia. In the broader sense, also counted among these stimulating particles are endogenous and exogenous cell constituents and cell fragments, as can occur in inflammation processes, sometimes in high concentration. The highly active enzyme is capable of degrading a multitude of binding tissue proteins, for example primarily the protein elastin (hence the name), and further proteins and proteoglycans such as collagen, fibronectin, laminin, chondroitin sulphate, heparan sulphate and others. This proteolytic activity of the enzyme makes macrophages capable of penetrating the basal membrane. Elastin, for example, occurs in high concentrations in all tissue types that exhibit high elasticity, for example in the lung and in arteries. In a large number of pathological processes, such as tissue damage, HME plays an important role in tissue degradation and remodelling. Furthermore, HME is an important modulator in inflammation processes. It is a key molecule in the recruitment of inflammation cells in that it, for example, releases the central inflammation mediator tumour necrosis factor alpha (TNF-α) and intervenes in the signal pathway mediated by transforming growth factor-beta (TGF-β) [Hydrolysis of a Broad Spectrum of Extracellular Matrix Proteins by Human Macrophage Elastase, Gronski et al., J. Biol. Chem. 272, 12189-12194 (1997)]. MMP-12 also plays a role in host defence, particularly in the regulation of antiviral immunity, presumably as a result of an intervention into the interferon-alpha (IFN-α)-mediated signal pathway [A new transcriptional role for matrix metalloproteinase-12 in antiviral immunity, Marchant et al., Nature Med. 20, 493-502 (2014)].

It is therefore assumed that HME plays an important role in many disorders, injuries and pathological lesions whose aetiology and/or progression is associated with an infectious or non-infectious event and/or proliferative and hypertrophic tissue and vessel remodelling. These may especially be diseases and/or damage to the lung, to the kidney or to the cardiovascular system, or they may be cancers or other inflammation disorders [Macrophage metalloelastase (MMP-12) as a target for inflammatory respiratory diseases, Lagente et al., Expert Opin. Ther. Targets 13, 287-295 (2009); Macrophage Metalloelastase as a major Factor for Glomerular Injury in Anti-Glomerular Basement Membrane Nephritis, Kaneko et al., J. Immunol. 170, 3377-3385 (2003); A Selective Matrix Metalloelastase-12 Inhibitor Retards Atherosclerotic Plaque Development in Apolipoprotein E Knock-out Mice, Johnson et al., Arterioscler. Thromb. Vasc. Biol. 31, 528-535 (2011); Impaired Coronary Collateral Growth in the Metabolic Syndrome Is in Part Mediated by Matrix Metalloelastase 12-dependent Production of Endostatin and Angiostatin, Dodd et al., Arterioscler. Thromb. Vasc. Biol. 33, 1339-1349 (2013); Matrix metalloproteinase pharmacogenomics in non-small-cell lung carcinoma, Chetty et al., Pharmacogenomics 12, 535-546 (2011)].

Diseases and damage to the lung that should be mentioned in this context are especially chronic obstructive pulmonary disease (COPD), pulmonary emphysema, interstitial lung diseases (ILD), for example idiopathic pulmonary fibrosis (IPF) and pulmonary sarcoidosis, acute lung injury (ALI), acute respiratory distress syndrome (ARDS), cystic fibrosis (CF; also called mucoviscidosis), asthma, and also infectious, particularly virally induced, respiratory disorders. Other fibrotic disorders that should be mentioned here by way of example include hepatic fibrosis and systemic sclerosis. Diseases and damage to the cardiovascular system in which HME is involved are, for example, tissue and vascular lesions in the event of arteriosclerosis, here in particular carotid arteriosclerosis, infective endocarditis, here in particular viral myocarditis, cardiomyopathy, heart failure, cardiogenic shock, acute coronary syndrome (ACS), aneurysms, reperfusion injuries following an acute myocardial infarct (AMI), ischaemic injuries to the kidneys or the retina, and also the chronic courses thereof, for example chronic kidney disease (CKD) and Alport's syndrome. Mention should also be made here of metabolic syndrome and obesity. Diseases connected to sepsis are, for example, systemic inflammatory response syndrome (SIRS), severe sepsis, septic shock and multiple organ failure (MOF)/multiorgan dysfunction (MODS) and also disseminated intravascular coagulation (DIC). Examples of tissue degradation and remodelling during neoplastic processes are the invasion of cancer cells into healthy tissue (formation of metastases) and neovascularization (neoangiogenesis). Other inflammatory diseases in which HME plays a role are rheumatoid diseases, for example rheumatoid arthritis, and also chronic intestinal inflammation (inflammatory bowel disease (IBD); Crohn's disease CD; ulcerative colitis UC).

In general, it is assumed that elastase-mediated pathological processes are based on a shifted equilibrium between free elastase (HME) and the endogenous tissue inhibitor of metalloproteinase (TIMP). In various pathological processes, particularly inflammation processes, the concentration of free elastase (HME) is elevated, such that there is a local shift in the balance between protease and anti-protease in favour of the protease. A similar (im)balance exists between the elastase of neutrophil cells (human neutrophil elastase, HNE, a member of the serine protease family) and endogenous anti-protease AAT (alpha-1 anti-trypsin, a member of the serine protease inhibitors, SERPINs). The two equilibria are coupled to one another since HME cleaves and inactivates the inhibitor of the HNE and, conversely, HNE cleaves and inactivates the HME inhibitor, which can result in an additional shift in the respective protease/anti-protease imbalances. Moreover, in the environment of local inflammation, strongly oxidizing conditions exist (an “oxidative burst”), which further strengthens the protease/anti-protease imbalance [Pathogenic triad in COPD: oxidative stress, protease-antiprotease imbalance, and inflammation, Fischer et al., Int. J. COPD 6, 413-421 (2011)].

Currently, more than 20 MMPs are known, which are historically roughly divided into different classes with regard to their most prominent substrates, e.g. gelatinases (MMP-2, MMP-9), collagenases (MMP-1, MMP-8, MMP-13), stromelysins (MMP-3, MMP-10, MMP-11) and matrilysins (MMP-7, MMP-26). HME (MMP-12) is hitherto the only representative of metalloelastases. Moreover, further MMPs are added to the group of so-called MT-MMPs (membrane-type MMPs) since these have a characteristic domain which anchors the protein in the membrane (MMP-14, MMP-15, MMP-16, MMP-17, MMP-24, MMP-25). A common feature of all the MMPs is a preserved zinc-binding region in the active centre of the enzyme which is important for the catalytic activity and which can also be found in other metalloproteins (e.g. a disintegrin and metalloproteinase, ADAM). The complexed zinc is masked by a sulphhydryl group in the N-terminal pro-peptide domain of the protein, which leads to an enzymatically inactive pro-form of the enzyme. It is only through detachment of this pro-peptide domain that the zinc in the active centre of the enzyme is freed from this coordination and hence the enzyme is activated (called activation by cysteine switch) [Matrix metalloproteinase inhibitors as therapy for inflammatory and vascular diseases, Hu et al., Nature Rev. Drug Discov. 6, 480-498 (2007)].

Most of the known synthetic MMP inhibitors have a zinc-complexing functional group, very frequently, for example, a hydroxamate, carboxylate or thiol [Recent Developments in the Design of Specific Matrix Metalloproteinase Inhibitors aided by Structural and Computational Studies, B. G. Rao, Curr. Pharm. Des. 11, 295-322 (2005)]. The scaffold of these inhibitors often still resembles peptides, in which case they are called “peptidomimetics” (generally with poor oral bioavailability), or it has no similarity to peptides, in which case they are more generally called small molecules (SMOLs). The physicochemical and pharmacokinetic properties of these inhibitors have, in quite general terms, a major influence on which target molecules (targets) and which undesired molecules (anti-targets, off-targets) are “encountered” in which tissue, in which period of time and to what extent.

It is a great challenge here to determine the specific role of a particular MMP in the course of a disease. This is made particularly difficult by the fact that there is a multitude of MMPs and further similar molecules (e.g. ADAMs), each associated with a multitude of possible physiological substrates and hence, under some circumstances, also with accompanying inhibiting or activating effects in various signal transduction pathways. Numerous in vitro and preclinical in vivo experiments have contributed to a better understanding of the MMPs in various disease models (e.g. transgenic animals, knockout animals and genetic data from human studies). A target can ultimately only be validated with respect to possible medicament therapy in clinical test series in humans or patients. In this context, the first generation of MMP inhibitors has been clinically examined in cancer studies. At this time, only a few representatives of the MMP protein family were known. None of the inhibitors examined were clinically convincing, since the side effects that occurred at effective dosages were intolerable. As emerged as further MMPs became known, the representatives of the first inhibitor generation were non-selective inhibitors, i.e. a large number of different MMPs was inhibited to the same extent (pan-MMP inhibitors, pan-MMPIs). Presumably, the desired effect on one or more MMP targets was concealed by an undesired effect on one or more MMP anti-targets or by means of an undesired effect at another target site (off-target) [Validating matrix metallo proteinases as drug targets and anti-targets for cancer therapy, Overall & Kleifeld, Nature Rev. Cancer 6, 227-239 (2006)].

Newer MMP inhibitors, which are characterized by increased selectivity, have now likewise been clinically tested, including compounds referred to explicitly as MMP-12 inhibitors, although hitherto likewise without compelling clinical success. On closer inspection, the inhibitors previously described as selective have not been found to be quite so selective here either.

For instance, for the clinical test compound “MMP408” as MMP-12 inhibitor, a certain to distinct selectivity in vitro with respect to MMP-13, MMP-3, MMP-14, MMP-9, Agg-1, MMP-1, Agg-2, MMP-7 and TACE is described [A Selective Matrix Metalloprotease 12 Inhibitor for Potential Treatment of Chronic Obstructive Pulmonary Disease (COPD): Discovery of (S)-2-(8-(Methoxycarbonylamino)dibenzo[b,d]furan-3-sulfonamido)-3-methylbutanoic acid (MMP408), Li et al., J. Med. Chem. 52, 1799-1802 (2009)]. In vitro efficacy data for MMP-2 and MMP-8 suggest less advantageous selectivity with respect to these two MMP representatives [Matrix metalloproteinase-12 is a therapeutic target for asthma in children and young adults, Mukhopadhyay et al., J. Allergy Clin. Immunol. 126, 70-76 (2010)].

Similar observations are made with the clinical test substance AZD1236 for treatment of COPD, which is described as a dual MMP-9/12 inhibitor [Effects of an oral MMP-9 and-12 inhibitor, AZD1236, on biomarkers in moderate/severe COPD: A randomised controlled trial, Dahl et al., Pulm. Pharmacol. Therap. 25, 169-177 (2012)]. The development of this compound was stopped in 2012; here too, noticeable inhibition of MMP-2 and MMP-13 is cited [http://www.wipo.int/research/en/details.jsp?id=2301].

In the assessment of MMP selectivity, moreover, a cautious assessment of the meaningfulness of animal models is appropriate. The test compound MMP408, for example, shows significantly reduced affinity for the orthologous MMP-12 target in mice: IC₅₀ 2 nM (human MMP-12), IC₅₀ 160 nM (murine MMP-12), IC₅₀ 320 nm (rat MMP-12) [see above Li et al., 2009; Mukhopadhyay et al., 2010]. No figures relating to potency with respect to other murine MMPs have been published. The situation seems to be similar for the test substance AZD1236 [see the information given relating to cross-reactivity in various animal species at http://www.wipo.int/research/en/details.jsp?id=2301].

As well as the selectivity profile across species boundaries, the potency on the MMP-12 target itself is very important. Given a comparatively similar pharmacokinetic profile, a compound of high potency will lead to a lower therapeutic dose than a less potent compound and, in general, a lower dose should be associated with a reduced probability of side effects. This is true particularly with regard to what is called the “free fraction” (fraction unbound, fᵤ) of a compound which can interact with the desired target and/or undesired anti- and off-targets (the “free fraction” is defined as the available amount of a compound which is not bound to constituents of blood plasma; these are primarily blood protein constituents, for example albumin). As well as MMP selectivity, specificity is also of major significance.

Novel active ingredients that inhibit macrophage elastase should accordingly have high selectivity and specificity in order to be able to selectively inhibit HME. For this purpose, good metabolic stability of the substances is also necessary (low clearance). Moreover, these compounds should be stable under oxidative conditions in order not to lose inhibitory potency in the course of the disease.

Chronic obstructive pulmonary disease (COPD) is a slowly progressing pulmonary disease characterized by an obstruction of respiratory flow which is caused by pulmonary emphysema and/or chronic bronchitis. The first symptoms of the disease generally manifest themselves during the fourth or fifth decade of life. In the subsequent years of life, shortness of breath frequently becomes worse, and there are instances of coughing combined with copious and purulent sputum, and stenotic respiration extending as far as breathlessness (dyspnoea). COPD is primarily a smokers' disease: smoking is the cause of 90% of all cases of COPD and of 80-90% of all COPD-related deaths. COPD is a big medical problem and constitutes the sixth most frequent cause of death worldwide. Of people over the age of 45, about 4-6% are affected.

Although the obstruction of the respiratory flow may only be partial and temporal, COPD cannot be cured. Accordingly, the aim of the treatment is to improve the quality of life, to alleviate the symptoms, to prevent acute worsening and to slow the progressive impairment of lung function. Existing pharmacotherapies, which have hardly changed over the last two or three decades, are the use of bronchodilators to open blocked respiratory passages, and in certain situations corticosteroids to control the inflammation of the lung [Chronic Obstructive Pulmonary Disease, P. J. Barnes, N. Engl. J. Med. 343, 269-280 (2000)]. The chronic inflammation of the lung, caused by cigarette smoke or other irritants, is the driving force of the development of the disease. The underlying mechanism includes immune cells that excrete various chemokines in the course of the inflammation reaction of the lung. As a result, neutrophil cells and, later on, alveolar macrophages are attracted to the connective tissue of the lung and lumen. Neutrophil cells secrete a protease cocktail containing mainly HNE and proteinase 3. Activated macrophages release HME. This results in a local shift in the protease/antiprotease balance in favour of the proteases, which leads, inter alia, to uncontrolled elastase activity and, as a result of this, to an overshoot in degradation of the alveolar elastin. This tissue degradation causes a collapse of the bronchi. This is associated with reduced elasticity of the lung, which leads to impairment of respiratory flow and impaired respiration. Moreover, frequent and prolonged inflammation of the lung can lead to remodelling of the bronchi and consequently to formation of lesions. Such lesions contribute to the occurrence of chronic coughing, which is an indication of chronic bronchitis.

It is known from studies with human sputum samples that the amount of HME protein is associated with the smoking or COPD status: The detectable amounts of HME are at their lowest in the case of non-smokers, somewhat elevated in the case of former smokers and smokers, and distinctly elevated in the case of COPD patients [Elevated MMP-12 protein levels in induced sputum from patients with COPD, Demedts et al., Thorax 61, 196-201 (2006)]. Similar data were obtained with human sputum samples and bronchial alveolar washing fluid (ALF). It was possible here to detect and quantify HME on activated macrophages: amount of HME in COPD patient/smoker>COPD patient/former smoker>former smoker>non-smoker [Patterns of airway inflammation and MMP-12 expression in smokers and ex-smokers with COPD, Babusyte et al., Respir. Res. 8, 81-90 (2007)].

An inflammatory lung disease having some degree of similarity to COPD is interstitial lung disease (ILD), particularly in the form of idiopathic pulmonary fibrosis (IPF) and sarcoidosis [Commonalities between the pro-fibrotic mechanisms in COPD and IPF, L. A. Murray, Pulm. Pharmacol. Therap. 25, 276-280 (2012); The pathogenesis of COPD and IPF: distinct horns of the same devil?, Chilosi et al., Respir. Res. 13:3 (2012)]. Here too, the homeostasis of the extracellular matrix is disturbed. Data from genome-wide association studies suggest a particular role of HME in the course of disease of such fibrotic disorders [Gene Expression Profiling Identifies MMP-12 and ADAMDEC1 as Potential Pathogenic Mediators of Pulmonary Sarcoidosis, Crouser et al., Am. J. Respir. Crit. Care Med. 179, 929-938 (2009); Association of a Functional Polymorphism in the Matrix Metalloproteinase-12 Promoter Region with Systemic Sclerosis in an Italian Population, Manetti et al., J. Rheumatol. 37, 1852-1857 (2010); Increased serum levels and tissue expression of matrix metalloproteinase-12 in patients with systemic sclerosis: correlation with severity of skin and pulmonary fibrosis and vascular damage, Manetti et al., Ann. Rheum. Dis. 71, 1064-1070 (2012)].

Furthermore, there is further preclinical evidence of a crucial role of HME in ischaemic-inflammatory disease processes [Macrophage Metalloelastase (MMP-12) Deficiency Mitigates Retinal Inflammation and Pathological Angiogenesis in Ischemic Retinopathy, Li et al., PLoS ONE 7 (12), e52699 (2012)]. Much higher MMP-12 expression is also known in the case of ischaemic kidney damage, as is the involvement of MMP-12 in further inflammatory kidney disorders [JNK signalling in human and experimental renal ischaemia/reperfusion injury, Kanellis et al., Nephrol. Dial. Transplant. 25, 2898-2908 (2010); Macrophage Metalloelastase as a Major Factor for Glomerular Injury in Anti-Glomerular Basement Membrane Nephritis, Kaneko et al., J. Immun 170, 3377-3385 (2003); Role for Macrophage Metalloelastase in Glomerular Basement Membrane Damage Associated with Alport Syndrome, Rao et al., Am. J. Pathol. 169, 32-46 (2006); Differential regulation of metzincins in experimental chronic renal allograft rejection: Potential markers and novel therapeutic targets, Berthier et al., Kidney Int. 69, 358-368 (2006); Macrophage infiltration and renal damage are independent of Matrix Metalloproteinase 12 (MMP-12) in the obstructed kidney, Abraham et al., Nephrology 17, 322-329 (2012)].

The problem addressed by the present invention was thus that of identifying and providing novel substances which act as potent, selective and specific inhibitors of human macrophage elastase (HME/MMP-12) and as such are suitable for treatment and/or prevention, particularly of disorders of the respiratory pathways, the lung and the cardiovascular system.

Patent applications WO 96/15096-A1, WO 97/43237-A1, WO 97/43238-A1, WO 97/43239-A1, WO 97/43240-A1, WO 97/43245-A1 and WO 97/43247-A1 disclose 4-aryl- and 4-biaryl-substituted 4-oxobutanoic acid derivatives with inhibitory activity towards MMP-2, MMP-3, MMP-9 and, to a lesser extent, MMP-1; on account of this activity profile, these compounds were considered to be suitable particularly for treatment of osteoarthritis, rheumatoid arthritis and tumour diseases. WO 98/09940-A1 and WO 99/18079-A1 disclose further biarylbutanoic acid derivatives as inhibitors of MMP-2, MMP-3 and/or MMP-13 which are suitable for treating a wide variety of diseases. WO 00/40539-A1 claims the use of 4-biaryl-4-oxobutanoic acids for treatment of pulmonary and respiratory disorders, based on a different extent of inhibition of MMP-2, MMP-3, MMP-8, MMP-9, MMP-12 and MMP-13 by these compounds. Furthermore, WO 2012/014114-A1 describes 3-hydroxypropionic acid derivatives and WO 2012/038942-A1 describes oxy- or sulphonylacetic acid derivatives as dual MMP 9/12 inhibitors.

Against the background of the problem described above, however, it was found that these MMP inhibitors from the prior art often have disadvantages such as, more particularly, inadequate inhibitory potency towards MMP-12, inadequate selectivity for MMP-12 compared to other MMPs and/or limited metabolic stability.

Further arylalkanecarboxylic acid derivatives are described in WO 2004/092146-A2, WO 2004/099168-A2, WO 2004/099170-A2, WO 2004/099171-A2, WO 2006/050097-A1 and WO 2006/055625-A2 as inhibitors of protein-tyrosine-phosphatase 1B (PTP-1B) for treatment of diabetes, cancer diseases and neurodegenerative diseases.

It has now been found that, surprisingly, particular 2,5-disubstituted cyclopentanecarboxylic acid derivatives have a significantly improved profile in terms of their potency and selectivity with respect to human macrophage elastase (HME/hMMP-12) compared to the compounds known from the prior art. Furthermore, many of the compounds according to the invention have low in vitro clearance and good metabolic stability. This profile of properties overall suggests, for the compounds according to the invention, low dosability and—as a result of the more specific mode of action—reduced risk of the occurrence of unwanted side effects in treatment.

The compounds according to the invention additionally feature significant inhibitory activity and selectivity with respect to the orthologous rodent MMP-12 peptidases such as murine MMP-12 (also referred to as murine macrophage elastase) and rat MMP-12. This enables more comprehensive preclinical evaluation of the substances in various establish animal models for the above-described diseases.

The present invention provides compounds of the general formula (I)

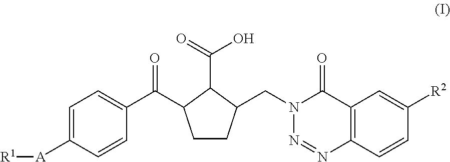

in which

### A is —O— or —S—,

R¹ is straight-chain or branched (C₃-C₇)-alkyl or a group of the formula

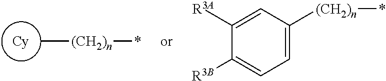

in which
* indicates the linkage to A,
n is the number 1, 2 or 3,
Cy is (C₃-C₆)-cycloalkyl,
R^{3A} is hydrogen, fluorine, chlorine, cyano, methyl, difluoromethyl, trifluoromethyl, methoxy, trifluoromethoxy, (trifluoromethyl)sulphanyl, methoxycarbonylamino or 2-oxo-1,3-oxazolidin-3-yl,
and
R^{3B} is hydrogen, fluorine, chlorine, methyl or trifluoromethyl,
   and
   R² is hydrogen, fluoromethyl, methyl, difluoromethyl or trifluoromethyl,
   and the salts, solvates and solvates of the salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are mentioned below as working examples and the salts, solvates and solvates of the salts thereof if the compounds that are encompassed by formula (I) and are mentioned below are not already salts, solvates and solvates of the salts.

Preferred salts in the context of the present invention are physiologically acceptable salts of the compounds according to the invention. Also encompassed are salts which are not themselves suitable for pharmaceutical applications but can be used, for example, for the isolation, purification or storage of the compounds according to the invention.

Physiologically acceptable salts of the compounds according to the invention include in particular the salts derived from conventional bases, by way of example and with preference alkali metal salts (e.g. sodium and potassium salts), alkaline earth metal salts (e.g. calcium and magnesium salts), zinc salts and ammonium salts derived from ammonia or organic amines having 1 to 16 carbon atoms, by way of example and with preference ethylamine, diethylamine, triethylamine, N,N-ethyldiisopropylamine, monoethanolamine, diethanolamine, triethanolamine, tromethamine, dimethylaminoethanol, diethylaminoethanol, choline, procaine, dicyclohexylamine, dibenzylamine, N-methylmorpholine, N-methylpiperidine, arginine, lysine and 1,2-ethylenediamine.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatography processes are preferably used for this purpose, especially HPLC chromatography on an achiral or chiral phase.

In the context of the present invention, the term “enantiomerically pure” is understood to the effect that the compound in question with respect to the absolute configuration of the chiral centres is present in an enantiomeric excess of more than 95%, preferably more than 98%. The enantiomeric excess, ee, is calculated here by evaluating an HPLC analysis chromatogram on a chiral phase using the formula below:

$$ee = \vert \frac{\begin{matrix}enantiomer 1 ( area per cent ) - \\ enantiomer 2 ( area per cent )\end{matrix}}{\begin{matrix}enantiomer 1 ( area per cent ) + \\ enantiomer 2 ( area per cent )\end{matrix}} \vert \times 100 \% .$$

If the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore possibly also constitute a preferred embodiment of the present invention. Isotopic variants of the compounds according to the invention can be prepared by commonly used processes known to those skilled in the art, for example by the methods described further down and the procedures described in the working examples, by using corresponding isotopic modifications of the respective reagents and/or starting compounds.

The present invention additionally also encompasses prodrugs of the compounds according to the invention. The term “prodrugs” refers here to compounds which may themselves be biologically active or inactive, but are converted while present in the body, for example by a metabolic or hydrolytic route, to compounds according to the invention.

The present invention comprises as prodrugs in particular hydrolysable ester derivatives of the carboxylic acids of the formula (I) according to the invention. These are understood to mean esters which can be hydrolysed to the free carboxylic acids, as the main biologically active compounds, in physiological media under the conditions of the biological tests described hereinbelow and in particular in vivo by enzymatic or chemical routes. (C₁-C₄)-Alkyl esters, in which the alkyl group can be straight-chain or branched, are preferred as such esters. Particular preference is given to methyl, ethyl or tert-butyl esters.

In the context of the present invention, unless specified otherwise, the substituents are defined as follows:

In the context of the invention, (C₃-C₇)-alkyl is a straight-chain or branched alkyl radical having 3 to 7 carbon atoms. Preferred examples include the following: n-propyl, isopropyl, n-butyl, isobutyl, sec-butyl, tert-butyl, n-pentyl, 2-pentyl, 3-pentyl, neopentyl, 3-methylbutyl, n-hexyl, 2-hexyl, 3-hexyl, 4-methylpentyl, n-heptyl and 5-methylhexyl. Preference is given to a straight-chain alkyl radical having 4 to 6 carbon atoms such as n-butyl, n-pentyl and n-hexyl.

(C₃-C₆)-Cycloalkyl in the context of the invention is a monocyclic saturated cycloalkyl group having 3 to 6 ring carbon atoms. Preferred examples include the following: cyclopropyl, cyclobutyl, cyclopentyl and cyclohexyl. Preference is given to a cycloalkyl radical having 5 or 6 ring carbon atoms such as cyclopentyl and cyclohexyl.

In the context of the present invention, all radicals which occur more than once are defined independently of one another. When radicals in the compounds according to the invention are substituted, the radicals may be mono- or polysubstituted, unless specified otherwise. Substitution by one substituent or by two identical or different substituents is preferred. Particular preference is given to substitution by one substituent.

Preference is given in the context of the present invention to compounds of the formula (I) in which

### A is —O— or —S—,

R¹ is n-butyl, n-pentyl or n-hexyl or a group of the formula

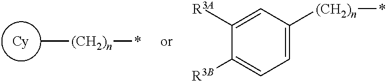

in which
indicates the linkage to A,
n is the number 1, 2 or 3,
Cy is cyclopentyl or cyclohexyl,
R^{3A} is hydrogen, chlorine, methyl, trifluoromethyl, methoxy, trifluoromethoxy, methoxycarbonylamino or 2-oxo-1,3-oxazolidin-3-yl,
and
R^{3B} is hydrogen, fluorine or chlorine,
   and
   R² is hydrogen, methyl or trifluoromethyl,
   and the salts, solvates and solvates of the salts thereof.

A particular embodiment of the present invention comprises compounds of the formula (I) in which

### A is —O—,

and the salts, solvates and solvates of the salts thereof.

A further particular embodiment of the present invention encompasses compounds of the formula (I) in which

R¹ is a group of the formula

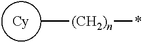

in which
* indicates the linkage to A,
n is the number 1 or 2
and
Cy is cyclopentyl or cyclohexyl,
   and the salts, solvates and solvates of the salts thereof.

A further particular embodiment of the present invention encompasses compounds of the formula (I) in which

R¹ is a group of the formula

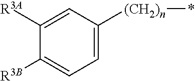

in which
* indicates the linkage to A,
n is the number 1,
R^{3A} is chlorine, methyl, trifluoromethyl, methoxy, trifluoromethoxy, methoxycarbonylamino or 2-oxo-1,3-oxazolidin-3-yl,
and
R^{3B} is hydrogen, fluorine or chlorine,
   and the salts, solvates and solvates of the salts thereof.

A further particular embodiment of the present invention encompasses compounds of the formula (I) in which

R² is methyl or trifluoromethyl,
and the salts, solvates and solvates of the salts thereof.

In the context of the present invention, particular preference is given to compounds of the formula (I) in which

### A is —O—,

R¹ is n-pentyl or n-hexyl or a group of the formula

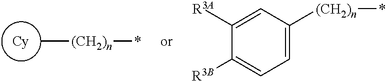

in which
* indicates the linkage to A,
n is the number 1 or 2,
Cy is cyclopentyl or cyclohexyl,
R^{3A} is hydrogen, chlorine, methyl, trifluoromethyl, methoxy or trifluoromethoxy,
and
R^{3B} is hydrogen, fluorine or chlorine,
   and
   R² is hydrogen, methyl or trifluoromethyl,
   and the salts, solvates and solvates of the salts thereof.

Of particular significance in the context of the present invention are compounds of the formulae (I-A) and (I-B)

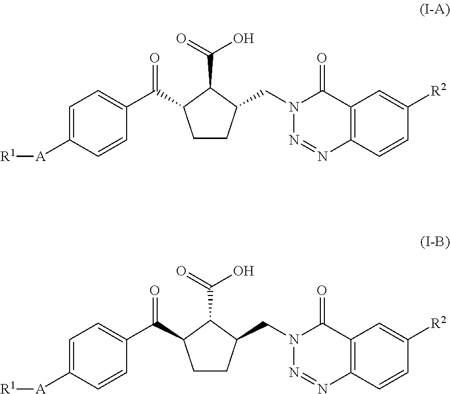

in which A, R¹ and R² have the definitions defined above or the groups bonded to the central cyclopentane ring have a relative trans arrangement, as are mixtures of these compounds where A, R¹ and/or R² are each identical in such a mixture of (I-A) and (I-B),
and the salts, solvates and solvates of the salts of these compounds and mixtures thereof.

In the context of the present invention, preference is given to the compounds of the formula (I-A)

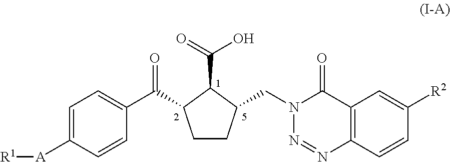

in which A, R¹ and R² have the definitions defined above and the groups bonded to the central cyclopentane ring, in enantiomerically pure form, have a (1S,2S,5R) arrangement relative to one another as shown,
and the salts, solvates and solvates of the salts of these compounds.

The individual radical definitions specified in the respective combinations or preferred combinations of radicals are, independently of the respective combinations of the radicals specified, also replaced as desired by radical definitions of other combinations.

Very particular preference is given to combinations of two or more of the abovementioned preferred ranges.

The invention further provides a process for preparing the compounds according to the invention, characterized in that

[A] a compound of the formula (II)

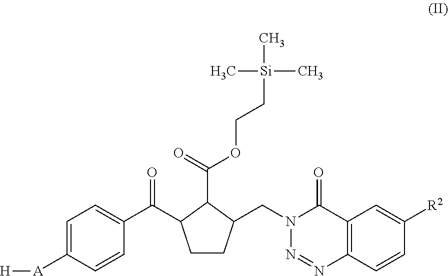

in which A and R² have the definitions given above,
is alkylated in the presence of a base with a compound of the formula (III)

R¹—X (III)
in which R¹ has the definition given above
and
X is a leaving group, for example chlorine, bromine, iodine, mesylate, triflate or tosylate,
to give a compound of the formula (IV)

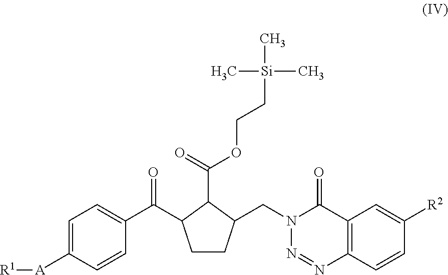

in which A, R¹ and R² have the definitions given above,
and then the 2-(trimethylsilyl)ethyl ester group is detached with the aid of an acid or a fluoride reagent to give the carboxylic acid of the formula (I)

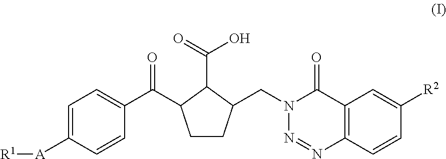

in which A, R¹ and R² have the definitions given above,

or alternatively, when A in formula (I) is —S—,
[B] a compound of the formula (II-A)

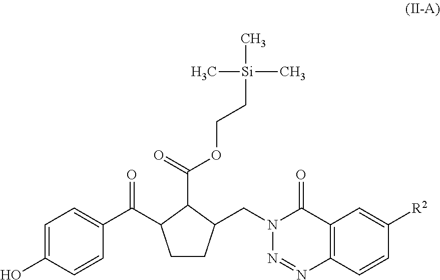

in which R¹ has the definition given above,
is converted to the corresponding trifluoromethanesulphonate of the formula (V)

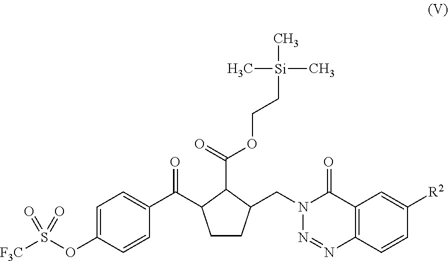

in which R¹ has the definition given above,
then the latter is reacted in the presence of a suitable palladium catalyst with a thiol of the formula (VI)

R¹—SH (VI)
in which R¹ has the definition given above
to give a compound of the formula (IV-A)

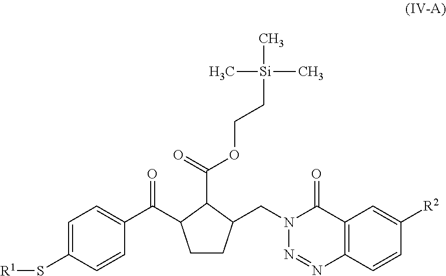

in which R¹ and R² have the definitions given above
and subsequently the 2-(trimethylsilyl)ethyl ester group is detached with the aid of an acid or a fluoride reagent to give the carboxylic acid of the formula (I-C)

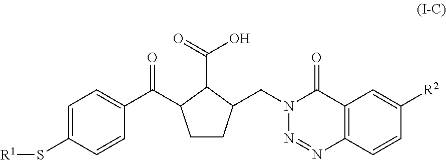

in which R¹ and R² have the definitions given above,
   and, if appropriate, the compounds of the formula (I) or (I-C) thus obtained are separated into their enantiomers and/or diastereomers and/or converted with the appropriate (i) solvents and/or (ii) bases to their solvates, salts and/or solvates of the salts.

Especially suitable bases for the alkylation reaction (II)+(III)→(IV) are alkali metal carbonates such as lithium carbonate, sodium carbonate, potassium carbonate or caesium carbonate, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amide bases such as lithium diisopropylamide or lithium bis(trimethylsilyl)amide, sodium bis(trimethylsilyl)amide or potassium bis(trimethylsilyl)amide, or standard organometallic bases such as phenyllithium or n-, sec- or tert-butyllithium. Preference is given to using potassium carbonate or potassium tert-butoxide.

Suitable inert solvents for this reaction are, for example, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons such as benzene, toluene, xylene, pentane, hexane or cyclohexane, or dipolar aprotic solvents such as acetonitrile, butyronitrile, N,N-dimethylformamide (DMF), N,N-dimethylacetamide (DMA), N,N′-dimethylpropyleneurea (DMPU), N-methylpyrrolidinone (NMP) or dimethyl sulphoxide (DMSO). It is also possible to use mixtures of such solvents. Preference is given to using acetonitrile or N,N-dimethylformamide (DMF).

The reaction (II)+(III)→(IV) is generally conducted, according to the reactivity of the components involved, within a temperature range from 0° C. to +120° C.

The detachment of the 2-(trimethylsilyl)ethyl ester moiety in the process step (IV)→(I) is effected by standard methods with the aid of a strong acid such as trifluoroacetic acid in particular in an inert solvent such as dichloromethane, or with the aid of a fluoride such as tetrabutylammonium fluoride (TBAF) in particular in an ethereal solvent such as tetrahydrofuran. The ester cleavage is generally conducted within a temperature range from −20° C. to +30° C.

The preparation of the trifluoromethanesulphonate (V) proceeding from the phenol (II-A) is effected in a customary manner by reaction with trifluoromethanesulphonic anhydride in the presence of an amine base, for example N,N-diisopropylethylamine or pyridine. Inert solvents used are generally chlorinated hydrocarbons such as dichloromethane or chloroform, and the reaction is generally conducted within a temperature range from −20° C. to +25° C.

The coupling reaction (V)+(VI)→(IV-A) is conducted with the aid of a palladium catalyst. Suitable examples for this purpose are palladium(II) acetate, palladium(II) chloride, bis(triphenylphosphine)palladium(II) chloride, bis(acetonitrile)palladium(II) chloride, tetrakis(triphenylphosphine)palladium(0), bis(dibenzylideneacetone)palladium(0), tris(dibenzylideneacetone)palladium(0) or [1,1¹-bis(diphenylphosphino)ferrocene]palladium(II) chloride, each in combination with a suitable phosphine ligand, for example 2-dicyclohexylphosphino-2′,4′,6′-triisopropylbiphenyl (X-Phos), 2-dicyclohexylphosphino-2′,6′-dimethoxybiphenyl (S-Phos), 1,2,3,4,5-pentaphenyl-1′-(di-tert-butylphosphino)ferrocene (Q-Phos), 4,5-bis(diphenylphosphino)-9,9-dimethylxanthene (Xantphos), 2,2′-bis(diphenylphosphino)-1,1′-binaphthyl (BINAP), 2-dicyclohexylphosphino-2′-(N,N-dimethylamino)biphenyl or 2-di-tert-butylphosphino-T-(N,N-dimethylamino)biphenyl.

The reaction is generally conducted in the presence of a base. Suitable bases are alkali metal carbonates such as sodium carbonate, potassium carbonate or caesium carbonate, alkali metal phosphates such as sodium phosphate or potassium phosphate, alkali metal fluorides such as potassium fluoride or caesium fluoride, alkali metal tert-butoxides such as sodium tert-butoxide or potassium tert-butoxide, tertiary amine bases such as triethylamine, N-methylmorpholine, N-methylpiperidine, N,N-diisopropylethylamine, pyridine or 4-N,N-dimethylaminopyridine, or amide bases such as lithium bis(trimethylsilyl)amide, sodium bis(trimethylsilyl)amide or potassium bis(trimethylsilyl)amide. The reaction is effected in an inert solvent, for example toluene, xylene, 1,2-dimethoxyethane, tetrahydrofuran, 1,4-dioxane, acetonitrile, dimethyl sulphoxide (DMSO), N,N-dimethylformamide (DMF) or N,N-dimethylacetamide (DMA) or mixtures thereof, within a temperature range from +50° C. to +150° C.; the use of a microwave apparatus may be advantageous.

For this coupling reaction, preference is given to using a catalyst/ligand/base system consisting of tris(dibenzylideneacetone)dipalladium(0), 4,5-bis(diphenylphosphino)-9,9-dimethylxanthene (Xantphos) and N,N-diisopropylethylamine, and 1,4-dioxane as solvent [cf. also M. S Chambers et al., Int. Pat. Appl. WO 2006/059149-A1, page 9; C.-K. Pei and M. Shi, Tetrahedron: Asymmetry 22 (11), 1239-1248 (2011)].

The detachment of the 2-(trimethylsilyl)ethyl ester moiety in the process step (IV-A)→(I-C) is effected in an analogous manner to that described above for the reaction (IV)→(I).

Compounds of the formula (II) in which A is —O— [i.e. compounds of the above formula (II-A)] can be prepared by reacting a compound of the formula (VII)

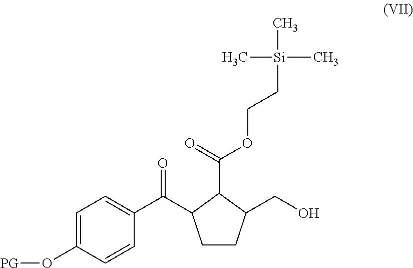

in which
PG is a temporary protecting group, for example benzyl,
in the presence of an alkyl- or arylphosphine and an azodicarboxylate, with a triazin-4(3H)-one derivative of the formula (VIII)

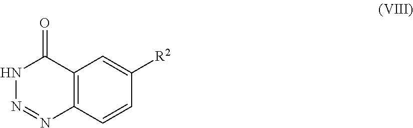

in which R² has the definition given above
to give a compound of the formula (IX)

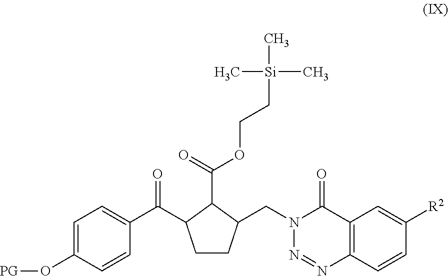

in which PG and R² have the definitions given above,
and then the protecting group PG is detached to obtain the compound of the formula (II-A)

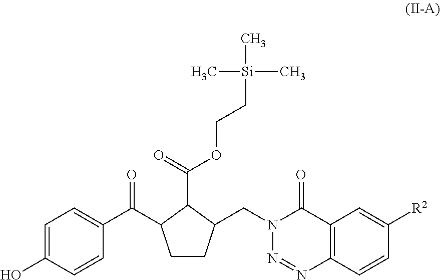

in which R² has the definition given above.

The reaction (VII)+(VIII)→(IX) is conducted under the customary conditions of a “Mitsunobu reaction” in the presence of a phosphine and an azodicarboxylate [see, for example, D. L. Hughes, Org. Reactions 42, 335 (1992); D. L. Hughes, Org. Prep. Proced. Int. 28 (2), 127 (1996)]. Examples of suitable phosphine components are triphenylphosphine, tri-n-butylphosphine, 1,2-bis(diphenylphosphino)ethane (DPPE), diphenyl(2-pyridyl)phosphine, (4-dimethylaminophenyl)diphenylphosphine or tris(4-dimethylaminophenyl)phosphine. The azodicarboxylate used may, for example, be diethyl azodicarboxylate (DEAD), diisopropyl azodicarboxylate (DIAD), di-tert-butyl azodicarboxylate, N,N,NN′-tetramethylazodicarboxamide (TMAD), 1,1′-(azodicarbonyl)dipiperidine (ADDP) or 4,7-dimethyl-3,5,7-hexahydro-1,2,4,7-tetrazocine-3,8-dione (DHTD). Preference is given here to using tri-n-butylphosphine in conjunction with diethyl azodicarboxylate (DEAD).

Inert solvents for this reaction are, for example, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons such as benzene, toluene, xylene, pentane, hexane or cyclohexane, or polar aprotic solvents such as acetonitrile, butyronitrile, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP). It is also possible to use mixtures of such solvents. Preference is given to using tetrahydrofuran, toluene or a mixture of the two.

The reaction (VII)+(VIII)→(IX) is generally effected within a temperature range from −20° C. to +60° C., preferably at 0° C. to +40° C. In some cases, the use of a microwave apparatus in this reaction may be advantageous.

The detachment of benzyl as temporary protecting group PG in the process step (IX)→(II-A) is effected in a customary manner by hydrogenation with gaseous hydrogen or, in the case of a transfer hydrogenation, with the aid of a hydrogen donor such as ammonium formate, cyclohexene or cyclohexadiene, in each case in the presence of a suitable hydrogenation catalyst such as palladium on activated carbon in particular. The reaction is preferably conducted in an alcoholic solvent such as methanol or ethanol, in ethyl acetate or tetrahydrofuran, or in a mixture of such solvents, optionally with addition of water, within a temperature range from +20° C. to +80° C. [with regard to possible alternative protecting groups and to the introduction and removal of such protecting groups see also: T. W. Greene and P. G. M. Wuts, Protective Groups in Organic Synthesis, Wiley, New York, 1999].

Compounds of the formula (II) in which A is —S— can be prepared by reacting the trifluoromethanesulphonate of the formula (V) already described above

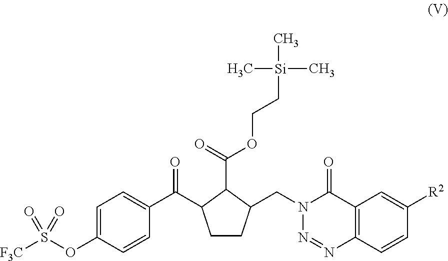

in which R² has the definition given above,
in the presence of a suitable palladium catalyst, with a trialkylsilanethiol, for example triisopropylsilanethiol, to give the compound of the formula (II-B)

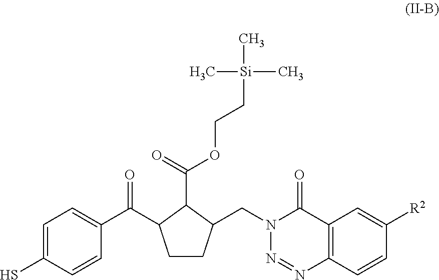

in which R² has the definition given above.

The transformation (V)→(II-B) is conducted in an analogous manner to that described in detail above for the coupling reaction (V)+(VI)→(IV-A). The trialkylsilyl sulphide formed at first here through coupling with the trialkylsilanethiol is cleaved again under the conditions of aqueous reaction workup and chromatographic product purification used, such that the free thiophenol (II-B) is obtained directly [cf. M. Kreis and S. Brase, Adv. Synth. Catal. 347 (2-3), 313-319 (2005)].

The individual process steps described above can be conducted at standard, elevated or reduced pressure (for example in the range from 0.5 to 5 bar); in general, standard pressure is employed in each case.

The compounds of the formula (VII) can in turn be obtained on the basis of published synthesis methods by various routes proceeding from compounds of the formula (X) or (XI)

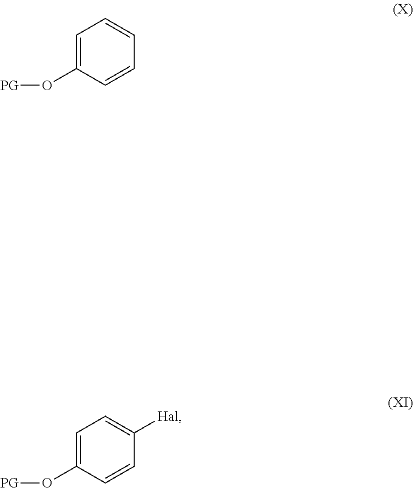

in which PG has the definition given above and Hal is a halogen atom
[see, for example, the general preparative methods described in WO 96/15096-A1, pages 26-44, especially methods A, G, H and K].

Compounds of the formula (VII) in particular that have a relative trans arrangement of the groups bonded to the central cyclopentane ring, i.e. compounds of the formulae (VII-A) and (VII-B)

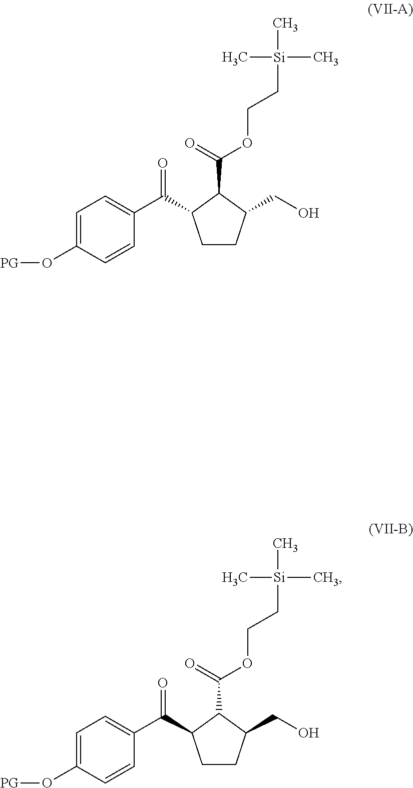

in which PG has the definition given above,
can be prepared in analogy to published synthesis methods, for example, by reacting exo-2-(trimethylsilyl)ethyl 2-oxobicyclo[2.2.1]heptane-7-carboxylate of the formula (XII)

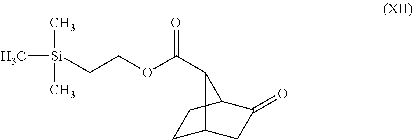 [SMILES: C[Si](C)(C)CCOC(=O)C1C2CCC1C(=O)C2]

with a phenyl Grignard compound of the formula (XIII)

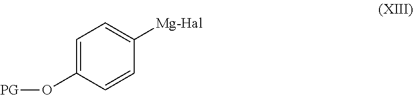

in which PG and Hal have the definitions given above
to give the adduct of the formula (XIV)

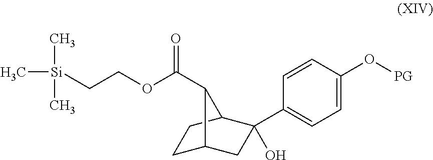

in which PG has the definition given above,
subsequently eliminating the tertiary hydroxyl group via the corresponding mesylate to give the olefin of the formula (XV)

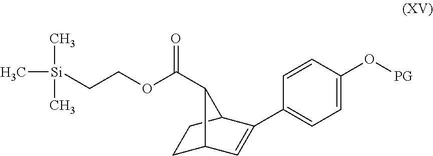

in which PG has the definition given above,
then oxidizing the latter with N-methylmorpholine N-oxide together with osmium tetroxide as catalyst to give the 1,2-diol of the formula (XVI)

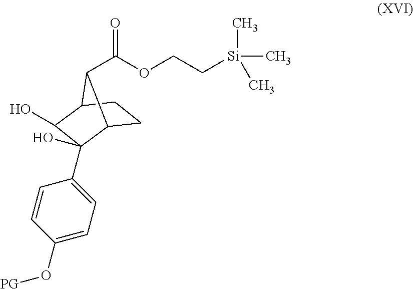

in which PG has the definition given above,
then cleaving this bicyclic diol with the aid of lead tetraacetate or sodium periodate to give the 2-formyl-5-ketocyclopentanecarboxylic ester of the formula (XVII)

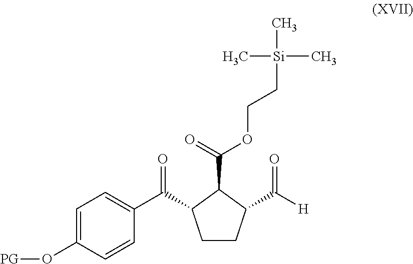

in which PG has the definition given above,
and finally reducing the latter with sodium borohydride to give the hydroxymethyl compound of the formula (VII-A)

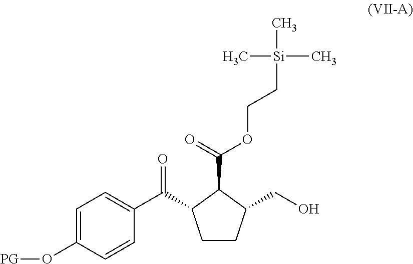

in which PG has the definition given above,
[cf. WO 96/15096-A1, preparative method K (pages 42-44)].

In the above-described synthesis sequence (XII)+(XIII)→(XIV)→(XV)→(XVI)→(XVII)→(VII-A), for simplified representation of the relative configuration of the chiral centres, only the structural formula of each enantiomer has been given, even though the compounds in question have been used or obtained in racemic form; the actual end product of a preparation process conducted in racemic form in such a way is the racemic mixture of the compounds (VII-A) and (VII-B).

The 1,2,3-triazin-4(3H)-one derivatives of the formula (VIII) are obtainable in a simple manner by treating ortho-aminobenzamides of the formula (XVIII)

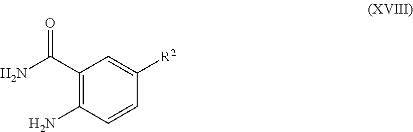

in which R² has the definition given above
with sodium nitrite in aqueous hydrochloric acid [see, for example, D. Fernandez-Forner et al., Tetrahedron 47 (42), 8917-8930 (1991)].

The separation of stereoisomers (enantiomers and/or diastereomers) of the inventive compounds of the formula (I) can be achieved by customary methods familiar to those skilled in the art. Preference is given to employing chromatographic methods on achiral or chiral separation phases for this purpose. Alternatively, separation can also be effected via diastereomeric salts of the carboxylic acids of the formula (I) with chiral amine bases.

Separation of the compounds according to the invention into the corresponding enantiomers and/or diastereomers can, if appropriate, also be conducted at the early stage of the intermediates (II), (IV), (II-A), (IV-A), (VII), (IX), (II-B) or (VII-A)/(VII-B), which are then reacted further in separated form in accordance with the reaction sequence described above. For such a separation of the stereoisomers of intermediates, preference is likewise given to employing chromatographic methods on achiral or chiral separation phases.

The compounds of the formulae (III), (VI), (X), (XI), (XII), (XIII) and (XVIII) are either commercially available or described as such in the literature, or they can be prepared from other commercially available compounds by literature methods familiar to those skilled in the art. Numerous detailed procedures and further literature references can also be found in the experimental section, in the section on the preparation of the starting compounds and intermediates.

The preparation of the inventive compounds can be illustrated by way of example by the following reaction schemes:

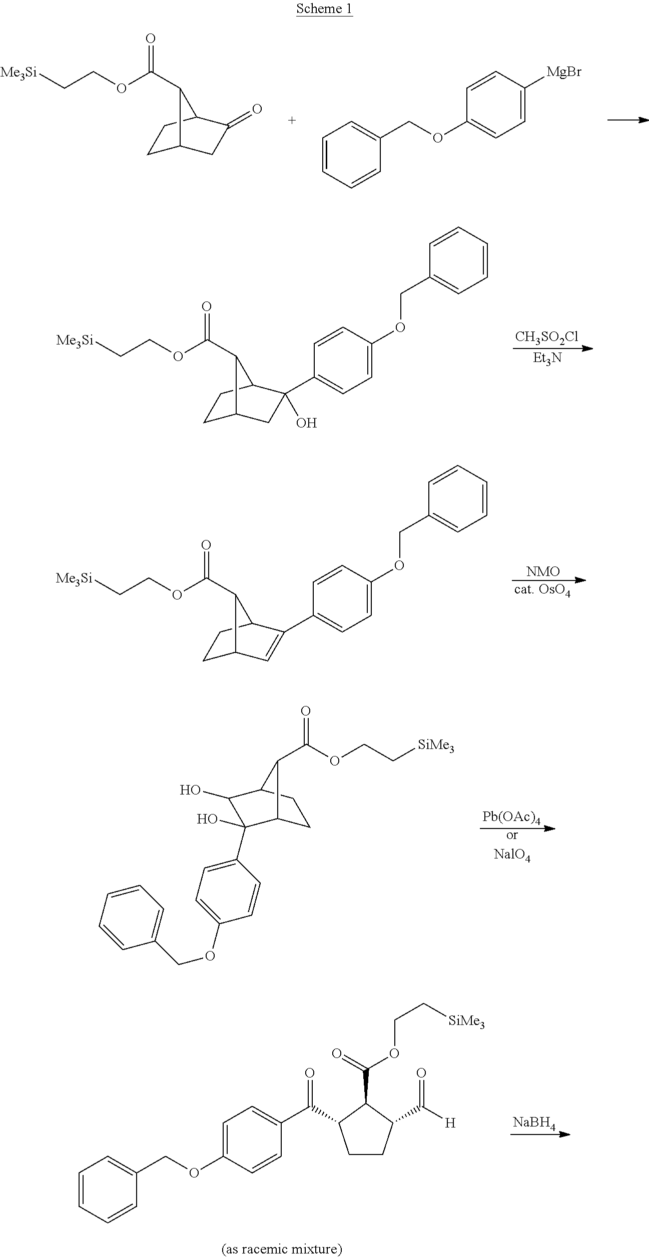

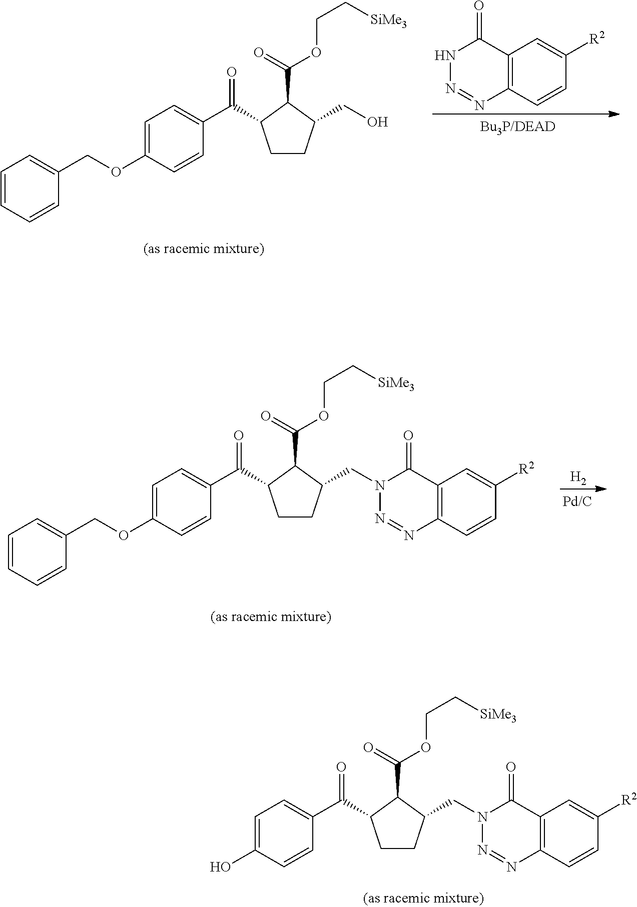

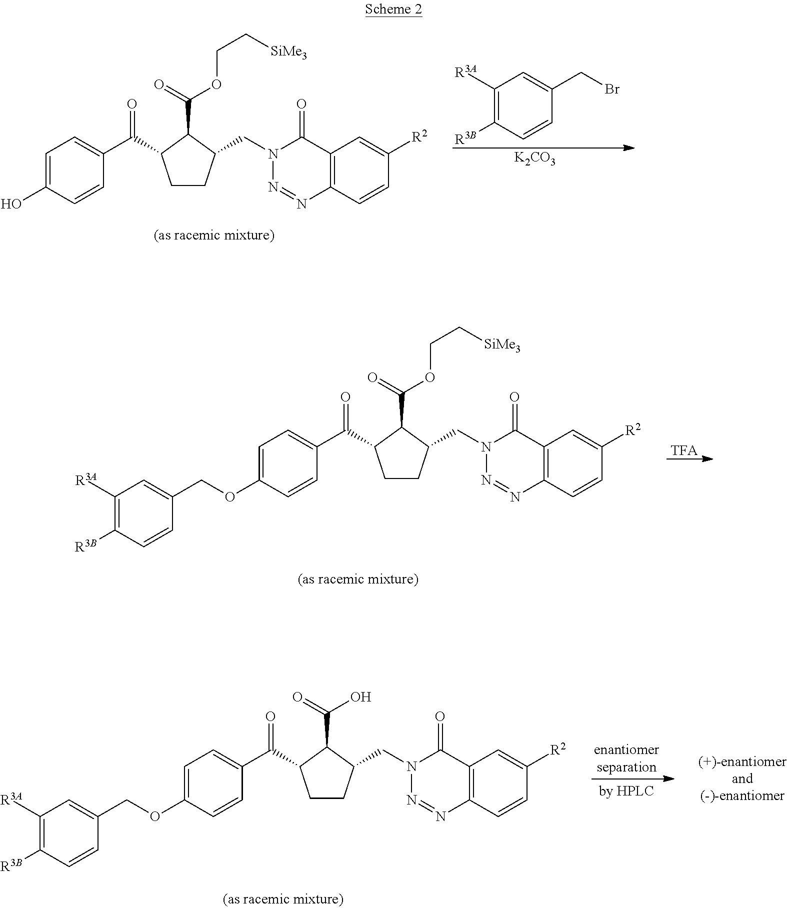

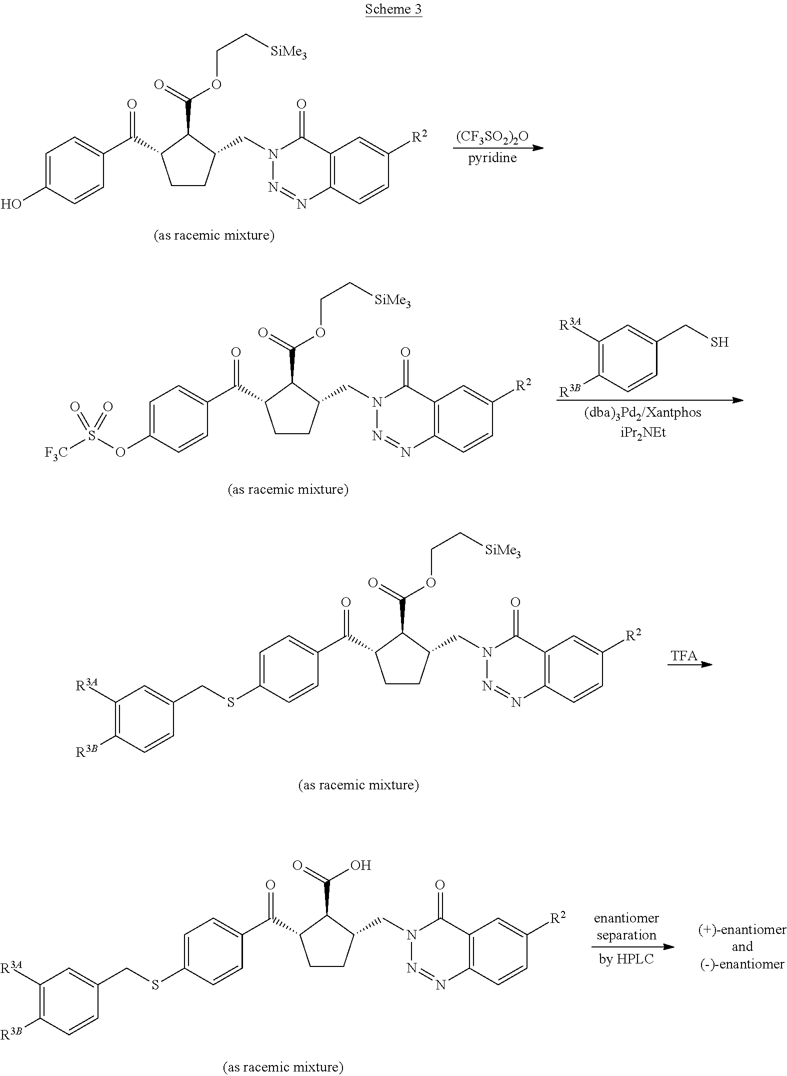

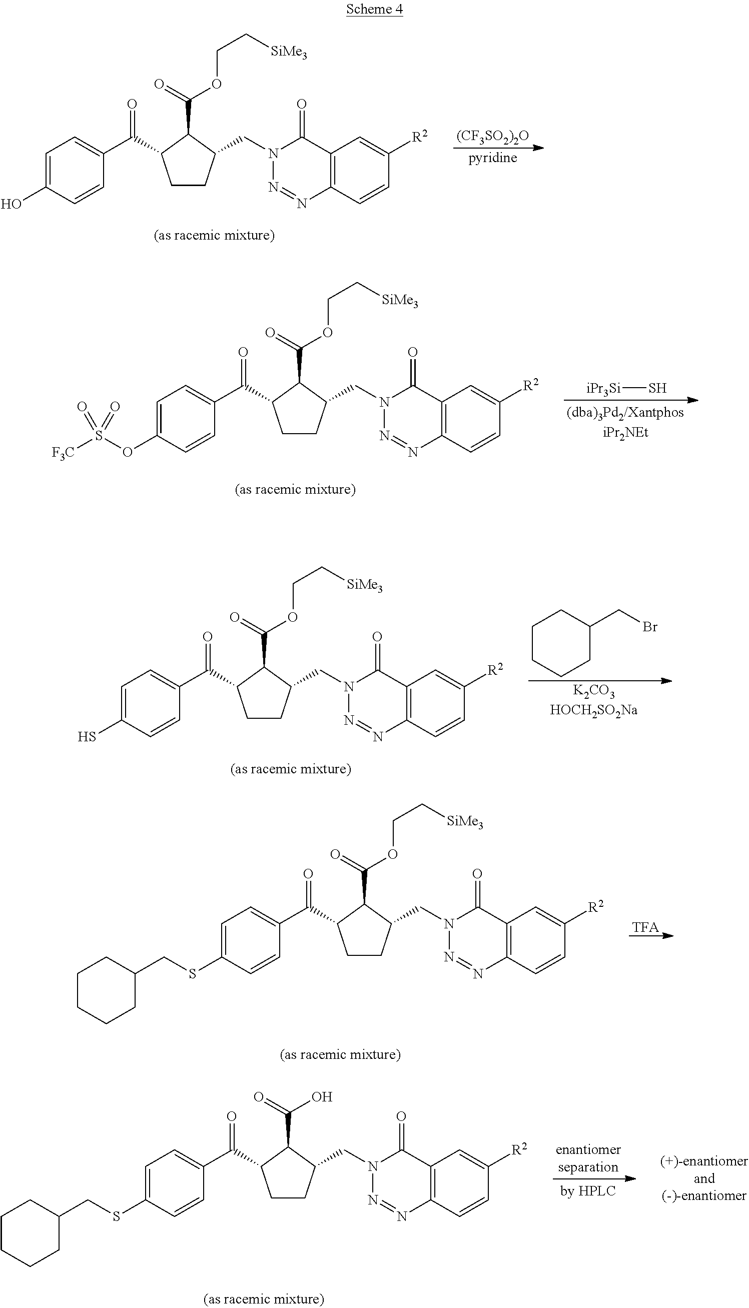

The compounds according to the invention have valuable pharmacological properties and can be used for prevention and treatment of diseases in humans and animals.

The compounds according to the invention are potent, non-reactive and selective inhibitors of human macrophage elastase (HME/hMMP-12) having a significantly improved profile with respect to potency and selectivity compared to the compounds known from the prior art. Furthermore, many of the compounds according to the invention have low in vitro clearance and good metabolic stability. This profile of properties overall suggests, for the compounds according to the invention, low dosability and—as a result of the more specific mode of action—reduced risk of the occurrence of unwanted side effects in treatment.

The compounds according to the invention are therefore suitable to a particular degree for treatment and/or prevention of diseases and pathological processes, in particular those in which macrophage elastase (HME/hMMP-12) is involved in the course of an infectious or noninfectious inflammatory event and/or tissue or vascular remodelling.

In the context of the present invention, these especially include disorders of the respiratory pathway and the lung, such as chronic obstructive pulmonary disease (COPD), asthma and the group of interstitial lung diseases (ILDs), and disorders of the cardiovascular system such as arteriosclerosis and aneurysms.

The forms of chronic obstructive lung disease (COPD) especially include pulmonary emphysema, for example the pulmonary emphysema induced by cigarette smoke, chronic bronchitis (CB), pulmonary hypertension in COPD (PH-COPD), bronchiectasis (BE) and combinations thereof, especially in acute exacerbating stages of the disease (AE-COPD).

The forms of asthma include asthmatic disorders of different severity with intermittent or persistent character, such as refractory asthma, bronchial asthma, allergic asthma, intrinsic asthma, extrinsic asthma and medicament- or dust-induced asthma.

The group of interstitial lung diseases (ILDs) includes idiopathic pulmonary fibrosis (IPF), pulmonary sarcoidosis and acute interstitial pneumonia, non-specific interstitial pneumonia, lymphoid interstitial pneumonia, respiratory bronchiolitis with interstitial pulmonary disorder, cryptogenic organizing pneumonia, desquamative interstitial pneumonia and non-classifiable idiopathic interstitial pneumonia, and also granulomatous interstitial pulmonary disorders, interstitial pulmonary disorders of known cause and other interstitial pulmonary disorders of unknown cause.

The compounds according to the invention can also be used for treatment and/or prevention of further disorders of the respiratory pathways and of the lung, for example of pulmonary arterial hypertension (PAH) and other forms of pulmonary hypertension (PH), of bronchiolitis obliterans syndrome (BOS), of acute respiratory distress syndrome (ARDS), of acute lung damage (ALI), alpha-1 antitrypsin deficiency (AATD) and cystic fibrosis (CF), of various forms of bronchitis (chronic bronchitis, infectious bronchitis, eosinophilic bronchitis), of bronchiectasis, farmer's lung and related diseases, cough- and cold-type diseases having infectious and non-infectious causes (chronic inflammatory cough, iatrogenic cough), mucous membrane inflammation (including medicamentous rhinitis, vasomotor rhinitis and seasonally dependent allergic rhinitis, for example hay fever), and polyps.

In the context of the present invention, the group of diseases of the cardiovascular system especially includes arteriosclerosis and its sequelae, for example stroke in the case of arteriosclerosis of the neck arteries (carotid arteriosclerosis), myocardial infarction in the case of arteriosclerosis of the coronary artery, peripheral arterial occlusive disease (pAOD) as a consequence of arteriosclerosis of arteries of the legs, and also aneurysms, especially aneurysms of the aorta, for example as a consequence of arteriosclerosis, high blood pressure, injuries and inflammations, infections (for example in the case of rheumatic fever, syphilis, Lyme borreliosis), inherited connective tissue weaknesses (for example in the case of Marfan syndrome and Ehlers-Danlos syndrome) or as a consequence of a volume load on the aorta in the case of inherited heart defects with right-left shunt or shunt-dependent perfusion of the lungs, and also aneurysms at coronary arteries in the course of suffering from Kawasaki syndrome and in areas of the brain in patients with a congenital malformation of the aortic valve.

In addition, the compounds according to the invention can be used for treatment and/or prevention of further cardiovascular disorders, for example high blood pressure (hypertension), heart failure, coronary heart disease, stable and unstable angina pectoris, renal hypertension, peripheral and cardiac vascular disorders, arrhythmias, atrial and ventricular arrhythmias and impaired conduction, for example atrioventricular blocks of degrees I-III, supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV junctional extrasystoles, sick sinus syndrome, syncopes, AV-nodal re-entry tachycardia, Wolff-Parkinson-White syndrome, acute coronary syndrome (ACS), autoimmune cardiac disorders (pericarditis, endocarditis, valvolitis, aortitis, cardiomyopathies), boxer cardiomyopathy, shock such as cardiogenic shock, septic shock and anaphylactic shock, and also for treatment and/or prevention of thromboembolic disorders and ischaemias such as myocardial ischaemia, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, micro- and macrovascular damage (vasculitis), and also to prevent restenoses, for example after thrombolysis therapies, percutaneous transluminal angioplasties (PTA), percutaneous transluminal coronary angioplasties (PTCA), heart transplants and bypass operations.

In the context of the present invention, the term “heart failure” encompasses both acute and chronic forms of heart failure, and also specific or related disease types thereof, such as acute decompensated heart failure, right heart failure, left heart failure, global failure, ischaemic cardiomyopathy, dilatative cardiomyopathy, hypertrophic cardiomyopathy, idiopathic cardiomyopathy, congenital heart defects, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders and diastolic and systolic heart failure.

The compounds according to the invention are also suitable for treatment and/or prevention of renal disorders, in particular renal insufficiency and kidney failure. In the context of the present invention, the terms “renal insufficiency” and “kidney failure” encompass both acute and chronic manifestations thereof and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and Alport's syndrome, immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds according to the invention for treatment and/or prevention of sequelae of renal insufficiency, for example hypertension, pulmonary oedema, heart failure, uraemia, anaemia, electrolyte disturbances (for example hyperkalaemia, hyponatraemia) and disturbances in bone and carbohydrate metabolism.

In addition, the compounds according to the invention are suitable for treatment and/or prevention of disorders of the urogenital system, for example benign prostate syndrome (BPS), benign prostate hyperplasia (BPH), benign prostate enlargement (BPE), bladder outlet obstruction (BOO), lower urinary tract syndromes (LUTS), neurogenic overactive bladder (OAB), incontinence, for example mixed urinary incontinence, urge urinary incontinence, stress urinary incontinence or overflow urinary incontinence (MUI, UUI, SUI, OUI), pelvic pain, and also erectile dysfunction and female sexual dysfunction.

In addition, the compounds according to the invention have antiinflammatory action and can therefore be used as antiinflammatory agents for treatment and/or prevention of sepsis (SIRS), multiple organ failure (MODS, MOF), inflammatory disorders of the kidney, chronic intestinal inflammations (IBD, Crohn's disease, ulcerative colitis), pancreatitis, peritonitis, cystitis, urethritis, prostatitis, epidimytitis, oophoritis, salpingitis, vulvovaginitis, rheumatoid disorders, inflammatory disorders of the central nervous system, multiple sclerosis, infammatory skin disorders and inflammatory eye disorders.

Furthermore, the compounds according to the invention are suitable for treatment and/or prevention of fibrotic disorders of the internal organs, for example the lung, the heart, the kidney, the bone marrow and in particular the liver, and also dermatological fibroses and fibrotic eye disorders. In the context of the present invention, the term “fibrotic disorders” includes in particular disorders such as hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, peritoneal fibrosis and similar fibrotic disorders, scleroderma, morphoea, keloids, hypertrophic scarring, naevi, diabetic retinopathy, proliferative vitroretinopathy and disorders of the connective tissue (for example sarcoidosis). The compounds according to the invention can likewise be used for promoting wound healing, for controlling postoperative scarring, for example following glaucoma operations and cosmetically for ageing or keratinized skin.

The compounds according to the invention can also be used for treatment and/or prevention of anaemias such as haemolytic anaemias, in particular haemoglobinopathies such as sickle cell anaemia and thalassaemias, megaloblastic anaemias, iron deficiency anaemias, anaemias owing to acute blood loss, displacement anaemias and aplastic anaemias.

Moreover, the compounds according to the invention are suitable for treatment of cancers, for example skin cancer, brain tumours, breast cancer, bone marrow tumours, leukaemias, liposarcomas, carcinomas of the gastrointestinal tract, of the liver, the pancreas, the lung, the kidney, the ureter, the prostate and the genital tract and also of malignant tumours of the lymphoproliferative system, for example Hodgkin's and non-Hodgkin's lymphoma.

In addition, the compounds according to the invention can be used for treatment and/or prevention of impaired lipid metabolism and dyslipidaemias (hypolipoproteinaemia, hypertriglyceridaemias, hyperlipidaemia, combined hyperlipidaemias, hypercholesterolaemia, abetalipoproteinaemia, sitosterolaemia), xanthomatosis, Tangier disease, adiposity, obesity, metabolic disorders (metabolic syndrome, hyperglycaemia, insulin-dependent diabetes, non-insulin-dependent diabetes, gestational diabetes, hyperinsulinaemia, insulin resistence, glucose intolerance and diabetic sequelae, such as retinopathy, nephropathy and neuropathy), of disorders of the gastrointestinal tract and the abdomen (glossitis, gingivitis, periodontitis, oesophagitis, eosinophilic gastroenteritis, mastocytosis, Crohn's disease, colitis, proctitis, anus pruritis, diarrhoea, coeliac disease, hepatitis, hepatic fibrosis, cirrhosis of the liver, pancreatitis and cholecystitis), of disorders of the central nervous system and neurodegenerative disorders (stroke, Alzheimer's disease, Parkinson's disease, dementia, epilepsy, depressions, multiple sclerosis), immune disorders, thyroid disorders (hyperthyreosis), skin disorders (psoriasis, acne, eczema, neurodermitis, various forms of dermatitis, for example dermatitis abacribus, actinic dermatitis, allergic dermatitis, ammonia dermatitis, facticial dermatitis, autogenic dermatitis, atopic dermatitis, dermatitis calorica, dermatitis combustionis, dermatitis congelationis, dermatitis cosmetica, dermatitis escharotica, exfoliative dermatitis, dermatitis gangraenose, stasis dermatitis, dermatitis herpetiformis, lichenoid dermatitis, dermatitis linearis, dermatitis maligna, medicinal eruption dermatitis, dermatitis palmaris and plantaris, parasitic dermatitis, photoallergic contact dermatitis, phototoxic dermatitis, dermatitis pustularis, seborrhoeic dermatitis, sunburn, toxic dermatitis, Meleney's ulcer, dermatitis veneata, infectious dermatitis, pyogenic dermatitis and rosacea-like dermatitis, and also keratitis, bullosis, vasculitis, cellulitis, panniculitis, lupus erythematosus, erythema, lymphomas, skin cancer, Sweet syndrome, Weber-Christian syndrome, scar formation, wart formation, chilblains), of inflammatory eye diseases (saccoidosis, blepharitis, conjunctivitis, iritis, uveitis, chorioiditis, ophthalmitis), viral diseases (caused by influenza, adeno and corona viruses, for example HPV, HCMV, HIV, SARS), of disorders of the skeletal bone and the joints and also the skeletal muscle (multifarious forms of arthritis, for example arthritis alcaptonurica, arthritis ankylosans, arthritis dysenterica, arthritis exsudativa, arthritis fungosa, arthritis gonorrhoica, arthritis mutilans, arthritis psoriatica, arthritis purulenta, arthritis rheumatica, arthritis serosa, arthritis syphilitica, arthritis tuberculosa, arthritis urica, arthritis villonodularis pigmentosa, atypical arthritis, haemophilic arthritis, juvenile chronic arthritis, rheumatoid arthritis and metastatic arthritis, furthermore Still syndrome, Felty syndrome, Sjörgen syndrome, Clutton syndrome, Poncet syndrome, Pott syndrome and Reiter syndrome, multifarious forms of arthropathies, for example arthropathia deformans, arthropathia neuropathica, arthropathia ovaripriva, arthropathia psoriatica and arthropathia tabica, systemic scleroses, multifarious forms of inflammatory myopathies, for example myopathie epidemica, myopathie fibrosa, myopathie myoglobinurica, myopathie ossificans, myopathie ossificans neurotica, myopathie ossificans progressiva multiplex, myopathie purulenta, myopathie rheumatica, myopathie trichinosa, myopathie tropica and myopathie typhosa, and also the Günther syndrome and the Münchmeyer syndrome), of inflammatory changes of the arteries (multifarious forms of arteritis, for example endarteritis, mesarteritis, periarteritis, panarteritis, arteritis rheumatica, arteritis deformans, arteritis temporalis, arteritis cranialis, arteritis gigantocellularis and arteritis granulomatosa, and also Horton syndrome, Churg-Strauss syndrome and Takayasu arteritis), of Muckle-Well syndrome, of Kikuchi disease, of polychondritis, dermatosclerosis and also other disorders having an inflammatory or immunological component, for example cataract, cachexia, osteoporosis, gout, incontinence, lepra, Sezary syndrome and paraneoplastic syndrome, for rejection reactions after organ transplants and for wound healing and angiogenesis in particular in the case of chronic wounds.

On account of their profile of properties, the compounds according to the invention are especially suitable for treatment and/or prevention of diseases of the respiratory tract and of the lung, primarily chronic obstructive pulmonary disorder (COPD), here in particular lung emphysema, chronic bronchitis (CB), pulmonary hypertension in COPD (PH-COPD) and bronchiectasis (BE), and also of combinations of these types of illnesses, particularly in acutely exacerbating stages of COPD disease (AE COPD), furthermore of asthma and of interstitial lung diseases, here in particular idiopathic pulmonary fibrosis (IPF) and pulmonary sarcoidosis, of diseases of the cardiovascular system, in particular of arteriosclerosis, specifically of carotid arteriosclerosis, and also viral myocarditis, cardiomyopathy and aneurysms, including their sequelae such as stroke, myocardial infarction and peripheral arterial occlusive disease (pAVK), and also of chronic kidney diseases and Alport's syndrome.

The aforementioned well-characterized diseases in humans can also occur with comparable aetiology in other mammals and can likewise be treated therein with the compounds of the present invention.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The present invention further provides for the use of the compounds according to the invention for treatment and/or prevention of disorders, especially of the aforementioned disorders.

The present invention further provides for the use of the compounds according to the invention for production of a medicament for treatment and/or prevention of disorders, especially of the aforementioned disorders.

The present invention further provides a medicament comprising at least one of the compounds according to the invention for treatment and/or prevention of disorders, especially of the aforementioned disorders.

The present invention further provides for the use of the compounds according to the invention in a method for treatment and/or prevention of disorders, especially of the aforementioned disorders.

The present invention further provides a process for treatment and/or prevention of disorders, especially of the aforementioned disorders, using an effective amount of at least one of the compounds according to the invention.

The compounds according to the invention can be used alone or, if required, in combination with one or more other pharmacologically active substances, provided that this combination does not lead to undesirable and unacceptable side effects. The present invention therefore further provides medicaments comprising at least one of the compounds according to the invention and one or more further active ingredients, especially for treatment and/or prevention of the aforementioned disorders. Preferred examples of combination active ingredients suitable for this purpose include:
anti-obstructive/bronchodilatory agents as used, for example, for treatment of chronic obstructive pulmonary disease (COPD) or bronchial asthma, by way of example and with preference from the group of the inhalatively or systemically administered agonists of the beta-adrenergic receptor (beta-mimetics), the inhalatively administered anti-muscarinergic substances and the PDE 4 inhibitors;
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 4 inhibitors such as roflumilast and PDE 5 inhibitors such as sildenafil, vardenafil, tadalafil, udenafil, dasantafil, avanafil, mirodenafil or lodenafil;
NO- and haem-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat and the compounds described in WO 00/06568, WO 00/06569, WO 02/42301, WO 03/095451, WO 2011/147809, WO 2012/004258, WO 2012/028647 and WO 2012/059549;
compounds which inhibit human neutrophil elastase (HNE), such as in particular sivelestat, DX-890 (Reltran) and the compounds described in WO 2004/020410, WO 2004/020412, WO 2004/024700, WO 2004/024701, WO 2005/080372, WO 2005/082863, WO 2005/082864, WO 2009/080199, WO 2009/135599, WO 2010/078953 and WO 2010/115548;
prostacyclin analogues and IP receptor agonists, by way of example and with preference iloprost, beraprost, treprostinil, epoprostenol or NS-304;
endothelin receptor antagonists, by way of example and with preference bosentan, darusentan, ambrisentan or sitaxsentan;
antiinflammatory, immunomodulating, immunosuppressive and/or cytotoxic agents, by way of example and with preference from the group of the systemically or inhalatively administered corticosteroids and also acetylcysteine, montelukast, azathioprine, cyclophosphamide, hydroxycarbamide, azithromycin, IFN-γ, pirfenidone or etanercept;
antifibrotic agents, by way of example and with preference lysophosphatidic acid receptor 1 (LPA-1) antagonists, lysyl oxidase (LOX) inhibitors, lysyl oxidase-like-2 inhibitors, vasoactive intestinal peptide (VIP), VIP analogues, α_{vβ6}m-integrin antagonists, cholchicine, IFN-β, D-penicillamine, inhibitors of the WNT signal path or CCR2 antagonists;
active ingredients that alter lipid metabolism, by way of example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists;
hypotensive active ingredients, by way of example and with preference from the group of the calcium antagonists, angiotensin AII antagonists, ACE inhibitors, vasopeptidase inhibitors, endothelin antagonists, renin inhibitors, alpha receptor blockers, beta receptor blockers, mineralocorticoid receptor antagonists and also the diuretics;
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
compounds which block the binding of serotonin to its receptors, by way of example and with preference antagonists of the 5-HT2B receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
compounds which influence the energy metabolism of the heart, by way of example and with preference etomoxir, dichloroacetate, ranolazine or trimetazidine;
antithrombotic agents, by way of example and with preference from the group of platelet aggregation inhibitors, the anticoagulants and the profibrinolytic substances;
chemotherapeutics as used, for example, for treatment of neoplasms in the lung or other organs; and/or
antibiotics, especially from the group of the fluoroquinolonecarboxylic acids, by way of example and with preference ciprofloxacin or moxifloxacin.

In a preferred embodiment of the invention, the compounds according to the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the compounds according to the invention are administered in combination with an antimuscarinergic substance, by way of example and with preference ipratropium bromide, tiotropium bromide or oxitropium bromide.

In a preferred embodiment of the invention, the compounds according to the invention are administered in combination with a corticosteroid, by way of example and with preference prednisone, prednisolone, methylprednisolone, triamcinolone, dexamethasone, beclomethasone, betamethasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the compounds according to the invention are administered in combination with a platelet aggregation inhibitor, by way of example and with preference aspirin, clopidogrel, ticlopidine or dipyridamole.

In a preferred embodiment of the invention, the compounds according to the invention are administered in combination with a thrombin inhibitor, by way of example and with preference ximelagatran, melagatran, dabigatran, bivalirudin or clexane.

In a preferred embodiment of the invention, the compounds according to the invention are administered in combination with a GPIIb/IIIa antagonist, by way of example and with preference tirofiban or abciximab.

In a preferred embodiment of the invention, the compounds according to the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embursatan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone, eplerenone or finerenone.

In a preferred embodiment of the invention, the compounds according to the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds according to the invention are administered in combination with a CETP inhibitor, by way of example and with preference torcetrapib (CP-529 414), HT-705 or CETP vaccine (Avant).

In a preferred embodiment of the invention, the compounds according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or JTT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorber, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, by way of example and with preference gemcabene calcium (CI-1027) or nicotinic acid.

Particular preference is given to combinations of the compounds according to the invention with one or more further active ingredients selected from the group consisting of corticosteroids, beta-adrenergic receptor agonists, anti-muscarinergic substances, PDE 4 inhibitors, PDE 5 inhibitors, sGC activators, sGC stimulators, HNE inhibitors, prostacyclin analogues, endothelin antagonists, statins, antifibrotic agents, anti-inflammatory agents, immunomodulating agents, immunosuppressive agents and cytotoxic agents.

The present invention further provides medicaments which comprise at least one compound according to the invention, typically together with one or more inert, non-toxic, pharmaceutically suitable excipients, and for the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in administration forms suitable for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. take place intravenously, intraarterially, intracardially, intraspinally or intralumbally) or include an absorption (e.g. take place inhalatively, intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers, metered aerosols), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral, intrapulmonary (inhalative) and intravenous administration.

The compounds according to the invention can be converted to the administration forms mentioned. This can be accomplished in a manner known per se by mixing with inert, non-toxic, pharmaceutically suitable excipient. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight. In the case of intrapulmonary administration, the amount is generally about 0.1 to 50 mg per inhalation.

It may nevertheless be necessary in some cases to deviate from the stated amounts, specifically as a function of body weight, route of administration, individual response to the active compound, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

### A. Examples

### Abbreviations and Acronyms

abs. absolute
Ac acetyl
aq. aqueous, aqueous solution
br. broad (in NMR signal)
Ex. Example
Bu butyl
c concentration
ca. circa, about
cat. catalytic
CI chemical ionization (in MS)
d doublet (in NMR)
d day(s)
(dba)₃Pd₂ tris(dibenzylideneacetone)dipalladium(0)
TLC thin-layer chromatography
DCI direct chemical ionization (in MS)
dd doublet of doublets (in NMR)
DEAD diethyl azodicarboxylate

### DMF N,N-dimethylformamide

DMSO dimethyl sulphoxide
dt doublet of triplets (in NMR)
ee enantiomeric excess
EI electron impact ionization (in MS)
ent enantiomerically pure, enantiomer
eq. equivalent(s)
ESI electrospray ionization (in MS)
Et ethyl
h hour(s)
HPLC high-pressure, high-performance liquid chromatography
iPr isopropyl
conc concentrated (in the case of a solution)
LC liquid chromatography
LC/MS liquid chromatography-coupled mass spectrometry
Lit. literature (reference)
m multiplet (in NMR)
Me methyl
min minute(s)
MPLC medium-pressure liquid chromatography (on silica gel; also referred to as flash chromatography)
Ms methanesulphonyl (mesyl)
MS mass spectrometry

### NMO N-methylmorpholine N-oxide

NMR nuclear magnetic resonance spectrometry
Pd/C palladium on activated carbon
Pr propyl
q (or quart) quartet (in NMR)
qd quartet of doublets (in NMR)
quant. quantitative (in chemical yield)
quint quintet (in NMR)
rac racemic, racemate
R_{f} retention index (in TLC)
RP reverse phase (in HPLC)
RT room temperature
R₁ retention time (in HPLC, LC/MS)
s singlet (in NMR)
sept septet (in NMR)
SFC supercritical liquid chromatography
t triplet (in NMR)
tBu tert-butyl
td triplet of doublets (in NMR)
TFA trifluoroacetic acid
THF tetrahydrofuran
UV ultraviolet spectrometry
v/v ratio by volume (of a solution)
Xantphos 4,5-bis(diphenylphosphino)-9,9-dimethylxanthene
HPLC- and LC/MS methods:

### Method 1 (LC/MS):

Instrument: Waters ACQUITY SQD UPLC System; column: Waters Acquity UPLC HSS T3 1.8μ 50×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 208-400 nm.

### Method 2 (LC/MS):

Instrument: Micromass Quattro Premier with Waters UPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 3 (LC/MS):

MS instrument: Waters Micromass QM; HPLC instrument: Agilent 1100 series; column: Agilent ZORBAX Extend-C18 3.5μ, 3.0×50 mm; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm.

### Method 4 (Preparative HPLC):

Column: Reprosil C18, 10μ, 250×30 mm; eluent: acetonitrile/water with 0.1% TFA; gradient: 0-5.00 min 10:90, sample injection at 3.00 min; 5.00-23.00 min to 95:5; 23.00-30.00 min 95:5; 30.00-30.50 min to 10:90; 30.50-31.20 min 10:90.

### Method 5 (Preparative HPLC):

Column: Reprosil C18, 10 μm, 250×30 mm; eluent: acetonitrile/water with 0.1% TFA; gradient: 0-5.00 min 10:90, sample injection at 3.00 min; 5.00-20.00 min to 95:5; 20.00-30.00 min 95:5; 30.00-30.50 min to 10:90; 30.50-31.20 min 10:90.

### Method 6 (Preparative HPLC):

Column: Reprosil C18, 10 μm, 125×30 mm; eluent: acetonitrile/water with 0.1% TFA; gradient: 0-6.00 min 35:65, sample injection at 3.00 min; 6.00-27.00 min to 80:20; 27.00-30.00 min 95:5; 30.00-33.00 min to 35:65.

### Method 7 (Preparative HPLC):

Column: Reprosil-Pur C18, 10 μm; eluent: water/methanol; gradient: 70:30→50:50 (to 6 min)→20:80 (to 22 min), to 75 min 20:80.

### Method 8 (Preparative HPLC):

Column: Reprosil-Pur C18, 10 μm; eluent: water/methanol; gradient: 70:30→50:50 (to 6 min)→20:80 (to 20 min), to 115 min 20:80.

### Method 9 (Preparative HPLC):

Column: Reprosil-Pur C18, 10 μm; eluent: water/methanol; gradient: 70:30→50:50 (to 6 min)→20:80 (to 21 min), to 75 min 20:80.

### Method 10 (Preparative HPLC):

Column: Reprosil-Pur C18, 10 μm; eluent: water/methanol; gradient: 70:30→50:50 (to 6 min)→20:80 (to 25 min), to 75 min 20:80.

### Method 11 (Preparative HPLC):

Column: Reprosil-Pur C18, 10 μm; eluent: water/methanol; gradient: 70:30→50:50 (to 6 min)→20:80 (to 20 min), to 75 min 20:80.

### Further Details:

The percentages in the example and test descriptions which follow are, unless indicated otherwise, percentages by weight; parts are parts by weight. Solvent ratios, dilution ratios and concentration data for the liquid/liquid solutions are based in each case on volume.

Purity figures are generally based on corresponding peak integrations in the LC/MS chromatogram, but may additionally also have been determined with the aid of the ¹H NMR spectrum. If no purity is indicated, the purity is generally 100% according to automated peak integration in the LC/MS chromatogram, or the purity has not been determined explicitly.

Stated yields in % of theory are generally corrected for purity if a purity of <100% is indicated. In solvent-containing or contaminated batches, the formal yield may be “>100%”; in these cases the yield is not corrected for solvent or purity.

The descriptions of the coupling patterns of ¹H NMR signals that follow have in some cases been taken directly from the suggestions of the ACD SpecManager (ACD/Labs Release 12.00, Product version 12.5) and have not necessarily been strictly scrutinized. In some cases, the suggestions of the SpecManager were adjusted manually. Manually adjusted or assigned descriptions are generally based on the optical appearance of the signals in question and do not necessarily correspond to a strict, physically correct interpretation. In general, the stated chemical shift refers to the centre of the signal in question. In the case of broad multiplets, an interval is given. Signals obscured by solvent or water were either tentatively assigned or have not been listed.

Melting points and melting-point ranges, if stated, are uncorrected.

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

In the intermediates and working examples described hereinafter, a “1RS,2RS,5SR” identifier in the IUPAC name of the example in question, in conjunction with the term “racemate”, means that this is a racemic mixture of the 1R,2R,5S enantiomer (→1st letter in each case after the position number in “1RS,2RS,5SR”) with the corresponding 1S,2S,5R enantiomers (→2nd letter in each case after the position number). The “1RS,2RS,5SR” identifier in conjunction with the statements “enantiomer 1” and “enantiomer 2” means that these are the two enantiomers in separate, isolated form, without having undertaken an assignment of the absolute configuration (1R,2R,5S or 1S,2S,5R) to these enantiomers. Similar identifiers such as “1RS,2SR,5RS” that arise from the altered priority and/or sequence of main constituents owing to the IUPAC nomenclature rules should be interpreted in an analogous manner according to these instructions.

For the simplified representation of the relative stereochemical configuration of chiral centres, the structural formulae of racemic example compounds hereinbelow show only the structural formula of one of the enantiomers involved; as is evident from the term “racemate” in the associated IUPAC name, the second enantiomer with the respective opposite absolute configuration is always included in these cases.

### Starting Compounds and Intermediates:

### Example 1A

### 6-(Trifluoromethyl)-1,2,3-benzotriazin-4(3H)-one

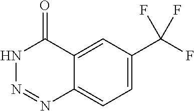 [SMILES: O=c1[nH]nnc2ccc(C(F)(F)F)cc12]

To a suspension of 24.4 g (119.51 mmol) of 2-amino-5-(trifluoromethyl)benzamide in 174 ml of a 2:1 mixture of water and conc. hydrochloric acid at 0° C. was gradually added a solution of 9.08 g (131.47 mmol) of sodium nitrite in 74 ml of water, in the course of which the internal temperature was kept below 5° C. After stirring at bath temperature 0° C. for 30 minutes, while continuing to cool with an ice bath, 74 ml (0.74 mol) of 10 M sodium hydroxide solution were added, in the course of which the internal temperature rose to about 20° C. A solution formed at first, from which a suspension then arose, which was diluted with 100 ml of water for better stirrability. After stirring at RT for 1.5 h, the mixture was cautiously acidified with conc. hydrochloric acid (pH=2). The precipitate formed was filtered off and washed three times with water. After drying under air and then under reduced pressure, 24.74 g (96% of theory) of the title compound were obtained.

¹H-NMR (400 MHz, DMSO-d₆): δ [ppm]=15.31 (br. s, 1H), 8.46 (s, 1H), 8.40 (d, 2H).

LC/MS (Method 1, ESIpos): Rₜ=0.78 min, m/z=216 [M+H]⁺.

### Example 2A

### 6-Methyl-1,2,3-benzotriazin-4(3H)-one

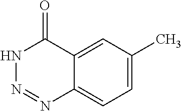

To a suspension of 32.0 g (213.08 mmol) of 2-amino-5-methylbenzamide in 300 ml of a 2:1 mixture of water and conc. hydrochloric acid at 0° C. was gradually added a solution of 16.17 g (234.38 mmol) of sodium nitrite in 120 ml of water, in the course of which the internal temperature was kept below 5° C. After stirring at bath temperature 0° C. for 30 minutes, while continuing to cool with an ice bath, 120 ml (1.2 mol) of 10 M sodium hydroxide solution were added, in the course of which the internal temperature rose to about 20° C. and solids that were present went into solution. After stirring at RT for 1 h, the mixture was cautiously acidified with conc. hydrochloric acid (pH=2). The precipitate formed was filtered off and washed three times with water. After drying under air and under reduced pressure, 33.80 g (98% of theory) of the title compound were obtained.

¹H-NMR (400 MHz, DMSO-d₆): δ [ppm]=14.85 (br. s, 1H), 8.08 (d, 1H), 8.02 (s, 1H), 7.90 (d, 1H).

LC/MS (Method 3, ESIpos): Rₜ=1.40 min, m/z=162 [M+H]⁺.

### Example 3A

### 2-(Trimethylsilyl)ethyl (1RS,2RS,5SR)-2-[4-(benzyloxy)benzoyl]-5-[(4-oxo-1,2,3-benzotriazin-3(4H)-yl)methyl]cyclopentanecarboxylate (racemate)

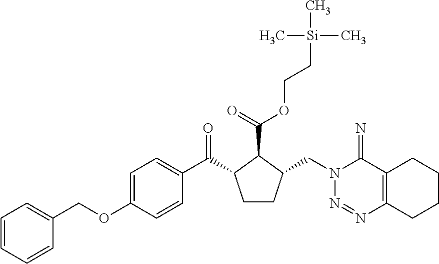 [SMILES: C[Si](C)(C)CCOC(=O)[C@H]1[C@H](Cn2nnc3c(c2=N)CCCC3)CC[C@@H]1C(=O)c1ccc(OCc2ccccc2)cc1]

### Step 1

### 2-(Trimethylsilyl)ethyl 2-[4-(benzyloxy)phenyl]-2-hydroxybicyclo[2.2.1]heptane-7-carboxylate

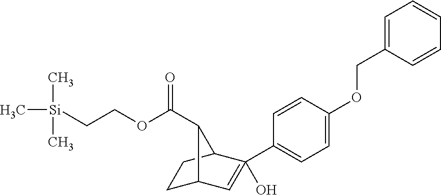

To a solution of 24.30 g (95.52 mmol) of exo-2-(trimethylsilyl)ethyl 2-oxobicyclo[2.2.1]heptane-7-carboxylate [WO 96/15096, Example 360/Stage 1] in 60 ml of THF were gradually added, at internal temperature about −5° C. under argon, 114.62 ml (114.62 mmol) of a 1 M solution of 4-(benzyloxy)phenylmagnesium bromide in THF, in the course of which the internal temperature rose to not more than 0° C. The cold bath was then removed and the mixture was stirred for a further 1 h. The mixture was then admixed with 200 ml of 5% citric acid solution and extracted twice with dichloromethane. The combined organic phases were dried over magnesium sulphate and concentrated. The residue was purified by means of flash chromatography on 1 kg of silica gel (eluent: cyclohexane/ethyl acetate 9:1). 28.70 g (66% of theory, 97% purity) of the title compound were obtained.

¹H-NMR (400 MHz, DMSO-d₆): δ [ppm]=7.49-7.27 (m, 7H), 6.95 (d, 2H), 5.09 (s, 2H), 5.05 (s, 1H), 4.10-4.00 (m, 2H), 2.44-2.37 (m, 1H), 2.33-2.24 (m, 1H), 2.23-2.11 (m, 1H), 1.78-1.60 (m, 1H), 1.52-1.26 (m, 4H), 0.95-0.80 (m, 2H), 0.00 (s, 9H).

LC/MS (Method 1, ESIpos): Rₜ=3.15 min, m/z=421 [M+H−H₂O]⁺.

### Step 2

### 2-(Trimethylsilyl)ethyl 2-[4-(benzyloxy)phenyl]bicyclo[2.2.1]hept-2-ene-7-carboxylate

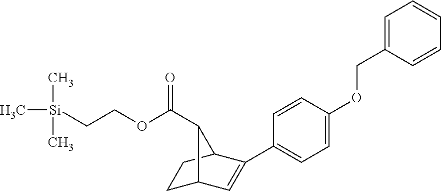 [SMILES: C[Si](C)(C)CCOC(=O)C1C2C=C(c3ccc(OCc4ccccc4)cc3)C1CC2]

To a solution of 28.70 g (63.466 mmol) of the compound from Example 3A/Step 1 in 150 ml of dichloromethane under argon were added, at about 0° C., first 26.50 ml (190.40 mmol) of triethylamine and then, gradually, 9.82 ml (126.93 mmol) of methanesulphonyl chloride, in the course of which the internal temperature did not exceed 5° C. This was followed by stirring at 0° C. for a further 1.5 h. Thereafter, the mixture was diluted with dichloromethane and extracted with water. The organic phase was dried over magnesium sulphate and concentrated, and the residue was purified by means of flash chromatography on 1 kg of silica gel (eluent: cyclohexane/ethyl acetate 95:5). 20.06 g (75% of theory) of the title compound were obtained.

¹H-NMR (400 MHz, DMSO-d₆): δ [ppm]=7.48-7.28 (m, 7H), 6.97 (d, 2H), 6.30 (d, 1H), 5.11 (s, 2H), 4.15-4.06 (m, 2H), 3.43 (br. s, 1H), 3.06 (br. s, 1H), 1.85-1.71 (m, 2H), 1.17-1.06 (m, 1H), 1.04-0.87 (m, 3H), 0.04 (s, 9H).

LC/MS (Method 1, ESIpos): Rₜ=1.61 min, m/z=421 [M+H]⁺.

### Step 3

### 2-(Trimethylsilyl)ethyl 2-[4-(benzyloxy)phenyl]-2,3-dihydroxybicyclo[2.2.1]heptane-7-carboxylate

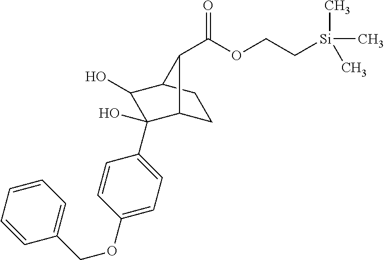 [SMILES: C[Si](C)(C)CCOC(=O)C1C2CCC1C(O)(c1ccc(OCc3ccccc3)cc1)C2O]

To a degassed solution of 25.37 g (60.314 mmol, not corrected for purity) of the compound from Example 3A/Step 2 in 150 ml of THF under argon was added, at 0° C., a degassed solution of 15.90 g (135.71 mmol) of N-methylmorpholine N-oxide (NMO) in 42 ml of water under argon. To this mixture were then gradually added, while stirring, 116 ml (9.05 mmol) of a 2.5% solution of osmium tetroxide in tert-butanol. This was followed by stirring at 0° C. for a further 1 h. After stirring at RT for a further 16 h, the mixture was diluted with 150 ml of ethyl acetate and extracted twice with 250 ml each time of 10% citric acid solution, twice with 300 ml each time of saturated sodium hydrogencarbonate solution and twice with 300 ml each time of saturated sodium chloride solution. The organic phase was then dried over sodium sulphate and concentrated. 27.51 g (75% of theory, 75% purity) of the title compound were obtained.

LC/MS (Method 1, ESIpos): Rₜ=1.40 min, m/z=437 [M+H−H₂O]⁺.

### Step 4

### 2-(Trimethylsilyl)ethyl (1RS,2RS,5SR)-2-[4-(benzyloxy)benzoyl]-5-formylcyclopentanecarboxylate (racemate)

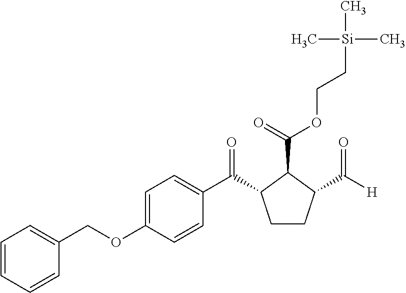 [SMILES: C[Si](C)(C)CCOC(=O)[C@H]1[C@H](C=O)CC[C@@H]1C(=O)c1ccc(OCc2ccccc2)cc1]

### Method A:

To a solution of 27.42 g (60.32 mmol, not corrected for purity) of the compound from Example 3A/Step 3 in 170 ml of methanol under argon were added gradually, at bath temperature −15° C., 30.96 g (66.34 mmol, 95% purity) of lead tetraacetate. The mixture was stirred at −15° C. for 1 h. After warming to RT, the mixture was filtered through Celite and the filtration residue was washed three times with 50 ml each time of methanol. The filtrate was concentrated and the residue was taken up in 500 ml of dichloromethane and 500 ml of water without onset of a phase separation. Thereafter, the mixture was filtered through silica gel and the silica gel was washed with dichloromethane After phase separation, the aqueous phase was extracted once again with 150 ml of dichloromethane. The combined organic phases were dried over sodium sulphate and concentrated. 27.1 g (86% of theory, 87% purity) of the title compound were obtained.

¹H-NMR (400 MHz, DMSO-d₆): δ [ppm]=9.72 (d, 1H), 8.02 (d, 2H), 7.53-7.34 (m, 5H), 7.18 (d, 2H), 5.25 (s, 2H), 4.17 (q, 1H), 4.09 (dd, 2H), 3.74 (t, 1H), 3.23-3.14 (m, 1H), 2.24-2.13 (m, 1H), 2.08-1.88 (m, 2H), 1.61-1.49 (m, 1H), 0.87-0.79 (m, 2H), 0.00 (s, 9H).

LC/MS (Method 1, ESIpos): Rₜ=1.45 min, m/z=425 [M+H−28]⁺.

### Method B:

To a solution of 69.0 g (131 mmol, about 80% purity) of the compound from Example 3A/Step 2 in a mixture of acetone/water/THF (3:1:1) were added, at 0° C. under argon, first 76.87 g (656 mmol) of N-methylmorpholine N-oxide (NMO) and then 2.09 g (8.20 mmol) of a 4% solution of osmium tetroxide in water. The mixture was stirred at RT for 3 days. Then 105.26 g (492 mmol) of sodium periodate were added and stirring of the mixture at RT continued overnight. After ethyl acetate and 10% aqueous citric acid had been added, the aqueous phase was removed and extracted once with ethyl acetate. The combined organic phases were washed once with saturated sodium hydrogencarbonate solution and then with magnesium silicate (Florisil). After filtration, the filter residue was washed with ethyl acetate. After the filtrate had been concentrated, the residue thus obtained was combined with the residues from two similarly conducted prior experiments [amounts of the compound from Example 3A used: 3.0 g (7.13 mmol) and 3.2 g (7.61 mmol)] and purified jointly by means of flash chromatography (silica gel, eluent: petroleum ether/ethyl acetate 8:2). In this way, a total of 53 g (58% of theory taking account of the prior experiments, 89% purity) of the title compound were obtained.

### Step 5

### 2-(Trimethylsilyl)ethyl (1RS,2RS,5SR)-2-[4-(benzyloxy)benzoyl]-5-(hydroxymethyl)cyclopentane-carboxylate (racemate)

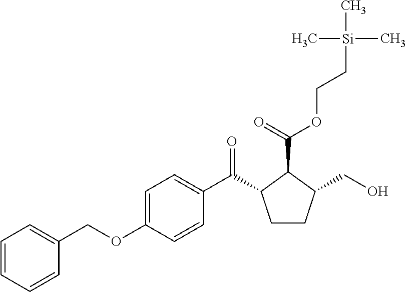 [SMILES: C[Si](C)(C)CCOC(=O)[C@H]1[C@H](CO)CC[C@@H]1C(=O)c1ccc(OCc2ccccc2)cc1]

To a solution of 27.0 g (59.65 mmol, not corrected for purity) of the compound from Example 3A/Step 4 in 135 ml of ethanol were added gradually, at RT, 677 mg (17.895 mmol) of sodium borohydride, and the mixture was stirred at RT for 30 min Subsequently, the mixture was admixed with 400 ml each of ammonium chloride solution and water and extracted twice with 300 ml each time of ethyl acetate. The combined organic phases were dried over sodium sulphate and concentrated. 21.90 g (70% of theory, 87% purity) of the title compound were obtained.

¹H-NMR (400 MHz, DMSO-d₆): δ [ppm]=7.95 (d, 2H), 7.48-7.31 (m, 5H), 7.12 (d, 2H), 5.20 (s, 2H), 4.64 (t, 1H), 4.07-3.98 (m, 3H), 3.53-3.45 (m, 1H), 3.40-3.34 (m, 1H), 2.94 (t, 1H), 2.34-2.23 (m, 1H), 2.12-2.01 (m, 1H), 1.90-1.78 (m, 1H), 1.67-1.47 (m, 2H), 0.82-0.75 (m, 2H), 0.00 (s, 9H).

LC/MS (Method 1, ESIpos): Rₜ=1.34 min, m/z=455 [M+H]⁺.

### Step 6

### 2-(Trimethylsilyl)ethyl (1RS,2RS,5SR)-2-[4-(benzyloxy)benzoyl]-5-[(4-oxo-1,2,3-benzotriazin-3(4H)-yl)methyl]cyclopentanecarboxylate (racemate)

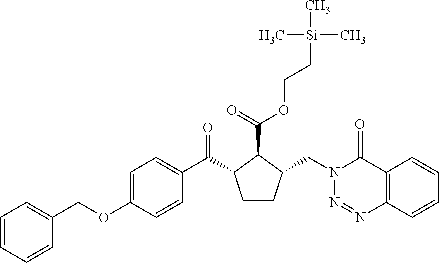 [SMILES: C[Si](C)(C)CCOC(=O)[C@H]1[C@H](Cn2nnc3ccccc3c2=O)CC[C@@H]1C(=O)c1ccc(OCc2ccccc2)cc1]

To a solution of 500 mg (1.10 mmol, not corrected for purity) of the compound from Example 3A/Step 5 in 6 ml of THF under argon were added 243 mg (1.65 mmol) of 1,2,3-benzotriazin-4(3H)-one and 1.11 g (5.50 mmol) of tributylphosphine. Subsequently, 1.50 ml (3.30 mmol) of a 40% solution of diethyl azodicarboxylate (DEAD) in toluene were added dropwise at 0° C. The mixture was stirred at RT for about 1 h, then diluted with ethyl acetate and extracted twice with 5 ml each time of water and twice with saturated sodium chloride solution. The organic phase was dried over magnesium sulphate and concentrated. The residue was purified by means of preparative HPLC (Method 6). 334 mg (52% of theory) of the title compound were obtained.

¹H-NMR (400 MHz, DMSO-d₆): δ [ppm]=8.44 (dd, 1H), 8.38 (d, 1H), 8.27 (td, 1H), 8.15-8.08 (m, 3H), 7.65-7.48 (m, 5H), 7.29 (d, 2H), 5.37 (s, 2H), 4.74-4.62 (m, 2H), 4.26 (q, 1H), 3.40 (t, 1H), 3.13-3.01 (m, 1H), 2.36-2.25 (m, 1H), 2.21-2.10 (m, 1H), 1.96-1.84 (m, 1H), 1.77-1.65 (m, 1H), 0.53-0.46 (m, 2H), 0.17 (s, 9H).

LC/MS (Method 1, ESIpos): Rₜ=1.51 min, m/z=584 [M+H]⁺.

### Example 4A

### 2-(Trimethylsilyl)ethyl (1RS,2RS,5SR)-2-(4-hydroxybenzoyl)-5-[(4-oxo-1,2,3-benzotriazin-3(4H)-yl)methyl]cyclopentanecarboxylate (racemate)

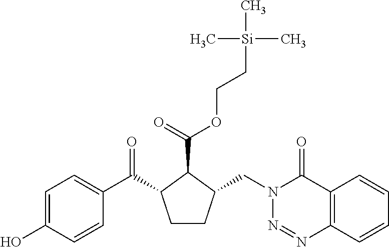 [SMILES: C[Si](C)(C)CCOC(=O)[C@H]1[C@H](Cn2nnc3ccccc3c2=O)CC[C@@H]1C(=O)c1ccc(O)cc1]

To a solution of 270 mg (0.46 mmol) of the compound from Example 3A in 12 ml of ethyl acetate under argon were added 25 mg (0.024 mmol) of palladium on activated carbon (10% Pd). This was followed by hydrogenation under standard pressure for 42 h. The mixture was then filtered through kieselguhr, the filter residue was washed with ethyl acetate and the filtrate was concentrated. The residue thus obtained was taken up in a little dichloromethane and purified by column chromatography (25 g of silica gel, eluent: cyclohexane/ethyl acetate 7:3). 165 mg (72% of theory, 100% purity) of the title compound were obtained.

¹H-NMR (400 MHz, CDCl₃): δ [ppm]=8.37 (d, 1H), 8.16 (d, 1H), 7.98-7.88 (m, 3H), 7.84-7.77 (m, 1H), 6.89 (d, 2H), 6.67 (br. s, 1H), 4.77-4.70 (m, 1H), 4.68-4.60 (m, 1H), 4.20-4.10 (m, 1H), 3.88-3.81 (m, 2H), 3.46 (t, 1H), 3.08-2.94 (m, 1H), 2.19-2.04 (m, 1H), 2.01-1.86 (m, 2H), 1.72-1.64 (m, partially hidden, 1H), 0.63-0.55 (m, 2H), −0.09 (s, 9H).

LC/MS (Method 1, ESIpos): Rₜ=1.23 min, m/z=494 [M+H]⁺.

### Example 5A

### 2-(Trimethylsilyl)ethyl (1RS,2RS,5SR)-2-{4-[(4-fluorobenzyl)oxy]benzoyl}-5-[(4-oxo-1,2,3-benzotriazin-3(4H)-yl)methyl]cyclopentanecarboxylate (racemate)

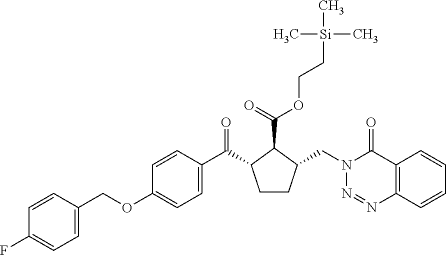 [SMILES: C[Si](C)(C)CCOC(=O)[C@H]1[C@H](Cn2nnc3ccccc3c2=O)CC[C@@H]1C(=O)c1ccc(OCc2ccc(F)cc2)cc1]

To a solution of 80 mg (0.16 mmol) of the compound from Example 4A in 1.8 ml of acetonitrile were added 45 mg (0.32 mmol) of potassium carbonate and 38 mg (0.19 mmol, 97% purity) of 4-fluorobenzyl bromide. Then the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 7). 50 mg (52% of theory, 100% purity) of the title compound were obtained.

LC/MS (Method 1, ESIpos): Rₜ=1.50 min, m/z=602 [M+H]⁺.

### Example 6A

### 2-(Trimethylsilyl)ethyl (1RS,2RS,5SR)-2-{4-[(3-chlorobenzyl)oxy]benzoyl}-5-[(4-oxo-1,2,3-benzotriazin-3(4H)-yl)methyl]cyclopentanecarboxylate (racemate)

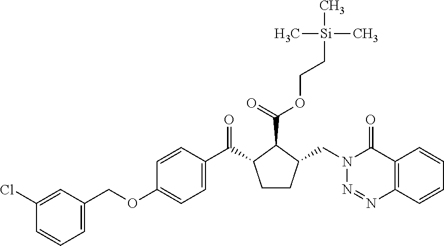 [SMILES: C[Si](C)(C)CCOC(=O)[C@H]1[C@H](Cn2nnc3ccccc3c2=O)CC[C@@H]1C(=O)c1ccc(OCc2cccc(Cl)c2)cc1]

To a solution of 80 mg (0.16 mmol) of the compound from Example 4A in 1.8 ml of acetonitrile were added 45 mg (0.32 mmol) of potassium carbonate and 41 mg (0.19 mmol, 97% purity) of 4-chlorobenzyl bromide. Then the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 8). 55 mg (55% of theory, 100% purity) of the title compound were obtained.

LC/MS (Method 1, ESIpos): Rₜ=1.55 min, m/z=618 [M+H]⁺.

### Example 7A

### 2-(Trimethylsilyl)ethyl (1RS,2RS,5SR)-2-{4-[(3,4-dichlorobenzyl)oxy]benzoyl}-5-[(4-oxo-1,2,3-benzotriazin-3(4H)-yl)methyl]cyclopentanecarboxylate (racemate)

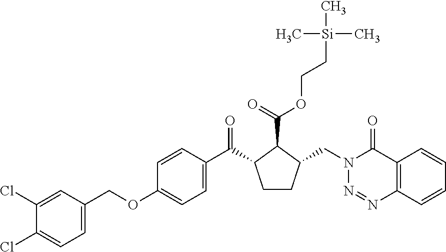 [SMILES: C[Si](C)(C)CCOC(=O)[C@H]1[C@H](Cn2nnc3ccccc3c2=O)CC[C@@H]1C(=O)c1ccc(OCc2ccc(Cl)c(Cl)c2)cc1]

To a solution of 80 mg (0.16 mmol) of the compound from Example 4A in 1.8 ml of acetonitrile were added 45 mg (0.32 mmol) of potassium carbonate and 48 mg (0.19 mmol, 98% purity) of 4-(bromomethyl)-1,2-dichlorobenzene. Then the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 9). 59 mg (55% of theory, 100% purity) of the title compound were obtained.

LC/MS (Method 1, ESIpos): Rₜ=1.59 min, m/z=652 [M+H]⁺.

### Example 8A

### 2-(Trimethylsilyl)ethyl (1RS,2RS,5SR)-2-[4-(cyclopentylmethoxy)benzoyl]-5-[(4-oxo-1,2,3-benzotriazin-3(4H)-yl)methyl]cyclopentanecarboxylate (racemate)

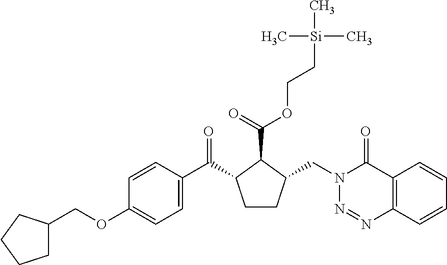 [SMILES: C[Si](C)(C)CCOC(=O)[C@H]1[C@H](Cn2nnc3ccccc3c2=O)CC[C@@H]1C(=O)c1ccc(OCC2CCCC2)cc1]

To a solution of 80 mg (0.16 mmol) of the compound from Example 4A in 1.8 ml of acetonitrile were added 45 mg (0.32 mmol) of potassium carbonate and 32 mg (0.19 mmol) of (bromomethyl)cyclopentane, and the mixture was stirred under reflux overnight. Thereafter, a further 32 mg (0.19 mmol) of (bromomethyl)cyclopentane were added, and the mixture was stirred under reflux once again for 4 h. After cooling to RT, the mixture was diluted with water and extracted twice with dichloromethane. The combined organic phases were dried over magnesium sulphate and concentrated. The residue was purified by means of preparative HPLC (Method 7). 33 mg (36% of theory, 100% purity) of the title compound were obtained.

LC/MS (Method 1, ESIpos): Rₜ=1.61 min, m/z=576 [M+H]⁺.

### Example 9A

### 2-(Trimethylsilyl)ethyl (1RS,2RS,5SR)-2-[4-(2-cyclohexylethoxy)benzoyl]-5-[(4-oxo-1,2,3-benzotriazin-3(4H)-yl)methyl]cyclopentanecarboxylate (racemate)

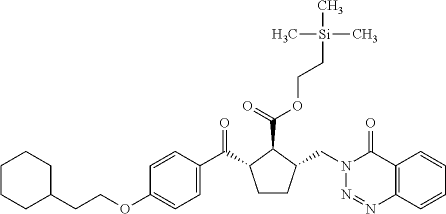 [SMILES: C[Si](C)(C)CCOC(=O)[C@H]1[C@H](Cn2nnc3ccccc3c2=O)CC[C@@H]1C(=O)c1ccc(OCCC2CCCCC2)cc1]

To a solution of 80 mg (0.16 mmol) of the compound from Example 4A in 1.8 ml of acetonitrile were added 45 mg (0.32 mmol) of potassium carbonate and 38 mg (0.19 mmol, 98% purity) of (2-bromoethyl)cyclohexane, and the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 7). 39 mg (39% of theory, 98% purity) of the title compound were obtained.

LC/MS (Method 1, ESIpos): Rₜ=1.68 min, m/z=604 [M+H]⁺.

### Example 10A

### 2-(Trimethylsilyl)ethyl (1RS,2RS,5SR)-2-[4-(hexyloxy)benzoyl]-5-[(4-oxo-1,2,3-benzotriazin-3(4H)-yl)methyl]cyclopentanecarboxylate (racemate)

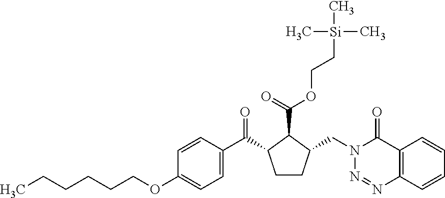 [SMILES: CCCCCCOc1ccc(C(=O)[C@H]2CC[C@@H](Cn3nnc4ccccc4c3=O)[C@@H]2C(=O)OCC[Si](C)(C)C)cc1]

To a solution of 80 mg (0.16 mmol) of the compound from Example 4A in 1.8 ml of acetonitrile were added 45 mg (0.32 mmol) of potassium carbonate and 33 mg (0.19 mmol, 98% purity) of 1-bromohexane, and the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 9). 41 mg (44% of theory, 100% purity) of the title compound were obtained.

LC/MS (Method 1, ESIpos): Rₜ=1.62 min, m/z=578 [M+H]⁺.

### Example 11A

### 2-(Trimethylsilyl)ethyl (1RS,2SR,5RS)-2-[(4-oxo-1,2,3-benzotriazin-3(4H)-yl)methyl]-5-[4-(pentyloxy)benzoyl]cyclopentanecarboxylate (racemate)

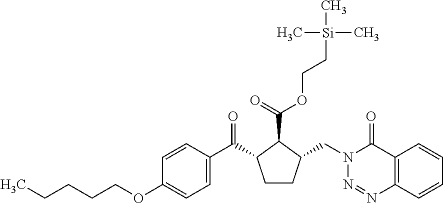 [SMILES: CCCCCOc1ccc(C(=O)[C@H]2CC[C@@H](Cn3nnc4ccccc4c3=O)[C@@H]2C(=O)OCC[Si](C)(C)C)cc1]

To a solution of 80 mg (0.16 mmol) of the compound from Example 4A in 1.8 ml of acetonitrile were added 45 mg (0.32 mmol) of potassium carbonate and 30 mg (0.19 mmol, 99% purity) of 1-bromopentane, and the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 10). 30 mg (33% of theory, 99% purity) of the title compound were obtained.

LC/MS (Method 1, ESIpos): Rₜ=1.59 min, m/z=564 [M+H]⁺.

### Example 12A

### 2-(Trimethylsilyl)ethyl (1RS,2RS,5SR)-2-(4-butoxybenzoyl)-5-[(4-oxo-1,2,3-benzotriazin-3(4H)-yl)methyl]cyclopentanecarboxylate (racemate)

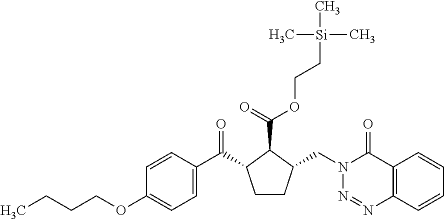 [SMILES: CCCCOc1ccc(C(=O)[C@H]2CC[C@@H](Cn3nnc4ccccc4c3=O)[C@@H]2C(=O)OCC[Si](C)(C)C)cc1]

To a solution of 80 mg (0.16 mmol) of the compound from Example 4A in 1.8 ml of acetonitrile were added 45 mg (0.32 mmol) of potassium carbonate and 27 mg (0.19 mmol) of 1-bromobutane, and the mixture was stirred under reflux overnight. After cooling to RT, the mixture was diluted with water and extracted twice with dichloromethane. The combined organic phases were dried over magnesium sulphate and concentrated. The residue was purified by means of preparative HPLC (Method 11). 33 mg (37% of theory, 100% purity) of the title compound were obtained.

LC/MS (Method 1, ESIpos): Rₜ=1.53 min, m/z=550 [M+H]⁺.

### Example 13A

### 2-(Trimethylsilyl)ethyl (1RS,2RS,5SR)-2-[4-(benzyloxy)benzoyl]-5-{[4-oxo-6-(trifluoromethyl)-1,2,3-benzotriazin-3(4H)-yl]methyl}cyclopentanecarboxylate (racemate)

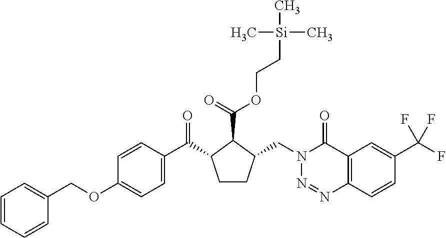 [SMILES: C[Si](C)(C)CCOC(=O)[C@H]1[C@H](Cn2nnc3ccc(C(F)(F)F)cc3c2=O)CC[C@@H]1C(=O)c1ccc(OCc2ccccc2)cc1]

To a solution of 13.88 g (30.53 mmol, not corrected for purity) of the compound from Example 3A Step 5 in 200 ml of toluene under argon were added 7.88 g (36.64 mmol) of the compound from Example 1A and 9.88 g (48.85 mmol) of tributylphosphine. Subsequently, 13.90 ml (30.53 mmol) of a 40% solution of diethyl azodicarboxylate in toluene was added dropwise at 0° C. The mixture was stirred at RT for 1 day and then concentrated. The residue was purified by means of flash chromatography (1 kg of silica gel, eluent: cyclohexane/ethyl acetate 9:1). 9.06 g (44% of theory, 98% purity) of the title compound were obtained.

¹H-NMR (400 MHz, DMSO-d₆): δ [ppm]=8.52 (s, 1H), 8.47-8.43 (m, 2H), 7.95 (d, 2H), 7.48-7.30 (m, 5H), 7.12 (d, 2H), 5.20 (s, 2H), 4.60-4.50 (m, 2H), 4.10 (q, 1H), 3.65-3.49 (m, 2H), 3.25 (t, 1H), 2.96-2.83 (m, 1H), 2.19-2.07 (m, 1H), 2.07-1.95 (m, 1H), 1.80-1.68 (m, 1H), 1.63-1.50 (m, 1H), 0.39-0.22 (m, 2H), −0.18 (s, 9H).

LC/MS (Method 1, ESIpos): Rₜ=1.57 min, m/z=652 [M+H]⁺.

### Example 14A

### 2-(Trimethylsilyl)ethyl (1RS,2RS,5SR)-2-(4-hydroxybenzoyl)-5-{[4-oxo-6-(trifluoromethyl)-1,2,3-benzotriazin-3(4H)-yl]methyl}cyclopentanecarboxylate (racemate)

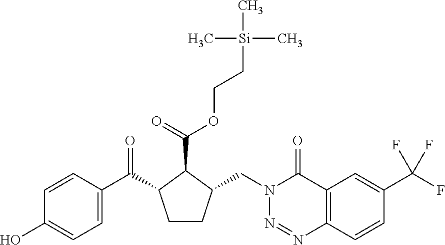 [SMILES: C[Si](C)(C)CCOC(=O)[C@H]1[C@H](Cn2nnc3ccc(C(F)(F)F)cc3c2=O)CC[C@@H]1C(=O)c1ccc(O)cc1]

To a solution of 9.05 g (13.89 mmol) of the compound from Example 13A in a mixture of 100 ml of ethyl acetate and 100 ml of ethanol under argon were added 1.05 g (16.66 mmol) of ammonium formate and 369 mg (0.35 mmol) of palladium on activated carbon (10% Pd). The mixture was then stirred at 75° C. for 1 h. Thereafter, a further 105 mg (1.67 mmol) of ammonium formate were added, and the mixture was stirred once again at 75° C. for 30 min. After cooling to RT, the mixture was filtered through kieselguhr, the filter residue was washed with ethyl acetate and ethanol, and the filtrate was concentrated. 7.86 g (100% of theory) of the title compound were obtained.

¹H-NMR (400 MHz, DMSO-d₆): δ [ppm]=10.40 (br. s, 1H), 8.52 (s, 1H), 8.47-8.42 (m, 2H), 7.85 (d, 2H), 6.84 (d, 2H), 4.60-4.51 (m, 2H), 4.05 (q, 1H), 3.64-3.48 (m, 2H), 3.23 (t, 1H), 2.96-2.82 (m, 1H), 2.17-2.05 (m, 1H), 2.05-1.94 (m, 1H), 1.79-1.67 (m, 1H), 1.62-1.49 (m, 1H), 0.38-0.22 (m, 2H), −0.18 (s, 9H).

LC/MS (Method 1, ESIpos): Rₜ=1.31 min, m/z=562 [M+H]⁺.

### Example 15A

### 2-(Trimethylsilyl)ethyl (1RS,2RS,5SR)-2-{4-[(4-fluorobenzyl)oxy]benzoyl}-5-{[4-oxo-6-(trifluoromethyl)-1,2,3-benzotriazin-3(4H)-yl]methyl}cyclopentanecarboxylate (racemate)

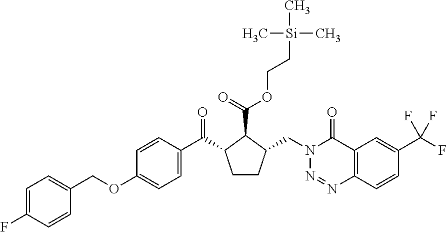 [SMILES: C[Si](C)(C)CCOC(=O)[C@H]1[C@H](Cn2nnc3ccc(C(F)(F)F)cc3c2=O)CC[C@@H]1C(=O)c1ccc(OCc2ccc(F)cc2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 93 mg (0.64 mmol) of 4-fluorobenzyl chloride were added, and the mixture was stirred at bath temperature 100° C. for 1.5 h. After cooling to RT, 60 ml of water and 60 ml of ethyl acetate were added to the mixture. After separating the phases, the aqueous phase was extracted once with 60 ml of ethyl acetate. The combined organic phases were dried over sodium sulphate, filtered and concentrated. The residue was taken up in dichloromethane and purified by column chromatography (25 g of silica gel, eluent: cyclohexane/ethyl acetate 85:15). 192 mg (54% of theory, 100% purity) of the title compound were obtained.

¹H-NMR (400 MHz, DMSO-d₆): δ [ppm]=8.52 (s, 1H), 8.45 (s, 2H), 7.96 (d, 2H), 7.54-7.48 (m, 2H), 7.27-7.19 (m, 2H), 7.12 (d, 2H), 5.18 (s, 2H), 4.58-4.53 (m, 2H), 4.10 (q, 1H), 3.64-3.49 (m, 2H), 3.25 (t, 1H), 2.94-2.86 (m, 1H), 2.18-2.07 (m, 1H), 2.06-1.97 (m, 1H), 1.79-1.68 (m, 1H), 1.63-1.50 (m, 1H), 0.38-0.23 (m, 2H), −0.18 (s, 9H).

LC/MS (Method 1, ESIpos): Rₜ=1.56 min, m/z=670 [M+H]⁺.

### Example 16A

### 2-(Trimethylsilyl)ethyl (1RS,2RS,5SR)-2-{4-[(3-methylbenzyl)oxy]benzoyl}-5-{[4-oxo-6-(trifluoromethyl)-1,2,3-benzotriazin-3(4H)-yl]methyl}cyclopentanecarboxylate (racemate)

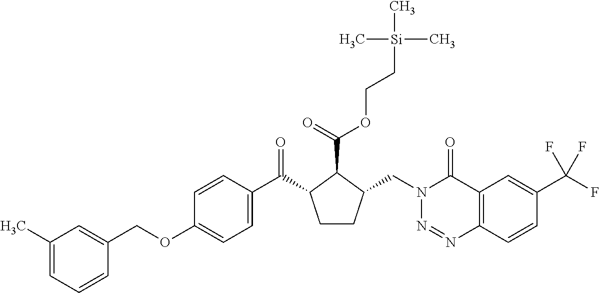 [SMILES: Cc1cccc(COc2ccc(C(=O)[C@H]3CC[C@@H](Cn4nnc5ccc(C(F)(F)F)cc5c4=O)[C@@H]3C(=O)OCC[Si](C)(C)C)cc2)c1]

To a solution of 300 mg (0.53 mmol) of the compound from Example 14A in 3 ml of DMF under argon were added 72 mg (0.64 mmol) of potassium tert-butoxide. After stirring at RT for 5 min, 118 mg (0.64 mmol) of 3-methylbenzyl bromide were added, and the mixture was stirred at bath temperature 100° C. for 10 min. After cooling to RT, 60 ml of water and 60 ml of ethyl acetate were added to the mixture. After separating the phases, the aqueous phase was extracted once with 30 ml of ethyl acetate. The combined organic phases were dried over sodium sulphate, filtered and concentrated. The residue was purified by means of column chromatography (40 g of silica gel, eluent: cyclohexane/ethyl acetate 9:1). 187 mg (53% of theory, 100% purity) of the title compound were obtained.

¹H-NMR (400 MHz, DMSO-d₆): δ [ppm]=8.52 (s, 1H), 8.45 (s, 2H), 7.95 (d, 2H), 7.26 (t, 3H), 7.18-7.08 (m, 3H), 5.16 (s, 2H), 4.59-4.52 (m, 2H), 4.10 (q, 1H), 3.65-3.50 (m, 2H), 3.25 (t, 1H), 2.94-2.86 (m, 1H), 2.32 (s, 3H), 2.18-2.07 (m, 1H), 2.06-1.95 (m, 1H), 1.79-1.68 (m, 1H), 1.62-1.50 (m, 1H), 0.38-0.23 (m, 2H), −0.18 (s, 9H).

LC/MS (Method 1, ESIpos): Rₜ=1.60 min, m/z=666 [M+H]⁺.

### Example 17A

### 2-(Trimethylsilyl)ethyl (1RS,2SR,5RS)-2-{[4-oxo-6-(trifluoromethyl)-1,2,3-benzotriazin-3(4H)-yl]methyl}-5-(4-{[3-(trifluoromethyl)benzyl]oxy}benzoyl)cyclopentanecarboxylate (racemate)

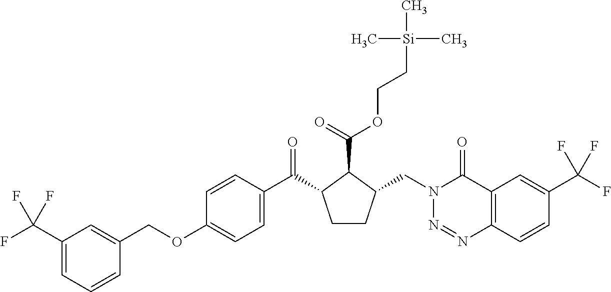 [SMILES: C[Si](C)(C)CCOC(=O)[C@H]1[C@H](Cn2nnc3ccc(C(F)(F)F)cc3c2=O)CC[C@@H]1C(=O)c1ccc(OCc2cccc(C(F)(F)F)c2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 153 mg (0.64 mmol) of 3-(trifluoromethyl)benzyl bromide were added, and the mixture was stirred at bath temperature 100° C. for 10 min. After cooling to RT, 60 ml of water and 60 ml of ethyl acetate were added to the mixture. After separating the phases, the aqueous phase was extracted once with 30 ml of ethyl acetate. The combined organic phases were dried over sodium sulphate, filtered and concentrated. The residue was purified by means of column chromatography (40 g of silica gel, eluent: cyclohexane/ethyl acetate 9:1). 184 mg of the title compound were obtained (48% of theory, still containing solvent and somewhat contaminated according to ¹H-NMR).

¹H-NMR (400 MHz, DMSO-d₆): δ [ppm]=8.52 (s, 1H), 8.45 (s, 2H), 7.97 (d, 2H), 7.83 (s, 1H), 7.78 (d, 1H), 7.74-7.70 (m, 1H), 7.68-7.62 (m, 1H), 7.15 (d, 2H), 5.32 (s, 2H), 4.59-4.52 (m, 2H), 4.14-4.07 (m, 1H), 3.64-3.50 (m, 2H), 3.24 (t, 1H), 2.95-2.86 (m, 1H), 2.18-2.07 (m, 1H), 2.06-1.96 (m, 1H), 1.79-1.68 (m, 1H), 1.68-1.60 (m, 1H), 0.36-0.25 (m, 2H), −0.19 (s, 9H).

LC/MS (Method 2, ESIpos): Rₜ=3.45 min, m/z=720 [M+H]⁺.

### Example 18A

### 2-(Trimethylsilyl)ethyl (1RS,2RS,5SR)-2-{4-[(3-chlorobenzyl)oxy]benzoyl}-5-{[4-oxo-6-(trifluoromethyl)-1,2,3-benzotriazin-3(4H)-yl]methyl}cyclopentanecarboxylate (racemate)

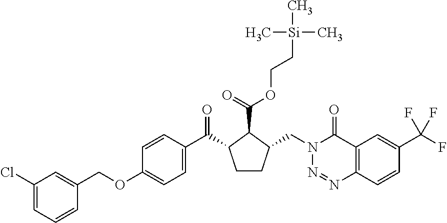 [SMILES: C[Si](C)(C)CCOC(=O)[C@H]1[C@H](Cn2nnc3ccc(C(F)(F)F)cc3c2=O)CC[C@@H]1C(=O)c1ccc(OCc2cccc(Cl)c2)cc1]

To a solution of 250 mg (0.45 mmol) of the compound from Example 14A in 2.5 ml of DMF under argon were added 60 mg (0.53 mmol) of potassium tert-butoxide. After stirring at RT for 5 min, 110 mg (0.53 mmol) of 3-chlorobenzyl bromide were added, and the mixture was stirred at bath temperature 100° C. for 15 min. After cooling to RT, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over sodium sulphate, filtered and concentrated. The residue was taken up in dichloromethane and purified by column chromatography (25 g of silica gel, eluent: cyclohexane/ethyl acetate 9:1). 113 mg of the title compound were obtained (37% of theory, still containing solvent and somewhat contaminated according to ¹H-NMR).

¹H-NMR (400 MHz, DMSO-d₆): δ [ppm]=8.52 (s, 1H), 8.45 (s, 2H), 7.96 (d, 2H), 7.55-7.52 (m, 1H), 7.45-7.40 (m, 3H), 7.13 (d, 2H), 5.23 (s, 2H), 4.59-4.53 (m, 2H), 4.14-4.06 (m, 1H), 3.63-3.51 (m, 2H), 3.28-3.21 (m, 1H), 2.95-2.86 (m, 1H), 2.18-2.07 (m, 1H), 2.07-1.95 (m, 1H), 1.78-1.69 (m, 1H), 1.69-1.60 (m, 1H), 0.36-0.25 (m, 2H), −0.18 (s, 9H).

LC/MS (Method 1, ESIpos): Rₜ=1.58 min, m/z=686 [M+H]⁺.

### Example 19A

### 2-(Trimethylsilyl)ethyl (1RS,2RS,5SR)-2-{4-[(4-chlorobenzyl)oxy]benzoyl}-5-{[4-oxo-6-(trifluoromethyl)-1,2,3-benzotriazin-3 (4H)-yl]methyl}cyclopentanecarboxylate (racemate)

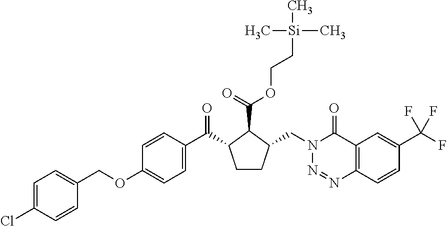 [SMILES: C[Si](C)(C)CCOC(=O)[C@H]1[C@H](Cn2nnc3ccc(C(F)(F)F)cc3c2=O)CC[C@@H]1C(=O)c1ccc(OCc2ccc(Cl)cc2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 153 mg (0.64 mmol) of 4-chlorobenzyl bromide were added, and the mixture was stirred at bath temperature 100° C. for 1.5 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (25 g of silica gel, eluent: cyclohexane/ethyl acetate 85:15). 112 mg (31% of theory, 100% purity) of the title compound were obtained.

¹H-NMR (400 MHz, DMSO-d₆): δ [ppm]=8.52 (s, 1H), 8.45 (s, 2H), 7.96 (d, 2H), 7.51-7.44 (m, 4H), 7.12 (d, 2H), 5.21 (s, 2H), 4.59-4.52 (m, 2H), 4.10 (q, 1H), 3.63-3.50 (m, 2H), 3.25 (t, 1H), 2.96-2.85 (m, 1H), 2.18-2.07 (m, 1H), 2.06-1.95 (m, 1H), 1.79-1.68 (m, 1H), 1.62-1.51 (m, 1H), 0.36-0.25 (m, 2H), −0.18 (s, 9H).

LC/MS (Method 1, ESIpos): Rₜ=1.60 min, m/z=686 [M+H]⁺.

### Example 20A

### 2-(Trimethylsilyl)ethyl (1RS,2RS,5SR)-2-{4-[(3,4-dichlorobenzyl)oxy]benzoyl}-5-{[4-oxo-6-(trifluoromethyl)-1,2,3-benzotriazin-3(4H)-yl]methyl}cyclopentanecarboxylate (racemate)

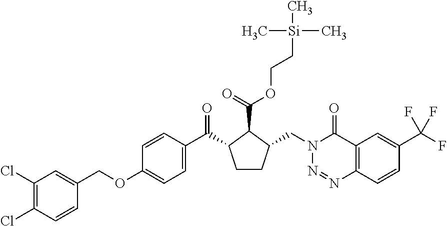 [SMILES: C[Si](C)(C)CCOC(=O)[C@H]1[C@H](Cn2nnc3ccc(C(F)(F)F)cc3c2=O)CC[C@@H]1C(=O)c1ccc(OCc2ccc(Cl)c(Cl)c2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 154 mg (0.64 mmol) of 3,4-dichlorobenzyl bromide were added, and the mixture was stirred at bath temperature 100° C. for 15 min. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was taken up in dichloromethane and purified by column chromatography (25 g of silica gel, eluent: cyclohexane/ethyl acetate 85:15). 222 mg (49% of theory, 85% purity) of the title compound were obtained.

¹H-NMR (400 MHz, DMSO-d₆): δ [ppm]=8.52 (s, 1H), 8.45 (s, 2H), 7.96 (d, 2H), 7.74 (d, 1H), 7.67 (d, 1H), 7.46 (dd, 1H), 7.13 (d, 2H), 5.23 (s, 2H), 4.59-4.53 (m, 2H), 4.10 (q, 1H), 3.64-3.50 (m, 2H), 3.25 (t, 1H), 2.96-2.85 (m, 1H), 2.18-2.07 (m, 1H), 2.07-1.95 (m, 1H), 1.79-1.68 (m, 1H), 1.64-1.50 (m, 1H), 0.38-0.23 (m, 2H), −0.19 (s, 9H).

LC/MS (Method 1, ESIpos): Rₜ=1.64 min, m/z=720 [M+H]⁺.

### Example 21A

### 2-(Trimethylsilyl)ethyl (1RS,2RS,5SR)-2-[4-({3-[(methoxycarbonyl)amino]benzyl}oxy)benzoyl]-5-{[4-oxo-6-(trifluoromethyl)-1,2,3-benzotriazin-3(4H)-yl]methyl}cyclopentanecarboxylate (racemate)

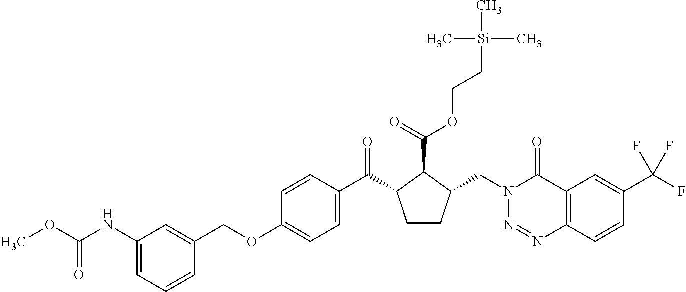 [SMILES: COC(=O)Nc1cccc(COc2ccc(C(=O)[C@H]3CC[C@@H](Cn4nnc5ccc(C(F)(F)F)cc5c4=O)[C@@H]3C(=O)OCC[Si](C)(C)C)cc2)c1]

To a solution of 300 mg (0.53 mmol) of the compound from Example 14A in 3 ml of DMF under argon were added 72 mg (0.64 mmol) of potassium tert-butoxide. After stirring at RT for 5 min, 156 mg (0.64 mmol) of methyl [3-(bromomethyl)phenyl]carbamate were added, and the mixture was stirred at bath temperature 100° C. for 10 min After cooling to RT, 60 ml of water and 60 ml of ethyl acetate were added to the mixture. After separating the phases, the aqueous phase was extracted once with 30 ml of ethyl acetate. The combined organic phases were dried over sodium sulphate, filtered and concentrated. The residue was taken up in dichloromethane and purified by column chromatography (25 g of silica gel, eluent: cyclohexane/ethyl acetate 7:3). 189 mg (43% of theory, 87% purity) of the title compound were obtained.

¹H-NMR (400 MHz, DMSO-d₆): δ [ppm]=9.70 (s, 1H), 8.52 (s, 1H), 8.45 (s, 2H), 7.95 (d, 2H), 7.57 (s, 1H), 7.41 (d, 1H), 7.29 (t, 1H), 7.13-7.04 (m, 3H), 5.16 (s, 2H), 4.59-4.52 (m, 2H), 4.14-4.06 (m, 1H), 3.65 (s, 3H), 3.62-3.52 (m, 2H), 3.25 (t, 1H), 2.95-2.85 (m, 1H), 2.17-2.06 (m, 1H), 2.05-1.94 (m, 1H), 1.79-1.67 (m, 1H), 1.62-1.51 (m, 1H), 0.37-0.25 (m, 2H), −0.18 (s, 9H).

LC/MS (Method 1, ESIpos): Rₜ=1.48 min, m/z=725 [M+H]⁺.

### Example 22A

### 2-(Trimethylsilyl)ethyl (1RS,2RS,5SR)-2-(4-{[3-(2-oxo-1,3-oxazolidin-3-yl)benzyl]oxy}benzoyl)-5-{[4-oxo-6-(trifluoromethyl)-1,2,3-benzotriazin-3(4H)-yl]methyl}cyclopentanecarboxylate (racemate)

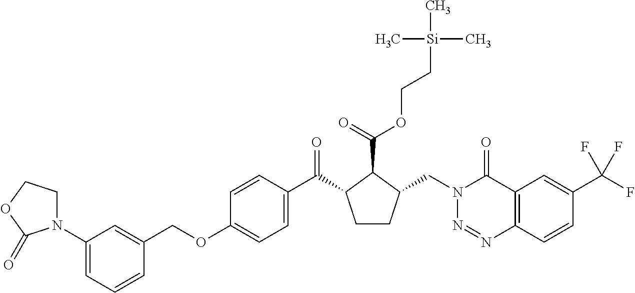 [SMILES: C[Si](C)(C)CCOC(=O)[C@H]1[C@H](Cn2nnc3ccc(C(F)(F)F)cc3c2=O)CC[C@@H]1C(=O)c1ccc(OCc2cccc(N3CCOC3=O)c2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 164 mg (0.64 mmol) of 3-[3-(bromomethyl)phenyl]-1,3-oxazolidin-2-one were added, and the mixture was stirred at bath temperature 100° C. for 20 min. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (40 g of silica gel, eluent: cyclohexane/ethyl acetate 6:4). 156 mg (39% of theory, 97% purity) of the title compound were obtained.

¹H-NMR (400 MHz, DMSO-d₆): δ [ppm]=8.52 (s, 1H), 8.45 (s, 2H), 7.96 (d, 2H), 7.70 (s, 1H), 7.52 (dd, 1H), 7.41 (t, 1H), 7.22 (d, 1H), 7.12 (d, 2H), 5.22 (s, 2H), 4.61-4.52 (m, 2H), 4.47-4.39 (m, 2H), 4.16-4.01 (m, 3H), 3.66-3.49 (m, 2H), 3.25 (t, 1H), 2.99-2.82 (m, 1H), 2.19-2.07 (m, 1H), 2.06-1.94 (m, 1H), 1.80-1.68 (m, 1H), 1.62-1.50 (m, 1H), 0.39-0.22 (m, 2H), −0.18 (s, 9H).

LC/MS (Method 1, ESIpos): Rₜ=1.41 min, m/z=737 [M+H]⁺.

### Example 23A

### 2-(Trimethylsilyl)ethyl (1RS,2SR,5RS)-2-{[4-oxo-6-(trifluoromethyl)-1,2,3-benzotriazin-3(4H)-yl]methyl}-5-[4-(2-phenylethoxy)benzoyl]cyclopentanecarboxylate (racemate)

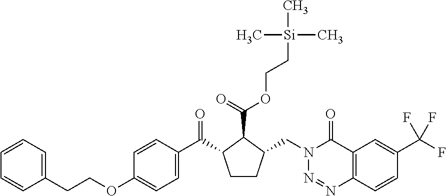 [SMILES: C[Si](C)(C)CCOC(=O)[C@H]1[C@H](Cn2nnc3ccc(C(F)(F)F)cc3c2=O)CC[C@@H]1C(=O)c1ccc(OCCc2ccccc2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 177 mg (0.64 mmol) of 2-phenylethyl 4-methylbenzenesulphonate were added, and the mixture was stirred at bath temperature 100° C. for 1.5 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (25 g of silica gel, eluent: cyclohexane/ethyl acetate 9:1). 240 mg (65% of theory, 96% purity) of the title compound were obtained.

¹H-NMR (400 MHz, DMSO-d₆): δ [ppm]=8.52 (s, 1H), 8.45 (s, 2H), 7.93 (d, 2H), 7.35-7.27 (m, 4H), 7.26-7.16 (m, 1H), 7.04 (d, 2H), 4.59-4.50 (m, 2H), 4.28 (t, 2H), 4.09 (q, 1H), 3.65-3.47 (m, 2H), 3.24 (t, 1H), 3.05 (t, 2H), 2.97-2.83 (m, 1H), 2.18-2.06 (m, 1H), 2.06-1.94 (m, 1H), 1.79-1.68 (m, 1H), 1.61-1.50 (m, 1H), 0.38-0.21 (m, 2H), −0.19 (s, 9H).

LC/MS (Method 2, ESIpos): Rₜ=3.43 min, m/z=666 [M+H]⁺.

### Example 24A

### 2-(Trimethylsilyl)ethyl (1RS,2SR,5RS)-2-{[4-oxo-6-(trifluoromethyl)-1,2,3-benzotriazin-3(4H)-yl]methyl}-5-[4-(3-phenylpropoxy)benzoyl]cyclopentanecarboxylate (racemate)

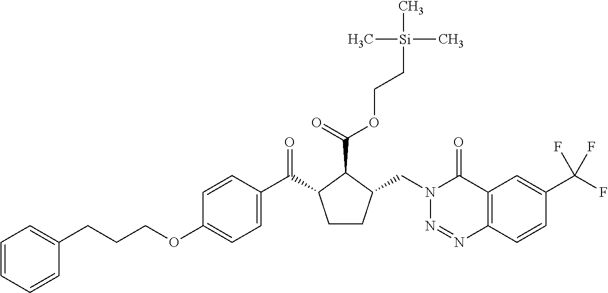 [SMILES: C[Si](C)(C)CCOC(=O)[C@H]1[C@H](Cn2nnc3ccc(C(F)(F)F)cc3c2=O)CC[C@@H]1C(=O)c1ccc(OCCCc2ccccc2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 319 mg (1.60 mmol) of (3-bromopropyl)benzene were added, and the mixture was stirred at bath temperature 100° C. for 2 h. After cooling to RT, 30 ml of water and 30 ml of ethyl acetate were added to the mixture. After separating the phases, the aqueous phase was extracted twice with 30 ml each time of ethyl acetate. The combined organic phases were dried over magnesium sulphate, filtered and concentrated. The residue was purified by means of column chromatography (40 g of silica gel, eluent: cyclohexane/ethyl acetate 9:1). 37 mg (10% of theory, 98% purity) of the title compound were obtained.

¹H-NMR (400 MHz, DMSO-d₆): δ [ppm]=8.52 (s, 1H), 8.45 (s, 2H), 7.94 (d, 2H), 7.32-7.14 (m, 5H), 7.03 (d, 2H), 4.59-4.52 (m, 2H), 4.05 (t, 3H), 3.65-3.49 (m, 2H), 3.24 (t, 1H), 2.97-2.85 (m, 1H), 2.74 (t, 2H), 2.18-1.95 (m, 4H), 1.80-1.69 (m, 1H), 1.63-1.51 (m, 1H), 0.38-0.21 (m, 2H), −0.19 (s, 9H).

LC/MS (Method 1, ESIpos): Rₜ=1.33 min, m/z=580 [M+H]⁺.

### Example 25A

### 2-(Trimethylsilyl)ethyl (1RS,2RS,5SR)-2-[4-(cyclohexylmethoxy)benzoyl]-5-{[4-oxo-6-(trifluoromethyl)-1,2,3-benzotriazin-3(4H)-yl]methyl}cyclopentanecarboxylate (racemate)

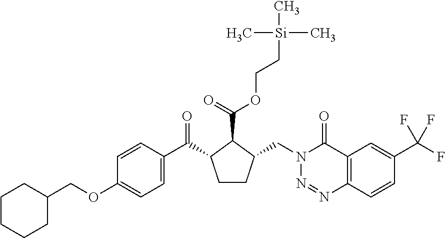 [SMILES: C[Si](C)(C)CCOC(=O)[C@H]1[C@H](Cn2nnc3ccc(C(F)(F)F)cc3c2=O)CC[C@@H]1C(=O)c1ccc(OCC2CCCCC2)cc1]

To a solution of 250 mg (0.45 mmol) of the compound from Example 14A in 4.5 ml of DMF under argon were added 60 mg (0.53 mmol) of potassium tert-butoxide. After stirring at RT for 5 min, 95 mg (0.53 mmol) of (bromomethyl)cyclohexane were added, and the mixture was stirred at bath temperature 100° C. for 1 h. Thereafter, a further 95 mg (0.53 mmol) of (bromomethyl)cyclohexane were added, and the mixture was stirred once again at bath temperature 100° C. for 90 min. After cooling to RT, 60 ml of water and 60 ml of tert-butyl methyl ether were added to the mixture. After separating the phases, the aqueous phase was extracted once with 30 ml of tert-butyl methyl ether and twice with 50 ml each time of ethyl acetate. The combined organic phases were dried over sodium sulphate, filtered and concentrated. The residue was taken up in dichloromethane and purified by column chromatography (25 g of silica gel, eluent: cyclohexane/ethyl acetate 9:1). 174 mg (59% of theory, 100% purity) of the title compound were obtained.

¹H-NMR (400 MHz, DMSO-d₆): δ [ppm]=8.52 (s, 1H), 8.45 (s, 2H), 7.92 (d, 2H), 7.02 (d, 2H), 4.61-4.50 (m, 2H), 4.09 (q, 1H), 3.87 (d, 2H), 3.64-3.47 (m, 2H), 3.22 (t, 1H), 2.97-2.84 (m, 1H), 2.18-1.96 (m, 2H), 1.85-1.61 (m, 8H), 1.33-1.13 (m, 3H), 1.11-0.95 (m, 2H), 0.35-0.23 (m, 2H), −0.19 (s, 9H).

LC/MS (Method 1, ESIpos): Rₜ=1.71 min, m/z=558 [M+H]⁺.

### Example 26A

### 2-(Trimethylsilyl)ethyl (1RS,2RS,5SR)-2-[4-(cyclopentylmethoxy)benzoyl]-5-{[4-oxo-6-(trifluoromethyl)-1,2,3-benzotriazin-3 (4H)-yl]methyl}cyclopentanecarboxylate (racemate)

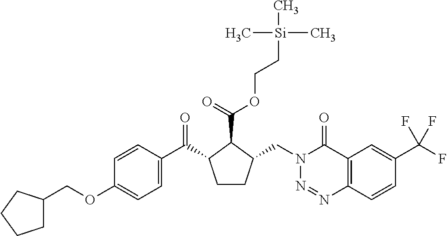 [SMILES: C[Si](C)(C)CCOC(=O)[C@H]1[C@H](Cn2nnc3ccc(C(F)(F)F)cc3c2=O)CC[C@@H]1C(=O)c1ccc(OCC2CCCC2)cc1]

To a solution of 300 mg (0.53 mmol) of the compound from Example 14A in 6 ml of acetonitrile under argon were added 180 mg (1.60 mmol) of potassium carbonate and 261 mg (1.60 mmol) of cyclopentylmethyl bromide, and the mixture was stirred under reflux for 6 h. Subsequently, a further 261 mg (1.60 mmol) of cyclopentylmethyl bromide were added, and stirring of the mixture was continued at RT overnight. Thereafter, 30 ml of water and 30 ml of ethyl acetate were added to the mixture. After separating the phases, the aqueous phase was extracted once with 30 ml of ethyl acetate. The combined organic phases were dried over magnesium sulphate, filtered and concentrated. The residue was purified by means of column chromatography (25 g of silica gel, eluent: cyclohexane/ethyl acetate 9:1). 217 mg (62% of theory, 98% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.60-4.50 (m, 2H), 4.09 (q, 1H), 3.93 (d, 2H), 3.63-3.48 (m, 2H), 3.22 (t, 1H), 2.97-2.84 (m, 1H), 2.38-2.24 (m, 1H), 2.19-1.95 (m, 2H), 1.82-1.69 (m, 3H), 1.67-1.47 (m, 5H), 1.38-1.26 (m, 2H), 0.37-0.21 (m, 2H), −0.18 (s, 9H).

LC/MS (Method 2, ESIpos): Rₜ=3.57 min, m/z=644 [M+H]⁺.

### Example 27A

### 2-(Trimethylsilyl)ethyl (1RS,2RS,5SR)-2-[4-(cyclobutylmethoxy)benzoyl]-5-{[4-oxo-6-(trifluoromethyl)-1,2,3-benzotriazin-3(4H)-yl]methyl}cyclopentanecarboxylate (racemate)

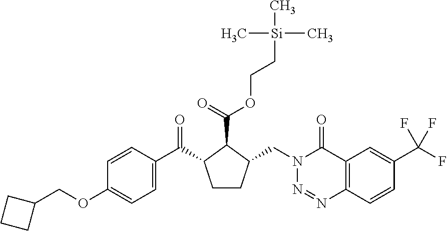 [SMILES: C[Si](C)(C)CCOC(=O)[C@H]1[C@H](Cn2nnc3ccc(C(F)(F)F)cc3c2=O)CC[C@@H]1C(=O)c1ccc(OCC2CCC2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 96 mg (0.64 mmol) of (bromomethyl)cyclobutane were added, and the mixture was stirred at bath temperature 100° C. for 1.5 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (25 g of silica gel, eluent: cyclohexane/ethyl acetate 9:1). 147 mg (41% of theory, 95% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.61-4.49 (m, 2H), 4.10 (q, 1H), 4.04 (d, 2H), 3.64-3.46 (m, 2H), 3.23 (t, 1H), 2.96-2.85 (m, 1H), 2.79-2.65 (m, 1H), 2.19-1.97 (m, 4H), 1.96-1.68 (m, 5H), 1.63-1.49 (m, 1H), 0.38-0.22 (m, 2H), −0.18 (s, 9H).

LC/MS (Method 2, ESIpos): Rₜ=3.50 min, m/z=630 [M+H]⁺.

### Example 28A

### 2-(Trimethylsilyl)ethyl (1RS,2RS,5SR)-2-[4-(cyclopropylmethoxy)benzoyl]-5-{[4-oxo-6-(trifluoromethyl)-1,2,3-benzotriazin-3(4H)-yl]methyl}cyclopentanecarboxylate (racemate)

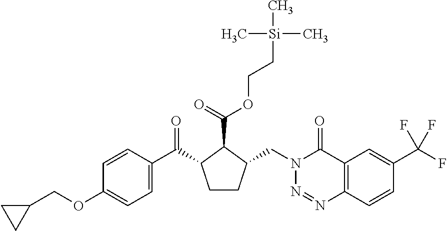 [SMILES: C[Si](C)(C)CCOC(=O)[C@H]1[C@H](Cn2nnc3ccc(C(F)(F)F)cc3c2=O)CC[C@@H]1C(=O)c1ccc(OCC2CC2)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 117 mg (0.64 mmol) of (iodomethyl)cyclopropane were added, and the mixture was stirred at bath temperature 100° C. for 1 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (40 g of silica gel, eluent: cyclohexane/ethyl acetate 85:15). 150 mg (46% of theory, 100% purity) of the title compound were obtained.

¹H-NMR (400 MHz, DMSO-d₆): δ [ppm]=8.52 (s, 1H), 8.45 (s, 2H), 7.93 (d, 2H), 7.02 (d, 2H), 4.61-4.49 (m, 2H), 4.09 (q, 1H), 3.91 (d, 2H), 3.66-3.47 (m, 2H), 3.23 (t, 1H), 2.97-2.83 (m, 1H), 2.19-2.07 (m, 1H), 2.06-1.96 (m, 1H), 1.80-1.68 (m, 1H), 1.62-1.49 (m, 1H), 1.29-1.18 (m, 1H), 0.62-0.54 (m, 2H), 0.37-0.24 (m, 4H), −0.19 (s, 9H).

LC/MS (Method 1, ESIpos): Rₜ=1.50 min, m/z=616 [M+H]⁺.

### Example 29A

### 2-(Trimethylsilyl)ethyl (1RS,2RS,5SR)-2-[4-(2-cyclohexylethoxy)benzoyl]-5-{[4-oxo-6-(trifluoromethyl)-1,2,3-benzotriazin-3(4H)-yl]methyl}cyclopentanecarboxylate (racemate)

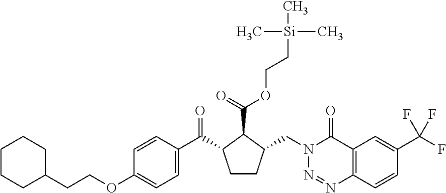 [SMILES: C[Si](C)(C)CCOC(=O)[C@H]1[C@H](Cn2nnc3ccc(C(F)(F)F)cc3c2=O)CC[C@@H]1C(=O)c1ccc(OCCC2CCCCC2)cc1]

To a solution of 293 mg (0.36 mmol, 70% purity) of the compound from Example 14A in 2 ml of DMF under argon were added 49 mg (0.43 mmol) of potassium tert-butoxide. After stirring at RT for 5 min, 83 mg (0.44 mmol) of (2-bromoethyl)cyclohexane were added, and the mixture was stirred at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was taken up in dichloromethane and purified by column chromatography (25 g of silica gel, eluent: cyclohexane/ethyl acetate 9:1). 124 mg (51% of theory, 100% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.64-4.48 (m, 2H), 4.14-3.98 (m, 3H), 3.65-3.45 (m, 2H), 3.22 (t, 1H), 2.97-2.84 (m, 1H), 2.18-2.06 (m, 1H), 2.05-1.94 (m, 1H), 1.83-1.52 (m, 8H), 1.50-1.38 (m, 1H), 1.30-1.08 (m, 4H), 1.02-0.87 (m, 2H), 0.36-0.20 (m, 2H), −0.19 (s, 9H).

LC/MS (Method 1, ESIpos): Rₜ=1.74 min, m/z=672 [M+H]⁺.

### Example 30A

### 2-(Trimethylsilyl)ethyl (1RS,2RS,5SR)-2-[4-(2-cyclopentylethoxy)benzoyl]-5-{[4-oxo-6-(trifluoromethyl)-1,2,3-benzotriazin-3 (4H)-yl]methyl}cyclopentanecarboxylate (racemate)

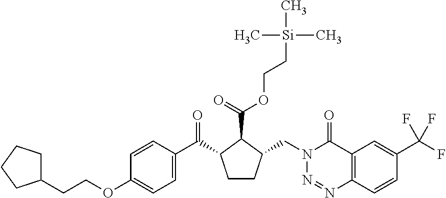 [SMILES: C[Si](C)(C)CCOC(=O)[C@H]1[C@H](Cn2nnc3ccc(C(F)(F)F)cc3c2=O)CC[C@@H]1C(=O)c1ccc(OCCC2CCCC2)cc1]

To a solution of 293 mg (0.36 mmol, 70% purity) of the compound from Example 14A in 2 ml of DMF under argon were added 49 mg (0.43 mmol) of potassium tert-butoxide. After stirring at RT for 5 min, 98 mg (0.44 mmol) of (2-iodoethyl)cyclopentane were added, and the mixture was stirred at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was taken up in dichloromethane and purified by column chromatography (25 g of silica gel, eluent: cyclohexane/ethyl acetate 9:1). 119 mg (50% of theory, 100% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.63-4.47 (m, 2H), 4.15-4.02 (m, 3H), 3.65-3.47 (m, 2H), 3.22 (t, 1H), 2.97-2.84 (m, 1H), 2.17-2.07 (m, 1H), 2.06-1.86 (m, 2H), 1.83-1.70 (m, 5H), 1.64-1.43 (m, 5H), 1.20-1.07 (m, 2H), 0.35-0.24 (m, 2H), −0.18 (s, 9H).

LC/MS (Method 1, ESIpos): Rₜ=1.69 min, m/z=658 [M+H]⁺.

### Example 31A

### 2-(Trimethylsilyl)ethyl (1RS,2RS,5SR)-2-[4-(3-cyclohexylpropoxy)benzoyl]-5-{[4-oxo-6-(trifluoromethyl)-1,2,3-benzotriazin-3 (4H)-yl]methyl}cyclopentanecarboxylate (racemate)

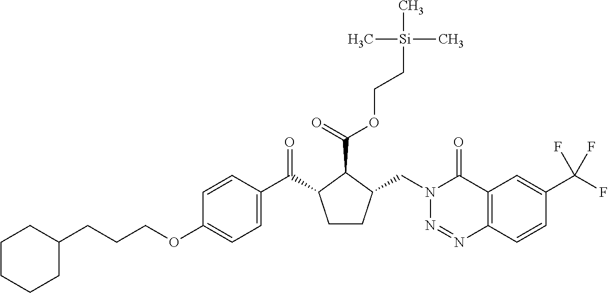 [SMILES: C[Si](C)(C)CCOC(=O)[C@H]1[C@H](Cn2nnc3ccc(C(F)(F)F)cc3c2=O)CC[C@@H]1C(=O)c1ccc(OCCCC2CCCCC2)cc1]

To a solution of 300 mg (0.53 mmol) of the compound from Example 14A in 2 ml of DMF under argon were added 180 mg (1.60 mmol) of potassium tert-butoxide. After stirring at RT for 5 min, 329 mg (1.60 mmol) of (3-bromopropyl)cyclohexane were added, and the mixture was stirred at bath temperature 100° C. for 1.5 h. After cooling to RT, 30 ml of water and 30 ml of ethyl acetate were added to the mixture. After separating the phases, the aqueous phase was extracted once with 30 ml of ethyl acetate. The combined organic phases were dried over magnesium sulphate, filtered and concentrated. The residue was purified by means of column chromatography (40 g of silica gel, eluent: cyclohexane/ethyl acetate 9:1). 84 mg (14% of theory, 63% purity) of the title compound were obtained.

LC/MS (Method 1, ESIpos): Rₜ=1.78 min, m/z=686 [M+H]⁺.

### Example 32A

### 2-(Trimethylsilyl)ethyl (1RS,2RS,5SR)-2-[4-(hexyloxy)benzoyl]-5-{[4-oxo-6-(trifluoromethyl)-1,2,3-benzotriazin-3(4H)-yl]methyl}cyclopentanecarboxylate (racemate)

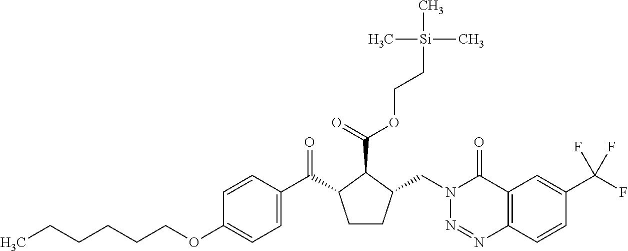 [SMILES: CCCCCCOc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 106 mg (0.64 mmol) of 1-bromohexane were added, and the mixture was stirred at bath temperature 100° C. for 1.5 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (25 g of silica gel, eluent: cyclohexane/ethyl acetate 85:15). 169 mg (49% of theory, 100% purity) of the title compound were obtained.

¹H-NMR (400 MHz, DMSO-d₆): δ [ppm]=8.52 (s, 1H), 8.45 (s, 2H), 7.93 (d, 2H), 7.02 (d, 2H), 4.62-4.50 (m, 2H), 4.15-4.00 (m, 3H), 3.65-3.45 (m, 2H), 3.22 (t, 1H), 2.97-2.83 (m, 1H), 2.18-2.06 (m, 1H), 2.06-1.95 (m, 1H), 1.82-1.67 (m, 3H), 1.63-1.50 (m, 1H), 1.46-1.36 (m, 2H), 1.35-1.25 (m, 4H), 0.91-0.84 (m, 3H), 0.37-0.22 (m, 2H), −0.19 (s, 9H).

LC/MS (Method 1, ESIpos): Rₜ=1.67 min, m/z=646 [M+H]⁺.

### Example 33A

### 2-(Trimethylsilyl)ethyl (1RS,2RS,5SR)-2-[4-(3-methylbutoxy)benzoyl]-5-{[4-oxo-6-(trifluoromethyl)-1,2,3-benzotriazin-3(4H)-yl]methyl}cyclopentanecarboxylate (racemate)

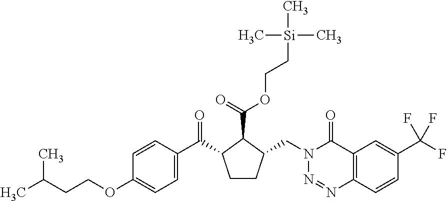 [SMILES: CC(C)CCOc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 97 mg (0.64 mmol) of 1-bromo-3-methylbutane were added, and the mixture was stirred at bath temperature 100° C. for 1 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was purified by means of column chromatography (40 g of silica gel, eluent: cyclohexane/ethyl acetate 8:2). 249 mg (74% of theory, 100% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.63-4.45 (m, 2H), 4.15-4.00 (m, 3H), 3.68-3.47 (m, 2H), 3.22 (t, 1H), 2.97-2.83 (m, 1H), 2.17-2.06 (m, 1H), 2.05-1.95 (m, 1H), 1.83-1.69 (m, 2H), 1.66-1.50 (m, 3H), 0.93 (s, 3H), 0.92 (s, 3H), 0.37-0.23 (m, 2H), −0.18 (s, 9H).

LC/MS (Method 1, ESIpos): Rₜ=1.61 min, m/z=632 [M+H]⁺.

### Example 34A

### 2-(Trimethylsilyl)ethyl (1RS,2SR,5RS)-2-{[4-oxo-6-(trifluoromethyl)-1,2,3-benzotriazin-3(4H)-yl]methyl}-5-[4-(pentyloxy)benzoyl]cyclopentanecarboxylate (racemate)

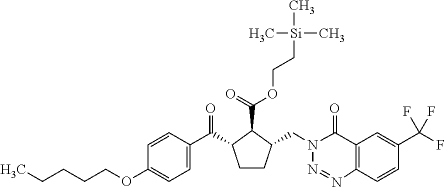 [SMILES: CCCCCOc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 97 mg (0.64 mmol) of 1-bromopentane were added, and the mixture was stirred at bath temperature 100° C. for 1.5 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was taken up in dichloromethane and purified by column chromatography (25 g of silica gel, eluent: cyclohexane/ethyl acetate 85:15). 181 mg (53% of theory, 99% purity) of the title compound were obtained.

¹H-NMR (400 MHz, DMSO-d₆): δ [ppm]=8.52 (s, 1H), 8.45 (s, 2H), 7.93 (d, 2H), 7.02 (d, 2H), 4.63-4.48 (m, 2H), 4.15-3.99 (m, 3H), 3.64-3.47 (m, 2H), 3.23 (t, 1H), 2.97-2.84 (m, 1H), 2.17-2.07 (m, 1H), 2.06-1.96 (m, 1H), 1.81-1.67 (m, 3H), 1.63-1.51 (m, 1H), 1.37 (d, 4H), 0.93-0.84 (m, 3H), 0.38-0.21 (m, 2H), −0.19 (s, 9H).

LC/MS (Method 1, ESIpos): Rₜ=1.64 min, m/z=632 [M+H]⁺.

### Example 35A

### 2-(Trimethylsilyl)ethyl (1RS,2RS,5SR)-2-(4-butoxybenzoyl)-5-{[4-oxo-6-(trifluoromethyl)-1,2,3-benzotriazin-3(4H)-yl]methyl}cyclopentanecarboxylate (racemate)

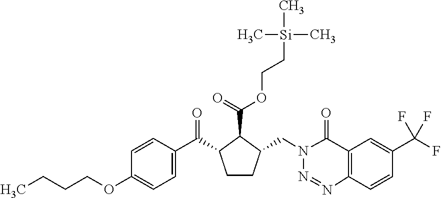 [SMILES: CCCCOc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 97 mg (0.64 mmol) of 1-bromobutane were added, and the mixture was stirred at bath temperature 100° C. for 1.5 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was taken up in dichloromethane and purified by column chromatography (25 g of silica gel, eluent: cyclohexane/ethyl acetate 9:1). 235 mg (68% of theory, 96% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.62-4.49 (m, 2H), 4.14-3.99 (m, 3H), 3.65-3.46 (m, 2H), 3.23 (t, 1H), 2.99-2.81 (m, 1H), 2.18-2.07 (m, 1H), 2.06-1.94 (m, 1H), 1.81-1.66 (m, 3H), 1.64-1.51 (m, 1H), 1.49-1.36 (m, 2H), 0.93 (t, 3H), 0.38-0.21 (m, 2H), −0.19 (s, 9H).

LC/MS (Method 2, ESIpos): Rₜ=3.47 min, m/z=618 [M+H]⁺.

### Example 36A

### 2-(Trimethylsilyl)ethyl (1RS,2SR,5RS)-2-{[4-oxo-6-(trifluoromethyl)-1,2,3-benzotriazin-3(4H)-yl]methyl}-5-(4-propoxybenzoyl)cyclopentanecarboxylate (racemate)

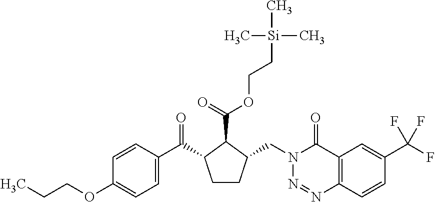 [SMILES: CCCOc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 103 mg (0.18 mmol) of the compound from Example 14A in 2.0 ml of acetonitrile were added 51 mg (0.37 mmol) of potassium carbonate and 28 mg (0.22 mmol, 98% purity) of 1-bromopropane. The mixture was stirred under reflux overnight. Thereafter, a further 8 mg (0.07 mmol) of 1-bromopropane were added and the mixture was stirred under reflux once again for 4 h. After cooling to RT, the mixture was diluted with water. The solids that precipitated out were filtered off and dried under vacuum. This crude product was then purified by means of preparative HPLC (Method 10). 69 mg (62% of theory, 100% purity) of the title compound were obtained.

LC/MS (Method 1, ESIpos): Rₜ=1.56 min, m/z=604 [M+H]⁺.

### Example 37A

### 2-(Trimethylsilyl)ethyl (1RS,2RS,5SR)-2-(4-isobutoxybenzoyl)-5-{[4-oxo-6-(trifluoromethyl)-1,2,3-benzotriazin-3(4H)-yl]methyl}cyclopentanecarboxylate (racemate)

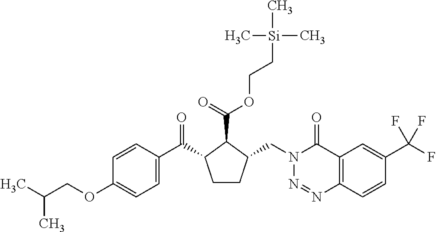 [SMILES: CC(C)COc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 300 mg (0.53 mmol) of the compound from Example 14A in 3 ml of DMF under argon were added 72 mg (0.64 mmol) of potassium tert-butoxide. After stirring at RT for 5 min, 88 mg (0.64 mmol) of 1-bromo-2-methylpropane were added, and the mixture was stirred at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with 80 ml of saturated sodium chloride solution, dried over sodium sulphate, filtered and concentrated. The residue was taken up in dichloromethane and purified by column chromatography (40 g of silica gel, eluent: cyclohexane/ethyl acetate 85:15). 92 mg (28% of theory, 100% purity) of the title compound were obtained.

¹H-NMR (400 MHz, DMSO-d₆): δ [ppm]=8.52 (s, 1H), 8.45 (s, 2H), 7.93 (d, 2H), 7.03 (d, 2H), 4.62-4.48 (m, 2H), 4.14-4.04 (m, 1H), 3.83 (d, 2H), 3.67-3.47 (m, 2H), 3.22 (t, 1H), 2.97-2.83 (m, 1H), 2.18-1.95 (m, 3H), 1.80-1.69 (m, 1H), 1.63-1.50 (m, 1H), 0.99 (s, 3H), 0.97 (s, 3H), 0.37-0.22 (m, 2H), −0.18 (s, 9H).

LC/MS (Method 1, ESIpos): Rₜ=1.57 min, m/z=618 [M+H]⁺.

### Example 38A

### 2-(Trimethylsilyl)ethyl (1RS,2SR,5RS)-2-{[4-oxo-6-(trifluoromethyl)-1,2,3-benzotriazin-3 (4H)-yl]methyl}-5-(4-{[(trifluoromethyl)sulphonyl]oxy}benzoyl)cyclopentanecarboxylate (racemate)

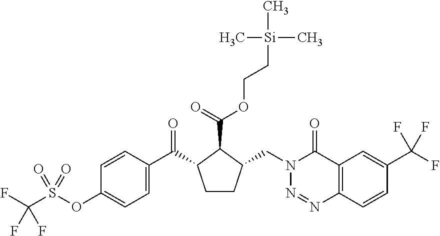 [SMILES: C[Si](C)(C)CCOC(=O)[C@H]1[C@H](Cn2nnc3ccc(C(F)(F)F)cc3c2=O)CC[C@@H]1C(=O)c1ccc(OS(=O)(=O)C(F)(F)F)cc1]

To a solution of 1.00 g (1.78 mmol) of the compound from Example 14A in 5.0 ml of dichloromethane under argon were added, at 0° C., first 0.25 ml (3.12 mmol) of pyridine and then, gradually, 0.45 ml (2.67 mmol) of trifluoromethanesulphonic anhydride. The mixture was stirred at 0° C. for 1 h, then dichloromethane was added and the mixture was washed once each with water and saturated sodium hydrogencarbonate solution. The organic phase was dried over magnesium sulphate, filtered and concentrated. 1.21 g (98% of theory, 100% purity) of the title compound were obtained.

LC/MS (Method 2, ESIpos): Rₜ=3.41 min, m/z=694 [M+H]⁺.

### Example 39A

### 2-(Trimethylsilyl)ethyl (1RS,2SR,5RS)-2-{[4-oxo-6-(trifluoromethyl)-1,2,3-benzotriazin-3(4H)-yl]methyl}-5-(4-sulphanylbenzoyl)cyclopentanecarboxylate (racemate)

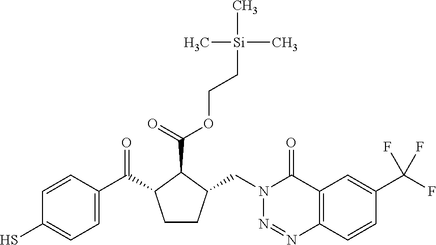 [SMILES: C[Si](C)(C)CCOC(=O)[C@H]1[C@H](Cn2nnc3ccc(C(F)(F)F)cc3c2=O)CC[C@@H]1C(=O)c1ccc(S)cc1]

To a solution of 800 mg (1.15 mmol) of the compound from Example 38A in 10 ml of dioxane were successively added 264 mg (1.38 mmol) of triisopropylsilanethiol, 298 mg (2.31 mmol) of N,N-diisopropylethylamine, 26 mg (0.03 mmol) of tris(dibenzylideneacetone)dipalladium and 33 mg (0.06 mmol) of 4,5-bis(diphenylphosphino)-9,9-dimethylxanthene (Xantphos). Subsequently, the mixture was degassed, purged with argon and stirred under reflux for 2.5 h. After cooling to RT, the mixture was admixed with ethyl acetate and washed once with water. After the aqueous phase had been extracted once with ethyl acetate, the combined organic phases were washed once with saturated sodium chloride solution, dried over magnesium sulphate, filtered and concentrated. The residue was purified by means of preparative HPLC (Method 4). The product-containing fractions were combined, neutralized with saturated aqueous sodium hydrogencarbonate solution and concentrated down to a small residual volume of water. After this aqueous phase had been extracted twice with dichloromethane, the combined organic phases were dried over magnesium sulphate, filtered and concentrated, and the residue was dried under reduced pressure. 350 mg (35% of theory, 67% purity) of the title compound were obtained. According to LC/MS, the corresponding disulphide (dimerized product, (+/−)-bis[2-(trimethylsilyl)ethyl]2,2′-[disulphanediylbis(benzene-4,1-diylcarbonyl)]bis(5-{[4-oxo-6-(trifluoromethyl)-1,2,3-benzotriazin-3(4H)-yl]methyl}cyclopentanecarboxylate) was present to an extent of 25%.

¹H-NMR (400 MHz, DMSO-d₆): δ [ppm]=8.52 (s, 1H), 8.44 (s, 2H), 7.83 (d, 2H), 7.42 (d, 2H), 6.03 (br. s, 1H), 4.60-4.48 (m, 2H), 4.08 (q, 1H), 3.64-3.48 (m, 2H), 3.23 (t, 1H), 2.97-2.81 (m, 1H), 2.19-2.06 (m, 1H), 2.06-1.94 (m, 1H), 1.79-1.67 (m, 1H), 1.62-1.48 (m, 1H), 0.37-0.22 (m, 2H), −0.18 (s, 9H).

LC/MS (Method 1, ESIpos): Rₜ=1.44 min, m/z=578 [M+H]⁺.

### Example 40A

### 2-(Trimethylsilyl)ethyl (1RS,2RS,5SR)-2-[4-(benzylsulphanyl)benzoyl]-5-{[4-oxo-6-(trifluoromethyl)-1,2,3-benzotriazin-3(4H)-yl]methyl}cyclopentanecarboxylate (racemate)

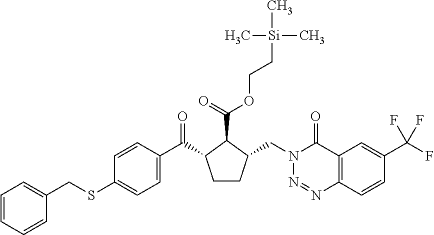 [SMILES: C[Si](C)(C)CCOC(=O)[C@H]1[C@H](Cn2nnc3ccc(C(F)(F)F)cc3c2=O)CC[C@@H]1C(=O)c1ccc(SCc2ccccc2)cc1]

To a solution of 245 mg (0.35 mmol) of the compound from Example 38A in 3 ml of dioxane were successively added 47 mg (0.38 mmol) of phenylmethanethiol, 91 mg (0.71 mmol) of N,N-diisopropylethylamine, 8 mg (0.01 mmol) of tris(dibenzylideneacetone)dipalladium and 10 mg (0.02 mmol) of 4,5-bis(diphenylphosphino)-9,9-dimethylxanthene (Xantphos). The mixture was degassed, purged with argon and then stirred under reflux for 2 h. After cooling to RT, the mixture was purified directly by means of preparative HPLC (Method 5). The product-containing fractions were combined, neutralized with saturated aqueous sodium hydrogencarbonate solution and concentrated down to a small residual volume of aqueous phase. After this aqueous phase had been extracted twice with dichloromethane, the combined organic phases were dried over magnesium sulphate, filtered and concentrated, and the residue was dried under reduced pressure. 203 mg (86% of theory, 100% purity) of the title compound were obtained.

¹H-NMR (400 MHz, DMSO-d₆): δ [ppm]=8.52 (s, 1H), 8.44 (s, 2H), 7.87 (d, 2H), 7.46-7.39 (m, 4H), 7.32 (t, 2H), 7.25 (d, 1H), 4.61-4.49 (m, 2H), 4.37 (s, 2H), 4.09 (q, 1H), 3.65-3.48 (m, 2H), 3.24 (t, 1H), 2.97-2.82 (m, 1H), 2.18-2.06 (m, 1H), 2.05-1.94 (m, 1H), 1.79-1.67 (m, 1H), 1.62-1.48 (m, 1H), 0.39-0.20 (m, 2H), −0.18 (s, 9H).

LC/MS (Method 1, ESIpos): Rₜ=1.55 min, m/z=668 [M+H]⁺.

### Example 41A

### 2-(Trimethylsilyl)ethyl (1RS,2RS,5SR)-2-{4-[(3-chlorobenzyl)sulphanyl]benzoyl}-5-{[4-oxo-6-(trifluoromethyl)-1,2,3-benzotriazin-3(4H)-yl]methyl}cyclopentanecarboxylate (racemate)

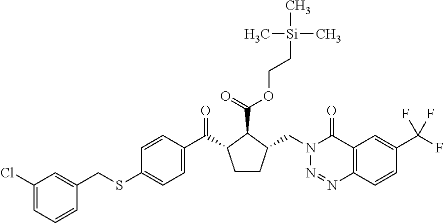 [SMILES: C[Si](C)(C)CCOC(=O)[C@H]1[C@H](Cn2nnc3ccc(C(F)(F)F)cc3c2=O)CC[C@@H]1C(=O)c1ccc(SCc2cccc(Cl)c2)cc1]

To a solution of 300 mg (0.43 mmol) of the compound from Example 38A in 4 ml of dioxane were successively added 73 mg (0.46 mmol) of (3-chlorophenyl)methanethiol, 112 mg (0.87 mmol) of N,N-diisopropylethylamine, 10 mg (0.01 mmol) of tris(dibenzylideneacetone)dipalladium and 13 mg (0.02 mmol) of 4,5-bis(diphenylphosphino)-9,9-dimethylxanthene (Xantphos). The mixture was degassed, purged with argon and then stirred under reflux for 2 h. After cooling to RT and standing overnight, the mixture was purified directly by means of preparative HPLC (Method 5). The product-containing fractions were combined, neutralized with saturated aqueous sodium hydrogencarbonate solution and concentrated down to a small residual volume of aqueous phase. After this aqueous phase had been extracted twice with dichloromethane, the combined organic phases were dried over magnesium sulphate, filtered and concentrated, and the residue was dried under reduced pressure. 255 mg (84% of theory, 100% purity) of the title compound were obtained.

¹H-NMR (400 MHz, DMSO-d₆): δ [ppm]=8.52 (s, 1H), 8.45 (s, 2H), 7.87 (d, 2H), 7.51-7.27 (m, 6H), 4.61-4.48 (m, 2H), 4.39 (s, 2H), 4.10 (q, 1H), 3.65-3.47 (m, 2H), 3.24 (t, 1H), 2.97-2.83 (m, 1H), 2.18-2.06 (m, 1H), 2.05-1.94 (m, 1H), 1.79-1.67 (m, 1H), 1.62-1.48 (m, 1H), 0.39-0.19 (m, 2H), −0.19 (s, 9H).

LC/MS (Method 1, ESIpos): Rₜ=1.59 min, m/z=702 [M+H]⁺.

### Example 42A

### 2-(Trimethylsilyl)ethyl (1RS,2RS,5SR)-2-{4-[(3,4-dichlorobenzyl)sulphanyl]benzoyl}-5-{[4-oxo-6-(trifluoromethyl)-1,2,3-benzotriazin-3(4H)-yl]methyl}cyclopentanecarboxylate (racemate)

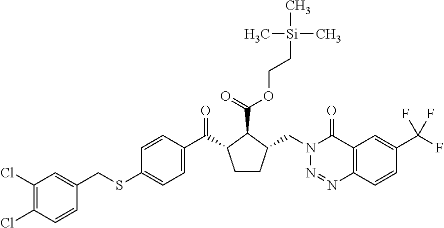 [SMILES: C[Si](C)(C)CCOC(=O)[C@H]1[C@H](Cn2nnc3ccc(C(F)(F)F)cc3c2=O)CC[C@@H]1C(=O)c1ccc(SCc2ccc(Cl)c(Cl)c2)cc1]

To a solution of 300 mg (0.43 mmol) of the compound from Example 38A in 4 ml of dioxane were successively added 89 mg (0.46 mmol) of (3,4-dichlorophenyl)methanethiol, 112 mg (0.87 mmol) of N,N-diisopropylethylamine, 10 mg (0.01 mmol) of tris(dibenzylideneacetone)dipalladium and 13 mg (0.02 mmol) of 4,5-bis(diphenylphosphino)-9,9-dimethylxanthene (Xantphos). The mixture was degassed, purged with argon and then stirred under reflux for 2 h. After cooling to RT and standing overnight, the mixture was purified directly by means of preparative HPLC (Method 5). The product-containing fractions were combined, neutralized with saturated aqueous sodium hydrogencarbonate solution and concentrated down to a small residual volume of aqueous phase. After this aqueous phase had been extracted twice with dichloromethane, the combined organic phases were dried over magnesium sulphate, filtered and concentrated, and the residue was dried under reduced pressure. 266 mg (84% of theory, 100% purity) of the title compound were obtained.

¹H-NMR (400 MHz, DMSO-d₆): δ [ppm]=8.52 (s, 1H), 8.45 (s, 2H), 7.87 (d, 2H), 7.69 (d, 1H), 7.57 (d, 1H), 7.47-7.39 (m, 3H), 4.59-4.50 (m, 2H), 4.39 (s, 2H), 4.09 (q, 1H), 3.64-3.48 (m, 2H), 3.23 (t, 1H), 2.96-2.82 (m, 1H), 2.17-2.06 (m, 1H), 2.05-1.94 (m, 1H), 1.80-1.67 (m, 1H), 1.62-1.49 (m, 1H), 0.37-0.21 (m, 2H), −0.20 (s, 9H).

LC/MS (Method 1, ESIpos): Rₜ=1.63 min, m/z=736 [M+H]⁺.

### Example 43A

### 2-(Trimethylsilyl)ethyl (1RS,2SR,5RS)-2-{[4-oxo-6-(trifluoromethyl)-1,2,3-benzotriazin-3(4H)-yl]methyl}-5-{4-[(2-phenylethyl)sulphanyl]benzoyl}cyclopentanecarboxylate (racemate)

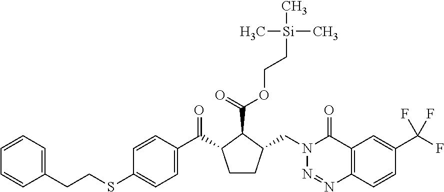 [SMILES: C[Si](C)(C)CCOC(=O)[C@H]1[C@H](Cn2nnc3ccc(C(F)(F)F)cc3c2=O)CC[C@@H]1C(=O)c1ccc(SCCc2ccccc2)cc1]

To a solution of 300 mg (0.43 mmol) of the compound from Example 38A in 4 ml of dioxane were successively added 64 mg (0.46 mmol) of 2-phenylethanethiol, 112 mg (0.87 mmol) of N,N-diisopropylethylamine, 10 mg (0.01 mmol) of tris(dibenzylideneacetone)dipalladium and 13 mg (0.02 mmol) of 4,5-bis(diphenylphosphino)-9,9-dimethylxanthene (Xantphos). The mixture was degassed, purged with argon and then stirred under reflux for 2 h. After cooling to RT and standing overnight, the mixture was purified directly by means of preparative HPLC (Method 5). The product-containing fractions were combined, neutralized with saturated aqueous sodium hydrogencarbonate solution and concentrated down to a small residual volume of aqueous phase. After this aqueous phase had been extracted twice with dichloromethane, the combined organic phases were dried over magnesium sulphate, filtered and concentrated, and the residue was dried under reduced pressure. 239 mg (81% of theory, 100% purity) of the title compound were obtained.

¹H-NMR (400 MHz, DMSO-d₆): δ [ppm]=8.52 (s, 1H), 8.45 (s, 2H), 7.90 (d, 2H), 7.43 (d, 2H), 7.34-7.26 (m, 4H), 7.25-7.18 (m, 1H), 4.61-4.50 (m, 2H), 4.11 (q, 1H), 3.66-3.49 (m, 2H), 3.37-3.30 (hidden, 2H), 3.25 (t, 1H), 2.96-2.84 (m, 3H), 2.19-2.08 (m, 1H), 2.07-1.96 (m, 1H), 1.81-1.70 (m, 1H), 1.62-1.51 (m, 1H), 0.40-0.23 (m, 2H), −0.19 (s, 9H).

LC/MS (Method 1, ESIpos): Rₜ=1.62 min, m/z=682 [M+H]⁺.

### Example 44A

### 2-(Trimethylsilyl)ethyl (1RS,2RS,5SR)-2-{4-[(cyclohexylmethyl)sulphanyl]benzoyl}-5-{[4-oxo-6-(trifluoromethyl)-1,2,3-benzotriazin-3(4H)-yl]methyl}cyclopentanecarboxylate (racemate)

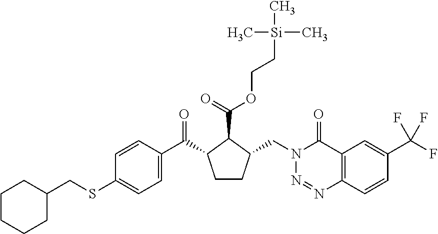 [SMILES: C[Si](C)(C)CCOC(=O)[C@H]1[C@H](Cn2nnc3ccc(C(F)(F)F)cc3c2=O)CC[C@@H]1C(=O)c1ccc(SCC2CCCCC2)cc1]

To a solution of 220 mg of the compound from Example 39A (0.38 mmol, not corrected for purity, containing about 25% corresponding disulphide) in 15 ml of DMF were added 105 mg (0.76 mmol) of potassium carbonate, and the mixture was stirred at RT for 2 min. Subsequently, 148 mg (0.84 mmol) of (bromomethyl)cyclohexane, 135 mg (1.14 mmol) of sodium hydroxymethanesulphinate and 0.2 ml (0.11 mmol) of water were added and the mixture was stirred at RT for a further 30 min. The mixture was then concentrated and the residue was admixed with water and extracted twice with ethyl acetate. The combined organic phases were washed once with saturated sodium chloride solution, dried over magnesium sulphate, filtered and concentrated. 236 mg (87% of theory, 95% purity) of the title compound were obtained.

LC/MS (Method 1, ESIpos): Rₜ=1.73 min, m/z=674 [M+H]⁺.

### Example 45A

### 2-(Trimethylsilyl)ethyl (1RS,2SR,5RS)-2-{[4-oxo-6-(trifluoromethyl)-1,2,3-benzotriazin-3(4H)-yl]methyl}-5-[4-(pentylsulphanyl)benzoyl]cyclopentanecarboxylate (racemate)

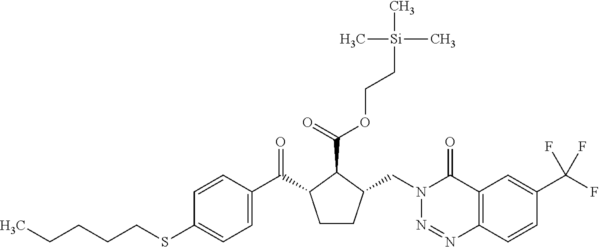 [SMILES: CCCCCSc1ccc(C(=O)[C@H]2CC[C@@H](Cn3nnc4ccc(C(F)(F)F)cc4c3=O)[C@@H]2C(=O)OCC[Si](C)(C)C)cc1]

To a solution of 300 mg (0.43 mmol) of the compound from Example 38A in 4 ml of dioxane were successively added 48 mg (0.46 mmol) of pentane-1-thiol, 112 mg (0.87 mmol) of N,N-diisopropylethylamine, 10 mg (0.01 mmol) of tris(dibenzylideneacetone)dipalladium and 13 mg (0.02 mmol) of 4,5-bis(diphenylphosphino)-9,9-dimethylxanthene (Xantphos). The mixture was degassed, purged with argon and then stirred under reflux for 2 h. After cooling to RT and standing overnight, the mixture was purified directly by means of preparative HPLC (Method 5). The product-containing fractions were combined, neutralized with saturated aqueous sodium hydrogencarbonate solution and concentrated down to a small residual volume of aqueous phase. After this aqueous phase had been extracted twice with dichloromethane, the combined organic phases were dried over magnesium sulphate, filtered and concentrated, and the residue was dried under reduced pressure. 233 mg (82% of theory, 98% purity) of the title compound were obtained.

¹H-NMR (400 MHz, DMSO-d₆): δ [ppm]=8.52 (s, 1H), 8.45 (s, 2H), 7.88 (d, 2H), 7.38 (d, 2H), 4.62-4.48 (m, 2H), 4.10 (q, 1H), 3.66-3.47 (m, 2H), 3.23 (t, 1H), 3.05 (t, 2H), 2.97-2.83 (m, 1H), 2.20-2.06 (m, 1H), 2.06-1.95 (m, 1H), 1.81-1.71 (m, 1H), 1.67-1.51 (m, 3H), 1.44-1.19 (m, 4H), 0.86 (t, 3H), 0.39-0.20 (m, 2H), −0.18 (s, 9H).

LC/MS (Method 1, ESIpos): Rₜ=1.67 min, m/z=648 [M+H]⁺.

### Example 46A

### 2-(Trimethylsilyl)ethyl (1RS,2RS,5SR)-2-[4-(benzyloxy)benzoyl]-5-[(6-methyl-4-oxo-1,2,3-benzotriazin-3(4H)-yl)methyl]cyclopentanecarboxylate (racemate)

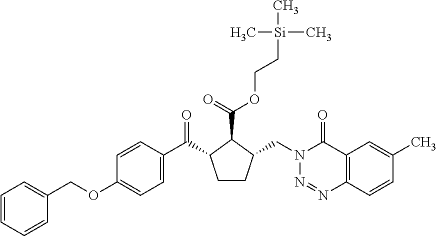 [SMILES: Cc1ccc2nnn(C[C@@H]3CC[C@H](C(=O)c4ccc(OCc5ccccc5)cc4)[C@H]3C(=O)OCC[Si](C)(C)C)c(=O)c2c1]

To a suspension of 9.60 g (20.06 mmol, 95% purity) of the compound from Example 3A/Step 5 in 110 ml of toluene under argon were added 3.88 g (24.07 mmol) of the compound from Example 2A.

Subsequently, 25.1 ml (100.30 mmol) of tributylphosphine and 27.4 ml (60.18 mmol) of a 40% solution of diethyl azodicarboxylate in toluene were added dropwise at 0° C. After stirring at RT for 2 h, the mixture was diluted with ethyl acetate and washed once with water. The aqueous phase was reextracted once with ethyl acetate. The combined organic phases were washed once with saturated sodium chloride solution, dried over magnesium sulphate, filtered and concentrated. The residue was purified by means of flash chromatography (silica gel, eluent: cyclohexane/ethyl acetate 85:15→80:20). 6.28 g (51% of theory, 98% purity) of the title compound were obtained.

¹H-NMR (400 MHz, DMSO-d₆): δ [ppm]=8.12-8.05 (m, 2H), 7.97-7.88 (m, 3H), 7.48-7.27 (m, 5H), 7.12 (d, 2H), 5.20 (s, 2H), 4.56-4.42 (m, 2H), 4.08 (q, 1H), 3.61-3.46 (m, 2H), 3.22 (t, 1H), 2.96-2.83 (m, 1H), 2.55 (s, 3H), 2.17-2.05 (m, 1H), 2.03-1.92 (m, 1H), 1.78-1.67 (m, 1H), 1.59-1.47 (m, 1H), 0.38-0.23 (m, 2H), −0.17 (s, 9H).

LC/MS (Method 1, ESIpos): Rₜ=1.49 min, m/z=598 [M+H]⁺.

### Example 47A

### 2-(Trimethylsilyl)ethyl (1RS,2RS,5SR)-2-(4-hydroxybenzoyl)-5-[(6-methyl-4-oxo-1,2,3-benzotriazin-3(4H)-yl)methyl]cyclopentanecarboxylate (racemate)

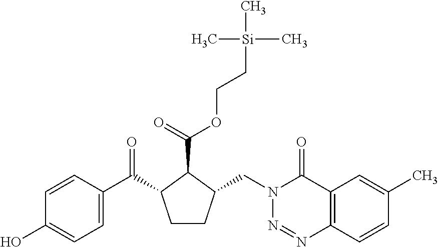 [SMILES: Cc1ccc2nnn(C[C@@H]3CC[C@H](C(=O)c4ccc(O)cc4)[C@H]3C(=O)OCC[Si](C)(C)C)c(=O)c2c1]

To a solution of 6.25 g (10.25 mmol, 98% purity) of the compound from Example 46A in a mixture of 50 ml of ethyl acetate and 50 ml of ethanol under argon were added 273 mg (0.26 mmol) of palladium on activated carbon (10% Pd) and 969 mg (15.37 mmol) of ammonium formate. The mixture was then stirred at 70° C. for 2 h. After cooling to RT, the mixture was filtered through kieselguhr, the filter residue was washed with ethyl acetate and ethanol, the filtrate was concentrated and the residue was dried under reduced pressure. 5.20 g (97% of theory, 97% purity) of the title compound were obtained.

¹H-NMR (400 MHz, DMSO-d₆): δ [ppm]=10.40 (br. s, 1H), 8.12-8.05 (m, 2H), 7.92 (dd, 1H), 7.84 (d, 2H), 6.84 (d, 2H), 4.57-4.42 (m, 2H), 4.03 (q, 1H), 3.62-3.46 (m, 2H), 3.21 (t, 1H), 2.96-2.83 (m, 1H), 2.55 (s, 3H), 2.17-2.04 (m, 1H), 2.03-1.91 (m, 1H), 1.78-1.66 (m, 1H), 1.60-1.48 (m, 1H), 0.39-0.25 (m, 2H), −0.17 (s, 9H).

LC/MS (Method 1, ESIpos): Rₜ=1.28 min, m/z=508 [M+H]⁺.

### Example 48A

### 2-(Trimethylsilyl)ethyl (1RS,2RS,5SR)-2-{4-[(4-fluorobenzyl)oxy]benzoyl}-5-[(6-methyl-4-oxo-1,2,3-benzotriazin-3(4H)-yl)methyl]cyclopentanecarboxylate (racemate)

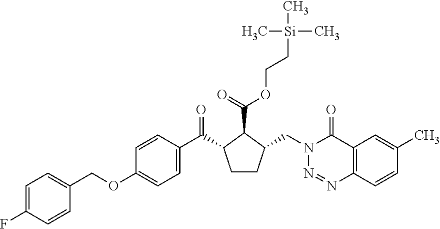 [SMILES: Cc1ccc2nnn(C[C@@H]3CC[C@H](C(=O)c4ccc(OCc5ccc(F)cc5)cc4)[C@H]3C(=O)OCC[Si](C)(C)C)c(=O)c2c1]

To a solution of 169 mg (0.27 mmol, 98% purity) of the compound from Example 47A in 2.1 ml of DMF under argon were added 51 mg (0.46 mmol) of potassium tert-butoxide. After stirring at RT for 5 min, 87 mg (0.46 mmol) of 4-fluorobenzyl bromide were added, and the mixture was stirred at bath temperature 100° C. for 1 h. After cooling to RT, 60 ml of water and 60 ml of ethyl acetate were added to the mixture. After separating the phases, the aqueous phase was extracted once with 60 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 169 mg (71% of theory, 98% purity) of the title compound were obtained.

LC/MS (Method 1, ESIpos): Rₜ=1.48 min, m/z=616 [M+H]⁺.

### Example 49A

### 2-(Trimethylsilyl)ethyl (1RS,2RS,5SR)-2-{4-[(3-methylbenzyl)oxy]benzoyl}-5-[(6-methyl-4-oxo-1,2,3-benzotriazin-3(4H)-yl)methyl]cyclopentanecarboxylate (racemate)

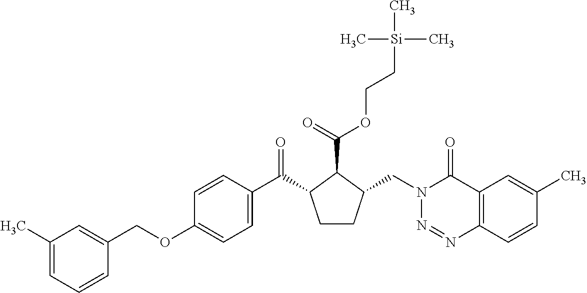

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 85 mg (0.46 mmol) of 3-methylbenzyl bromide were added, and the mixture was stirred at bath temperature 100° C. for 1 h. After cooling to RT, 60 ml of water and 60 ml of ethyl acetate were added to the mixture. After separating the phases, the aqueous phase was extracted once with 60 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 120 mg (51% of theory, 100% purity) of the title compound were obtained.

LC/MS (Method 1, ESIpos): Rₜ=1.53 min, m/z=612 [M+H]⁺.

### Example 50A

### 2-(Trimethylsilyl)ethyl (1RS,2SR,5RS)-2-[(6-methyl-4-oxo-1,2,3-benzotriazin-3(4H)-yl)methyl]-5-(4-{[3-(trifluoromethyl)benzyl]oxy}benzoyl)cyclopentanecarboxylate (racemate)

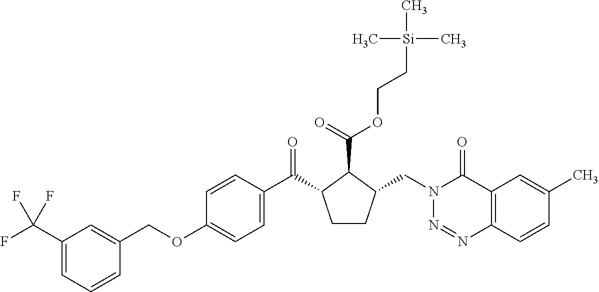 [SMILES: Cc1ccc2nnn(C[C@@H]3CC[C@H](C(=O)c4ccc(OCc5cccc(C(F)(F)F)c5)cc4)[C@H]3C(=O)OCC[Si](C)(C)C)c(=O)c2c1]

To a solution of 500 mg (0.96 mmol, 97% purity) of the compound from Example 47A in 5.3 ml of DMF under argon were added 129 mg (1.15 mmol) of potassium tert-butoxide. After stirring at RT for 5 min, 274 mg (1.15 mmol) of 3-(trifluoromethyl)benzyl bromide were added, and the mixture was stirred at bath temperature 100° C. for 2 h. Subsequently, a further 274 mg (1.15 mmol) of 3-(trifluoromethyl)benzyl bromide were added, and the mixture was stirred at bath temperature 100° C. for 2 h. After cooling to RT, 60 ml of water and 60 ml of ethyl acetate were added to the mixture. After separating the phases, the aqueous phase was extracted once with 60 ml of ethyl acetate. The combined organic phases were washed once with saturated sodium chloride solution, dried over magnesium sulphate, filtered and concentrated. The residue was purified by means of column chromatography (90 g of silica gel, eluent: cyclohexane/ethyl acetate 8:2). 345 mg (53% of theory, 97% purity) of the title compound were obtained.

LC/MS (Method 1, ESIpos): Rₜ=1.58 min, m/z=666 [M+H]⁺.

### Example 51A

### 2-(Trimethylsilyl)ethyl (1RS,2RS,5SR)-2-{4-[(3-chlorobenzyl)oxy]benzoyl}-5-[(6-methyl-4-oxo-1,2,3-benzotriazin-3(4H)-yl)methyl]cyclopentanecarboxylate (racemate)

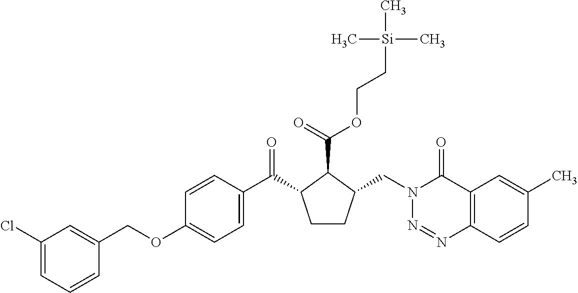 [SMILES: Cc1ccc2nnn(C[C@@H]3CC[C@H](C(=O)c4ccc(OCc5cccc(Cl)c5)cc4)[C@H]3C(=O)OCC[Si](C)(C)C)c(=O)c2c1]

To a solution of 500 mg (0.96 mmol, 97% purity) of the compound from Example 47A in 5.3 ml of DMF under argon were added 129 mg (1.15 mmol) of potassium tert-butoxide. After stirring at RT for 5 min, 236 mg (1.15 mmol) of 3-chlorobenzyl bromide were added, and the mixture was stirred at bath temperature 100° C. for 1 h. After cooling and being left to stand at RT overnight, the mixture was stirred once again at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (90 g of silica gel, eluent: cyclohexane/ethyl acetate 85:15). 392 mg (65% of theory, 100% purity) of the title compound were obtained.

LC/MS (Method 1, ESIpos): Rₜ=1.58 min, m/z=632 [M+H]⁺.

### Example 52A

### 2-(Trimethylsilyl)ethyl (1RS,2RS,5SR)-2-{4-[(3-methoxybenzyl)oxy]benzoyl}-5-[(6-methyl-4-oxo-1,2,3-benzotriazin-3(4H)-yl)methyl]cyclopentanecarboxylate (racemate)

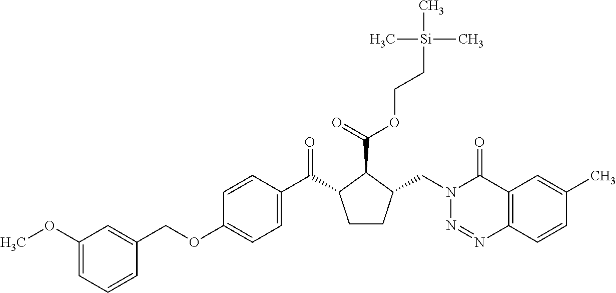

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 92 mg (0.46 mmol) of 1-(bromomethyl)-3-methoxybenzene were added, and the mixture was stirred at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 115 mg (47% of theory, 98% purity) of the title compound were obtained.

LC/MS (Method 1, ESIpos): Rₜ=1.50 min, m/z=628 [M+H]⁺.

### Example 53A

### 2-(Trimethylsilyl)ethyl (1RS,2SR,5RS)-2-[(6-methyl-4-oxo-1,2,3-benzotriazin-3(4H)-yl)methyl]-5-(4-{[3-(trifluoromethoxy)benzyl]oxy}benzoyl)cyclopentanecarboxylate (racemate)

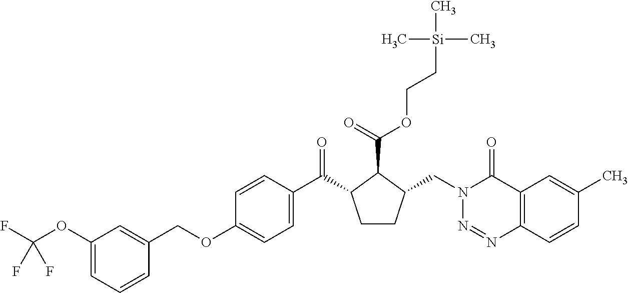 [SMILES: Cc1ccc2nnn(C[C@@H]3CC[C@H](C(=O)c4ccc(OCc5cccc(OC(F)(F)F)c5)cc4)[C@H]3C(=O)OCC[Si](C)(C)C)c(=O)c2c1]

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 117 mg (0.46 mmol) of 1-(bromomethyl)-3-(trifluoromethoxy)benzene were added, and the mixture was stirred at bath temperature 100° C. for 2 h. Thereafter, a further 117 mg (0.46 mmol) of 1-(bromomethyl)-3-(trifluoromethoxy)benzene were added, and the mixture was stirred once again at bath temperature 100° C. for 2 h. Subsequently, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 95 mg (36% of theory, 100% purity) of the title compound were obtained.

LC/MS (Method 1, ESIpos): Rₜ=1.56 min, m/z=682 [M+H]⁺.

### Example 54A

### 2-(Trimethylsilyl)ethyl (1RS,2SR,5RS)-2-[(6-methyl-4-oxo-1,2,3-benzotriazin-3(4H)-yl)methyl]-5-[4-(2-phenylethoxy)benzoyl]cyclopentanecarboxylate (racemate)

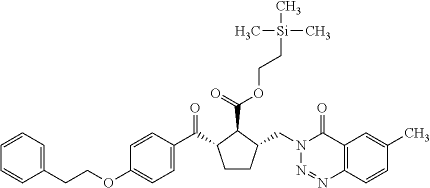 [SMILES: Cc1ccc2nnn(C[C@@H]3CC[C@H](C(=O)c4ccc(OCCc5ccccc5)cc4)[C@H]3C(=O)OCC[Si](C)(C)C)c(=O)c2c1]

To a solution of 500 mg (0.96 mmol, 97% purity) of the compound from Example 47A in 5 ml of acetonitrile under argon were added 214 mg (1.91 mmol) of potassium carbonate and 324 mg (1.15 mmol, 90% purity) of 2-phenylethyl trifluoromethanesulphonate. The mixture was stirred at RT for 4 h. The mixture was then admixed with water and extracted twice with ethyl acetate. The combined organic phases were dried over magnesium sulphate, filtered and concentrated. The residue was purified by means of column chromatography (90 g of silica gel, eluent: cyclohexane/ethyl acetate 8:2). 347 mg (59% of theory, 100% purity) of the title compound were obtained.

LC/MS (Method 1, ESIpos): Rₜ=1.58 min, m/z=612 [M+H]⁺.

### Example 55A

### 2-(Trimethylsilyl)ethyl (1RS,2SR,5RS)-2-[(6-methyl-4-oxo-1,2,3-benzotriazin-3(4H)-yl)methyl]-5-[4-(3-phenylpropoxy)benzoyl]cyclopentanecarboxylate (racemate)

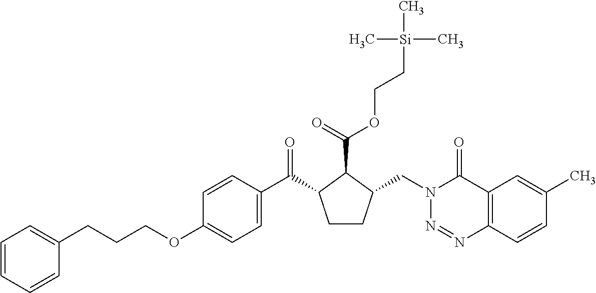 [SMILES: Cc1ccc2nnn(C[C@@H]3CC[C@H](C(=O)c4ccc(OCCCc5ccccc5)cc4)[C@H]3C(=O)OCC[Si](C)(C)C)c(=O)c2c1]

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 91 mg (0.46 mmol) of (3-bromopropyl)benzene were added, and the mixture was stirred at bath temperature 100° C. for 2 h. Subsequently, a further 75 mg (0.38 mmol) of (3-bromopropyl)benzene were added, and the mixture was stirred once again at bath temperature 100° C. for 2 h. Thereafter, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 44 mg (18% of theory, 100% purity) of the title compound were obtained.

LC/MS (Method 1, ESIpos): Rₜ=1.60 min, m/z=626 [M+H]⁺.

### Example 56A

### 2-(Trimethylsilyl)ethyl (1RS,2RS,5SR)-2-[4-(cyclohexylmethoxy)benzoyl]-5-[(6-methyl-4-oxo-1,2,3-benzotriazin-3(4H)-yl)methyl]cyclopentanecarboxylate (racemate)

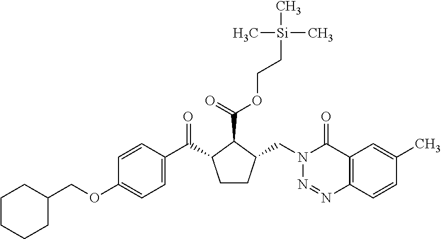 [SMILES: Cc1ccc2nnn(C[C@@H]3CC[C@H](C(=O)c4ccc(OCC5CCCCC5)cc4)[C@H]3C(=O)OCC[Si](C)(C)C)c(=O)c2c1]

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 81 mg (0.46 mmol) of (bromomethyl)cyclohexane were added, and the mixture was stirred at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 163 mg (71% of theory, 100% purity) of the title compound were obtained.

LC/MS (Method 1, ESIpos): Rₜ=1.65 min, m/z=604 [M+H]⁺.

### Example 57A

### 2-(Trimethylsilyl)ethyl (1RS,2RS,5SR)-2-[4-(cyclopentylmethoxy)benzoyl]-5-[(6-methyl-4-oxo-1,2,3-benzotriazin-3(4H)-yl)methyl]cyclopentanecarboxylate (racemate)

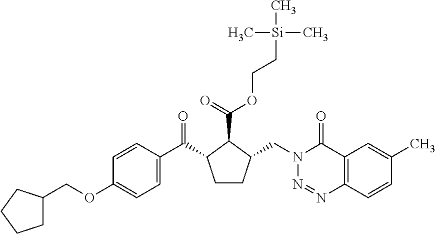 [SMILES: Cc1ccc2nnn(C[C@@H]3CC[C@H](C(=O)c4ccc(OCC5CCCC5)cc4)[C@H]3C(=O)OCC[Si](C)(C)C)c(=O)c2c1]

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 75 mg (0.46 mmol) of (bromomethyl)cyclopentane were added, and the mixture was stirred at bath temperature 100° C. for 2 h. Thereafter, a further 75 mg (0.46 mmol) of (bromomethyl)cyclopentane were added, and the mixture was stirred once again at bath temperature 100° C. for 3.5 h. Subsequently, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 49 mg (13% of theory, 58% purity) of the title compound were obtained.

LC/MS (Method 1, ESIpos): Rₜ=1.64 min, m/z=590 [M+H]⁺.

### Example 58A

### 2-(Trimethylsilyl)ethyl (1RS,2RS,5SR)-2-[4-(hexyloxy)benzoyl]-5-[(6-methyl-4-oxo-1,2,3-benzotriazin-3(4H)-yl)methyl]cyclopentanecarboxylate (racemate)

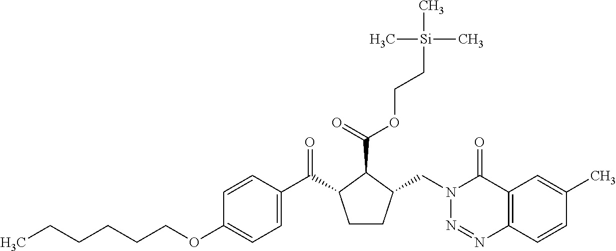 [SMILES: CCCCCCOc1ccc(C(=O)[C@H]2CC[C@@H](Cn3nnc4ccc(C)cc4c3=O)[C@@H]2C(=O)OCC[Si](C)(C)C)cc1]

To a solution of 200 mg (0.38 mmol, 97% purity) of the compound from Example 47A in 2.1 ml of DMF under argon were added 51 mg (0.46 mmol) of potassium tert-butoxide. After stirring at RT for 5 min, 76 mg (0.46 mmol) of 1-bromohexane were added, and the mixture was stirred at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (40 g of silica gel, eluent: cyclohexane/ethyl acetate 85:15). 140 mg (62% of theory, 100% purity) of the title compound were obtained.

LC/MS (Method 1, ESIpos): Rₜ=1.66 min, m/z=592 [M+H]⁺.

### Example 59A

### 2-(Trimethylsilyl)ethyl (1RS,2SR,5RS)-2-[(6-methyl-4-oxo-1,2,3-benzotriazin-3(4H)-yl)methyl]-5-[4-(pentyloxy)benzoyl]cyclopentanecarboxylate (racemate)

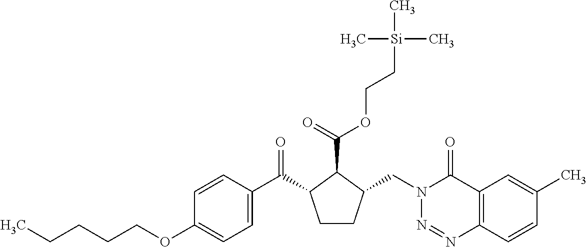 [SMILES: CCCCCOc1ccc(C(=O)[C@H]2CC[C@@H](Cn3nnc4ccc(C)cc4c3=O)[C@@H]2C(=O)OCC[Si](C)(C)C)cc1]

To a solution of 500 mg (0.96 mmol, 97% purity) of the compound from Example 47A in 5.3 ml of DMF under argon were added 129 mg (1.15 mmol) of potassium tert-butoxide. After stirring at RT for 5 min, 173 mg (1.15 mmol) of 1-bromopentane were added, and the mixture was stirred at bath temperature 100° C. for 1 h. After cooling and being left to stand at RT overnight, the mixture was stirred once again at bath temperature 100° C. for 2 h. After cooling to RT, 60 ml of water and 60 ml of ethyl acetate were added to the mixture. After separating the phases, the aqueous phase was extracted once with 30 ml of ethyl acetate. The combined organic phases were washed once with saturated sodium chloride solution, dried over magnesium sulphate, filtered and concentrated. The residue was purified by means of column chromatography (90 g of silica gel, eluent: cyclohexane/ethyl acetate 85:15). 378 mg (60% of theory, 88% purity) of the title compound were obtained.

LC/MS (Method 1, ESIpos): Rₜ=1.58 min, m/z=578 [M+H]⁺.

### WORKING EXAMPLES

### Example 1

(+/−)-(1RS,2RS,5SR)-2-{4-[(4-Fluorobenzyl)oxy]benzoyl}-5-[(4-oxo-1,2,3-benzotriazin-3(4H)-yl)methyl]cyclopentanecarboxylic acid (racemate)

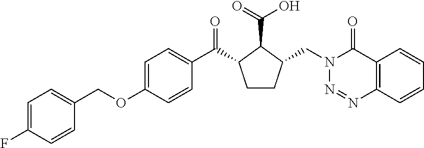 [SMILES: O=C(O)[C@H]1[C@H](Cn2nnc3ccccc3c2=O)CC[C@@H]1C(=O)c1ccc(OCc2ccc(F)cc2)cc1]

To a solution of 49.8 mg (0.08 mmol) of the compound from Example 5A in 0.3 ml of dichloromethane was added, at 0° C., 0.16 ml (2.07 mmol) of trifluoroacetic acid. The mixture was stirred at 0° C. for 3 h and then stored at 5° C. for about 72 h. Thereafter, the mixture was concentrated, the residue was taken up in dichloromethane and the solution was concentrated again. This procedure was repeated several times. In this way, 37 mg (87% of theory, 99% purity) of the title compound were obtained.

¹H-NMR (400 MHz, DMSO-d₆): δ [ppm]=12.14 (br. s, 1H), 8.26 (d, 1H), 8.20 (d, 1H), 8.08 (dd, 1H), 7.98 (d, 2H), 7.93 (dd, 1H), 7.52 (dd, 2H), 7.23 (t, 2H), 7.12 (d, 2H), 5.19 (s, 2H), 4.58-4.48 (m, 2H), 4.11 (dd, 1H), 3.24 (dd, 1H), 2.92 (m, 1H), 2.15-2.06 (m, 1H), 1.93-1.85 (m, 1H), 1.70-1.62 (m, 1H), 1.56-1.46 (m, 1H).

LC/MS (Method 1, ESIpos): Rₜ=1.17 min, m/z=502 [M+H]⁺.

### Example 2

(+/−)-(1RS,2RS,5SR)-2-{4-[(3-Chlorobenzyl)oxy]benzoyl}-5-[(4-oxo-1,2,3-benzotriazin-3(4H)-yl)methyl]cyclopentanecarboxylic acid (racemate)

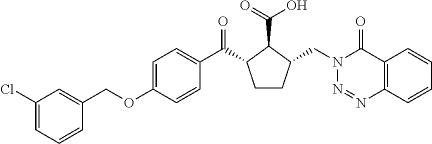 [SMILES: O=C(O)[C@H]1[C@H](Cn2nnc3ccccc3c2=O)CC[C@@H]1C(=O)c1ccc(OCc2cccc(Cl)c2)cc1]

To a solution of 57 mg (0.09 mmol) of the compound from Example 6A in 0.36 ml of dichloromethane was added 0.18 ml (2.29 mmol) of trifluoroacetic acid. The mixture was stirred at RT for 5.5 h. Thereafter, the mixture was concentrated, the residue was taken up in dichloromethane and the solution was concentrated again. This procedure was repeated several times. In this way, 48 mg (100% of theory, 100% purity) of the title compound were obtained.

¹H-NMR (400 MHz, DMSO-d₆): δ [ppm]=12.15 (br. s, 1H), 8.26 (d, 1H), 8.20 (d, 1H), 8.11-8.05 (m, 1H), 7.99 (d, 2H), 7.96-7.90 (m, 1H), 7.54 (s, 1H), 7.47-7.38 (m, 3H), 7.13 (d, 2H), 5.23 (s, 2H), 4.59-4.46 (m, 2H), 4.16-4.04 (m, 1H), 3.24 (t, 1H), 2.94-2.80 (m, 1H), 2.18-2.03 (m, 1H), 1.95-1.82 (m, 1H), 1.73-1.61 (m, 1H), 1.58-1.44 (m, 1H).

LC/MS (Method 1, ESIpos): Rₜ=1.22 min, m/z=518 [M+H]⁺.

### Example 3

### (+/−)-(1RS,2RS,5SR)-2-{4-[(3,4-Dichlorobenzyl)oxy]benzoyl}-5-[(4-oxo-1,2,3-benzotriazin-3(4H)-yl)methyl]cyclopentanecarboxylic acid (racemate)

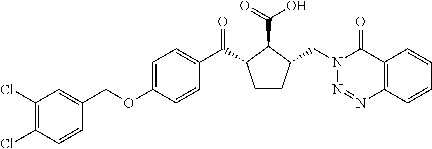 [SMILES: O=C(O)[C@H]1[C@H](Cn2nnc3ccccc3c2=O)CC[C@@H]1C(=O)c1ccc(OCc2ccc(Cl)c(Cl)c2)cc1]

To a solution of 54 mg (0.08 mmol) of the compound from Example 7A in 0.33 ml of dichloromethane was added 0.16 ml (2.06 mmol) of trifluoroacetic acid. The mixture was stirred at RT for 5.5 h. Thereafter, the mixture was concentrated, the residue was taken up in dichloromethane and the solution was concentrated again. This procedure was repeated several times. In this way, 43 mg (95% of theory, 100% purity) of the title compound were obtained.

¹H-NMR (400 MHz, DMSO-d₆): δ [ppm]=12.14 (br. s, 1H), 8.26 (d, 1H), 8.20 (d, 1H), 8.08 (dd, 1H), 7.99 (d, 2H), 7.93 (t, 1H), 7.76 (d, 1H), 7.68 (d, 1H), 7.46 (dd, 1H), 7.13 (d, 2H), 5.23 (s, 2H), 4.58-4.48 (m, 2H), 4.11 (q, 1H), 3.23 (t, 1H), 2.92-2.82 (m, 1H), 2.16-2.06 (m, 1H), 1.93-1.82 (m, 1H), 1.70-1.62 (m, 1H), 1.56-1.46 (m, 1H).

LC/MS (Method 1, ESIpos): Rₜ=1.28 min, m/z=552 [M+H]⁺.

### Example 4

### (+/−)-(1RS,2RS,5SR)-2-[4-(Cyclopentylmethoxy)benzoyl]-5-[(4-oxo-1,2,3-benzotriazin-3(4H)-yl)methyl]cyclopentanecarboxylic acid (racemate)

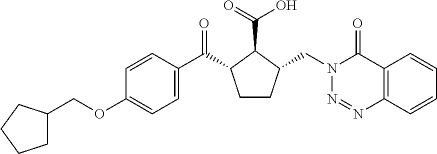 [SMILES: O=C(O)[C@H]1[C@H](Cn2nnc3ccccc3c2=O)CC[C@@H]1C(=O)c1ccc(OCC2CCCC2)cc1]

To a solution of 33 mg (0.06 mmol) of the compound from Example 8A in 0.22 ml of dichloromethane was added, at 0° C., 0.11 ml (1.42 mmol) of trifluoroacetic acid. The mixture was stirred at 0° C. for 3 h and then stored at 5° C. for about 72 h. Then a further 0.04 ml of trifluoroacetic acid was added and the mixture was stirred at RT for another 3 h. Thereafter, the mixture was concentrated, the residue was taken up in dichloromethane and the solution was concentrated again. This procedure was repeated several times. In this way, 26 mg (93% of theory, 97% purity) of the title compound were obtained.

¹H-NMR (400 MHz, DMSO-d₆): δ [ppm]=12.14 (br. s, 1H), 8.26 (d, 1H), 8.20 (d, 1H), 8.08 (dd, 1H), 7.96-7.91 (m, 3H), 7.03 (d, 2H), 4.57-4.48 (m, 2H), 4.10 (dd, 1H), 3.94 (d, 2H), 3.23 (t, 1H), 2.92-2.82 (m, 1H), 2.37-2.28 (m, 1H), 2.15-2.06 (m, 1H), 1.93-1.85 (m, 1H), 1.81-1.73 (m, 2H), 1.70-1.46 (m, 6H), 1.37-1.29 (m, 2H).

LC/MS (Method 1, ESIpos): Rₜ=1.29 min, m/z=476 [M+H]⁺.

### Example 5

### (+/−)-(1RS,2RS,5SR)-2-[4-(2-Cyclohexylethoxy)benzoyl]-5-[(4-oxo-1,2,3-benzotriazin-3(4H)-yl)methyl]cyclopentanecarboxylic acid (racemate)

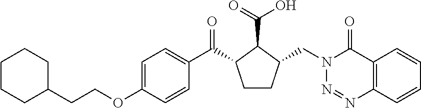 [SMILES: O=C(O)[C@H]1[C@H](Cn2nnc3ccccc3c2=O)CC[C@@H]1C(=O)c1ccc(OCCC2CCCCC2)cc1]

To a solution of 38 mg (0.06 mmol) of the compound from Example 9A in 0.25 ml of dichloromethane was added, at 0° C., 0.12 ml (1.58 mmol) of trifluoroacetic acid. The mixture was stirred at 0° C. for 3 h, then stored at 5° C. for about 72 h, and subsequently stirred at RT for another 3 h. Then the mixture was concentrated, the residue was taken up in dichloromethane and the solution was concentrated again. This procedure was repeated several times. In this way, 31 mg (94% of theory, 96% purity) of the title compound were obtained.

¹H-NMR (400 MHz, DMSO-d₆): δ [ppm]=12.14 (br. s, 1H), 8.26 (d, 1H), 8.20 (d, 1H), 8.08 (dd, 1H), 7.96-7.91 (m, 3H), 7.03 (d, 2H), 4.58-4.48 (m, 2H), 4.13-4.08 (m, 3H), 3.24 (dd, 1H), 2.92-2.82 (m, 1H), 2.15-2.06 (m, 1H), 1.93-1.82 (m, 1H), 1.75-1.61 (m, 8H), 1.56-1.46 (m, 2H), 1.26-1.11 (m, 3H), 0.95 (dd, 2H).

LC/MS (Method 1, ESIpos): Rₜ=1.41 min, m/z=504 [M+H]⁺.

### Example 6

### (+/−)-(1RS,2RS,5SR)-2-[4-(Hexyloxy)benzoyl]-5-[(4-oxo-1,2,3-benzotriazin-3(4H)-yl)methyl]cyclopentanecarboxylic acid (racemate)

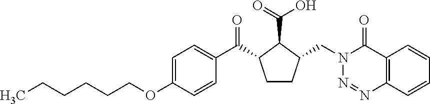 [SMILES: CCCCCCOc1ccc(C(=O)[C@H]2CC[C@@H](Cn3nnc4ccccc4c3=O)[C@@H]2C(=O)O)cc1]

To a solution of 41 mg (0.07 mmol) of the compound from Example 10A in 0.28 ml of dichloromethane was added 0.14 ml (1.76 mmol) of trifluoroacetic acid. The mixture was stirred at RT for 5.5 h. Thereafter, the mixture was concentrated, the residue was taken up in dichloromethane and the solution was concentrated again. This procedure was repeated several times. In this way, 32 mg (95% of theory, 100% purity) of the title compound were obtained.

¹H-NMR (400 MHz, DMSO-d₆): δ [ppm]=12.14 (br. s, 1H), 8.26 (d, 2H), 8.20 (d, 2H), 8.08 (dd, 1H), 7.97-7.91 (m, 2H), 7.03 (d, 1H), 4.58-4.48 (m, 2H), 4.13-4.04 (m, 3H), 3.24 (t, 1H), 2.90-2.84 (m, 1H), 2.15-2.06 (m, 1H), 1.93-1.85 (m, 1H), 1.76-1.62 (m, 3H), 1.56-1.51 (m, 1H), 1.44-1.37 (m, 2H), 1.33-1.23 (m, 4H), 0.88 (t, 3H).

LC/MS (Method 1, ESIpos): Rₜ=1.30 min, m/z=478 [M+H]⁺.

### Example 7

### (+/−)-(1RS,2SR,5RS)-2-[(4-Oxo-1,2,3-benzotriazin-3(4H)-yl)methyl]-5-[4-(pentyloxy)benzoyl]cyclopentanecarboxylic acid (racemate)

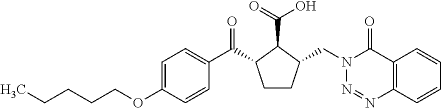 [SMILES: CCCCCOc1ccc(C(=O)[C@H]2CC[C@@H](Cn3nnc4ccccc4c3=O)[C@@H]2C(=O)O)cc1]

To a solution of 30 mg (0.05 mmol) of the compound from Example 11A in 0.21 ml of dichloromethane was added 0.10 ml (1.33 mmol) of trifluoroacetic acid. The mixture was stirred at RT for 5.5 h. Thereafter, the mixture was concentrated, the residue was taken up in dichloromethane and the solution was concentrated again. This procedure was repeated several times. In this way, 26 mg (>100% of theory, 99% purity) of the title compound were obtained.

¹H-NMR (400 MHz, DMSO-d₆): δ [ppm]=12.14 (s, 1H), 8.26 (d, 1H), 8.20 (d, 1H), 8.08 (dd, 1H), 7.97-7.91 (m, 3H), 7.03 (d, 2H), 4.58-4.48 (m, 2H), 4.14-4.01 (m, 3H), 3.24 (t, 1H), 2.92-2.82 (m, 1H), 2.15-2.06 (m, 1H), 1.93-1.85 (m, 1H), 1.77-1.62 (m, 3H), 1.56-1.46 (m, 1H), 1.43-1.32 (m, 4H), 0.89 (t, 3H).

LC/MS (Method 1, ESIpos): Rₜ=1.25 min, m/z=464 [M+H]⁺.

### Separation of the Enantiomers:

18 mg of the racemic compound from Example 7 were dissolved in 7 ml of ethanol/isohexane and separated into the enantiomers by means of preparative HPLC on a chiral phase (see Examples 8 and 9) [column: Daicel Chiralpak AY-H, 5 μm, 250 mm×20 mm; flow rate: 20 ml/min; detection: 210 nm; injection volume: 3.5 ml; temperature: 40° C.; eluent: 30% isohexane/70% ethanol+0.2% TFA+1% water; total run time 14.1 min].

### Example 8

### (+)-(1RS,2SR,5RS)-2-[(4-Oxo-1,2,3-benzotriazin-3(4H)-yl)methyl]-5-[4-(pentyloxy)benzoyl]cyclopentanecarboxylic acid (enantiomer 1)

Yield: 6 mg; chem. purity=100%; ee=100%
[α]_{D}²⁰=+51.4°, 589 nm, c=0.07 g/100 ml, chloroform

¹H-NMR (400 MHz, DMSO-d₆): δ [ppm]=12.11 (br. s, 1H), 8.26 (d, 1H), 8.20 (d, 1H), 8.08 (t, 1H), 8.00-7.89 (m, 3H), 7.03 (d, 2H), 4.59-4.46 (m, 2H), 4.14-4.00 (m, 3H), 3.24 (t, 1H), 2.95-2.81 (m, 1H), 2.17-2.03 (m, 1H), 1.95-1.82 (m, 1H), 1.79-1.60 (m, 3H), 1.57-1.45 (m, 1H), 1.45-1.27 (m, 4H), 0.89 (t, 3H).

LC/MS (Method 1, ESIpos): Rₜ=1.23 min, m/z=464 [M+H]⁺.

### Example 9

### (−)-(1RS,2SR,5RS)-2-[(4-Oxo-1,2,3-benzotriazin-3(4H)-yl)methyl]-5-[4-(pentyloxy)benzoyl]cyclopentanecarboxylic acid (enantiomer 2)

Yield: 6 mg; chem. purity=100%; ee=100%
[α]_{D}²⁰°=−51.9°, 589 nm, c=0.09 g/100 ml, chloroform

¹H-NMR (400 MHz, DMSO-d₆): δ [ppm]=12.12 (br. s, 1H), 8.26 (d, 1H), 8.20 (d, 1H), 8.08 (t, 1H), 7.99-7.88 (m, 3H), 7.03 (d, 2H), 4.59-4.46 (m, 2H), 4.15-4.01 (m, 3H), 3.23 (t, 1H), 2.94-2.79 (m, 1H), 2.18-2.03 (m, 1H), 1.95-1.82 (m, 1H), 1.78-1.61 (m, 3H), 1.57-1.45 (m, 1H), 1.44-1.28 (m, 4H), 0.89 (t, 3H).

LC/MS (Method 1, ESIpos): Rₜ=1.23 min, m/z=464 [M+H]⁺.

### Example 10

### (+/−)-(1RS,2RS,5SR)-2-(4-Butoxybenzoyl)-5-[(4-oxo-1,2,3-benzotriazin-3 (4H)-yl)methyl]cyclopentanecarboxylic acid (racemate)

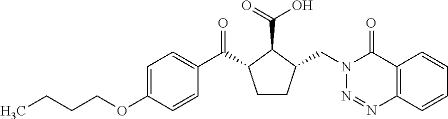 [SMILES: CCCCOc1ccc(C(=O)[C@H]2CC[C@@H](Cn3nnc4ccccc4c3=O)[C@@H]2C(=O)O)cc1]

To a solution of 32 mg (0.06 mmol) of the compound from Example 12A in 0.23 ml of dichloromethane was added 0.11 ml (1.45 mmol) of trifluoroacetic acid. The mixture was stirred at RT for 5.5 h. Thereafter, the mixture was concentrated, the residue was taken up in dichloromethane and the solution was concentrated again. This procedure was repeated several times. In this way, 26 mg (100% of theory, 100% purity) of the title compound were obtained.

¹H-NMR (400 MHz, DMSO-d₆): δ [ppm]=12.14 (br. s, 1H), 8.26 (d, 1H), 8.20 (d, 1H), 8.08 (td, 1H), 7.99-7.89 (m, 3H), 7.03 (d, 2H), 4.53 (dd, 2H), 4.14-4.03 (m, 3H), 3.24 (t, 1H), 2.94-2.80 (m, 1H), 2.17-2.04 (m, 1H), 1.95-1.82 (m, 1H), 1.77-1.60 (m, 3H), 1.57-1.37 (m, 3H), 0.93 (t, 3H).

LC/MS (Method 1, ESIpos): Rₜ=1.19 min, m/z=450 [M+H]⁺.

### Example 11

### (+/−)-(1RS,2RS,5SR)-2-[4-(Benzyloxy)benzoyl]-5-{[4-oxo-6-(trifluoromethyl)-1,2,3-benzotriazin-3(4H)-yl]methyl}cyclopentanecarboxylic acid (racemate)

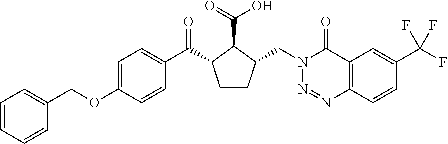 [SMILES: O=C(O)[C@H]1[C@H](Cn2nnc3ccc(C(F)(F)F)cc3c2=O)CC[C@@H]1C(=O)c1ccc(OCc2ccccc2)cc1]

To a solution of 82 mg (0.11 mmol, 85% purity) of the compound from Example 13A in 0.5 ml of dichloromethane was added, at 0° C., 0.25 ml (3.24 mmol) of trifluoroacetic acid. The mixture was stirred at 0° C. for 4.5 h and then concentrated. The residue was taken up in acetonitrile and purified by means of preparative HPLC (method 4). 42 mg (70% of theory, 98% purity) of the title compound were obtained.

¹H-NMR (400 MHz, DMSO-d₆): δ [ppm]=12.14 (br. s, 1H), 8.51 (s, 1H), 8.46-8.37 (m, 2H), 7.98 (d, 2H), 7.49-7.44 (m, 2H), 7.44-7.37 (m, 2H), 7.37-7.31 (m, 1H), 7.13 (d, 2H), 5.21 (s, 2H), 4.57 (d, 2H), 4.11 (dd, 1H), 3.24 (t, 1H), 2.95-2.79 (m, 1H), 2.17-2.05 (m, 1H), 1.99-1.87 (m, 1H), 1.72-1.61 (m, 1H), 1.59-1.45 (m, 1H).

LC/MS (Method 1, ESIpos): Rₜ=1.29 min, m/z=552 [M+H]⁺.

### Example 12

### (+/−)-(1RS,2RS,5SR)-2-{4-[(4-Fluorobenzyl)oxy]benzoyl}-5-{[4-oxo-6-(trifluoromethyl)-1,2,3-benzotriazin-3(4H)-yl]methyl}cyclopentanecarboxylic acid (racemate)

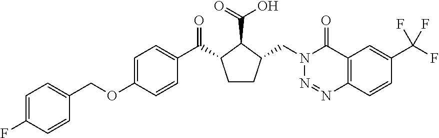 [SMILES: O=C(O)[C@H]1[C@H](Cn2nnc3ccc(C(F)(F)F)cc3c2=O)CC[C@@H]1C(=O)c1ccc(OCc2ccc(F)cc2)cc1]

To a solution of 189 mg (0.28 mmol) of the compound from Example 15A in 1.6 ml of dichloromethane was added, at 0° C., 0.8 ml (10.46 mmol) of trifluoroacetic acid. The mixture was stirred at 0° C. for 2.5 h and then concentrated. The residue was taken up in 2 ml of acetonitrile and purified by means of preparative HPLC (Method 4). 140 mg (87% of theory, 100% purity) of the title compound were obtained.

¹H-NMR (400 MHz, DMSO-d₆): δ [ppm]=12.14 (s, 1H), 8.51 (s, 1H), 8.46-8.37 (m, 2H), 7.98 (d, 2H), 7.52 (dd, 2H), 7.23 (t, 2H), 7.12 (d, 2H), 5.19 (s, 2H), 4.57 (d, 2H), 4.11 (dd, 1H), 3.24 (t, 1H), 2.94-2.79 (m, 1H), 2.17-2.04 (m, 1H), 2.00-1.87 (m, 1H), 1.73-1.61 (m, 1H), 1.59-1.45 (m, 1H).

LC/MS (Method 1, ESIpos): Rₜ=1.25 min, m/z=570 [M+H]⁺.

### Separation of the Enantiomers:

131 mg of the racemic compound from Example 12 were dissolved in 6 ml of ethanol and separated into the enantiomers by means of preparative SFC on a chiral phase (see Examples 13 and 14) [column: Daicel Chiralpak AY-H, 5 μm, 250 mm×20 mm; flow rate: 80 ml/min; detection: 220 nm; injection volume: 0.75 ml; temperature: 40° C.; eluent: 70% carbon dioxide/30% ethanol; total run time 10 min].

### Example 13

### (+)-(1RS,2RS,5SR)-2-{4-[(4-Fluorobenzyl)oxy]benzoyl}-5-{[4-oxo-6-(trifluoromethyl)-1,2,3-benzotriazin-3(4H)-yl]methyl}cyclopentanecarboxylic acid (enantiomer 1)

Yield: 47 mg; ee=100%
[α]_{D}²⁰=+79.1°, 589 nm, c=0.32 g/100 ml, chloroform

¹H-NMR (400 MHz, DMSO-d₆): δ [ppm]=12.13 (br. s, 1H), 8.51 (s, 1H), 8.46-8.38 (m, 2H), 7.98 (d, 2H), 7.52 (dd, 2H), 7.23 (t, 2H), 7.12 (d, 2H), 5.19 (s, 2H), 4.57 (d, 2H), 4.11 (dd, 1H), 3.23 (t, 1H), 2.94-2.79 (m, 1H), 2.17-2.04 (m, 1H), 1.99-1.86 (m, 1H), 1.73-1.61 (m, 1H), 1.59-1.46 (m, 1H).

LC/MS (Method 1, ESIpos): Rₜ=1.26 min, m/z=570 [M+H]⁺.

### Example 14

### (−)-(1RS,2RS,5SR)-2-{4-[(4-Fluorobenzyl)oxy]benzoyl}-5-{[4-oxo-6-(trifluoromethyl)-1,2,3-benzotriazin-3(4H)-yl]methyl}cyclopentanecarboxylic acid (enantiomer 2)

Yield: 49 mg; ee=100%
[α]_{D}²⁰=−81.2°, 589 nm, c=0.38 g/100 ml, chloroform

¹H-NMR (400 MHz, DMSO-d₆): δ [ppm]=12.14 (br. s, 1H), 8.51 (s, 1H), 8.46-8.37 (m, 2H), 7.98 (d, 2H), 7.52 (dd, 2H), 7.23 (t, 2H), 7.12 (d, 2H), 5.19 (s, 2H), 4.57 (d, 2H), 4.11 (dd, 1H), 3.24 (t, 1H), 2.94-2.78 (m, 1H), 2.17-2.04 (m, 1H), 2.02-1.86 (m, 1H), 1.74-1.60 (m, 1H), 1.59-1.45 (m, 1H).

LC/MS (Method 1, ESIpos): Rₜ=1.26 min, m/z=570 [M+H]⁺.

### Example 15

### (+/−)-(1RS,2RS,5SR)-2-{4-[(3-Methylbenzyl)oxy]benzoyl}-5-{[4-oxo-6-(trifluoromethyl)-1,2,3-benzotriazin-3(4H)-yl]methyl}cyclopentanecarboxylic acid (racemate)

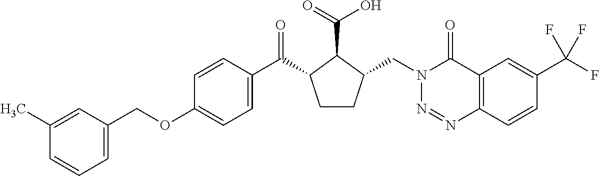 [SMILES: Cc1cccc(COc2ccc(C(=O)[C@H]3CC[C@@H](Cn4nnc5ccc(C(F)(F)F)cc5c4=O)[C@@H]3C(=O)O)cc2)c1]

To a solution of 182 mg (0.27 mmol) of the compound from Example 16A in 3 ml of dichloromethane was added, at 0° C., 0.78 ml (10.12 mmol) of trifluoroacetic acid. The mixture was stirred at 0° C. for 1.5 h and then concentrated. The residue was taken up in 3 ml of acetonitrile and purified by means of preparative HPLC (Method 4). 110 mg (71% of theory, 100% purity) of the title compound were obtained.

¹H-NMR (400 MHz, DMSO-d₆): δ [ppm]=12.13 (br. s, 1H), 8.51 (s, 1H), 8.47-8.36 (m, 2H), 7.97 (d, 2H), 7.32-7.21 (m, 3H), 7.18-7.08 (m, 3H), 5.16 (s, 2H), 4.57 (d, 2H), 4.15-4.04 (m, 1H), 3.24 (t, 1H), 2.94-2.80 (m, 1H), 2.32 (s, 3H), 2.18-2.05 (m, 1H), 1.99-1.87 (m, 1H), 1.72-1.61 (m, 1H), 1.59-1.43 (m, 1H).

LC/MS (Method 1, ESIpos): Rₜ=1.34 min, m/z=566 [M+H]⁺.

### Separation of the Enantiomers:

101 mg of the racemic compound from Example 15 were dissolved in 4 ml of ethanol and separated into the enantiomers by means of preparative SFC on a chiral phase (see Examples 16 and 17) [column: Daicel Chiralpak AZ-H, 5 μm, 250 mm×20 mm; flow rate: 80 ml/min; detection: 220 nm; injection volume: 0.75 ml; temperature: 35° C.; eluent: 65% carbon dioxide/35% ethanol; total run time 9 min].

### Example 16

### (+)-(1RS,2RS,5SR)-2-{4-[(3-Methylbenzyl)oxy]benzoyl}-5-{[4-oxo-6-(trifluoromethyl)-1,2,3-benzotriazin-3(4H)-yl]methyl}cyclopentanecarboxylic acid (enantiomer 1)

Yield: 43 mg; ee=100%
[α]_{D}²⁰=+75.5°, 589 nm, c=0.38 g/100 ml, chloroform

¹H-NMR (400 MHz, DMSO-d₆): δ [ppm]=12.14 (br. s, 1H), 8.51 (s, 1H), 8.46-8.38 (m, 2H), 7.97 (d, 2H), 7.31-7.22 (m, 3H), 7.18-7.09 (m, 3H), 5.16 (s, 2H), 4.57 (d, 2H), 4.15-4.06 (m, 1H), 3.24 (t, 1H), 2.93-2.81 (m, 1H), 2.32 (s, 3H), 2.17-2.05 (m, 1H), 1.99-1.87 (m, 1H), 1.72-1.61 (m, 1H), 1.58-1.46 (m, 1H).

LC/MS (Method 1, ESIpos): Rₜ=1.29 min, m/z=566 [M+H]⁺.

### Example 17

### (−)-(1RS,2RS,5SR)-2-{4-[(3-Methylbenzyl)oxy]benzoyl}-5-{[4-oxo-6-(trifluoromethyl)-1,2,3-benzotriazin-3(4H)-yl]methyl}cyclopentanecarboxylic acid (enantiomer 2)

Yield: 44 mg; ee=100%
[α]_{D}²⁰=−94.1°, 589 nm, c=0.36 g/100 ml, chloroform

¹H-NMR (400 MHz, DMSO-d₆): δ [ppm]=12.14 (br. s, 1H), 8.51 (s, 1H), 8.46-8.38 (m, 2H), 7.97 (d, 2H), 7.32-7.22 (m, 3H), 7.18-7.09 (m, 3H), 5.16 (s, 2H), 4.57 (d, 2H), 4.15-4.05 (m, 1H), 3.24 (t, 1H), 2.93-2.80 (m, 1H), 2.32 (s, 3H), 2.17-2.05 (m, 1H), 2.00-1.87 (m, 1H), 1.73-1.60 (m, 1H), 1.58-1.46 (m, 1H).

LC/MS (Method 1, ESIpos): Rₜ=1.29 min, m/z=566 [M+H]⁺.

### Example 18

### (+/−)-(1RS,2SR,5RS)-2-{[4-Oxo-6-(trifluoromethyl)-1,2,3-benzotriazin-3(4H)-yl]methyl}-5-(4-{[3-(trifluoromethyl)benzyl]oxy}benzoyl)cyclopentanecarboxylic acid (racemate)

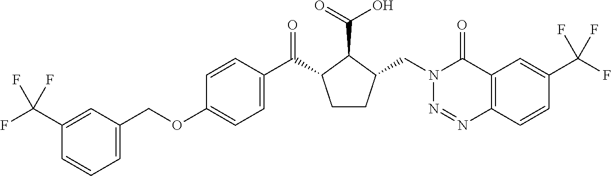 [SMILES: O=C(O)[C@H]1[C@H](Cn2nnc3ccc(C(F)(F)F)cc3c2=O)CC[C@@H]1C(=O)c1ccc(OCc2cccc(C(F)(F)F)c2)cc1]

To a solution of 180 mg (0.25 mmol) of the compound from Example 17A in 1.4 ml of dichloromethane was added, at 0° C., 0.71 ml (9.23 mmol) of trifluoroacetic acid. The mixture was stirred at 0° C. for 1.5 h and then concentrated. The residue was taken up in 3 ml of acetonitrile and purified by means of preparative HPLC (Method 4). 128 mg (83% of theory, 100% purity) of the title compound were obtained.

¹H-NMR (400 MHz, DMSO-d₆): δ [ppm]=12.14 (br. s, 1H), 8.51 (s, 1H), 8.46-8.37 (m, 2H), 7.99 (d, 2H), 7.84 (s, 1H), 7.79 (d, 1H), 7.75-7.70 (m, 1H), 7.68-7.61 (m, 1H), 7.15 (d, 2H), 5.32 (s, 2H), 4.57 (d, 2H), 4.16-4.06 (m, 1H), 3.24 (t, 1H), 2.95-2.80 (m, 1H), 2.18-2.05 (m, 1H), 1.99-1.87 (m, 1H), 1.72-1.61 (m, 1H), 1.58-1.44 (m, 1H).

LC/MS (Method 1, ESIpos): Rₜ=1.35 min, m/z=620 [M+H]⁺.

### Separation of the Enantiomers:

120 mg of the racemic compound from Example 18 were dissolved in 4 ml of ethanol and separated into the enantiomers by means of preparative SFC on a chiral phase (see Examples 19 and 20) [column: Daicel Chiralpak AY-H, 5 μm, 250 mm×20 mm; flow rate: 220 ml/min; detection: 220 nm; injection volume: 0.50 ml; temperature: 35° C.; eluent: 70% carbon dioxide/30% ethanol; total run time 6 min].

### Example 19

### (+)-(1RS,2SR,5RS)-2-{[4-Oxo-6-(trifluoromethyl)-1,2,3-benzotriazin-3(4H)-yl]methyl}-5-(4-{[3-(trifluoromethyl)benzyl]oxy}benzoyl)cyclopentanecarboxylic acid (enantiomer 1)

Yield: 52 mg; ee=100%
[α]_{D}²⁰=+73.1°, 589 nm, c=0.37 g/100 ml, chloroform

¹H-NMR (400 MHz, DMSO-d₆): δ [ppm]=12.14 (br. s, 1H), 8.51 (s, 1H), 8.46-8.37 (m, 2H), 7.99 (d, 2H), 7.84 (s, 1H), 7.79 (d, 1H), 7.75-7.69 (m, 1H), 7.68-7.61 (m, 1H), 7.15 (d, 2H), 5.32 (s, 2H), 4.57 (d, 2H), 4.16-4.05 (m, 1H), 3.24 (t, 1H), 2.93-2.80 (m, 1H), 2.17-2.06 (m, 1H), 2.00-1.87 (m, 1H), 1.73-1.61 (m, 1H), 1.59-1.45 (m, 1H).

LC/MS (Method 1, ESIpos): Rₜ=1.30 min, m/z=620 [M+H]⁺.

### Example 20

### (−)-(1RS,2SR,5RS)-2-{[4-Oxo-6-(trifluoromethyl)-1,2,3-benzotriazin-3(4H)-yl]methyl}-5-(4-{[3-(trifluoromethyl)benzyl]oxy}benzoyl)cyclopentanecarboxylic acid (enantiomer 2)

Yield: 52 mg; ee=100%
[α]_{D}²⁰=−77.5°, 589 nm, c=0.37 g/100 ml, chloroform

¹H-NMR (400 MHz, DMSO-d₆): δ [ppm]=12.14 (br. s, 1H), 8.51 (s, 1H), 8.46-8.38 (m, 2H), 7.99 (d, 2H), 7.84 (s, 1H), 7.79 (d, 1H), 7.75-7.70 (m, 1H), 7.68-7.63 (m, 1H), 7.15 (d, 2H), 5.32 (s, 2H), 4.57 (d, 2H), 4.16-4.07 (m, 1H), 3.24 (t, 1H), 2.93-2.82 (m, 1H), 2.18-2.05 (m, 1H), 2.02-1.87 (m, 1H), 1.73-1.61 (m, 1H), 1.59-1.46 (m, 1H).

LC/MS (Method 1, ESIpos): Rₜ=1.30 min, m/z=620 [M+H]⁺.

### Example 21

(+/−)-(1RS,2RS,5SR)-2-{4-[(3-Chlorobenzyl)oxy]benzoyl}-5-{[4-oxo-6-(trifluoromethyl)-1,2,3-benzotriazin-3(4H)-yl]methyl}cyclopentanecarboxylic acid (racemate)

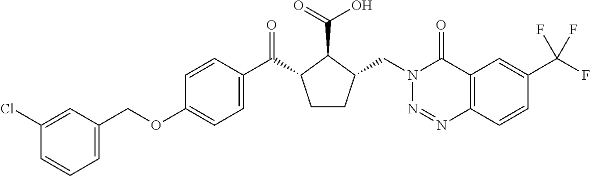 [SMILES: O=C(O)[C@H]1[C@H](Cn2nnc3ccc(C(F)(F)F)cc3c2=O)CC[C@@H]1C(=O)c1ccc(OCc2cccc(Cl)c2)cc1]

To a solution of 108 mg (0.16 mmol) of the compound from Example 18A in 0.5 ml of dichloromethane was added, at 0° C., 0.25 ml of trifluoroacetic acid. The mixture was stirred at 0° C. for 1.5 h and then concentrated. The residue was purified by means of preparative HPLC (Method 4). 71 mg (77% of theory, 100% purity) of the title compound were obtained.

¹H-NMR (400 MHz, DMSO-d₆): δ [ppm]=12.14 (s, 1H), 8.51 (s, 1H), 8.46-8.38 (m, 2H), 7.98 (d, 2H), 7.54 (s, 1H), 7.47-7.39 (m, 3H), 7.13 (d, 2H), 5.23 (s, 2H), 4.57 (d, 2H), 4.16-4.07 (m, 1H), 3.24 (t, 1H), 2.93-2.81 (m, 1H), 2.17-2.05 (m, 1H), 1.99-1.87 (m, 1H), 1.72-1.62 (m, 1H), 1.58-1.46 (m, 1H).

LC/MS (Method 1, ESIpos): Rₜ=1.31 min, m/z=586 [M+H]⁺.

### Separation of the Enantiomers:

62 mg of the racemic compound from Example 21 were dissolved in 6 ml of ethanol and separated into the enantiomers by means of preparative SFC on a chiral phase (see Examples 22 and 23) [column: Daicel Chiralpak IC, 5 μm, 250 mm×20 mm; flow rate: 80 ml/min; detection: 220 nm; injection volume: 0.6 ml; temperature: 40° C.; eluent: 70% carbon dioxide/30% ethanol; total run time 15 min].

### Example 22

### (−)-(1RS,2RS,5SR)-2-{4-[(3-Chlorobenzyl)oxy]benzoyl}-5-{[4-oxo-6-(trifluoromethyl)-1,2,3-benzotriazin-3(4H)-yl]methyl}cyclopentanecarboxylic acid (enantiomer 1)

Yield: 26 mg; ee=100%
[α]_{D}²⁰=−88.5°, 589 nm, c=0.39 g/100 ml, chloroform

¹H-NMR (400 MHz, DMSO-d₆): δ [ppm]=12.14 (s, 1H), 8.51 (s, 1H), 8.46-8.38 (m, 2H), 7.98 (d, 2H), 7.54 (s, 1H), 7.48-7.37 (m, 3H), 7.13 (d, 2H), 5.23 (s, 2H), 4.57 (d, 2H), 4.15-4.05 (m, 1H), 3.24 (t, 1H), 2.94-2.80 (m, 1H), 2.17-2.04 (m, 1H), 1.99-1.87 (m, 1H), 1.72-1.62 (m, 1H), 1.58-1.44 (m, 1H).

LC/MS (Method 1, ESIpos): Rₜ=1.31 min, m/z=586 [M+H]⁺.

### Example 23

### (+)-(1RS,2RS,5SR)-2-{4-[(3-Chlorobenzyl)oxy]benzoyl}-5-{[4-oxo-6-(trifluoromethyl)-1,2,3-benzotriazin-3(4H)-yl]methyl}cyclopentanecarboxylic acid (enantiomer 2)

Yield: 25 mg; ee=97%
[α]_{D}²⁰=+80.0°, 589 nm, c=0.37 g/100 ml, chloroform

¹H-NMR (400 MHz, DMSO-d₆): δ [ppm]=12.14 (br. s, 1H), 8.51 (s, 1H), 8.46-8.37 (m, 2H), 7.98 (d, 2H), 7.54 (s, 1H), 7.47-7.37 (m, 3H), 7.13 (d, 2H), 5.23 (s, 2H), 4.57 (d, 2H), 4.16-4.05 (m, 1H), 3.24 (t, 1H), 2.94-2.79 (m, 1H), 2.17-2.05 (m, 1H), 1.99-1.87 (m, 1H), 1.72-1.61 (m, 1H), 1.58-1.46 (m, 1H).

LC/MS (Method 1, ESIpos): Rₜ=1.31 min, m/z=586 [M+H]⁺.

### Example 24

### (+/−)-(1RS,2RS,5SR)-2-{4-[(4-Chlorobenzyl)oxy]benzoyl}-5-{[4-oxo-6-(trifluoromethyl)-1,2,3-benzotriazin-3(4H)-yl]methyl}cyclopentanecarboxylic acid (racemate)

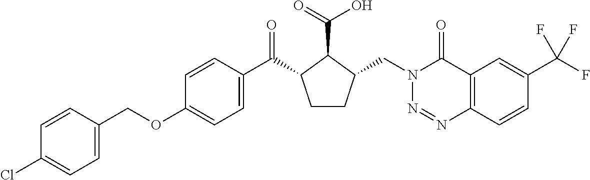 [SMILES: O=C(O)[C@H]1[C@H](Cn2nnc3ccc(C(F)(F)F)cc3c2=O)CC[C@@H]1C(=O)c1ccc(OCc2ccc(Cl)cc2)cc1]

To a solution of 111.1 mg (0.16 mmol) of the compound from Example 19A in 0.9 ml of dichloromethane was added, at 0° C., 0.46 ml (5.99 mmol) of trifluoroacetic acid. The mixture was stirred at 0° C. for 2.5 h and then concentrated. The residue was taken up in 2 ml of acetonitrile and purified by means of preparative HPLC (Method 4). 72 mg (75% of theory, 100% purity) of the title compound were obtained.

¹H-NMR (400 MHz, DMSO-d₆): δ [ppm]=12.14 (s, 1H), 8.51 (s, 1H), 8.46-8.38 (m, 2H), 7.98 (d, 2H), 7.51-7.44 (m, 4H), 7.12 (d, 2H), 5.21 (s, 2H), 4.57 (d, 2H), 4.14-4.07 (m, 1H), 3.24 (t, 1H), 2.93-2.79 (m, 1H), 2.17-2.05 (m, 1H), 1.98-1.88 (m, 1H), 1.72-1.61 (m, 1H), 1.58-1.46 (m, 1H).

LC/MS (Method 1, ESIpos): Rₜ=1.30 min, m/z=586 [M+H]⁺.

### Separation of the Enantiomers:

63 mg of the racemic compound from Example 24 were dissolved in 3 ml of acetonitrile/ethanol (2:1) and separated into the enantiomers by means of preparative HPLC on a chiral phase (see Examples 25 and 26) [column: Daicel Chiralpak AD-H, 5 μm, 250 mm×20 mm; flow rate: 20 ml/min; detection: 220 nm; injection volume: 0.25 ml; temperature: 20° C.; eluent: t=0-8 min 30% acetonitrile/70% ethanol+0.2% acetic acid].

### Example 25

### (+)-(1RS,2RS,5SR)-2-{4-[(4-Chlorobenzyl)oxy]benzoyl}-5-{[4-oxo-6-(trifluoromethyl)-1,2,3-benzotriazin-3(4H)-yl]methyl}cyclopentanecarboxylic acid (enantiomer 1)

Yield: 22 mg; ee=100%
[α]_{D}²⁰=+80.0°, 589 nm, c=0.28 g/100 ml, chloroform

¹H-NMR (400 MHz, DMSO-d₆): δ [ppm]=12.14 (br. s, 1H), 8.51 (s, 1H), 8.46-8.37 (m, 2H), 7.98 (d, 2H), 7.52-7.43 (m, 4H), 7.12 (d, 2H), 5.21 (s, 2H), 4.57 (d, 2H), 4.14-4.06 (m, 1H), 3.23 (t, 1H), 2.93-2.80 (m, 1H), 2.17-2.05 (m, 1H), 1.99-1.86 (m, 1H), 1.72-1.61 (m, 1H), 1.58-1.46 (m, 1H).

LC/MS (Method 1, ESIpos): Rₜ=1.35 min, m/z=586 [M+H]⁺.

### Example 26

### (−)-(1RS,2RS,5SR)-2-{4-[(4-Chlorobenzyl)oxy]benzoyl}-5-{[4-oxo-6-(trifluoromethyl)-1,2,3-benzotriazin-3(4H)-yl]methyl}cyclopentanecarboxylic acid (enantiomer 2)

Yield: 27 mg; ee=97%

[α]_{D}²⁰=−85.5°, 589 nm, c=0.22 g/100 ml, chloroform

¹H-NMR (400 MHz, DMSO-d₆): δ [ppm]=12.14 (br. s, 1H), 8.51 (s, 1H), 8.46-8.37 (m, 2H), 7.98 (d, 2H), 7.52-7.43 (m, 4H), 7.12 (d, 2H), 5.21 (s, 2H), 4.57 (d, 2H), 4.15-4.06 (m, 1H), 3.23 (t, 1H), 2.93-2.79 (m, 1H), 2.17-2.05 (m, 1H), 1.99-1.86 (m, 1H), 1.72-1.61 (m, 1H), 1.58-1.45 (m, 1H).

LC/MS (Method 1, ESIpos): Rₜ=1.35 min, m/z=586 [M+H]⁺.

### Example 27

### (+/−)-(1RS,2RS,5SR)-2-{4-[(3,4-Dichlorobenzyl)oxy]benzoyl}-5-{[4-oxo-6-(trifluoromethyl)-1,2,3-benzotriazin-3(4H)-yl]methyl}cyclopentanecarboxylic acid (racemate)

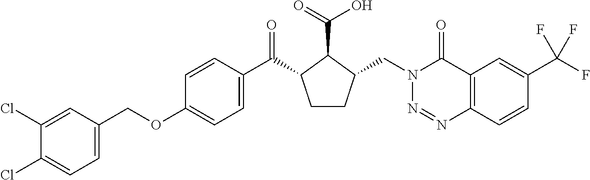 [SMILES: O=C(O)[C@H]1[C@H](Cn2nnc3ccc(C(F)(F)F)cc3c2=O)CC[C@@H]1C(=O)c1ccc(OCc2ccc(Cl)c(Cl)c2)cc1]

To a solution of 218 mg (0.30 mmol) of the compound from Example 20A in 1.7 ml of dichloromethane was added, at 0° C., 0.9 ml (11.20 mmol) of trifluoroacetic acid. The mixture was stirred at 0° C. for 2.5 h and then concentrated. The residue was taken up in 2 ml of acetonitrile and purified by means of preparative HPLC (Method 4). 170 mg (91% of theory, 100% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.99 (d, 2H), 7.75 (d, 1H), 7.68 (d, 1H), 7.46 (dd, 1H), 7.13 (d, 2H), 5.23 (s, 2H), 4.57 (d, 2H), 4.15-4.07 (m, 1H), 3.24 (t, 1H), 2.94-2.80 (m, 1H), 2.17-2.04 (m, 1H), 2.00-1.88 (m, 1H), 1.72-1.60 (m, 1H), 1.58-1.45 (m, 1H).

LC/MS (Method 1, ESIpos): Rₜ=1.35 min, m/z=520 [M+H]⁺.

### Separation of the Enantiomers:

162 mg of the racemic compound from Example 27 were dissolved in 5 ml of methanol and separated into the enantiomers by means of preparative SFC on a chiral phase (see Examples 28 and 29) [column: Daicel Chiralpak AY-H, 5 μm, 250 mm×20 mm; flow rate: 80 ml/min; detection: 220 nm; injection volume: 0.75 ml; temperature: 40° C.; eluent: 70% carbon dioxide/30% ethanol; total run time 10 min].

### Example 28

### (+)-(1RS,2RS,5SR)-2-{4-[(3,4-Dichlorobenzyl)oxy]benzoyl}-5-{[4-oxo-6-(trifluoromethyl)-1,2,3-benzotriazin-3(4H)-yl]methyl}cyclopentanecarboxylic acid (enantiomer 1)

Yield: 72 mg; ee=100%
[α]_{D}²⁰=+77.4°, 589 nm, c=0.37 g/100 ml, chloroform

¹H-NMR (400 MHz, DMSO-d₆): δ [ppm]=12.14 (br. s, 1H), 8.51 (s, 1H), 8.46-8.38 (m, 2H), 7.99 (d, 2H), 7.75 (d, 1H), 7.68 (d, 1H), 7.46 (dd, 1H), 7.13 (d, 2H), 5.23 (s, 2H), 4.57 (d, 2H), 4.15-4.06 (m, 1H), 3.24 (t, 1H), 2.93-2.80 (m, 1H), 2.18-2.04 (m, 1H), 2.01-1.87 (m, 1H), 1.73-1.61 (m, 1H), 1.59-1.45 (m, 1H).

LC/MS (Method 1, ESIpos): Rₜ=1.36 min, m/z=520 [M+H]⁺.

### Example 29

### (−)-(1RS,2RS,5SR)-2-{4-[(3,4-Dichlorobenzyl)oxy]benzoyl}-5-{[4-oxo-6-(trifluoromethyl)-1,2,3-benzotriazin-3(4H)-yl]methyl}cyclopentanecarboxylic acid (enantiomer 2)

Yield: 73 mg; ee=100%
[α]_{D}²⁰=−80.8°, 589 nm, c=0.36 g/100 ml, chloroform

¹H-NMR (400 MHz, DMSO-d₆): δ [ppm]=12.14 (br. s, 1H), 8.51 (s, 1H), 8.46-8.38 (m, 2H), 7.99 (d, 2H), 7.75 (d, 1H), 7.67 (d, 1H), 7.46 (dd, 1H), 7.13 (d, 2H), 5.23 (s, 2H), 4.57 (d, 2H), 4.16-4.06 (m, 1H), 3.24 (t, 1H), 2.94-2.80 (m, 1H), 2.19-2.04 (m, 1H), 1.99-1.87 (m, 1H), 1.73-1.61 (m, 1H), 1.58-1.45 (m, 1H).

LC/MS (Method 1, ESIpos): Rₜ=1.35 min, m/z=520 [M+H]⁺.

### Example 30

### (+/−)-(1RS,2RS,5SR)-2-{4-({3-[(Methoxycarbonyl)amino]benzyl}oxy)benzoyl}-5-{[4-oxo-6-(trifluoromethyl)-1,2,3-benzotriazin-3(4H)-yl]methyl}cyclopentanecarboxylic acid (racemate)

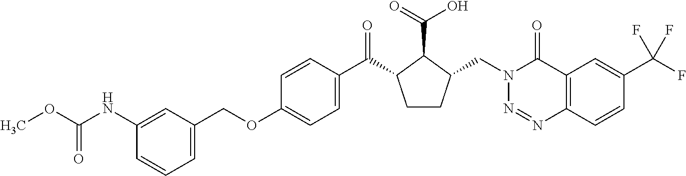 [SMILES: COC(=O)Nc1cccc(COc2ccc(C(=O)[C@H]3CC[C@@H](Cn4nnc5ccc(C(F)(F)F)cc5c4=O)[C@@H]3C(=O)O)cc2)c1]

To a solution of 185 mg (0.26 mmol) of the compound from Example 21A in 1.5 ml of dichloromethane was added, at 0° C., 0.73 ml (9.44 mmol) of trifluoroacetic acid. The mixture was stirred at 0° C. for 1.5 h and then concentrated. The residue was taken up in 2 ml of acetonitrile and purified by means of preparative HPLC (Method 4). 113 mg (71% of theory, 100% purity) of the title compound were obtained.

¹H-NMR (400 MHz, DMSO-d₆): δ [ppm]=12.13 (br. s, 1H), 9.71 (s, 1H), 8.51 (s, 1H), 8.46-8.38 (m, 2H), 7.97 (d, 2H), 7.57 (s, 1H), 7.42 (d, 1H), 7.30 (t, 1H), 7.14-7.05 (m, 3H), 5.17 (s, 2H), 4.57 (d, 2H), 4.15-4.06 (m, 1H), 3.66 (s, 3H), 3.24 (t, 1H), 2.94-2.81 (m, 1H), 2.18-2.05 (m, 1H), 1.99-1.87 (m, 1H), 1.73-1.61 (m, 1H), 1.58-1.45 (m, 1H).

LC/MS (Method 1, ESIpos): Rₜ=1.19 min, m/z=625 [M+H]⁺.

### Separation of the Enantiomers:

104 mg of the racemic compound from Example 30 were dissolved in 5 ml of ethanol and separated into the enantiomers by means of preparative HPLC on a chiral phase (see Examples 31 and 32) [column: Daicel Chiralpak AY-H, 5 μm, 250 mm×20 mm; flow rate: 12 ml/min; detection: 220 nm; injection volume: 1 ml; temperature: 45° C.; eluent: t=0-15 min 100% ethanol+0.2% acetic acid].

### Example 31

### (+)-(1RS,2RS,5SR)-2-[4-({3-[(Methoxycarbonyl)amino]benzyl}oxy)benzoyl]-5-{[4-oxo-6-(trifluoromethyl)-1,2,3-benzotriazin-3(4H)-yl]methyl}cyclopentanecarboxylic acid (enantiomer 1)

Yield: 39 mg; ee=99%
[α]_{D}²⁰=+74.0°, 589 nm, c=0.38 g/100 ml, chloroform

¹H-NMR (400 MHz, DMSO-d₆): δ [ppm]=12.13 (br. s, 1H), 9.70 (s, 1H), 8.51 (s, 1H), 8.45-8.38 (m, 2H), 7.97 (d, 2H), 7.56 (s, 1H), 7.42 (d, 1H), 7.30 (t, 1H), 7.14-7.05 (m, 3H), 5.17 (s, 2H), 4.57 (d, 2H), 4.15-4.06 (m, 1H), 3.66 (s, 3H), 3.23 (t, 1H), 2.93-2.81 (m, 1H), 2.16-2.05 (m, 1H), 1.99-1.87 (m, 1H), 1.73-1.61 (m, 1H), 1.59-1.45 (m, 1H).

LC/MS (Method 1, ESIpos): Rₜ=1.19 min, m/z=625 [M+H]⁺.

### Example 32

### (−)-(1RS,2RS,5SR)-2-[4-({3-[(Methoxycarbonyl)amino]benzyl}oxy)benzoyl]-5-{[4-oxo-6-(trifluoromethyl)-1,2,3-benzotriazin-3(4H)-yl]methyl}cyclopentanecarboxylic acid (enantiomer 2)

Yield: 35 mg; ee=99%
[α]_{D}²⁰=−75.3°, 589 nm, c=0.32 g/100 ml, chloroform

¹H-NMR (400 MHz, DMSO-d₆): δ [ppm]=12.13 (br. s, 1H), 9.70 (s, 1H), 8.51 (s, 1H), 8.46-8.37 (m, 2H), 7.97 (d, 2H), 7.56 (s, 1H), 7.42 (d, 1H), 7.30 (t, 1H), 7.14-7.05 (m, 3H), 5.17 (s, 2H), 4.57 (d, 2H), 4.15-4.06 (m, 1H), 3.66 (s, 3H), 3.23 (t, 1H), 2.94-2.79 (m, 1H), 2.17-2.04 (m, 1H), 1.99-1.87 (m, 1H), 1.73-1.61 (m, 1H), 1.58-1.44 (m, 1H).

LC/MS (Method 1, ESIpos): Rₜ=1.19 min, m/z=625 [M+H]⁺.

### Example 33

### (+/−)-(1RS,2RS,5SR)-2-(4-{[3-(2-Oxo-1,3-oxazolidin-3-yl)benzyl]oxy}benzoyl)-5-{[4-oxo-6-(trifluoromethyl)-1,2,3-benzotriazin-3 (4H)-yl]methyl}cyclopentanecarboxylic acid (racemate)

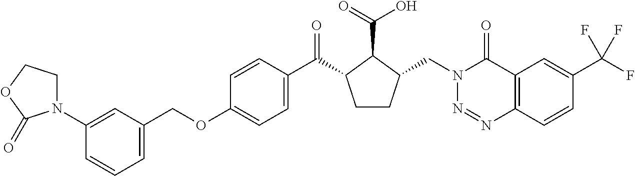 [SMILES: O=C(O)[C@H]1[C@H](Cn2nnc3ccc(C(F)(F)F)cc3c2=O)CC[C@@H]1C(=O)c1ccc(OCc2cccc(N3CCOC3=O)c2)cc1]

To a solution of 157 mg (0.21 mmol) of the compound from Example 22A in 1.2 ml of dichloromethane was added, at 0° C., 0.6 ml (7.87 mmol) of trifluoroacetic acid. The mixture was stirred at 0° C. for 1.5 h and then concentrated. The residue was taken up in 2 ml of acetonitrile and purified by means of preparative HPLC (Method 4). 117 mg (87% of theory, 100% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.98 (d, 2H), 7.70 (s, 1H), 7.53 (d, 1H), 7.42 (t, 1H), 7.22 (d, 1H), 7.13 (d, 2H), 5.23 (s, 2H), 4.57 (d, 2H), 4.44 (t, 2H), 4.15-4.02 (m, 3H), 3.24 (t, 1H), 2.94-2.80 (m, 1H), 2.17-2.05 (m, 1H), 1.99-1.87 (m, 1H), 1.73-1.61 (m, 1H), 1.59-1.45 (m, 1H).

LC/MS (Method 1, ESIpos): Rₜ=1.15 min, m/z=637 [M+H]⁺.

### Separation of the Enantiomers:

108 mg of the racemic compound from Example 33 were dissolved in 2 ml of acetonitrile/4 ml of ethanol and separated into the enantiomers by means of preparative HPLC on a chiral phase (see Examples 34 and 35) [column: Daicel Chiralpak IC, 5 μm, 250 mm×20 mm; flow rate: 12 ml/min; detection: 220 nm; injection volume: 1.5 ml; temperature: 45° C.; eluent: t=0-25 min 100% ethanol+0.2% acetic acid].

### Example 34

### (−)-(1RS,2RS,5SR)-2-(4-{[3-(2-Oxo-1,3-oxazolidin-3-yl)benzyl]oxy}benzoyl)-5-{[4-oxo-6-(trifluoromethyl)-1,2,3-benzotriazin-3(4H)-yl]methyl}cyclopentanecarboxylic acid (enantiomer 1)

Yield: 46 mg; ee=99%
[α]_{D}²⁰=−70.9°, 589 nm, c=0.42 g/100 ml, chloroform

¹H-NMR (400 MHz, DMSO-d₆): δ [ppm]=12.14 (br. s, 1H), 8.51 (s, 1H), 8.46-8.37 (m, 2H), 7.98 (d, 2H), 7.70 (s, 1H), 7.53 (dd, 1H), 7.42 (t, 1H), 7.22 (d, 1H), 7.12 (d, 2H), 5.22 (s, 2H), 4.57 (d, 2H), 4.44 (t, 2H), 4.15-4.02 (m, 3H), 3.23 (t, 1H), 2.93-2.80 (m, 1H), 2.17-2.03 (m, 1H), 1.99-1.86 (m, 1H), 1.72-1.61 (m, 1H), 1.59-1.46 (m, 1H).

LC/MS (Method 1, ESIpos): Rₜ=1.16 min, m/z=637 [M+H]⁺.

### Example 35

### (+)-(1RS,2RS,5SR)-2-(4-{[3-(2-Oxo-1,3-oxazolidin-3-yl)benzyl]oxy}benzoyl)-5-{[4-oxo-6-(trifluoromethyl)-1,2,3-benzotriazin-3(4H)-yl]methyl}cyclopentanecarboxylic acid (enantiomer 2)

Yield: 50 mg; ee=99%
[α]_{D}²⁰=+70.3°, 589 nm, c=0.39 g/100 ml, chloroform

¹H-NMR (400 MHz, DMSO-d₆): δ [ppm]=12.14 (br. s, 1H), 8.51 (s, 1H), 8.46-8.37 (m, 2H), 7.98 (d, 2H), 7.70 (s, 1H), 7.53 (dd, 1H), 7.42 (t, 1H), 7.22 (d, 1H), 7.12 (d, 2H), 5.22 (s, 2H), 4.57 (d, 2H), 4.47-4.40 (m, 2H), 4.14-4.02 (m, 3H), 3.22 (t, 1H), 2.93-2.80 (m, 1H), 2.17-2.04 (m, 1H), 1.98-1.86 (m, 1H), 1.72-1.61 (m, 1H), 1.58-1.45 (m, 1H).

LC/MS (Method 1, ESIpos): Rₜ=1.16 min, m/z=637 [M+H]⁺.

### Example 36

### (+/−)-(1RS,2SR,5RS)-2-{[4-Oxo-6-(trifluoromethyl)-1,2,3-benzotriazin-3(4H)-yl]methyl}-5-[4-(2-phenylethoxy)benzoyl]cyclopentanecarboxylic acid (racemate)

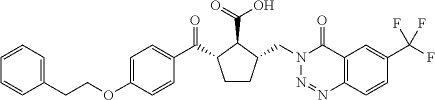 [SMILES: O=C(O)[C@H]1[C@H](Cn2nnc3ccc(C(F)(F)F)cc3c2=O)CC[C@@H]1C(=O)c1ccc(OCCc2ccccc2)cc1]

To a solution of 236 mg (0.35 mmol) of the compound from Example 23A in 2 ml of dichloromethane was added, at 0° C., 1 ml (13.12 mmol) of trifluoroacetic acid. The mixture was stirred at 0° C. for 2 h and then concentrated. The residue was taken up in 3 ml of acetonitrile and purified by means of preparative HPLC (Method 4). 150 mg (75% of theory, 100% purity) of the title compound were obtained.

¹H-NMR (400 MHz, DMSO-d₆): δ [ppm]=12.13 (br. s, 1H), 8.50 (s, 1H), 8.46-8.38 (m, 2H), 7.95 (d, 2H), 7.36-7.27 (m, 4H), 7.26-7.19 (m, 1H), 7.05 (d, 2H), 4.57 (d, 2H), 4.29 (t, 2H), 4.14-4.05 (m, 1H), 3.23 (t, 1H), 3.06 (t, 2H), 2.93-2.81 (m, 1H), 2.16-2.05 (m, 1H), 1.99-1.87 (m, 1H), 1.72-1.60 (m, 1H), 1.58-1.46 (m, 1H).

LC/MS (Method 1, ESIpos): Rₜ=1.33 min, m/z=566 [M+H]⁺.

### Separation of the Enantiomers:

142 mg of the racemic compound from Example 36 were dissolved in 3 ml of ethanol and separated into the enantiomers by means of preparative HPLC on a chiral phase (see Examples 37 and 38) [column: Daicel Chiralpak AD-H, 5 μm, 250 mm×20 mm; flow rate: 20 ml/min; detection: 230 nm; injection volume: 0.05 ml; temperature: 25° C.; eluent: t=0-12 min 86% acetonitrile/10% ethanol/4% of 5% acetic acid in acetonitrile].

### Example 37

### (+)-(1RS,2SR,5RS)-2-{[4-Oxo-6-(trifluoromethyl)-1,2,3-benzotriazin-3(4H)-yl]methyl}-5-[4-(2-phenylethoxy)benzoyl]cyclopentanecarboxylic acid (enantiomer 1)

Yield: 43 mg; ee=100%
[α]_{D}²⁰=+77.5°, 589 nm, c=0.39 g/100 ml, chloroform

¹H-NMR (400 MHz, DMSO-d₆): δ [ppm]=12.13 (br. s, 1H), 8.50 (s, 1H), 8.46-8.38 (m, 2H), 7.95 (d, 2H), 7.36-7.27 (m, 4H), 7.26-7.19 (m, 1H), 7.05 (d, 2H), 4.56 (d, 2H), 4.29 (t, 2H), 4.14-4.05 (m, 1H), 3.23 (t, 1H), 3.06 (t, 2H), 2.93-2.80 (m, 1H), 2.17-2.04 (m, 1H), 2.00-1.87 (m, 1H), 1.73-1.61 (m, 1H), 1.58-1.45 (m, 1H).

LC/MS (Method 1, ESIpos): Rₜ=1.28 min, m/z=566 [M+H]⁺.

### Example 38

### (−)-(1RS,2SR,5RS)-2-{[4-Oxo-6-(trifluoromethyl)-1,2,3-benzotriazin-3(4H)-yl]methyl}-5-[4-(2-phenylethoxy)benzoyl]cyclopentanecarboxylic acid (enantiomer 2)

Yield: 34 mg; ee=99%
[α]_{D}²⁰=−84.3°, 589 nm, c=0.40 g/100 ml, chloroform

¹H-NMR (400 MHz, DMSO-d₆): δ [ppm]=12.13 (br. s, 1H), 8.50 (s, 1H), 8.46-8.37 (m, 2H), 7.95 (d, 2H), 7.36-7.28 (m, 4H), 7.26-7.19 (m, 1H), 7.05 (d, 2H), 4.56 (d, 2H), 4.29 (t, 2H), 4.15-4.04 (m, 1H), 3.23 (t, 1H), 3.06 (t, 2H), 2.93-2.80 (m, 1H), 2.17-2.04 (m, 1H), 2.00-1.87 (m, 1H), 1.72-1.60 (m, 1H), 1.58-1.43 (m, 1H).

LC/MS (Method 1, ESIpos): Rₜ=1.28 min, m/z=566 [M+H]⁺.

### Example 39

### (+/−)-(1RS,2SR,5RS)-2-{[4-Oxo-6-(trifluoromethyl)-1,2,3-benzotriazin-3(4H)-yl]methyl}-5-[4-(3-phenylpropoxy)benzoyl]cyclopentanecarboxylic acid (racemate)

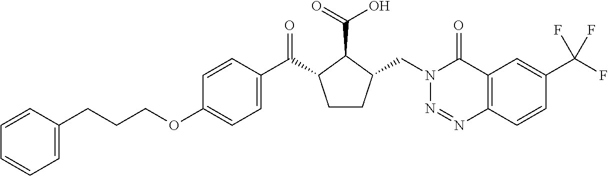 [SMILES: O=C(O)[C@H]1[C@H](Cn2nnc3ccc(C(F)(F)F)cc3c2=O)CC[C@@H]1C(=O)c1ccc(OCCCc2ccccc2)cc1]

To a solution of 37 mg (0.08 mmol) of the compound from Example 24A in 0.17 ml of dichloromethane was added, at 0° C., 0.09 ml of trifluoroacetic acid. The mixture was stirred first at 0° C. for 15 min, then at RT for 1 h, and then concentrated. The residue was purified by means of preparative HPLC (Method 4). 18 mg (58% of theory, 100% purity) of the title compound were obtained.

¹H-NMR (400 MHz, DMSO-d₆): δ [ppm]=12.13 (s, 1H), 8.51 (s, 1H), 8.47-8.37 (m, 2H), 7.96 (d, 2H), 7.32-7.14 (m, 5H), 7.04 (d, 2H), 4.57 (d, 2H), 4.14-4.02 (m, 3H), 3.24 (t, 1H), 2.94-2.81 (m, 1H), 2.75 (t, 2H), 2.18-1.99 (m, 3H), 1.99-1.88 (m, 1H), 1.73-1.61 (m, 1H), 1.59-1.45 (m, 1H).

LC/MS (Method 1, ESIpos): Rₜ=1.33 min, m/z=580 [M+H]⁺.

### Example 40

### (+/−)-(1RS,2RS,5SR)-2-[4-(Cyclohexylmethoxy)benzoyl]-5-{[4-oxo-6-(trifluoromethyl)-1,2,3-benzotriazin-3(4H)-yl]methyl}cyclopentanecarboxylic acid (racemate)

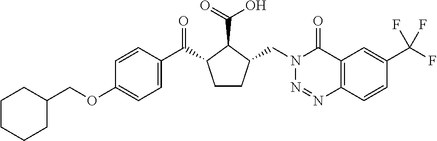 [SMILES: O=C(O)[C@H]1[C@H](Cn2nnc3ccc(C(F)(F)F)cc3c2=O)CC[C@@H]1C(=O)c1ccc(OCC2CCCCC2)cc1]

To a solution of 175 mg (0.27 mmol) of the compound from Example 25A in 0.9 ml of dichloromethane was added, at 0° C., 0.45 ml (5.84 mmol) of trifluoroacetic acid. The mixture was stirred at 0° C. for 2.5 h and then concentrated. The residue was taken up in 2 ml of acetonitrile. A solid precipitated out, which was filtered off and dried under reduced pressure. 97 mg (65% of theory, 99% purity) of the title compound were obtained.

¹H-NMR (400 MHz, DMSO-d₆): δ [ppm]=12.13 (s, 1H), 8.51 (s, 1H), 8.46-8.38 (m, 2H), 7.95 (d, 2H), 7.03 (d, 2H), 4.57 (d, 2H), 4.14-4.04 (m, 1H), 3.87 (d, 2H), 3.24 (t, 1H), 2.93-2.80 (m, 1H), 2.18-2.04 (m, 1H), 1.99-1.87 (m, 1H), 1.85-1.60 (m, 7H), 1.58-1.46 (m, 1H), 1.32-1.11 (m, 3H), 1.11-0.97 (m, 2H).

LC/MS (Method 1, ESIpos): Rₜ=1.40 min, m/z=558 [M+H]⁺.

### Separation of the Enantiomers:

89 mg of the racemic compound from Example 40 were dissolved in 4 ml of ethanol while heating and separated into the enantiomers by means of preparative HPLC on a chiral phase (see Examples 41 and 42) [column: Daicel Chiralpak AY-H, 5 μm, 250 mm×20 mm; flow rate: 15 ml/min; detection: 220 nm; injection volume: 1.4 ml; temperature: 45° C.; eluent: t=0-12 min 25% isohexane/75% ethanol+0.2% acetic acid].

### Example 41

### (+)-(1RS,2RS,5SR)-2-[4-(Cyclohexylmethoxy)benzoyl]-5-{[4-oxo-6-(trifluoromethyl)-1,2,3-benzotriazin-3(4H)-yl]methyl}cyclopentanecarboxylic acid (enantiomer 1)

Yield: 38 mg; ee=100%
[α]_{D}²⁰=+93.0°, 589 nm, c=0.11 g/100 ml, chloroform

¹H-NMR (400 MHz, DMSO-d₆): δ [ppm]=12.14 (br. s, 1H), 8.51 (s, 1H), 8.46-8.37 (m, 2H), 7.95 (d, 2H), 7.03 (d, 2H), 4.57 (d, 2H), 4.14-4.05 (m, 1H), 3.87 (d, 2H), 3.23 (t, 1H), 2.93-2.80 (m, 1H), 2.17-2.03 (m, 1H), 2.00-1.87 (m, 1H), 1.84-1.60 (m, 7H), 1.58-1.46 (m, 1H), 1.32-1.12 (m, 3H), 1.11-0.97 (m, 2H).

LC/MS (Method 1, ESIpos): Rₜ=1.45 min, m/z=558 [M+H]⁺.

### Example 42

### (−)-(1RS,2RS,5SR)-2-[4-(Cyclohexylmethoxy)benzoyl]-5-{[4-oxo-6-(trifluoromethyl)-1,2,3-benzotriazin-3(4H)-yl]methyl}cyclopentanecarboxylic acid (enantiomer 2)

Yield: 39 mg; ee=100%
[α]_{D}²⁰=−86.6°, 589 nm, c=0.38 g/100 ml, chloroform

¹H-NMR (400 MHz, DMSO-d₆): δ [ppm]=12.14 (br. s, 1H), 8.51 (s, 1H), 8.46-8.38 (m, 2H), 7.95 (d, 2H), 7.03 (d, 2H), 4.57 (d, 2H), 4.14-4.05 (m, 1H), 3.87 (d, 2H), 3.23 (t, 1H), 2.93-2.79 (m, 1H), 2.17-2.04 (m, 1H), 1.99-1.86 (m, 1H), 1.85-1.60 (m, 7H), 1.58-1.44 (m, 1H), 1.22 (d, 3H), 1.11-0.97 (m, 2H).

LC/MS (Method 1, ESIpos): Rₜ=1.45 min, m/z=558 [M+H]⁺.

### Example 43

### (+/−)-(1RS,2RS,5SR)-2-[4-(Cyclopentylmethoxy)benzoyl]-5-{[4-oxo-6-(trifluoromethyl)-1,2,3-benzotriazin-3(4H)-yl]methyl}cyclopentanecarboxylic acid (racemate)

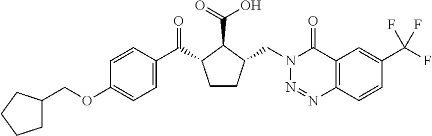 [SMILES: O=C(O)[C@H]1[C@H](Cn2nnc3ccc(C(F)(F)F)cc3c2=O)CC[C@@H]1C(=O)c1ccc(OCC2CCCC2)cc1]

To a solution of 206 mg (0.31 mmol) of the compound from Example 26A in 1 ml of dichloromethane was added, at 0° C., 0.5 ml (6.88 mmol) of trifluoroacetic acid. The mixture was stirred first at 0° C. for 15 min, then at RT for 1 h, and then concentrated. The residue was purified by means of preparative HPLC (Method 4). 173 mg (>100% of theory, ˜100% purity, still containing solvent) of the title compound were obtained.

¹H-NMR (400 MHz, DMSO-d₆): δ [ppm]=12.13 (br. s, 1H), 8.51 (s, 1H), 8.46-8.37 (m, 2H), 7.95 (d, 2H), 7.03 (d, 2H), 4.57 (d, 2H), 4.15-4.05 (m, 1H), 3.94 (d, 2H), 3.24 (t, 1H), 2.93-2.80 (m, 1H), 2.39-2.23 (m, 1H), 2.18-2.04 (m, 1H), 2.00-1.87 (m, 1H), 1.83-1.72 (m, 2H), 1.72-1.46 (m, 6H), 1.38-1.26 (m, 2H).

LC/MS (Method 1, ESIpos): Rₜ=1.34 min, m/z=544 [M+H]⁺.

### Separation of the Enantiomers:

166 mg of the racemic compound from Example 43 were dissolved in 6 ml of acetonitrile/ethanol (1:1) and separated into the enantiomers by means of preparative SFC on a chiral phase (see Examples 44 and 45) [column: Daicel Chiralpak AD SFC, 5 μm, 250 mm×20 mm; flow rate: 100 ml/min; detection: 220 nm; injection volume: 0.2 ml; temperature: 30° C.; eluent: 70% carbon dioxide/30% ethanol; total run time 11 min].

### Example 44

### (+)-(1RS,2RS,5SR)-2-[4-(Cyclopentylmethoxy)benzoyl]-5-{[4-oxo-6-(trifluoromethyl)-1,2,3-benzotriazin-3(4H)-yl]methyl}cyclopentanecarboxylic acid (enantiomer 1)

Yield: 76 mg; ee=100%
[α]_{D}²⁰=+85.1°, 589 nm, c=0.41 g/100 ml, chloroform

¹H-NMR (400 MHz, DMSO-d₆): δ [ppm]=12.13 (br. s, 1H), 8.51 (s, 1H), 8.46-8.37 (m, 2H), 7.95 (d, 2H), 7.03 (d, 2H), 4.57 (d, 2H), 4.14-4.06 (m, 1H), 3.94 (d, 2H), 3.24 (t, 1H), 2.94-2.80 (m, 1H), 2.38-2.25 (m, 1H), 2.16-2.04 (m, 1H), 2.00-1.87 (m, 1H), 1.83-1.72 (m, 2H), 1.72-1.45 (m, 6H), 1.38-1.27 (m, 2H).

LC/MS (Method 1, ESIpos): Rₜ=1.34 min, m/z=544 [M+H]⁺.

### Example 45

### (−)-(1RS,2RS,5SR)-2-[4-(Cyclopentylmethoxy)benzoyl]-5-{[4-oxo-6-(trifluoromethyl)-1,2,3-benzotriazin-3(4H)-yl]methyl}cyclopentanecarboxylic acid (enantiomer 2)

Yield: 76 mg; ee=100%
[α]_{D}²⁰=−84.5°, 589 nm, c=0.39 g/100 ml, chloroform

¹H-NMR (400 MHz, DMSO-d₆): δ [ppm]=12.13 (br. s, 1H), 8.51 (s, 1H), 8.46-8.37 (m, 2H), 7.95 (d, 2H), 7.03 (d, 2H), 4.57 (d, 2H), 4.15-4.04 (m, 1H), 3.94 (d, 2H), 3.23 (t, 1H), 2.95-2.80 (m, 1H), 2.39-2.24 (m, 1H), 2.17-2.04 (m, 1H), 2.00-1.86 (m, 1H), 1.82-1.73 (m, 2H), 1.72-1.46 (m, 6H), 1.38-1.27 (m, 2H).

LC/MS (Method 1, ESIpos): Rₜ=1.34 min, m/z=544 [M+H]⁺.

### Example 46

### (+/−)-(1RS,2RS,5SR)-2-[4-(Cyclobutylmethoxy)benzoyl]-5-{[4-oxo-6-(trifluoromethyl)-1,2,3-benzotriazin-3(4H)-yl]methyl}cyclopentanecarboxylic acid (racemate)

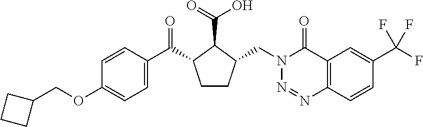 [SMILES: O=C(O)[C@H]1[C@H](Cn2nnc3ccc(C(F)(F)F)cc3c2=O)CC[C@@H]1C(=O)c1ccc(OCC2CCC2)cc1]

To a solution of 141 mg (0.22 mmol) of the compound from Example 27A in 1.3 ml of dichloromethane was added, at 0° C., 0.64 ml (8.26 mmol) of trifluoroacetic acid. The mixture was stirred at 0° C. for 1.5 h and then concentrated. The residue was taken up in 3 ml of acetonitrile and purified by means of preparative HPLC (Method 5). 93 mg (78% of theory, 100% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.95 (d, 2H), 7.03 (d, 2H), 4.57 (d, 2H), 4.14-4.06 (m, 1H), 4.04 (d, 2H), 3.24 (t, 1H), 2.93-2.81 (m, 1H), 2.79-2.66 (m, 1H), 2.17-2.02 (m, 3H), 1.99-1.77 (m, 5H), 1.72-1.61 (m, 1H), 1.58-1.46 (m, 1H).

LC/MS (Method 1, ESIpos): Rₜ=1.34 min, m/z=530 [M+H]⁺.

### Separation of the Enantiomers:

83 mg of the racemic compound from Example 46 were dissolved in 4 ml of ethanol and separated into the enantiomers by means of preparative SFC on a chiral phase (see Examples 47 and 48) [column: Daicel Chiralpak AD-H, 5 μm, 250 mm×20 mm; flow rate: 80 ml/min; detection: 220 nm; injection volume: 0.75 ml; temperature: 40° C.; eluent: 65% carbon dioxide/35% ethanol; total run time 8 min].

### Example 47

### (+)-(1RS,2RS,5SR)-2-[4-(Cyclobutylmethoxy)benzoyl]-5-{[4-oxo-6-(trifluoromethyl)-1,2,3-benzotriazin-3(4H)-yl]methyl}cyclopentanecarboxylic acid (enantiomer 1)

Yield: 35 mg; ee=100%
[α]_{D}²⁰=+86.7°, 589 nm, c=0.32 g/100 ml, chloroform

¹H-NMR (400 MHz, DMSO-d₆): δ [ppm]=12.14 (br. s, 1H), 8.51 (s, 1H), 8.46-8.36 (m, 2H), 7.95 (d, 2H), 7.03 (d, 2H), 4.57 (d, 2H), 4.14-4.06 (m, 1H), 4.04 (d, 2H), 3.23 (t, 1H), 2.93-2.81 (m, 1H), 2.80-2.67 (m, 1H), 2.16-2.01 (m, 3H), 1.99-1.76 (m, 5H), 1.72-1.61 (m, 1H), 1.58-1.46 (m, 1H).

LC/MS (Method 1, ESIpos): Rₜ=1.29 min, m/z=530 [M+H]⁺.

### Example 48

### (−)-(1RS,2RS,5SR)-2-[4-(Cyclobutylmethoxy)benzoyl]-5-{[4-oxo-6-(trifluoromethyl)-1,2,3-benzotriazin-3(4H)-yl]methyl}cyclopentanecarboxylic acid (enantiomer 2)

Yield: 35 mg; ee=100%
[α]_{D}²⁰=−90.1°, 589 nm, c=0.33 g/100 ml, chloroform

¹H-NMR (400 MHz, DMSO-d₆): δ [ppm]=12.14 (br. s, 1H), 8.51 (s, 1H), 8.46-8.38 (m, 2H), 7.95 (d, 2H), 7.03 (d, 2H), 4.57 (d, 2H), 4.14-4.06 (m, 1H), 4.04 (d, 2H), 3.23 (t, 1H), 2.93-2.80 (m, 1H), 2.78-2.65 (m, 1H), 2.17-2.00 (m, 3H), 1.99-1.76 (m, 5H), 1.67 (d, 1H), 1.58-1.46 (m, 1H).

LC/MS (Method 1, ESIpos): Rₜ=1.29 min, m/z=530 [M+H]⁺.

### Example 49

### (+/−)-(1RS,2RS,5SR)-2-[4-(Cyclopropylmethoxy)benzoyl]-5-{[4-oxo-6-(trifluoromethyl)-1,2,3-benzotriazin-3(4H)-yl]methyl}cyclopentanecarboxylic acid (racemate)

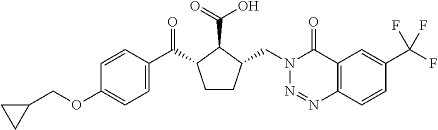 [SMILES: O=C(O)[C@H]1[C@H](Cn2nnc3ccc(C(F)(F)F)cc3c2=O)CC[C@@H]1C(=O)c1ccc(OCC2CC2)cc1]

To a solution of 150 mg (0.24 mmol) of the compound from Example 28A in 0.82 ml of dichloromethane was added, at 0° C., 0.41 ml (5.33 mmol) of trifluoroacetic acid. The mixture was stirred first at 0° C. for 15 min, then at RT for 1 h, and then concentrated. The residue was purified by means of preparative HPLC (Method 4). 79 mg (63% of theory, 100% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.95 (d, 2H), 7.02 (d, 2H), 4.57 (d, 2H), 4.14-4.05 (m, 1H), 3.91 (d, 2H), 3.23 (t, 1H), 2.93-2.80 (m, 1H), 2.17-2.04 (m, 1H), 2.00-1.86 (m, 1H), 1.73-1.61 (m, 1H), 1.58-1.46 (m, 1H), 1.29-1.19 (m, 1H), 0.61-0.54 (m, 2H), 0.37-0.30 (m, 2H).

LC/MS (Method 1, ESIpos): Rₜ=1.21 min, m/z=516 [M+H]⁺.

### Example 50

### (+/−)-(1RS,2RS,5SR)-2-[4-(2-Cyclohexylethoxy)benzoyl]-5-{[4-oxo-6-(trifluoromethyl)-1,2,3-benzotriazin-3(4H)-yl]methyl}cyclopentanecarboxylic acid (racemate)

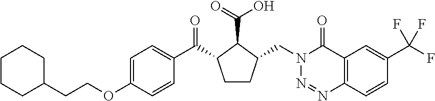 [SMILES: O=C(O)[C@H]1[C@H](Cn2nnc3ccc(C(F)(F)F)cc3c2=O)CC[C@@H]1C(=O)c1ccc(OCCC2CCCCC2)cc1]

To a solution of 121 mg (0.18 mmol) of the compound from Example 29A in 1 ml of dichloromethane was added, at 0° C., 0.5 ml (6.49 mmol) of trifluoroacetic acid. The mixture was stirred at 0° C. for 2.5 h and then concentrated. The residue was taken up in 2 ml of acetonitrile and purified by means of preparative HPLC (Method 4). 97 mg (94% of theory, 100% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.95 (d, 2H), 7.03 (d, 2H), 4.57 (d, 2H), 4.14-4.05 (m, 3H), 3.24 (t, 1H), 2.94-2.81 (m, 1H), 2.17-2.05 (m, 1H), 2.00-1.87 (m, 1H), 1.78-1.40 (m, 9H), 1.29-1.07 (m, 4H), 1.01-0.88 (m, 2H).

LC/MS (Method 1, ESIpos): Rₜ=1.43 min, m/z=572 [M+H]⁺.

### Separation of the Enantiomers:

89 mg of the racemic compound from Example 50 were dissolved in 5 ml of isopropanol and separated into the enantiomers by means of preparative HPLC on a chiral phase (see Examples 51 and 52) [column: Daicel Chiralcel OZ-H, 5 μm, 250 mm×20 mm; flow rate: 20 ml/min; detection: 230 nm; injection volume: 0.3 ml; temperature: 25° C.; eluent: t=0-20 min 50% isohexane/50% isopropanol+0.2% acetic acid].

### Example 51

### (−)-(1RS,2RS,5SR)-2-[4-(2-Cyclohexylethoxy)benzoyl]-5-{[4-oxo-6-(trifluoromethyl)-1,2,3-benzotriazin-3(4H)-yl]methyl}cyclopentanecarboxylic acid (enantiomer 1)

Yield: 38 mg; ee=100%
[α]_{D}²⁰=−76.3°, 589 nm, c=0.34 g/100 ml, chloroform

¹H-NMR (400 MHz, DMSO-d₆): δ [ppm]=12.14 (br. s, 1H), 8.51 (s, 1H), 8.46-8.38 (m, 2H), 7.95 (d, 2H), 7.03 (d, 2H), 4.57 (d, 2H), 4.14-4.05 (m, 3H), 3.23 (t, 1H), 2.94-2.80 (m, 1H), 2.17-2.04 (m, 1H), 2.00-1.86 (m, 1H), 1.78-1.40 (m, 9H), 1.31-1.07 (m, 4H), 1.04-0.86 (m, 2H).

LC/MS (Method 2, ESIpos): Rₜ=3.20 min, m/z=572 [M+H]⁺.

### Example 52

### (+)-(1RS,2RS,5SR)-2-[4-(2-Cyclohexylethoxy)benzoyl]-5-{[4-oxo-6-(trifluoromethyl)-1,2,3-benzotriazin-3(4H)-yl]methyl}cyclopentanecarboxylic acid (enantiomer 2)

Yield: 38 mg; ee=100%
[α]_{D}²⁰=+72.3°, 589 nm, c=0.39 g/100 ml, chloroform

¹H-NMR (400 MHz, DMSO-d₆): δ [ppm]=12.13 (br. s, 1H), 8.51 (s, 1H), 8.46-8.38 (m, 2H), 7.95 (d, 2H), 7.03 (d, 2H), 4.57 (d, 2H), 4.14-4.05 (m, 3H), 3.23 (t, 1H), 2.93-2.81 (m, 1H), 2.17-2.04 (m, 1H), 1.99-1.87 (m, 1H), 1.78-1.40 (m, 9H), 1.31-1.07 (m, 4H), 1.03-0.87 (m, 2H).

LC/MS (Method 2, ESIpos): Rₜ=3.20 min, m/z=572 [M+H]⁺.

### Example 53

### (+/−)-(1RS,2RS,5SR)-2-[4-(2-Cyclopentylethoxy)benzoyl]-5-{[4-oxo-6-(trifluoromethyl)-1,2,3-benzotriazin-3(4H)-yl]methyl}cyclopentanecarboxylic acid (racemate)

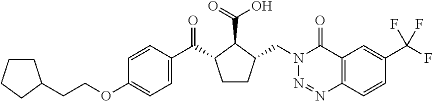 [SMILES: O=C(O)[C@H]1[C@H](Cn2nnc3ccc(C(F)(F)F)cc3c2=O)CC[C@@H]1C(=O)c1ccc(OCCC2CCCC2)cc1]

To a solution of 115.4 mg (0.18 mmol) of the compound from Example 30A in 1 ml of dichloromethane was added, at 0° C., 0.5 ml (6.49 mmol) of trifluoroacetic acid. The mixture was stirred at 0° C. for 2.5 h and then concentrated. The residue was taken up in 2 ml of acetonitrile and purified by means of preparative HPLC (Method 4). 92 mg (94% of theory, 100% purity) of the title compound were obtained.

¹H-NMR (400 MHz, DMSO-d₆): δ [ppm]=12.13 (br. s, 1H), 8.51 (s, 1H), 8.45-8.38 (m, 2H), 7.96 (d, 2H), 7.03 (d, 2H), 4.57 (d, 2H), 4.14-4.03 (m, 3H), 3.24 (t, 1H), 2.93-2.81 (m, 1H), 2.17-2.05 (m, 1H), 2.00-1.86 (m, 2H), 1.83-1.41 (m, 10H), 1.25-1.06 (m, 2H).

LC/MS (Method 1, ESIpos): Rₜ=1.39 min, m/z=558 [M+H]⁺.

### Separation of the Enantiomers:

84 mg of the racemic compound from Example 53 were dissolved in 3 ml of ethanol and separated into the enantiomers by means of preparative SFC on a chiral phase (see Examples 54 and 55) [column: Daicel Chiralpak IC, 5 μm, 250 mm×20 mm; flow rate: 80 ml/min; detection: 220 nm; injection volume: 0.25 ml; temperature: 40° C.; eluent: 60% carbon dioxide/40% ethanol; total run time 5 min].

### Example 54

### (−)-(1RS,2RS,5SR)-2-[4-(2-Cyclopentylethoxy)benzoyl]-5-{[4-oxo-6-(trifluoromethyl)-1,2,3-benzotriazin-3(4H)-yl]methyl}cyclopentanecarboxylic acid (enantiomer 1)

Yield: 30 mg; chem. purity=100%; ee=100%
[α]_{D}²⁰=−87.7°, 589 nm, c=0.37 g/100 ml, chloroform

¹H-NMR (400 MHz, DMSO-d₆): δ [ppm]=12.13 (br. s, 1H), 8.51 (s, 1H), 8.46-8.36 (m, 2H), 7.96 (d, 2H), 7.03 (d, 2H), 4.57 (d, 2H), 4.15-4.03 (m, 3H), 3.23 (t, 1H), 2.95-2.78 (m, 1H), 2.18-2.03 (m, 1H), 2.00-1.85 (m, 2H), 1.84-1.42 (m, 10H), 1.22-1.07 (m, 2H).

LC/MS (Method 2, ESIpos): Rₜ=3.11 min, m/z=558 [M+H]⁺.

### Example 55

### (+)-(1RS,2RS,5SR)-2-[4-(2-Cyclopentylethoxy)benzoyl]-5-{[4-oxo-6-(trifluoromethyl)-1,2,3-benzotriazin-3(4H)-yl]methyl}cyclopentanecarboxylic acid (enantiomer 2)

Yield: 31 mg; chem. purity=99%; ee=100%
[α]_{D}²⁰=+77.2°, 589 nm, c=0.47 g/100 ml, chloroform

¹H-NMR (400 MHz, DMSO-d₆): δ [ppm]=12.14 (br. s, 1H), 8.50 (s, 1H), 8.46-8.37 (m, 2H), 7.96 (d, 2H), 7.02 (d, 2H), 4.61-4.51 (m, 2H), 4.16-4.00 (m, 3H), 3.22-3.13 (m, 1H), 2.91-2.80 (m, 1H), 2.18-2.01 (m, 1H), 2.00-1.85 (m, 2H), 1.75 (d, 10H), 1.34-1.08 (m, 2H).

LC/MS (Method 2, ESIpos): Rₜ=3.11 min, m/z=558 [M+H]⁺.

### Example 56

### (+/−)-(1RS,2RS,5SR)-2-{4-(3-Cyclohexylpropoxy)benzoyl}-5-{[4-oxo-6-(trifluoromethyl)-1,2,3-benzotriazin-3(4H)-yl]methyl}cyclopentanecarboxylic acid (racemate)

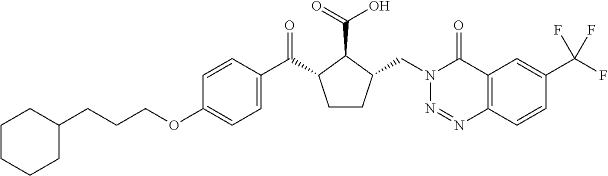 [SMILES: O=C(O)[C@H]1[C@H](Cn2nnc3ccc(C(F)(F)F)cc3c2=O)CC[C@@H]1C(=O)c1ccc(OCCCC2CCCCC2)cc1]

To a solution of 84 mg (0.08 mmol, 63% purity) of the compound from Example 31A in 0.24 ml of dichloromethane was added, at 0° C., 0.12 ml of trifluoroacetic acid. The mixture was stirred first at 0° C. for 15 min, then at RT for 1 h, and then concentrated. The residue was purified by means of preparative HPLC (Method 4). 5 mg (12% of theory, 100% purity) of the title compound were obtained.

¹H-NMR (400 MHz, DMSO-d₆): δ [ppm]=12.14 (br. s, 1H), 8.51 (s, 1H), 8.46-8.38 (m, 2H), 7.95 (d, 2H), 7.02 (d, 2H), 4.57 (d, 2H), 4.15-3.99 (m, 3H), 3.24 (t, 1H), 2.93-2.81 (m, 1H), 2.17-2.04 (m, 1H), 1.98-1.88 (m, 1H), 1.79-1.04 (m, 15H), 0.94-0.81 (m, 2H).

LC/MS (Method 1, ESIpos): Rₜ=1.52 min, m/z=586 [M+H]⁺.

### Example 57

### (+/−)-(1RS,2RS,5SR)-2-[4-(Hexyloxy)benzoyl]-5-{[4-oxo-6-(trifluoromethyl)-1,2,3-benzotriazin-3(4H)-yl]methyl}cyclopentanecarboxylic acid (racemate)

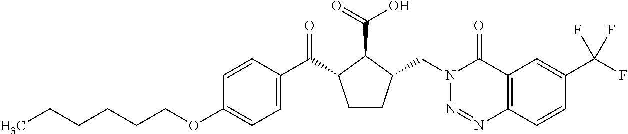 [SMILES: CCCCCCOc1ccc(C(=O)[C@H]2CC[C@@H](Cn3nnc4ccc(C(F)(F)F)cc4c3=O)[C@@H]2C(=O)O)cc1]

To a solution of 164 mg (0.25 mmol) of the compound from Example 32A in 1.5 ml of dichloromethane was added, at 0° C., 0.72 ml (9.39 mmol) of trifluoroacetic acid. The mixture was stirred at 0° C. for 2.5 h and then concentrated. The residue was taken up in 2 ml of acetonitrile and purified by means of preparative HPLC (Method 4). 112 mg (81% of theory, 100% purity) of the title compound were obtained.

¹H-NMR (400 MHz, DMSO-d₆): δ [ppm]=12.13 (s, 1H), 8.51 (s, 1H), 8.46-8.38 (m, 2H), 7.95 (d, 2H), 7.03 (d, 2H), 4.57 (d, 2H), 4.14-4.02 (m, 3H), 3.24 (t, 1H), 2.93-2.81 (m, 1H), 2.17-2.05 (m, 1H), 2.00-1.87 (m, 1H), 1.77-1.61 (m, 3H), 1.58-1.46 (m, 1H), 1.46-1.36 (m, 2H), 1.35-1.22 (m, 4H), 0.90-0.83 (m, 3H).

LC/MS (Method 1, ESIpos): Rₜ=1.37 min, m/z=546 [M+H]⁺.

### Separation of the Enantiomers:

103 mg of the racemic compound from Example 57 were dissolved in 6 ml of ethanol and separated into the enantiomers by means of preparative SFC on a chiral phase (see Examples 58 and 59) [column: Daicel Chiralpak AY-H, 5 μm, 250 mm×20 mm; flow rate: 80 ml/min; detection: 220 nm; injection volume: 0.75 ml; temperature: 40° C.; eluent: 70% carbon dioxide/30% ethanol; total run time 5 min].

### Example 58

### (+)-(1RS,2RS,5SR)-2-[4-(Hexyloxy)benzoyl]-5-{[4-oxo-6-(trifluoromethyl)-1,2,3-benzotriazin-3(4H)-yl]methyl}cyclopentanecarboxylic acid (enantiomer 1)

Yield: 48 mg; ee=100%
[α]_{D}²⁰=+83.3°, 589 nm, c=0.29 g/100 ml, chloroform

¹H-NMR (400 MHz, DMSO-d₆): δ [ppm]=12.13 (br. s, 1H), 8.51 (s, 1H), 8.46-8.37 (m, 2H), 7.95 (d, 2H), 7.03 (d, 2H), 4.57 (d, 2H), 4.14-4.01 (m, 3H), 3.23 (t, 1H), 2.94-2.80 (m, 1H), 2.17-2.02 (m, 1H), 2.00-1.87 (m, 1H), 1.78-1.60 (m, 3H), 1.58-1.47 (m, 1H), 1.46-1.36 (m, 2H), 1.35-1.25 (m, 4H), 0.90-0.83 (m, 3H).

LC/MS (Method 1, ESIpos): Rₜ=1.43 min, m/z=546 [M+H]⁺.

### Example 59

### (−)-(1RS,2RS,5SR)-2-[4-(Hexyloxy)benzoyl]-5-{[4-oxo-6-(trifluoromethyl)-1,2,3-benzotriazin-3(4H)-yl]methyl}cyclopentanecarboxylic acid (enantiomer 2)

Yield: 51 mg; ee=100%
[α]_{D}²⁰=−91.8°, 589 nm, c=0.36 g/100 ml, chloroform

¹H-NMR (400 MHz, DMSO-d₆): δ [ppm]=12.13 (br. s, 1H), 8.51 (s, 1H), 8.46-8.38 (m, 2H), 7.95 (d, 2H), 7.03 (d, 2H), 4.57 (d, 2H), 4.15-3.99 (m, 3H), 3.23 (t, 1H), 2.94-2.80 (m, 1H), 2.17-2.04 (m, 1H), 2.00-1.87 (m, 1H), 1.78-1.60 (m, 3H), 1.59-1.47 (m, 1H), 1.46-1.37 (m, 2H), 1.35-1.21 (m, 4H), 0.91-0.82 (m, 3H).

LC/MS (Method 1, ESIpos): Rₜ=1.43 min, m/z=546 [M+H]⁺.

### Example 60

### (+/−)-(1RS,2RS,5SR)-2-[4-(3-Methylbutoxy)benzoyl]-5-{[4-oxo-6-(trifluoromethyl)-1,2,3-benzotriazin-3(4H)-yl]methyl}cyclopentanecarboxylic acid (racemate)

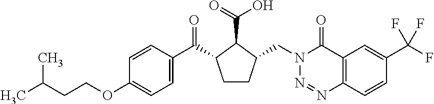 [SMILES: CC(C)CCOc1ccc(C(=O)[C@H]2CC[C@@H](Cn3nnc4ccc(C(F)(F)F)cc4c3=O)[C@@H]2C(=O)O)cc1]

To a solution of 249 mg (0.39 mmol) of the compound from Example 33A in 1.5 ml of dichloromethane was added, at 0° C., 0.75 ml (9.89 mmol) of trifluoroacetic acid. The mixture was stirred at 0° C. for 2 h and then concentrated. The residue was taken up in 2 ml of acetonitrile and purified by means of preparative HPLC (Method 4). 168 mg (73% of theory, 91% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.96 (d, 2H), 7.04 (d, 2H), 4.57 (d, 2H), 4.14-4.05 (m, 3H), 3.24 (t, 1H), 2.93-2.81 (m, 1H), 2.18-2.04 (m, 1H), 2.01-1.87 (m, 1H), 1.84-1.72 (m, 1H), 1.72-1.59 (m, 3H), 1.58-1.46 (m, 1H), 0.94 (s, 3H), 0.92 (s, 3H).

LC/MS (Method 1, ESIpos): Rₜ=1.33 min, m/z=532 [M+H]⁺.

### Separation of the Enantiomers:

164 mg of the racemic compound from Example 60 were dissolved in 6 ml of ethanol and separated into the enantiomers by means of preparative HPLC on a chiral phase (see Examples 61 and 62) [column: Daicel Chiralpak AY-H, 5 μm, 250 mm×20 mm; flow rate: 12 ml/min; detection: 220 nm; injection volume: 1.0 ml; temperature: 45° C.; eluent: t=0-12 min 100% ethanol+0.2% acetic acid].

### Example 61

### (+)-(1RS,2RS,5SR)-2-[4-(3-Methylbutoxy)benzoyl]-5-{[4-oxo-6-(trifluoromethyl)-1,2,3-benzotriazin-3(4H)-yl]methyl}cyclopentanecarboxylic acid (enantiomer 1)

Yield: 78 mg; chem. purity=100%; ee=99%
[α]_{D}²⁰=+85.6°, 589 nm, c=0.33 g/100 ml, chloroform

¹H-NMR (400 MHz, DMSO-d₆): δ [ppm]=12.13 (s, 1H), 8.51 (s, 1H), 8.46-8.38 (m, 2H), 7.96 (d, 2H), 7.04 (d, 2H), 4.57 (d, 2H), 4.14-4.04 (m, 3H), 3.24 (t, 1H), 2.94-2.80 (m, 1H), 2.17-2.04 (m, 1H), 2.00-1.87 (m, 1H), 1.85-1.72 (m, 1H), 1.72-1.59 (m, 3H), 1.59-1.46 (m, 1H), 0.94 (s, 3H), 0.92 (s, 3H).

LC/MS (Method 1, ESIpos): Rₜ=1.37 min, m/z=532 [M+H]⁺.

### Example 62

### (−)-(1RS,2RS,5SR)-2-[4-(3-Methylbutoxy)benzoyl]-5-{[4-oxo-6-(trifluoromethyl)-1,2,3-benzotriazin-3(4H)-yl]methyl}cyclopentanecarboxylic acid (enantiomer 2)

Yield: 79 mg; chem. purity=100%; ee=99%
[α]_{D}²⁰=−92.8°, 589 nm, c=0.35 g/100 ml, chloroform

¹H-NMR (400 MHz, DMSO-d₆): δ [ppm]=12.13 (s, 1H), 8.51 (s, 1H), 8.46-8.38 (m, 2H), 7.96 (d, 2H), 7.04 (d, 2H), 4.57 (d, 2H), 4.14-4.05 (m, 3H), 3.23 (t, 1H), 2.93-2.80 (m, 1H), 2.17-2.03 (m, 1H), 1.99-1.87 (m, 1H), 1.85-1.72 (m, 1H), 1.72-1.59 (m, 3H), 1.59-1.46 (m, 1H), 0.94 (s, 3H), 0.92 (s, 3H).

LC/MS (Method 1, ESIpos): Rₜ=1.37 min, m/z=532 [M+H]⁺.

### Example 63

### (+/−)-(1RS,2SR,5RS)-2-{[4-Oxo-6-(trifluoromethyl)-1,2,3-benzotriazin-3(4H)-yl]methyl}-5-[4-(pentyloxy)benzoyl]cyclopentanecarboxylic acid (racemate)

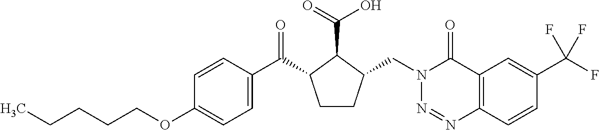 [SMILES: CCCCCOc1ccc(C(=O)[C@H]2CC[C@@H](Cn3nnc4ccc(C(F)(F)F)cc4c3=O)[C@@H]2C(=O)O)cc1]

To a solution of 177 mg (0.28 mmol) of the compound from Example 34A in 1.6 ml of dichloromethane was added, at 0° C., 0.8 ml (10.38 mmol) of trifluoroacetic acid. The mixture was stirred at 0° C. for 2.5 h and then concentrated. The residue was taken up in 2 ml of acetonitrile and purified by means of preparative HPLC (Method 4). 122 mg (82% of theory, 100% purity) of the title compound were obtained.

¹H-NMR (400 MHz, DMSO-d₆): δ [ppm]=12.13 (br. s, 1H), 8.51 (s, 1H), 8.46-8.38 (m, 2H), 7.95 (d, 2H), 7.03 (d, 2H), 4.57 (d, 2H), 4.14-4.02 (m, 3H), 3.24 (t, 1H), 2.94-2.79 (m, 1H), 2.17-2.04 (m, 1H), 1.99-1.87 (m, 1H), 1.79-1.60 (m, 3H), 1.58-1.45 (m, 1H), 1.45-1.28 (m, 4H), 0.92-0.86 (m, 3H).

LC/MS (Method 1, ESIpos): Rₜ=1.33 min, m/z=532 [M+H]⁺.

### Separation of the Enantiomers:

114 mg of the racemic compound from Example 63 were dissolved in 6 ml of ethanol and separated into the enantiomers by means of preparative SFC on a chiral phase (see Examples 64 and 65) [column: Daicel Chiralpak AY-H, 5 μm, 250 mm×20 mm; flow rate: 80 ml/min; detection: 220 nm; injection volume: 0.75 ml; temperature: 40° C.; eluent: 60% carbon dioxide/40% ethanol; total run time 8 min].

### Example 64

### (+)-(1RS,2SR,5RS)-2-{[4-Oxo-6-(trifluoromethyl)-1,2,3-benzotriazin-3(4H)-yl]methyl}-5-[4-(pentyloxy)benzoyl]cyclopentanecarboxylic acid (enantiomer 1)

Yield: 52 mg; ee=100%
[α]_{D}²⁰=+82.3°, 589 nm, c=0.35 g/100 ml, chloroform

¹H-NMR (400 MHz, DMSO-d₆): δ [ppm]=12.13 (br. s, 1H), 8.51 (s, 1H), 8.46-8.37 (m, 2H), 7.95 (d, 2H), 7.03 (d, 2H), 4.57 (d, 2H), 4.14-4.01 (m, 3H), 3.23 (t, 1H), 2.93-2.81 (m, 1H), 2.17-2.04 (m, 1H), 1.99-1.87 (m, 1H), 1.78-1.61 (m, 3H), 1.59-1.46 (m, 1H), 1.44-1.27 (m, 4H), 0.93-0.85 (m, 3H).

LC/MS (Method 1, ESIpos): Rₜ=1.32 min, m/z=532 [M+H]⁺.

### Example 65

### (−)-(1RS,2SR,5RS)-2-{[4-Oxo-6-(trifluoromethyl)-1,2,3-benzotriazin-3(4H)-yl]methyl}-5-[4-(pentyloxy)benzoyl]cyclopentanecarboxylic acid (enantiomer 2)

Yield: 51 mg; ee=100%
[α]_{D}²⁰=−75.7°, 589 nm, c=0.34 g/100 ml, chloroform

¹H-NMR (400 MHz, DMSO-d₆): δ [ppm]=12.13 (br. s, 1H), 8.51 (s, 1H), 8.46-8.37 (m, 2H), 7.95 (d, 2H), 7.03 (d, 2H), 4.57 (d, 2H), 4.15-4.00 (m, 3H), 3.23 (t, 1H), 2.95-2.77 (m, 1H), 2.17-2.03 (m, 1H), 2.00-1.85 (m, 1H), 1.79-1.60 (m, 3H), 1.59-1.46 (m, 1H), 1.45-1.27 (m, 4H), 0.93-0.84 (m, 3H).

LC/MS (Method 1, ESIpos): Rₜ=1.32 min, m/z=532 [M+H]⁺.

### Example 66

### (+/−)-(1RS,2RS,5SR)-2-(4-Butoxybenzoyl)-5-{[4-oxo-6-(trifluoromethyl)-1,2,3-benzotriazin-3 (4H)-yl]methyl}cyclopentanecarboxylic acid (racemate)

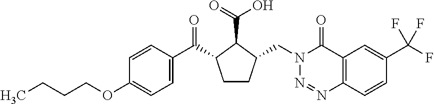 [SMILES: CCCCOc1ccc(C(=O)[C@H]2CC[C@@H](Cn3nnc4ccc(C(F)(F)F)cc4c3=O)[C@@H]2C(=O)O)cc1]

To a solution of 230 mg (0.37 mmol) of the compound from Example 35A in 2 ml of dichloromethane was added, at 0° C., 1 ml (13.79 mmol) of trifluoroacetic acid. The mixture was stirred at 0° C. for 1.5 h and then concentrated. The residue was taken up in 3 ml of acetonitrile and purified by means of preparative HPLC (Method 4). 152 mg (79% of theory, 100% purity) of the title compound were obtained.

¹H-NMR (400 MHz, DMSO-d₆): δ [ppm]=12.13 (s, 1H), 8.51 (s, 1H), 8.46-8.38 (m, 2H), 7.96 (d, 2H), 7.03 (d, 2H), 4.57 (d, 2H), 4.15-4.01 (m, 3H), 3.24 (t, 1H), 2.94-2.80 (m, 1H), 2.17-2.05 (m, 1H), 2.00-1.87 (m, 1H), 1.77-1.61 (m, 3H), 1.59-1.37 (m, 3H), 0.93 (t, 3H).

LC/MS (Method 1, ESIpos): Rₜ=1.32 min, m/z=518 [M+H]⁺.

### Separation of the Enantiomers:

142 mg of the racemic compound from Example 66 were dissolved in 4 ml of ethanol and separated into the enantiomers by means of preparative SFC on a chiral phase (see Examples 67 and 68) [column: Daicel Chiralpak AD-H, 5 μm, 250 mm×20 mm; flow rate: 80 ml/min; detection: 220 nm; injection volume: 0.75 ml; temperature: 40° C.; eluent: 70% carbon dioxide/30% ethanol; total run time 7 min].

### Example 67

### (+)-(1RS,2RS,5SR)-2-(4-Butoxybenzoyl)-5-{[4-oxo-6-(trifluoromethyl)-1,2,3-benzotriazin-3(4H)-yl]methyl}cyclopentanecarboxylic acid (enantiomer 1)

Yield: 65 mg; ee=100%
[α]_{D}²⁰=+89.3°, 589 nm, c=0.40 g/100 ml, chloroform

¹H-NMR (400 MHz, DMSO-d₆): δ [ppm]=12.14 (br. s, 1H), 8.51 (s, 1H), 8.46-8.37 (m, 2H), 7.96 (d, 2H), 7.03 (d, 2H), 4.57 (d, 2H), 4.14-4.02 (m, 3H), 3.23 (t, 1H), 2.94-2.80 (m, 1H), 2.17-2.03 (m, 1H), 2.00-1.86 (m, 1H), 1.77-1.60 (m, 3H), 1.58-1.38 (m, 3H), 0.93 (t, 3H).

LC/MS (Method 1, ESIpos): Rₜ=1.27 min, m/z=518 [M+H]⁺.

### Example 68

### (−)-(1RS,2RS,5SR)-2-(4-Butoxybenzoyl)-5-{[4-oxo-6-(trifluoromethyl)-1,2,3-benzotriazin-3(4H)-yl]methyl}cyclopentanecarboxylic acid (enantiomer 2)

Yield: 56 mg; ee=100%
[α]_{D}²⁰=−94.2°, 589 nm, c=0.37 g/100 ml, chloroform

¹H-NMR (400 MHz, DMSO-d₆): δ [ppm]=12.13 (br. s, 1H), 8.51 (s, 1H), 8.46-8.37 (m, 2H), 7.96 (d, 2H), 7.03 (d, 2H), 4.57 (d, 2H), 4.16-4.00 (m, 4H), 3.24 (t, 1H), 2.94-2.79 (m, 1H), 2.17-2.04 (m, 1H), 1.99-1.86 (m, 1H), 1.78-1.60 (m, 3H), 1.59-1.37 (m, 3H), 0.93 (t, 3H).

LC/MS (Method 1, ESIpos): Rₜ=1.27 min, m/z=518 [M+H]⁺.

### Example 69

### (+/−)-(1RS,2SR,5RS)-2-{[4-Oxo-6-(trifluoromethyl)-1,2,3-benzotriazin-3(4H)-yl]methyl}-5-(4-propoxybenzoyl)cyclopentanecarboxylic acid (racemate)

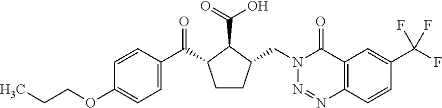 [SMILES: CCCOc1ccc(C(=O)[C@H]2CC[C@@H](Cn3nnc4ccc(C(F)(F)F)cc4c3=O)[C@@H]2C(=O)O)cc1]

To a solution of 63 mg (0.10 mmol) of the compound from Example 36A in 0.4 ml of dichloromethane was added, at 0° C., 0.2 ml (2.61 mmol) of trifluoroacetic acid. The mixture was stirred at 0° C. for 8 h. Thereafter, the mixture was concentrated, the residue was taken up in dichloromethane and the solution was concentrated again. This procedure was repeated several times. In this way, 51 mg (94% of theory, 96% purity) of the title compound were obtained.

¹H-NMR (400 MHz, DMSO-d₆): δ [ppm]=12.13 (br. s, 1H), 8.51 (s, 1H), 8.45-8.39 (m, 2H), 7.96 (d, 2H), 7.03 (d, 2H), 4.57 (d, 2H), 4.10 (dd, 1H), 4.02 (t, 2H), 3.24 (dd, 1H), 2.92-2.82 (m, 1H), 2.16-2.07 (m, 1H), 1.97-1.89 (m, 1H), 1.79-1.70 (m, 1H), 1.70-1.63 (m, 2H), 1.57-1.48 (m, 1H), 0.98 (t, 3H).

LC/MS (Method 1, ESIpos): Rₜ=1.24 min, m/z=504 [M+H]⁺.

### Separation of the Enantiomers:

33 mg of the racemic compound from Example 69 were dissolved in 4 ml of methanol/isopropanol and separated into the enantiomers by means of preparative HPLC on a chiral phase (see Examples 70 and 71) [column: Daicel Chiralpak AD-H, 5 μm, 250 mm×20 mm; flow rate: 20 ml/min; detection: 280 nm; injection volume: 1 ml; temperature: 20° C.; eluent: t=0-18 min 50% isohexane/50% isopropanol+0.2% trifluoroacetic acid].

### Example 70

### (+)-(1RS,2SR,5RS)-2-{[4-Oxo-6-(trifluoromethyl)-1,2,3-benzotriazin-3(4H)-yl]methyl}-5-(4-propoxybenzoyl)cyclopentanecarboxylic acid (enantiomer 1)

Yield: 9 mg; chem. purity=97%; ee=100%
[α]_{D}²⁰=+83.1°, 589 nm, c=0.24 g/100 ml, chloroform

¹H-NMR (400 MHz, DMSO-d₆): δ [ppm]=12.13 (br. s, 1H), 8.51 (s, 1H), 8.45-8.39 (m, 2H), 7.96 (d, 2H), 7.03 (d, 2H), 4.57 (d, 2H), 4.10 (dd, 1H), 4.02 (t, 2H), 3.24 (dd, 1H), 2.92-2.84 (m, 1H), 2.16-2.07 (m, 1H), 1.97-1.89 (m, 1H), 1.79-1.70 (m, 1H), 1.70-1.63 (m, 2H), 1.57-1.48 (m, 1H), 0.98 (t, 3H).

LC/MS (Method 1, ESIpos): Rₜ=1.25 min, m/z=504 [M+H]⁺.

### Example 71

### (−)-(1RS,2SR,5RS)-2-{[4-Oxo-6-(trifluoromethyl)-1,2,3-benzotriazin-3(4H)-yl]methyl}-5-(4-propoxybenzoyl)cyclopentanecarboxylic acid (enantiomer 2)

Yield: 9 mg; chem. purity=97%; ee=98%
[α]_{D}²⁰=−71.3°, 589 nm, c=0.28 g/100 ml, chloroform

¹H-NMR (400 MHz, DMSO-d₆): δ [ppm]=12.12 (br. s, 1H), 8.51 (s, 1H), 8.45-8.39 (m, 2H), 7.96 (d, 2H), 7.03 (d, 2H), 4.57 (d, 2H), 4.10 (dd, 1H), 4.02 (t, 2H), 3.24 (dd, 1H), 2.92-2.84 (m, 1H), 2.16-2.07 (m, 1H), 1.97-1.89 (m, 1H), 1.79-1.70 (m, 1H), 1.70-1.63 (m, 2H), 1.57-1.48 (m, 1H), 0.98 (t, 3H).

LC/MS (Method 1, ESIpos): Rₜ=1.25 min, m/z=504 [M+H]⁺.

### Example 72

### (+/−)-(1RS,2RS,5SR)-2-(4-Isobutoxybenzoyl)-5-{[4-oxo-6-(trifluoromethyl)-1,2,3-benzotriazin-3(4H)-yl]methyl}cyclopentanecarboxylic acid (racemate)

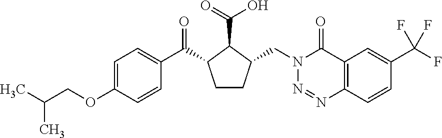 [SMILES: CC(C)COc1ccc(C(=O)[C@H]2CC[C@@H](Cn3nnc4ccc(C(F)(F)F)cc4c3=O)[C@@H]2C(=O)O)cc1]

To a solution of 92 mg (0.15 mmol) of the compound from Example 37A in 0.5 ml of dichloromethane was added, at 0° C., 0.25 ml (3.26 mmol) of trifluoroacetic acid. The mixture was stirred first at 0° C. for 15 min, then at RT for 1.5 h, and then concentrated. The residue was purified by means of preparative HPLC (Method 4). 62 mg (81% of theory, 100% purity) of the title compound were obtained.

¹H-NMR (400 MHz, DMSO-d₆): δ [ppm]=12.13 (br. s, 1H), 8.51 (s, 1H), 8.46-8.37 (m, 2H), 7.96 (d, 2H), 7.04 (d, 2H), 4.57 (d, 2H), 4.15-4.05 (m, 1H), 3.84 (d, 2H), 3.24 (t, 1H), 2.94-2.80 (m, 1H), 2.17-1.87 (m, 3H), 1.73-1.61 (m, 1H), 1.59-1.45 (m, 1H), 0.99 (s, 3H), 0.97 (s, 3H).

LC/MS (Method 1, ESIpos): Rₜ=1.28 min, m/z=518 [M+H]⁺.

### Example 73

### (+/−)-(1RS,2RS,5SR)-2-[4-(Benzylsulphanyl)benzoyl]-5-{[4-oxo-6-(trifluoromethyl)-1,2,3-benzotriazin-3(4H)-yl]methyl}cyclopentanecarboxylic acid (racemate)

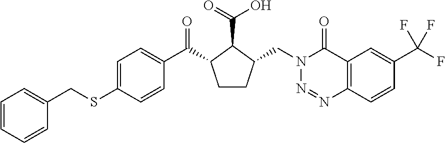 [SMILES: O=C(O)[C@H]1[C@H](Cn2nnc3ccc(C(F)(F)F)cc3c2=O)CC[C@@H]1C(=O)c1ccc(SCc2ccccc2)cc1]

To a solution of 150 mg (0.23 mmol) of the compound from Example 40A in 2 ml of dichloromethane was added, at 0° C., 1 ml of trifluoroacetic acid. The mixture was stirred first at 0° C. for 15 min, then at RT for 1.5 h, and then concentrated. The residue was purified by means of preparative HPLC (Method 4). 117 mg (92% of theory, 100% purity) of the title compound were obtained.

¹H-NMR (400 MHz, DMSO-d₆): δ [ppm]=12.15 (s, 1H), 8.50 (s, 1H), 8.46-8.37 (m, 2H), 7.89 (d, 2H), 7.47-7.39 (m, 4H), 7.35-7.29 (m, 2H), 7.28-7.21 (m, 1H), 4.59-4.54 (m, 2H), 4.37 (s, 2H), 4.14-4.04 (m, 1H), 3.23 (t, 1H), 2.94-2.79 (m, 1H), 2.17-2.04 (m, 1H), 1.99-1.87 (m, 1H), 1.72-1.60 (m, 1H), 1.58-1.45 (m, 1H).

LC/MS (Method 1, ESIpos): Rₜ=1.29 min, m/z=568 [M+H]⁺.

### Separation of the Enantiomers:

98 mg of the racemic compound from Example 73 were dissolved in 4 ml of ethanol and separated into the enantiomers by means of preparative SFC on a chiral phase (see Examples 74 and 75) [column: Daicel Chiralpak IC, 5 μm, 250 mm×20 mm; flow rate: 80 ml/min; detection: 220 nm; injection volume: 0.75 ml; temperature: 40° C.; eluent: 70% carbon dioxide/30% ethanol; total run time 10 min].

### Example 74

### (−)-(1RS,2RS,5SR)-2-[4-(Benzylsulphanyl)benzoyl]-5-{[4-oxo-6-(trifluoromethyl)-1,2,3-benzotriazin-3(4H)-yl]methyl}cyclopentanecarboxylic acid (enantiomer 1)

Yield: 43 mg; ee=100%
[α]_{D}²⁰=−87.6°, 589 nm, c=0.36 g/100 ml, chloroform

¹H-NMR (400 MHz, DMSO-d₆): δ [ppm]=12.15 (br. s, 1H), 8.50 (s, 1H), 8.46-8.37 (m, 2H), 7.89 (d, 2H), 7.46-7.40 (m, 4H), 7.35-7.29 (m, 2H), 7.28-7.21 (m, 1H), 4.59-4.53 (m, 2H), 4.37 (s, 2H), 4.14-4.05 (m, 1H), 3.23 (t, 1H), 2.94-2.79 (m, 1H), 2.17-2.02 (m, 1H), 2.00-1.87 (m, 1H), 1.72-1.59 (m, 1H), 1.58-1.45 (m, 1H).

LC/MS (Method 1, ESIpos): Rₜ=1.29 min, m/z=568 [M+H]⁺.

### Example 75

### (+)-(1RS,2RS,5SR)-2-[4-(Benzylsulphanyl)benzoyl]-5-{[4-oxo-6-(trifluoromethyl)-1,2,3-benzotriazin-3(4H)-yl]methyl}cyclopentanecarboxylic acid (enantiomer 2)

Yield: 43 mg; ee=100%
[α]_{D}²⁰=+85.2°, 589 nm, c=0.38 g/100 ml, chloroform

¹H-NMR (400 MHz, DMSO-d₆): δ [ppm]=12.15 (br. s, 1H), 8.50 (s, 1H), 8.45-8.37 (m, 2H), 7.89 (d, 2H), 7.47-7.40 (m, 4H), 7.36-7.29 (m, 2H), 7.28-7.21 (m, 1H), 4.60-4.53 (m, 2H), 4.37 (s, 2H), 4.15-4.05 (m, 1H), 3.23 (t, 1H), 2.93-2.80 (m, 1H), 2.16-2.04 (m, 1H), 1.99-1.86 (m, 1H), 1.72-1.60 (m, 1H), 1.58-1.44 (m, 1H).

LC/MS (Method 1, ESIpos): Rₜ=1.29 min, m/z=568 [M+H]⁺.

### Example 76

### (+/−)-(1RS,2RS,5SR)-2-{4-[(3-Chlorobenzyl)sulphanyl]benzoyl}-5-{[4-oxo-6-(trifluoromethyl)-1,2,3-benzotriazin-3(4H)-yl]methyl}cyclopentanecarboxylic acid (racemate)

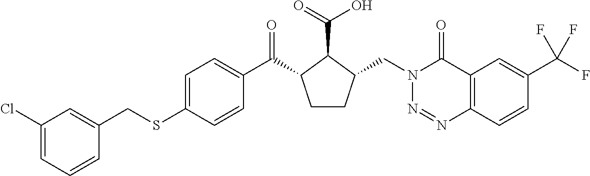 [SMILES: O=C(O)[C@H]1[C@H](Cn2nnc3ccc(C(F)(F)F)cc3c2=O)CC[C@@H]1C(=O)c1ccc(SCc2cccc(Cl)c2)cc1]

To a solution of 255 mg (0.36 mmol) of the compound from Example 41A in 1.2 ml of dichloromethane was added, at 0° C., 0.6 ml (7.98 mmol) of trifluoroacetic acid. The mixture was stirred at RT for 2 h and then concentrated. The residue was purified by means of preparative HPLC (Method 4). 182 mg (83% of theory, 100% purity) of the title compound were obtained.

¹H-NMR (400 MHz, DMSO-d₆): δ [ppm]=12.15 (s, 1H), 8.50 (s, 1H), 8.46-8.38 (m, 2H), 7.90 (d, 2H), 7.50 (s, 1H), 7.44 (d, 2H), 7.42-7.28 (m, 3H), 4.59-4.53 (m, 2H), 4.39 (s, 2H), 4.15-4.05 (m, 1H), 3.23 (t, 1H), 2.93-2.80 (m, 1H), 2.17-2.03 (m, 1H), 2.00-1.86 (m, 1H), 1.73-1.60 (m, 1H), 1.58-1.45 (m, 1H).

LC/MS (Method 1, ESIpos): Rₜ=1.38 min, m/z=602 [M+H]⁺.

### Separation of the Enantiomers:

174 mg of the racemic compound from Example 76 were dissolved in 7 ml of ethanol and separated into the enantiomers by means of preparative SFC on a chiral phase (see Examples 77 and 78) [column: Daicel Chiralpak AY-H, 5 μm, 250 mm×20 mm; flow rate: 80 ml/min; detection: 220 nm; injection volume: 0.75 ml; temperature: 40° C.; eluent: 60% carbon dioxide/40% ethanol; total run time 8 min].

### Example 77

### (+)-(1RS,2RS,5SR)-2-{4-[(3-Chlorobenzyl)sulphanyl]benzoyl}-5-{[4-oxo-6-(trifluoromethyl)-1,2,3-benzotriazin-3(4H)-yl]methyl}cyclopentanecarboxylic acid (enantiomer 1)

Yield: 80 mg; chem. purity=100%; ee=100%
[α]_{D}²⁰=+72.9°, 589 nm, c=0.36 g/100 ml, chloroform

¹H-NMR (400 MHz, DMSO-d₆): δ [ppm]=12.13 (br. s, 1H), 8.50 (s, 1H), 8.46-8.37 (m, 2H), 7.89 (d, 2H), 7.50 (s, 1H), 7.44 (d, 2H), 7.41-7.37 (m, 1H), 7.37-7.34 (m, 1H), 7.33-7.28 (m, 2H), 4.58-4.54 (m, 2H), 4.39 (s, 2H), 4.14-4.06 (m, 1H), 3.22 (t, 1H), 2.91-2.83 (m, 1H), 2.17-2.04 (m, 1H), 1.98-1.87 (m, 1H), 1.72-1.61 (m, 1H), 1.58-1.44 (m, 1H).

LC/MS (Method 1, ESIpos): Rₜ=1.33 min, m/z=602 [M+H]⁺.

### Example 78

### (−)-(1RS,2RS,5SR)-2-{4-[(3-Chlorobenzyl)sulphanyl]benzoyl}-5-{[4-oxo-6-(trifluoromethyl)-1,2,3-benzotriazin-3(4H)-yl]methyl}cyclopentanecarboxylic acid (enantiomer 2)

Yield: 80 mg; chem. purity=100%; ee=100%
[α]_{D}²⁰=−80.7°, 589 nm, c=0.39 g/100 ml, chloroform

¹H-NMR (400 MHz, DMSO-d₆): δ [ppm]=12.15 (br. s, 1H), 8.50 (s, 1H), 8.46-8.37 (m, 2H), 7.90 (d, 2H), 7.50 (s, 1H), 7.44 (d, 2H), 7.42-7.38 (m, 1H), 7.37-7.34 (m, 1H), 7.34-7.28 (m, 2H), 4.60-4.52 (m, 2H), 4.39 (s, 2H), 4.15-4.05 (m, 1H), 3.23 (t, 1H), 2.93-2.80 (m, 1H), 2.17-2.04 (m, 1H), 1.99-1.86 (m, 1H), 1.71-1.60 (m, 1H), 1.58-1.45 (m, 1H).

LC/MS (Method 1, ESIpos): Rₜ=1.33 min, m/z=602 [M+H]⁺.

### Example 79

### (+/−)-(1RS,2RS,5SR)-2-{4-[(3,4-Dichlorobenzyl)sulphanyl]benzoyl}-5-{[4-oxo-6-(trifluoromethyl)-1,2,3-benzotriazin-3(4H)-yl]methyl}cyclopentanecarboxylic acid (racemate)

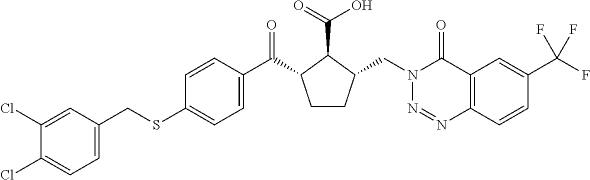 [SMILES: O=C(O)[C@H]1[C@H](Cn2nnc3ccc(C(F)(F)F)cc3c2=O)CC[C@@H]1C(=O)c1ccc(SCc2ccc(Cl)c(Cl)c2)cc1]

To a solution of 266 mg (0.36 mmol) of the compound from Example 42A in 1.2 ml of dichloromethane was added, at 0° C., 0.6 ml (7.94 mmol) of trifluoroacetic acid. The mixture was stirred at RT for 1.5 h and then concentrated. The residue was purified by means of preparative HPLC (Method 4). 204 mg (89% of theory, 100% purity) of the title compound were obtained.

¹H-NMR (400 MHz, DMSO-d₆): δ [ppm]=12.15 (br. s, 1H), 8.50 (s, 1H), 8.46-8.37 (m, 2H), 7.90 (d, 2H), 7.70 (d, 1H), 7.58 (d, 1H), 7.47-7.40 (m, 3H), 4.59-4.53 (m, 2H), 4.40 (s, 2H), 4.16-4.04 (m, 1H), 3.23 (t, 1H), 2.93-2.79 (m, 1H), 2.16-2.04 (m, 1H), 1.99-1.87 (m, 1H), 1.72-1.60 (m, 1H), 1.58-1.45 (m, 1H).

LC/MS (Method 1, ESIpos): Rₜ=1.42 min, m/z=636 [M+H]⁺.

### Separation of the Enantiomers:

195 mg of the racemic compound from Example 79 were dissolved in 7 ml of ethanol and separated into the enantiomers by means of preparative SFC on a chiral phase (see Examples 80 and 81) [column: Daicel Chiralpak AY-H, 5 μm, 250 mm×20 mm; flow rate: 80 ml/min; detection: 220 nm; injection volume: 0.15 ml; temperature: 40° C.; eluent: 60% carbon dioxide/40% ethanol; total run time 8 min].

### Example 80

### (+)-(1RS,2RS,5SR)-2-{4-[(3,4-Dichlorobenzyl)sulphanyl]benzoyl}-5-{[4-oxo-6-(trifluoromethyl)-1,2,3-benzotriazin-3(4H)-yl]methyl}cyclopentanecarboxylic acid (enantiomer 1)

Yield: 86 mg; chem. purity=100%; ee=100%
[α]_{D}²⁰=+52.9°, 589 nm, c=0.21 g/100 ml, chloroform

¹H-NMR (400 MHz, DMSO-d₆): δ [ppm]=12.15 (br. s, 1H), 8.50 (s, 1H), 8.45-8.38 (m, 2H), 7.90 (d, 2H), 7.70 (d, 1H), 7.58 (d, 1H), 7.47-7.39 (m, 3H), 4.59-4.53 (m, 2H), 4.40 (s, 2H), 4.14-4.05 (m, 1H), 3.23 (t, 1H), 2.93-2.80 (m, 1H), 2.17-2.03 (m, 1H), 2.00-1.87 (m, 1H), 1.71-1.60 (m, 1H), 1.58-1.44 (m, 1H).

LC/MS (Method 1, ESIpos): Rₜ=1.42 min, m/z=636 [M+H]⁺.

### Example 81

### (−)-(1RS,2RS,5SR)-2-{4-[(3,4-Dichlorobenzyl)sulphanyl]benzoyl}-5-{[4-oxo-6-(trifluoromethyl)-1,2,3-benzotriazin-3(4H)-yl]methyl}cyclopentanecarboxylic acid (enantiomer 2)

Yield: 92 mg; chem. purity=100%; ee=100%
[α]_{D}²⁰=−73.7°, 589 nm, c=0.35 g/100 ml, chloroform

¹H-NMR (400 MHz, DMSO-d₆): δ [ppm]=12.13 (br. s, 1H), 8.50 (s, 1H), 8.46-8.36 (m, 2H), 7.90 (d, 2H), 7.70 (d, 1H), 7.57 (d, 1H), 7.47-7.38 (m, 3H), 4.60-4.52 (m, 2H), 4.39 (s, 2H), 4.15-4.05 (m, 1H), 3.22 (t, 1H), 2.93-2.80 (m, 1H), 2.16-2.03 (m, 1H), 1.98-1.87 (m, 1H), 1.72-1.61 (m, 1H), 1.58-1.44 (m, 1H).

LC/MS (Method 1, ESIpos): Rₜ=1.38 min, m/z=636 [M+H]⁺.

### Example 82

### (+/−)-(1RS,2SR,5RS)-2-{[4-Oxo-6-(trifluoromethyl)-1,2,3-benzotriazin-3(4H)-yl]methyl}-5-{4-[(2-phenylethyl)sulphanyl]benzoyl}cyclopentanecarboxylic acid (racemate)

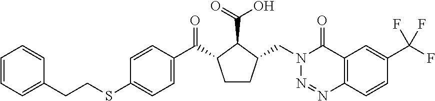 [SMILES: O=C(O)[C@H]1[C@H](Cn2nnc3ccc(C(F)(F)F)cc3c2=O)CC[C@@H]1C(=O)c1ccc(SCCc2ccccc2)cc1]

To a solution of 239 mg (0.35 mmol) of the compound from Example 43A in 1.2 ml of dichloromethane was added, at 0° C., 0.6 ml (7.69 mmol) of trifluoroacetic acid. The mixture was stirred at RT for 1.5 h and then concentrated. The residue was purified by means of preparative HPLC (Method 4). 92 mg (45% of theory, 100% purity) of the title compound were obtained.

¹H-NMR (400 MHz, DMSO-d₆): δ [ppm]=12.16 (br. s, 1H), 8.51 (s, 1H), 8.46-8.37 (m, 2H), 7.92 (d, 2H), 7.43 (d, 2H), 7.34-7.27 (m, 4H), 7.25-7.16 (m, 1H), 4.61-4.53 (m, 2H), 4.17-4.05 (m, 1H), 3.34 (t, 2H), 3.25 (t, 1H), 2.97-2.81 (m, 3H), 2.19-2.07 (m, 1H), 2.01-1.87 (m, 1H), 1.74-1.62 (m, 1H), 1.59-1.46 (m, 1H).

LC/MS (Method 1, ESIpos): Rₜ=1.37 min, m/z=582 [M+H]⁺.

### Separation of the Enantiomers:

80 mg of the racemic compound from Example 82 were dissolved in 4 ml of ethanol and separated into the enantiomers by means of preparative SFC on a chiral phase (see Examples 83 and 84) [column: Daicel Chiralpak AY-H, 5 μm, 250 mm×20 mm; flow rate: 80 ml/min; detection: 220 nm; injection volume: 0.75 ml; temperature: 40° C.; eluent: 60% carbon dioxide/40% ethanol; total run time 7 min].

### Example 83

### (+)-(1RS,2SR,5RS)-2-{[4-Oxo-6-(trifluoromethyl)-1,2,3-benzotriazin-3(4H)-yl]methyl}-5-{4-[(2-phenylethy)sulphanyl]benzoyl}cyclopentanecarboxylic acid (enantiomer 1)

Yield: 35 mg; chem. purity=97%; ee=100%
[α]_{D}²⁰=+77.8°, 589 nm, c=0.32 g/100 ml, chloroform

¹H-NMR (400 MHz, DMSO-d₆): δ [ppm]=12.16 (br. s, 1H), 8.51 (s, 1H), 8.46-8.37 (m, 2H), 7.92 (d, 2H), 7.43 (d, 2H), 7.34-7.26 (m, 4H), 7.25-7.18 (m, 1H), 4.62-4.52 (m, 2H), 4.16-4.06 (m, 1H), 3.37-3.30 (hidden, 2H), 3.24 (t, 1H), 2.96-2.81 (m, 3H), 2.18-2.06 (m, 1H), 2.00-1.86 (m, 1H), 1.74-1.62 (m, 1H), 1.60-1.45 (m, 1H).

LC/MS (Method 1, ESIpos): Rₜ=1.33 min, m/z=582 [M+H]⁺.

### Example 84

### (−)-(1RS,2SR,5RS)-2-{[4-Oxo-6-(trifluoromethyl)-1,2,3-benzotriazin-3(4H)-yl]methyl}-5-{4-[(2-phenylethy)sulphanyl]benzoyl}cyclopentanecarboxylic acid (enantiomer 2)

Yield: 36 mg; chem. purity=100%; ee=100%
[α]_{D}²⁰=−67.7°, 589 nm, c=0.30 g/100 ml, chloroform

¹H-NMR (400 MHz, DMSO-d₆): δ [ppm]=12.16 (br. s, 1H), 8.51 (s, 1H), 8.46-8.37 (m, 2H), 7.92 (d, 2H), 7.43 (d, 2H), 7.34-7.27 (m, 4H), 7.25-7.19 (m, 1H), 4.60-4.54 (m, 2H), 4.16-4.04 (m, 1H), 3.37-3.29 (hidden, 2H), 3.24 (t, 1H), 2.96-2.81 (m, 3H), 2.19-2.05 (m, 1H), 2.00-1.86 (m, 1H), 1.75-1.62 (m, 1H), 1.59-1.45 (m, 1H).

LC/MS (Method 1, ESIpos): Rₜ=1.33 min, m/z=582 [M+H]⁺.

### Example 85

### (+/−)-(1RS,2RS,5SR)-2-{4-[Cyclohexylmethyl)sulphanyl]benzoyl}-5-{[4-oxo-6-(trifluoromethyl)-1,2,3-benzotriazin-3(4H)-yl]methyl}cyclopentanecarboxylic acid (racemate)

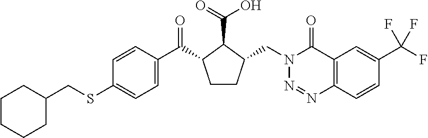 [SMILES: O=C(O)[C@H]1[C@H](Cn2nnc3ccc(C(F)(F)F)cc3c2=O)CC[C@@H]1C(=O)c1ccc(SCC2CCCCC2)cc1]

To a solution of 235 mg (0.33 mmol, 95% purity) of the compound from Example 44A in 3.5 ml of dichloromethane was added, at 0° C., 1.75 ml of trifluoroacetic acid. The mixture was stirred first at 0° C. for 15 min, then at RT for 2 h, and then concentrated. The residue was purified by means of preparative HPLC (Method 4). 184 mg (88% of theory, 91% purity) of the title compound were obtained.

¹H-NMR (400 MHz, DMSO-d₆): δ [ppm]=12.16 (br. s, 1H), 8.51 (s, 1H), 8.46-8.38 (m, 2H), 7.90 (d, 2H), 7.38 (d, 2H), 4.61-4.53 (m, 2H), 4.15-4.01 (m, 1H), 3.24 (t, 1H), 2.96 (d, 2H), 2.94-2.81 (m, 1H), 2.18-2.06 (m, 1H), 1.99-1.89 (m, 1H), 1.88-1.80 (m, 2H), 1.77-1.42 (m, 6H), 1.36-0.79 (m, 5H).

LC/MS (Method 1, ESIpos): Rₜ=1.43 min, m/z=574 [M+H]⁺.

### Separation of the Enantiomers:

174 mg of the racemic compound from Example 85 were dissolved in 3 ml of acetonitrile/30 ml of ethanol and separated into the enantiomers by means of preparative HPLC on a chiral phase (see Examples 86 and 87) [column: Daicel Chiralpak AY-H, 5 μm, 250 mm×20 mm; flow rate: 12 ml/min; detection: 220 nm; injection volume: 0.75 ml; temperature: 45° C.; eluent: t=0-16 min 100% ethanol+0.2% acetic acid].

### Example 86

### (+)-(1RS,2RS,5SR)-2-{4-[Cyclohexyl)methyl)sulphanyl]benzoyl}-5-{[4-oxo-6-(trifluoromethyl)-1,2,3-benzotriazin-3(4H)-yl]methyl}cyclopentanecarboxylic acid (enantiomer 1)

Yield: 64 mg; chem. purity=100%; ee=99%
[α]_{D}²⁰=+84.9°, 589 nm, c=0.36 g/100 ml, chloroform

¹H-NMR (400 MHz, DMSO-d₆): δ [ppm]=12.15 (br. s, 1H), 8.51 (s, 1H), 8.47-8.37 (m, 2H), 7.90 (d, 2H), 7.38 (d, 2H), 4.63-4.50 (m, 2H), 4.16-4.03 (m, 1H), 3.24 (t, 1H), 2.96 (d, 2H), 2.93-2.82 (m, 1H), 2.17-2.06 (m, 1H), 2.00-1.89 (m, 1H), 1.88-1.79 (m, 2H), 1.73-1.45 (m, 6H), 1.26-0.93 (m, 5H).

LC/MS (Method 1, ESIpos): Rₜ=1.47 min, m/z=574 [M+H]⁺.

### Example 87

### (−)-(1RS,2RS,5SR)-2-{4-[Cyclohexyl)methyl)sulphanyl]benzoyl}-5-{[4-oxo-6-(trifluoromethyl)-1,2,3-benzotriazin-3(4H)-yl]methyl}cyclopentanecarboxylic acid (enantiomer 2)

Yield: 61 mg; chem. purity=100%; ee=99%
[α]_{D}²⁰=−87.3°, 589 nm, c=0.38 g/100 ml, chloroform

¹H-NMR (400 MHz, DMSO-d₆): δ [ppm]=12.15 (br. s, 1H), 8.51 (s, 1H), 8.46-8.37 (m, 2H), 7.90 (d, 2H), 7.38 (d, 2H), 4.63-4.51 (m, 2H), 4.15-4.05 (m, 1H), 3.24 (t, 1H), 2.96 (d, 2H), 2.93-2.80 (m, 1H), 2.18-2.05 (m, 1H), 1.99-1.89 (m, 1H), 1.89-1.79 (m, 2H), 1.74-1.46 (m, 6H), 1.26-0.94 (m, 5H).

LC/MS (Method 1, ESIpos): Rₜ=1.47 min, m/z=574 [M+H]⁺.

### Example 88

### (+/−)-(1RS,2SR,5RS)-2-{[4-Oxo-6-(trifluoromethyl)-1,2,3-benzotriazin-3(4H)-yl]methyl}-5-[4-(pentylsulphanyl)benzoyl]cyclopentanecarboxylic acid (racemate)

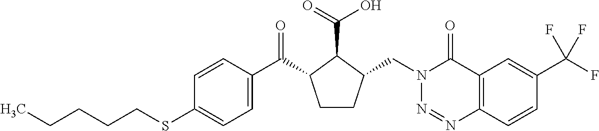 [SMILES: CCCCCSc1ccc(C(=O)[C@H]2CC[C@@H](Cn3nnc4ccc(C(F)(F)F)cc4c3=O)[C@@H]2C(=O)O)cc1]

To a solution of 233 mg (0.35 mmol) of the compound from Example 45A in 1.2 ml of dichloromethane was added, at 0° C., 0.6 ml (7.75 mmol) of trifluoroacetic acid. The mixture was stirred at RT for 1 h and then concentrated. The residue was purified by means of preparative HPLC (Method 4). 150 mg (78% of theory, 100% purity) of the title compound were obtained.

¹H-NMR (400 MHz, DMSO-d₆): δ [ppm]=12.16 (br. s, 1H), 8.51 (s, 1H), 8.46-8.38 (m, 2H), 7.90 (d, 2H), 7.39 (d, 2H), 4.63-4.51 (m, 2H), 4.16-4.05 (m, 1H), 3.24 (t, 1H), 3.06 (t, 2H), 2.94-2.79 (m, 1H), 2.18-2.03 (m, 1H), 2.00-1.86 (m, 1H), 1.74-1.46 (m, 4H), 1.45-1.20 (m, 4H), 0.86 (t, 3H).

LC/MS (Method 1, ESIpos): Rₜ=1.42 min, m/z=548 [M+H]⁺.

### Separation of the Enantiomers:

140 mg of the racemic compound from Example 88 were dissolved in 7 ml of ethanol and separated into the enantiomers by means of preparative SFC on a chiral phase (see Examples 89 and 90) [column: Daicel Chiralpak AY-H, 5 μm, 250 mm×20 mm; flow rate: 80 ml/min; detection: 220 nm; injection volume: 0.25 ml; temperature: 40° C.; eluent: 65% carbon dioxide/35% ethanol; total run time 7 min].

### Example 89

### (+)-(1RS,2SR,5RS)-2-{[4-Oxo-6-(trifluoromethyl)-1,2,3-benzotriazin-3(4H)-yl]methyl}-5-[4-(pentylsulphanyl)benzoyl]cyclopentanecarboxylic acid (enantiomer 1)

Yield: 64 mg; chem. purity=100%; ee=100%
[α]_{D}²⁰=+91.2°, 589 nm, c=0.35 g/100 ml, chloroform

¹H-NMR (400 MHz, DMSO-d₆): δ [ppm]=12.16 (br. s, 1H), 8.51 (s, 1H), 8.46-8.38 (m, 2H), 7.91 (d, 2H), 7.39 (d, 2H), 4.62-4.51 (m, 2H), 4.15-4.05 (m, 1H), 3.24 (t, 1H), 3.06 (t, 2H), 2.93-2.80 (m, 1H), 2.18-2.05 (m, 1H), 2.00-1.87 (m, 1H), 1.73-1.46 (m, 4H), 1.45-1.21 (m, 4H), 0.86 (t, 3H).

LC/MS (Method 1, ESIpos): Rₜ=1.37 min, m/z=548 [M+H]⁺.

### Example 90

### (−)-(1RS,2SR,5RS)-2-{[4-Oxo-6-(trifluoromethyl)-1,2,3-benzotriazin-3(4H)-yl]methyl}-5-[4-(pentylsulphanyl)benzoyl]cyclopentanecarboxylic acid (enantiomer 2)

Yield: 65 mg; chem. purity=100%; ee=100%
[α]_{D}²⁰=−90.8°, 589 nm, c=0.41 g/100 ml, chloroform

¹H-NMR (400 MHz, DMSO-d₆): δ [ppm]=12.16 (br. s, 1H), 8.51 (s, 1H), 8.46-8.37 (m, 2H), 7.90 (d, 2H), 7.39 (d, 2H), 4.63-4.51 (m, 2H), 4.15-4.06 (m, 1H), 3.24 (t, 1H), 3.06 (t, 2H), 2.94-2.80 (m, 1H), 2.18-2.05 (m, 1H), 1.94 (d, 1H), 1.74-1.46 (m, 4H), 1.45-1.21 (m, 4H), 0.86 (t, 3H).

LC/MS (Method 1, ESIpos): Rₜ=1.37 min, m/z=548 [M+H]⁺.

### Example 91

### (+/−)-(1RS,2RS,5SR)-2-[4-(Benzyloxy)benzoyl]-5-[(6-methyl-4-oxo-1,2,3-benzotriazin-3(4H)-yl)methyl]cyclopentanecarboxylic acid (racemate)

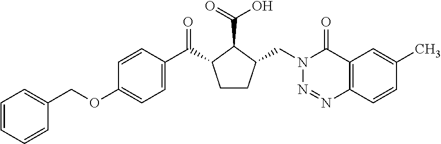 [SMILES: Cc1ccc2nnn(C[C@@H]3CC[C@H](C(=O)c4ccc(OCc5ccccc5)cc4)[C@H]3C(=O)O)c(=O)c2c1]

To a solution of 155 mg (0.25 mmol, 98% purity) of the compound from Example 46A in 0.9 ml of dichloromethane was added, at 0° C., 0.4 ml (5.58 mmol) of trifluoroacetic acid. The mixture was stirred at RT for 1.5 h and then concentrated. The residue was purified by means of preparative HPLC (Method 6). 102 mg (80% of theory, 100% purity) of the title compound were obtained.

¹H-NMR (400 MHz, DMSO-d₆): δ [ppm]=12.12 (s, 1H), 8.12-8.04 (m, 2H), 7.98 (d, 2H), 7.90 (d, 1H), 7.49-7.31 (m, 5H), 7.13 (d, 2H), 5.21 (s, 2H), 4.51 (d, 2H), 4.15-4.04 (m, 1H), 3.23 (t, 1H), 2.93-2.80 (m, 1H), 2.54 (s, 3H), 2.17-2.04 (m, 1H), 1.93-1.82 (m, 1H), 1.71-1.60 (m, 1H), 1.56-1.44 (m, 1H).

LC/MS (Method 1, ESIpos): Rₜ=1.18 min, m/z=498 [M+H]⁺.

### Separation of the Enantiomers:

92 mg of the racemic compound from Example 91 were dissolved in a mixture of 1 ml of DMSO and 11 ml of ethanol/acetonitrile and separated into the enantiomers by means of preparative SFC on a chiral phase (see Examples 92 and 93) [column: Phenomenex Amylose II, 5 μm, 250 mm×20 mm; flow rate: 100 ml/min; detection: 210 nm; injection volume: 0.95 ml; temperature: 40° C.; eluent: 70% carbon dioxide/30% ethanol; total run time 23 min].

### Example 92

### (+)-(1RS,2RS,5SR)-2-[4-(Benzyloxy)benzoyl]-5-[(6-methyl-4-oxo-1,2,3-benzotriazin-3(4H)-yl)methyl]cyclopentanecarboxylic acid (enantiomer 1)

Yield: 37 mg; chem. purity=100%; ee=100%
[α]_{D}²⁰=+64.4°, 589 nm, c=0.33 g/100 ml, chloroform

¹H-NMR (400 MHz, DMSO-d₆): δ [ppm]=12.11 (br. s, 1H), 8.12-8.05 (m, 2H), 7.97 (d, 2H), 7.90 (dd, 1H), 7.49-7.31 (m, 5H), 7.13 (d, 2H), 5.21 (s, 2H), 4.51 (d, 2H), 4.14-4.05 (m, 1H), 3.23 (t, 1H), 2.93-2.80 (m, 1H), 2.55 (s, 3H), 2.16-2.04 (m, 1H), 1.93-1.82 (m, 1H), 1.71-1.60 (m, 1H), 1.56-1.43 (m, 1H).

LC/MS (Method 1, ESIpos): Rₜ=1.18 min, m/z=498 [M+H]⁺.

### Example 93

### (−)-(1RS,2RS,5SR)-2-[4-(Benzyloxy)benzoyl]-5-[(6-methyl-4-oxo-1,2,3-benzotriazin-3(4H)-yl)methyl]cyclopentanecarboxylic acid (enantiomer 2)

Yield: 34 mg; chem. purity=100%; ee=100%
[α]_{D}²⁰=−66.4°, 589 nm, c=0.34 g/100 ml, chloroform

¹H-NMR (400 MHz, DMSO-d₆): δ [ppm]=12.10 (br. s, 1H), 8.11-8.04 (m, 2H), 7.97 (d, 2H), 7.90 (dd, 1H), 7.49-7.31 (m, 5H), 7.13 (d, 2H), 5.21 (s, 2H), 4.51 (d, 2H), 4.14-4.04 (m, 1H), 3.23 (t, 1H), 2.93-2.80 (m, 1H), 2.55 (s, 3H), 2.16-2.03 (m, 1H), 1.93-1.82 (m, 1H), 1.71-1.60 (m, 1H), 1.56-1.44 (m, 1H).

LC/MS (Method 1, ESIpos): Rₜ=1.18 min, m/z=498 [M+H]⁺.

### Example 94

### (+/−)-(1RS,2RS,5SR)-2-{4-[(4-Fluorobenzyl)oxy]benzoyl}-5-[(6-methyl-4-oxo-1,2,3-benzotriazin-3(4H)-yl)methyl]cyclopentanecarboxylic acid (racemate)

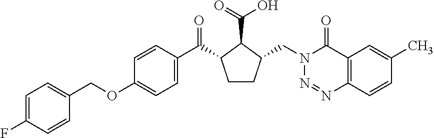 [SMILES: Cc1ccc2nnn(C[C@@H]3CC[C@H](C(=O)c4ccc(OCc5ccc(F)cc5)cc4)[C@H]3C(=O)O)c(=O)c2c1]

To a solution of 170 mg (0.27 mmol, 98% purity) of the compound from Example 48A in 0.9 ml of dichloromethane was added, at 0° C., 0.5 ml (5.95 mmol) of trifluoroacetic acid. The mixture was stirred at RT for 1.5 h and then concentrated. The residue was purified by means of preparative HPLC (Method 6). 102 mg (73% of theory, 100% purity) of the title compound were obtained.

¹H-NMR (400 MHz, DMSO-d₆): δ [ppm]=12.12 (br. s, 1H), 8.12-8.04 (m, 2H), 7.98 (d, 2H), 7.90 (dd, 1H), 7.55-7.48 (m, 2H), 7.23 (t, 2H), 7.12 (d, 2H), 5.19 (s, 2H), 4.51 (d, 2H), 4.14-4.06 (m, 1H), 3.23 (t, 1H), 2.92-2.79 (m, 1H), 2.55 (s, 3H), 2.17-2.03 (m, 1H), 1.93-1.81 (m, 1H), 1.71-1.60 (m, 1H), 1.56-1.42 (m, 1H).

LC/MS (Method 1, ESIpos): Rₜ=1.18 min, m/z=516 [M+H]⁺.

### Separation of the Enantiomers:

92 mg of the racemic compound from Example 94 were dissolved in 1 ml of DMSO and 9 ml of ethanol and separated into the enantiomers by means of preparative SFC on a chiral phase (see Examples 95 and 96) [column: Phenomenex Amylose II, 5 μm, 250 mm×20 mm; flow rate: 100 ml/min; detection: 210 nm; injection volume: 0.8 ml; temperature: 40° C.; eluent: 70% carbon dioxide/30% ethanol; total run time 18 min].

### Example 95

### (+)-(1RS,2RS,5SR)-2-{4-[(4-Fluorobenzyl)oxy]benzoyl}-5-[(6-methyl-4-oxo-1,2,3-benzotriazin-3(4H)-yl)methyl]cyclopentanecarboxylic acid (enantiomer 1)

Yield: 34 mg; chem. purity=100%; ee=100%
[α]_{D}²⁰=+56.5°, 589 nm, c=0.32 g/100 ml, chloroform

¹H-NMR (400 MHz, DMSO-d₆): δ [ppm]=12.14 (br. s, 1H), 8.11-8.04 (m, 2H), 7.98 (d, 2H), 7.90 (dd, 1H), 7.52 (dd, 2H), 7.23 (t, 2H), 7.12 (d, 2H), 5.19 (s, 2H), 4.51 (d, 2H), 4.14-4.05 (m, 1H), 3.23 (t, 1H), 2.93-2.79 (m, 1H), 2.55 (s, 3H), 2.17-2.04 (m, 1H), 1.94-1.81 (m, 1H), 1.72-1.60 (m, 1H), 1.57-1.43 (m, 1H).

LC/MS (Method 1, ESIpos): Rₜ=1.18 min, m/z=516 [M+H]⁺.

### Example 96

### (−)-(1RS,2RS,5SR)-2-{4-[(4-Fluorobenzyl)oxy]benzoyl}-5-[(6-methyl-4-oxo-1,2,3-benzotriazin-3(4H)-yl)methyl]cyclopentanecarboxylic acid (enantiomer 2)

Yield: 37 mg; chem. purity=100%; ee=100%
[α]_{D}²⁰=−64.6°, 589 nm, c=0.31 g/100 ml, chloroform.

### Example 97

### (+/−)-(1RS,2RS,5SR)-2-{4-[(3-Methylbenzyl)oxy]benzoyl}-5-[(6-methyl-4-oxo-1,2,3-benzotriazin-3(4H)-yl)methyl]cyclopentanecarboxylic acid (racemate)

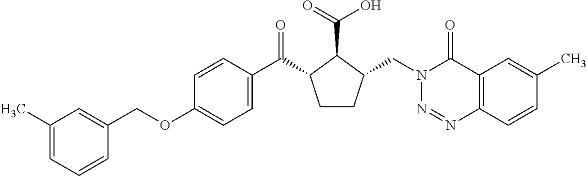

To a solution of 120 mg (0.20 mmol) of the compound from Example 49A in 0.7 ml of dichloromethane was added, at 0° C., 0.3 ml (4.30 mmol) of trifluoroacetic acid. The mixture was stirred at RT for 1.5 h and then concentrated. The residue was purified by means of preparative HPLC (Method 6). 68 mg (68% of theory, 100% purity) of the title compound were obtained.

¹H-NMR (400 MHz, DMSO-d₆): δ [ppm]=12.12 (br. s, 1H), 8.12-8.04 (m, 2H), 7.97 (d, 2H), 7.90 (dd, 1H), 7.31-7.21 (m, 3H), 7.18-7.08 (m, 3H), 5.16 (s, 2H), 4.51 (d, 2H), 4.14-4.05 (m, 1H), 3.23 (t, 1H), 2.93-2.78 (m, 1H), 2.55 (s, 3H), 2.32 (s, 3H), 2.17-2.03 (m, 1H), 1.93-1.81 (m, 1H), 1.71-1.60 (m, 1H), 1.56-1.43 (m, 1H).

LC/MS (Method 1, ESIpos): Rₜ=1.23 min, m/z=512 [M+H]⁺.

### Separation of the Enantiomers:

56 mg of the racemic compound from Example 97 were dissolved in 5 ml of acetonitrile/ethanol and separated into the enantiomers by means of preparative SFC on a chiral phase (see Examples 98 and 99) [column: Daicel Chiralpak AY-H, 5 μm, 250 mm×20 mm; flow rate: 100 ml/min; detection: 210 nm; injection volume: 0.70 ml; temperature: 40° C.; eluent: 70% carbon dioxide/30% ethanol; total run time 25 min].

### Example 98

### (+)-(1RS,2RS,5SR)-2-{4-[(3-Methylbenzyl)oxy]benzoyl}-5-[(6-methyl-4-oxo-1,2,3-benzotriazin-3(4H)-yl)methyl]cyclopentanecarboxylic acid (enantiomer 1)

Yield: 42 mg; chem. purity=100%; ee=100%
[α]_{D}²⁰=+57.7°, 589 nm, c=0.3 g/100 ml, chloroform

¹H-NMR (400 MHz, DMSO-d₆): δ [ppm]=12.11 (br. s, 1H), 8.11-8.04 (m, 2H), 7.97 (d, 2H), 7.90 (dd, 1H), 7.31-7.22 (m, 3H), 7.18-7.08 (m, 3H), 5.16 (s, 2H), 4.51 (d, 2H), 4.14-4.05 (m, 1H), 3.23 (t, 1H), 2.93-2.80 (m, 1H), 2.55 (s, 3H), 2.32 (s, 3H), 2.17-2.03 (m, 1H), 1.93-1.81 (m, 1H), 1.72-1.59 (m, 1H), 1.57-1.43 (m, 1H).

LC/MS (Method 1, ESIpos): Rₜ=1.25 min, m/z=512 [M+H]⁺.

### Example 99

### (−)-(1RS,2RS,5SR)-2-{4-[(3-Methylbenzyl)oxy]benzoyl}-5-[(6-methyl-4-oxo-1,2,3-benzotriazin-3(4H)-yl)methyl]cyclopentanecarboxylic acid (enantiomer 2)

Yield: 25 mg; chem. purity=100%; ee=100%
[α]_{D}²⁰=−59.0°, 589 nm, c=0.35 g/100 ml, chloroform

¹H-NMR (400 MHz, DMSO-d₆): δ [ppm]=12.12 (br. s, 1H), 8.12-8.04 (m, 2H), 7.97 (d, 2H), 7.91 (d, 1H), 7.32-7.22 (m, 3H), 7.18-7.08 (m, 3H), 5.16 (s, 2H), 4.51 (d, 2H), 4.15-4.04 (m, 1H), 3.23 (t, 1H), 2.94-2.78 (m, 1H), 2.55 (s, 3H), 2.32 (s, 3H), 2.17-2.03 (m, 1H), 1.93-1.81 (m, 1H), 1.72-1.59 (m, 1H), 1.56-1.43 (m, 1H).

LC/MS (Method 1, ESIpos): Rₜ=1.25 min, m/z=512 [M+H]⁺.

### Example 100

### (+/−)-(1RS,2SR,5RS)-2-[(6-Methyl-4-oxo-1,2,3-benzotriazin-3(4H)-yl)methyl]-5-(4-{[3-(trifluoromethyl)benzyl]oxy}benzoyl)cyclopentanecarboxylic acid (racemate)

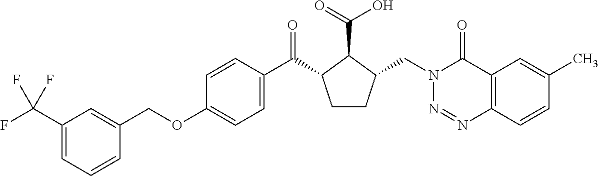 [SMILES: Cc1ccc2nnn(C[C@@H]3CC[C@H](C(=O)c4ccc(OCc5cccc(C(F)(F)F)c5)cc4)[C@H]3C(=O)O)c(=O)c2c1]

To a solution of 345 mg (0.50 mmol) of the compound from Example 50A in 1.7 ml of dichloromethane was added, at 0° C., 0.85 ml (11.03 mmol) of trifluoroacetic acid. The mixture was stirred at RT for 1.5 h and then concentrated. The residue was purified by means of preparative HPLC (Method 4). 203 mg (71% of theory, 100% purity) of the title compound were obtained.

¹H-NMR (400 MHz, DMSO-d₆): δ [ppm]=12.12 (br. s, 1H), 8.12-8.04 (m, 2H), 7.99 (d, 2H), 7.90 (dd, 1H), 7.84 (s, 1H), 7.79 (d, 1H), 7.74-7.70 (m, 1H), 7.68-7.61 (m, 1H), 7.15 (d, 2H), 5.32 (s, 2H), 4.51 (d, 2H), 4.15-4.05 (m, 1H), 3.23 (t, 1H), 2.93-2.80 (m, 1H), 2.55 (s, 3H), 2.17-2.03 (m, 1H), 1.94-1.80 (m, 1H), 1.71-1.61 (m, 1H), 1.57-1.44 (m, 1H).

LC/MS (Method 1, ESIpos): Rₜ=1.25 min, m/z=566 [M+H]⁺.

### Separation of the Enantiomers:

194 mg of the racemic compound from Example 100 were dissolved in 11 ml of ethanol and separated into the enantiomers by means of preparative SFC on a chiral phase (see Examples 101 and 102) [column: Phenomenex Amylose II, 5 μm, 250 mm×20 mm; flow rate: 100 ml/min; detection: 210 nm; injection volume: 0.30 ml; temperature: 40° C.; eluent: 65% carbon dioxide/35% ethanol; total run time 7 min].

### Example 101

### (+)-(1RS,2SR,5RS)-2-[(6-Methyl-4-oxo-1,2,3-benzotriazin-3(4H)-yl)methyl]-5-(4-{[3-(trifluoromethyl)benzyl]oxy}benzoyl)cyclopentanecarboxylic acid (enantiomer 1)

Yield: 76 mg; chem. purity=100%; ee=100%
[α]_{D}²⁰=+61.0°, 589 nm, c=0.35 g/100 ml, chloroform

¹H-NMR (400 MHz, DMSO-d₆): δ [ppm]=12.13 (br. s, 1H), 8.11-8.04 (m, 2H), 7.99 (d, 2H), 7.90 (dd, 1H), 7.85 (s, 1H), 7.79 (d, 1H), 7.75-7.70 (m, 1H), 7.69-7.62 (m, 1H), 7.15 (d, 2H), 5.32 (s, 2H), 4.51 (d, 2H), 4.16-4.05 (m, 1H), 3.23 (t, 1H), 2.94-2.79 (m, 1H), 2.56 (s, 3H), 2.17-2.04 (m, 1H), 1.94-1.80 (m, 1H), 1.72-1.60 (m, 1H), 1.58-1.43 (m, 1H).

LC/MS (Method 1, ESIpos): Rₜ=1.25 min, m/z=566 [M+H]⁺.

### Example 102

### (−)-(1RS,2SR,5RS)-2-[(6-Methyl-4-oxo-1,2,3-benzotriazin-3(4H)-yl)methyl]-5-(4-{[3-(trifluoromethyl)benzyl]oxy}benzoyl)cyclopentanecarboxylic acid (enantiomer 2)

Yield: 77 mg; chem. purity=100%; ee=92%
[α]_{D}²⁰=−57.8°, 589 nm, c=0.36 g/100 ml, chloroform

¹H-NMR (400 MHz, DMSO-d₆): δ [ppm]=12.13 (br. s, 1H), 8.12-8.04 (m, 2H), 7.99 (d, 2H), 7.90 (d, 1H), 7.84 (s, 1H), 7.79 (d, 1H), 7.75-7.70 (m, 1H), 7.69-7.63 (m, 1H), 7.15 (d, 2H), 5.32 (s, 2H), 4.51 (d, 2H), 4.16-4.05 (m, 1H), 3.23 (t, 1H), 2.93-2.80 (m, 1H), 2.17-2.04 (m, 1H), 1.95-1.81 (m, 1H), 1.73-1.60 (m, 1H), 1.56-1.44 (m, 1H).

LC/MS (Method 1, ESIpos): Rₜ=1.25 min, m/z=566 [M+H]⁺.

### Example 103

### (+/−)-(1RS,2RS,5SR)-2-{4-[(3-Chlorobenzyl)oxy]benzoyl}-5-[(6-methyl-4-oxo-1,2,3-benzotriazin-3(4H)-yl)methyl]cyclopentanecarboxylic acid (racemate)

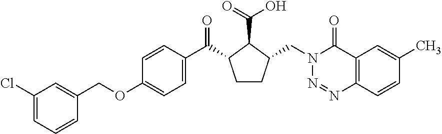 [SMILES: Cc1ccc2nnn(C[C@@H]3CC[C@H](C(=O)c4ccc(OCc5cccc(Cl)c5)cc4)[C@H]3C(=O)O)c(=O)c2c1]

To a solution of 392 mg (0.62 mmol) of the compound from Example 51A in 2.1 ml of dichloromethane was added, at 0° C., 1.0 ml (13.62 mmol) of trifluoroacetic acid. The mixture was stirred at RT for 1 h and then concentrated. The residue was purified by means of preparative HPLC (Method 4). 234 mg (71% of theory, 100% purity) of the title compound were obtained.

¹H-NMR (400 MHz, DMSO-d₆): δ [ppm]=12.12 (s, 1H), 8.12-8.04 (m, 2H), 7.98 (d, 2H), 7.90 (dd, 1H), 7.54 (s, 1H), 7.47-7.38 (m, 3H), 7.13 (d, 2H), 5.23 (s, 2H), 4.51 (d, 2H), 4.14-4.06 (m, 1H), 3.23 (t, 1H), 2.93-2.80 (m, 1H), 2.16-2.04 (m, 1H), 1.93-1.82 (m, 1H), 1.71-1.60 (m, 1H), 1.56-1.44 (m, 1H).

LC/MS (Method 1, ESIpos): Rₜ=1.25 min, m/z=532 [M+H]⁺.

### Separation of the Enantiomers:

122 mg of the racemic compound from Example 103 were dissolved in 20 ml of methanol and 3 ml of dioxane and separated into the enantiomers by means of preparative SFC on a chiral phase (see Examples 104 and 105) [column: Phenomenex Amylose II, 5 μm, 250 mm×30 mm; flow rate: 100 ml/min; detection: 210 nm; injection volume: 0.90 ml; temperature: 40° C.; eluent: 60% carbon dioxide/40% ethanol; total run time 15 min].

### Example 104

### (+)-(1RS,2RS,5SR)-2-{4-[(3-Chlorobenzyl)oxy]benzoyl}-5-[(6-methyl-4-oxo-1,2,3-benzotriazin-3(4H)-yl)methyl]cyclopentanecarboxylic acid (enantiomer 1)

Yield: 95 mg; chem. purity=100%; ee=100%
[α]_{D}²⁰=+64.6°, 589 nm, c=0.40 g/100 ml, chloroform

¹H-NMR (400 MHz, DMSO-d₆): δ [ppm]=12.13 (br. s, 1H), 8.11-8.04 (m, 2H), 7.98 (d, 2H), 7.90 (dd, 1H), 7.54 (s, 1H), 7.47-7.38 (m, 3H), 7.13 (d, 2H), 5.23 (s, 2H), 4.51 (d, 2H), 4.14-4.05 (m, 1H), 3.23 (t, 1H), 2.93-2.80 (m, 1H), 2.55 (s, 3H), 2.17-2.03 (m, 1H), 1.93-1.81 (m, 1H), 1.72-1.60 (m, 1H), 1.56-1.44 (m, 1H).

LC/MS (Method 1, ESIpos): Rₜ=1.24 min, m/z=532 [M+H]⁺.

### Example 105

### (−)-(1RS,2RS,5SR)-2-{4-[(3-Chlorobenzyl)oxy]benzoyl}-5-[(6-methyl-4-oxo-1,2,3-benzotriazin-3(4H)-yl)methyl]cyclopentanecarboxylic acid (enantiomer 2)

Yield: 91 mg; chem. purity=100%; ee=97%
[α]_{D}²⁰=−62.4°, 589 nm, c=0.27 g/100 ml, chloroform

¹H-NMR (400 MHz, DMSO-d₆): δ [ppm]=12.13 (br. s, 1H), 8.11-8.04 (m, 2H), 7.98 (d, 2H), 7.90 (d, 1H), 7.54 (s, 1H), 7.47-7.39 (m, 3H), 7.13 (d, 2H), 5.23 (s, 2H), 4.51 (d, 2H), 4.15-4.04 (m, 1H), 3.23 (t, 1H), 2.94-2.79 (m, 1H), 2.55 (s, 3H), 2.17-2.03 (m, 1H), 1.93-1.81 (m, 1H), 1.72-1.60 (m, 1H), 1.56-1.43 (m, 1H).

LC/MS (Method 1, ESIpos): Rₜ=1.24 min, m/z=532 [M+H]⁺.

### Example 106

### (+/−)-(1RS,2RS,5SR)-2-{4-[(3-Methoxybenzyl)oxy]benzoyl}-5-[(6-methyl-4-oxo-1,2,3-benzotriazin-3(4H)-yl)methyl]cyclopentanecarboxylic acid (racemate)

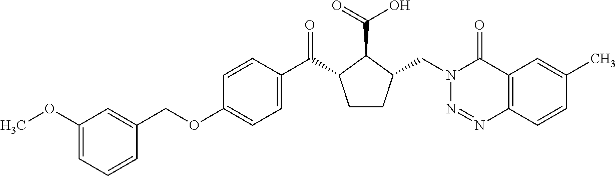

To a solution of 115 mg (0.18 mmol) of the compound from Example 52A in 0.6 ml of dichloromethane was added, at 0° C., 0.3 ml (3.93 mmol) of trifluoroacetic acid. The mixture was stirred at RT for 1 h and then concentrated. The residue was purified by means of preparative HPLC (Method 6). 72 mg (75% of theory, 99% purity) of the title compound were obtained.

¹H-NMR (400 MHz, DMSO-d₆): δ [ppm]=12.12 (br. s, 1H), 8.12-8.04 (m, 2H), 7.97 (d, 2H), 7.90 (d, 1H), 7.34-7.28 (m, 1H), 7.12 (d, 2H), 7.05-7.00 (m, 2H), 6.93-6.87 (m, 1H), 5.18 (s, 2H), 4.51 (d, 2H), 4.14-4.04 (m, 1H), 3.76 (s, 3H), 3.23 (t, 1H), 2.93-2.79 (m, 1H), 2.55 (s, 3H), 2.16-2.04 (m, 1H), 1.93-1.81 (m, 1H), 1.71-1.59 (m, 1H), 1.57-1.43 (m, 1H).

LC/MS (Method 1, ESIpos): Rₜ=1.17 min, m/z=528 [M+H]⁺.

### Separation of the Enantiomers:

64 mg of the racemic compound from Example 106 were dissolved in 11 ml of ethanol/acetonitrile and separated into the enantiomers by means of preparative SFC on a chiral phase (see Examples 107 and 108) [column: Phenomenex Amylose II, 5 μm, 250 mm×20 mm; flow rate: 100 ml/min; detection: 210 nm; injection volume: 0.95 ml; temperature: 40° C.; eluent: 70% carbon dioxide/30% ethanol; total run time 23 min].

### Example 107

### (+)-(1RS,2RS,5SR)-2-{4-[(3-Methoxybenzyl)oxy]benzoyl}-5-[(6-methyl-4-oxo-1,2,3-benzotriazin-3(4H)-yl)methyl]cyclopentanecarboxylic acid (enantiomer 1)

Yield: 25 mg; chem. purity=100%; ee=100%
[α]_{D}²⁰=+61.8°, 589 nm, c=0.32 g/100 ml, chloroform

¹H-NMR (400 MHz, DMSO-d₆): δ [ppm]=12.10 (br. s, 1H), 8.12-8.04 (m, 2H), 7.97 (d, 2H), 7.90 (d, 1H), 7.31 (t, 1H), 7.12 (d, 2H), 7.05-7.00 (m, 2H), 6.91 (d, 1H), 5.18 (s, 2H), 4.51 (d, 2H), 4.14-4.05 (m, 1H), 3.76 (s, 3H), 3.23 (t, 1H), 2.94-2.79 (m, 1H), 2.55 (s, 3H), 2.16-2.04 (m, 1H), 1.93-1.82 (m, 1H), 1.72-1.60 (m, 1H), 1.56-1.43 (m, 1H).

LC/MS (Method 1, ESIpos): Rₜ=1.17 min, m/z=528 [M+H]⁺.

### Example 108

### (−)-(1RS,2RS,5SR)-2-{4-[(3-Methoxybenzyl)oxy]benzoyl}-5-[(6-methyl-4-oxo-1,2,3-benzotriazin-3(4H)-yl)methyl]cyclopentanecarboxylic acid (enantiomer 2)

Yield: 26 mg; chem. purity=100%; ee=100%
[α]_{D}²⁰=−62.5°, 589 nm, c=0.32 g/100 ml, chloroform

¹H-NMR (400 MHz, DMSO-d₆): δ [ppm]=12.10 (br. s, 1H), 8.12-8.04 (m, 2H), 7.97 (d, 2H), 7.90 (dd, 1H), 7.31 (t, 1H), 7.12 (d, 2H), 7.05-7.00 (m, 2H), 6.93-6.88 (m, 1H), 5.18 (s, 2H), 4.51 (d, 2H), 4.14-4.04 (m, 1H), 3.76 (s, 3H), 3.23 (t, 1H), 2.93-2.80 (m, 1H), 2.55 (s, 3H), 2.16-2.04 (m, 1H), 1.93-1.82 (m, 1H), 1.72-1.59 (m, 1H), 1.56-1.43 (m, 1H).

LC/MS (Method 1, ESIpos): Rₜ=1.17 min, m/z=528 [M+H]⁺.

### Example 109

### (+/−)-(1RS,2SR,5RS)-2-[(6-Methyl-4-oxo-1,2,3-benzotriazin-3(4H)-yl)methyl]-5-(4-{[3-(trifluoromethoxy)benzyl]oxy}benzoyl)cyclopentanecarboxylic acid (racemate)

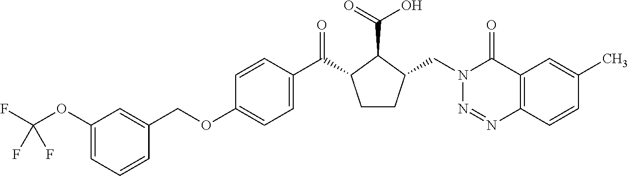 [SMILES: Cc1ccc2nnn(C[C@@H]3CC[C@H](C(=O)c4ccc(OCc5cccc(OC(F)(F)F)c5)cc4)[C@H]3C(=O)O)c(=O)c2c1]

To a solution of 95 mg (0.14 mmol) of the compound from Example 53A in 0.5 ml of dichloromethane was added, at 0° C., 0.2 ml (3.06 mmol) of trifluoroacetic acid. The mixture was stirred at RT for 1 h and then concentrated. The residue was purified by means of preparative HPLC (Method 6). 56 mg (69% of theory, 100% purity) of the title compound were obtained.

¹H-NMR (400 MHz, DMSO-d₆): δ [ppm]=12.12 (s, 1H), 8.12-8.04 (m, 2H), 7.99 (d, 2H), 7.90 (dd, 1H), 7.58-7.44 (m, 3H), 7.37-7.33 (m, 1H), 7.14 (d, 2H), 5.28 (s, 2H), 4.51 (d, 2H), 4.15-4.06 (m, 1H), 3.23 (t, 1H), 2.94-2.79 (m, 1H), 2.55 (s, 3H), 2.17-2.05 (m, 1H), 1.93-1.82 (m, 1H), 1.71-1.60 (m, 1H), 1.56-1.43 (m, 1H).

LC/MS (Method 1, ESIpos): Rₜ=1.31 min, m/z=582 [M+H]⁺.

### Separation of the Enantiomers:

48 mg of the racemic compound from Example 109 were dissolved in 8 ml of ethanol/acetonitrile and separated into the enantiomers by means of preparative SFC on a chiral phase (see Examples 110 and 111) [column: Phenomenex Amylose II, 5 μm, 250 mm×20 mm; flow rate: 100 ml/min; detection: 210 nm; injection volume: 0.70 ml; temperature: 40° C.; eluent: 70% carbon dioxide/30% ethanol; total run time 10 min].

### Example 110

### (+)-(1RS,2SR,5RS)-2-[(6-Methyl-4-oxo-1,2,3-benzotriazin-3(4H)-yl)methyl]-5-(4-{[3-(trifluoromethoxy)benzyl]oxy}benzoyl)cyclopentanecarboxylic acid (enantiomer 1)

Yield: 22 mg; chem. purity=100%; ee=100%
[α]_{D}²⁰=+51.4°, 589 nm, c=0.33 g/100 ml, chloroform

¹H-NMR (400 MHz, DMSO-d₆): δ [ppm]=12.10 (br. s, 1H), 8.12-8.04 (m, 2H), 7.99 (d, 2H), 7.90 (dd, 1H), 7.58-7.46 (m, 3H), 7.35 (d, 1H), 7.14 (d, 2H), 5.28 (s, 2H), 4.51 (d, 2H), 4.16-4.05 (m, 1H), 3.23 (t, 1H), 2.93-2.79 (m, 1H), 2.55 (s, 3H), 2.17-2.04 (m, 1H), 1.93-1.82 (m, 1H), 1.72-1.60 (m, 1H), 1.56-1.43 (m, 1H).

LC/MS (Method 1, ESIpos): Rₜ=1.27 min, m/z=582 [M+H]⁺.

### Example 111

### (−)-(1RS,2SR,5RS)-2-[(6-Methyl-4-oxo-1,2,3-benzotriazin-3(4H)-yl)methyl]-5-(4-{[3-(trifluoromethoxy)benzyl]oxy}benzoyl)cyclopentanecarboxylic acid (enantiomer 2)

Yield: 21 mg; chem. purity=100%; ee=100%
[α]_{D}²⁰=−34.5°, 589 nm, c=0.31 g/100 ml, chloroform.

### Example 112

### (+/−)-(1RS,2SR,5RS)-2-[(6-Methyl-4-oxo-1,2,3-benzotriazin-3(4H)-yl)methyl]-5-[4-(2-phenylethoxy)benzoyl]cyclopentanecarboxylic acid (racemate)

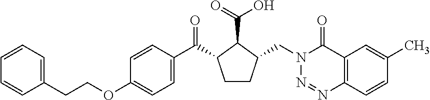 [SMILES: Cc1ccc2nnn(C[C@@H]3CC[C@H](C(=O)c4ccc(OCCc5ccccc5)cc4)[C@H]3C(=O)O)c(=O)c2c1]

To a solution of 347 mg (0.57 mmol) of the compound from Example 54A in 1.9 ml of dichloromethane was added, at 0° C., 1.0 ml (12.46 mmol) of trifluoroacetic acid. The mixture was stirred at RT for 1 h and then concentrated. The residue was purified by means of preparative HPLC (Method 4). 253 mg (87% of theory, 100% purity) of the title compound were obtained.

¹H-NMR (400 MHz, DMSO-d₆): δ [ppm]=12.11 (br. s, 1H), 8.12-8.04 (m, 2H), 7.95 (d, 2H), 7.90 (dd, 1H), 7.36-7.28 (m, 4H), 7.25-7.19 (m, 1H), 7.04 (d, 2H), 4.51 (d, 2H), 4.29 (t, 2H), 4.13-4.05 (m, 1H), 3.23 (t, 1H), 3.06 (t, 2H), 2.93-2.79 (m, 1H), 2.55 (s, 3H), 2.16-2.03 (m, 1H), 1.93-1.82 (m, 1H), 1.71-1.59 (m, 1H), 1.56-1.43 (m, 1H).

LC/MS (Method 1, ESIpos): Rₜ=1.22 min, m/z=512 [M+H]⁺.

### Separation of the Enantiomers:

243 mg of the racemic compound from Example 112 were dissolved in 21 ml of methanol and separated into the enantiomers by means of preparative SFC on a chiral phase (see Examples 113 and 114) [column: Phenomenex Amylose II, 5 μm, 250 mm×20 mm; flow rate: 100 ml/min; detection: 210 nm; injection volume: 0.40 ml; temperature: 40° C.; eluent: 65% carbon dioxide/35% ethanol; total run time 17 min].

### Example 113

### (+)-(1RS,2SR,5RS)-2-[(6-Methyl-4-oxo-1,2,3-benzotriazin-3(4H)-yl)methyl]-5-[4-(2-phenylethoxy)benzoyl]cyclopentanecarboxylic acid (enantiomer 1)

Yield: 108 mg; chem. purity=97%; ee=100%
[α]_{D}²⁰=+66.8°, 589 nm, c=0.39 g/100 ml, chloroform

¹H-NMR (400 MHz, DMSO-d₆): δ [ppm]=12.13 (br. s, 1H), 8.11-8.04 (m, 2H), 7.95 (d, 2H), 7.89 (dd, 1H), 7.36-7.28 (m, 4H), 7.26-7.18 (m, 1H), 7.04 (d, 2H), 4.51 (d, 2H), 4.29 (t, 2H), 4.13-4.05 (m, 1H), 3.22 (t, 1H), 3.06 (t, 2H), 2.93-2.79 (m, 1H), 2.55 (s, 3H), 2.16-2.02 (m, 1H), 1.93-1.80 (m, 1H), 1.71-1.59 (m, 1H), 1.57-1.43 (m, 1H).

LC/MS (Method 1, ESIpos): Rₜ=1.22 min, m/z=512 [M+H]⁺.

### Example 114

### (−)-(1RS,2SR,5RS)-2-[(6-Methyl-4-oxo-1,2,3-benzotriazin-3(4H)-yl)methyl]-5-[4-(2-phenylethoxy)benzoyl]cyclopentanecarboxylic acid (enantiomer 2)

Yield: 112 mg; chem. purity=100%; ee=99%
[α]_{D}²⁰=−63.7°, 589 nm, c=0.40 g/100 ml, chloroform

¹H-NMR (400 MHz, DMSO-d₆): δ [ppm]=12.13 (br. s, 1H), 8.11-8.04 (m, 2H), 7.95 (d, 2H), 7.89 (dd, 1H), 7.36-7.27 (m, 4H), 7.26-7.19 (m, 1H), 7.04 (d, 2H), 4.51 (d, 2H), 4.29 (t, 2H), 4.13-4.04 (m, 1H), 3.22 (t, 1H), 3.06 (t, 2H), 2.93-2.79 (m, 1H), 2.55 (s, 3H), 2.16-2.02 (m, 1H), 1.93-1.80 (m, 1H), 1.71-1.59 (m, 1H), 1.56-1.43 (m, 1H).

LC/MS (Method 1, ESIpos): Rₜ=1.22 min, m/z=512 [M+H]⁺.

### Example 115

### (+/−)-(1RS,2SR,5RS)-2-[(6-Methyl-4-oxo-1,2,3-benzotriazin-3(4H)-yl)methyl]-5-[4-(3-phenylpropoxy)benzoyl]cyclopentanecarboxylic acid (racemate)

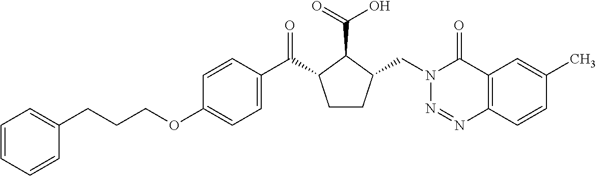 [SMILES: Cc1ccc2nnn(C[C@@H]3CC[C@H](C(=O)c4ccc(OCCCc5ccccc5)cc4)[C@H]3C(=O)O)c(=O)c2c1]

To a solution of 44 mg (0.07 mmol) of the compound from Example 55A in 0.2 ml of dichloromethane was added, at 0° C., 0.11 ml (1.55 mmol) of trifluoroacetic acid. The mixture was stirred at RT for 1 h and then concentrated. The residue was purified by means of preparative HPLC (Method 6). 23 mg (62% of theory, 100% purity) of the title compound were obtained.

¹H-NMR (400 MHz, DMSO-d₆): δ [ppm]=12.12 (s, 1H), 8.12-8.05 (m, 2H), 7.96 (d, 2H), 7.90 (dd, 1H), 7.32-7.15 (m, 5H), 7.04 (d, 2H), 4.51 (d, 2H), 4.14-4.03 (m, 3H), 3.23 (t, 1H), 2.87 (d, 1H), 2.75 (t, 2H), 2.55 (s, 3H), 2.17-1.98 (m, 3H), 1.94-1.82 (m, 1H), 1.72-1.60 (m, 1H), 1.57-1.44 (m, 1H).

LC/MS (Method 1, ESIpos): Rₜ=1.32 min, m/z=526 [M+H]⁺.

### Example 116

### (+/−)-(1RS,2RS,5SR)-2-[4-(Cyclohexylmethoxy)benzoyl]-5-[(6-methyl-4-oxo-1,2,3-benzotriazin-3(4H)-yl)methyl]cyclopentanecarboxylic acid (racemate)

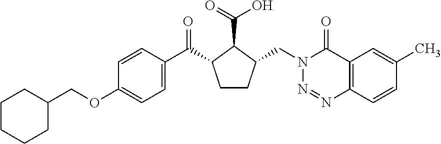 [SMILES: Cc1ccc2nnn(C[C@@H]3CC[C@H](C(=O)c4ccc(OCC5CCCCC5)cc4)[C@H]3C(=O)O)c(=O)c2c1]

To a solution of 163 mg (0.27 mmol) of the compound from Example 56A in 0.9 ml of dichloromethane was added, at 0° C., 0.5 ml (5.92 mmol) of trifluoroacetic acid. The mixture was stirred at RT for 1 h and then concentrated. The residue was purified by means of preparative HPLC (Method 6). 106 mg (78% of theory, 100% purity) of the title compound were obtained.

¹H-NMR (400 MHz, DMSO-d₆): δ [ppm]=12.12 (br. s, 1H), 8.11-8.04 (m, 2H), 7.95 (d, 2H), 7.90 (dd, 1H), 7.03 (d, 2H), 4.51 (d, 2H), 4.13-4.04 (m, 1H), 3.87 (d, 2H), 3.23 (t, 1H), 2.93-2.80 (m, 1H), 2.55 (s, 3H), 2.17-2.02 (m, 1H), 1.93-1.59 (m, 7H), 1.56-1.44 (m, 1H), 1.32-0.97 (m, 6H).

LC/MS (Method 1, ESIpos): Rₜ=1.35 min, m/z=504 [M+H]⁺.

### Separation of the Enantiomers:

98 mg of the racemic compound from Example 116 were dissolved in 10 ml of acetonitrile/ethanol and separated into the enantiomers by means of preparative SFC on a chiral phase (see Examples 117 and 118) [column: Phenomenex Amylose II, 5 μm, 250 mm×20 mm; flow rate: 100 ml/min; detection: 210 nm; injection volume: 0.80 ml; temperature: 40° C.; eluent: 70% carbon dioxide/30% ethanol; total run time 17 min].

### Example 117

### (+)-(1RS,2RS,5SR)-2-[4-(Cyclohexylmethoxy)benzoyl]-5-[(6-methyl-4-oxo-1,2,3-benzotriazin-3(4H)-yl)methyl]cyclopentanecarboxylic acid (enantiomer 1)

Yield: 40 mg; chem. purity=100%; ee=100%
[α]_{D}²⁰=+60.0°, 589 nm, c=0.32 g/100 ml, chloroform

¹H-NMR (400 MHz, DMSO-d₆): δ [ppm]=12.11 (br. s, 1H), 8.12-8.03 (m, 2H), 7.95 (d, 2H), 7.90 (dd, 1H), 7.03 (d, 2H), 4.51 (d, 2H), 4.13-4.03 (m, 1H), 3.87 (d, 2H), 3.23 (t, 1H), 2.93-2.80 (m, 1H), 2.55 (s, 3H), 2.10 (d, 1H), 1.93-1.59 (m, 7H), 1.57-1.42 (m, 1H), 1.35-0.97 (m, 6H).

LC/MS (Method 1, ESIpos): Rₜ=1.35 min, m/z=504 [M+H]⁺.

### Example 118

### (−)-(1RS,2RS,5SR)-2-[4-(Cyclohexylmethoxy)benzoyl]-5-[(6-methyl-4-oxo-1,2,3-benzotriazin-3(4H)-yl)methyl]cyclopentanecarboxylic acid (enantiomer 2)

Yield: 37 mg; chem. purity=100%; ee=100%
[α]_{D}²⁰=−68.5°, 589 nm, c=0.33 g/100 ml, chloroform

¹H-NMR (400 MHz, DMSO-d₆): δ [ppm]=12.12 (br. s, 1H), 8.12-8.04 (m, 2H), 7.95 (d, 2H), 7.90 (dd, 1H), 7.03 (d, 2H), 4.51 (d, 2H), 4.13-4.04 (m, 1H), 3.87 (d, 2H), 3.23 (t, 1H), 2.93-2.79 (m, 1H), 2.55 (s, 3H), 2.15-2.03 (m, 1H), 1.93-1.59 (m, 7H), 1.56-1.44 (m, 1H), 1.35-0.96 (m, 6H).

LC/MS (Method 1, ESIpos): Rₜ=1.35 min, m/z=504 [M+H]⁺.

### Example 119

### (+/−)-(1RS,2RS,5SR)-2-[4-(Cyclopentylmethoxy)benzoyl]-5-[(6-methyl-4-oxo-1,2,3-benzotriazin-3(4H)-yl)methyl]cyclopentanecarboxylic acid (racemate)

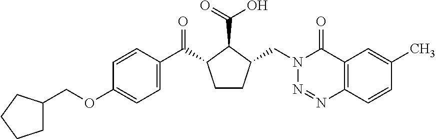 [SMILES: Cc1ccc2nnn(C[C@@H]3CC[C@H](C(=O)c4ccc(OCC5CCCC5)cc4)[C@H]3C(=O)O)c(=O)c2c1]

To a solution of 49 mg (0.049 mmol, 58% purity) of the compound from Example 57A in 0.16 ml of dichloromethane was added, at 0° C., 0.08 ml (1.07 mmol) of trifluoroacetic acid. The mixture was stirred at RT for 1 h and then concentrated. The residue was purified by means of preparative HPLC (Method 6). 18 mg (76% of theory, 100% purity) of the title compound were obtained.

¹H-NMR (400 MHz, DMSO-d₆): δ [ppm]=12.12 (br. s, 1H), 8.11-8.04 (m, 2H), 7.95 (d, 2H), 7.90 (dd, 1H), 7.03 (d, 2H), 4.51 (d, 2H), 4.13-4.04 (m, 1H), 3.94 (d, 2H), 3.23 (t, 1H), 2.93-2.79 (m, 1H), 2.55 (s, 3H), 2.38-2.24 (m, 1H), 2.17-2.03 (m, 1H), 1.93-1.43 (m, 9H), 1.40-1.25 (m, 2H).

LC/MS (Method 1, ESIpos): Rₜ=1.29 min, m/z=490 [M+H]⁺.

### Example 120

### (+/−)-(1RS,2RS,5SR)-2-[4-(Hexyloxy)benzoyl]-5-[(6-methyl-4-oxo-1,2,3-benzotriazin-3(4H)-yl)methyl]cyclopentanecarboxylic acid (racemate)

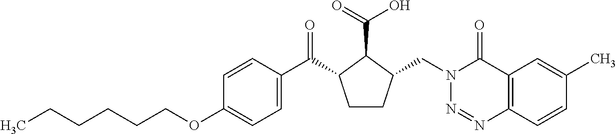 [SMILES: CCCCCCOc1ccc(C(=O)[C@H]2CC[C@@H](Cn3nnc4ccc(C)cc4c3=O)[C@@H]2C(=O)O)cc1]

To a solution of 140 mg (0.24 mmol) of the compound from Example 58A in 0.8 ml of dichloromethane was added, at 0° C., 0.4 ml (5.21 mmol) of trifluoroacetic acid. The mixture was stirred at RT for 1 h and then concentrated. The residue was purified by means of preparative HPLC (Method 6). 69 mg (59% of theory, 100% purity) of the title compound were obtained.

¹H-NMR (400 MHz, DMSO-d₆): δ [ppm]=12.12 (br. s, 1H), 8.12-8.04 (m, 2H), 7.95 (d, 2H), 7.90 (dd, 1H), 7.03 (d, 2H), 4.51 (d, 2H), 4.13-4.02 (m, 3H), 3.23 (t, 1H), 2.93-2.80 (m, 1H), 2.55 (s, 3H), 2.16-2.03 (m, 1H), 1.93-1.82 (m, 1H), 1.77-1.60 (m, 3H), 1.56-1.24 (m, 7H), 0.91-0.83 (m, 3H).

LC/MS (Method 1, ESIpos): Rₜ=1.33 min, m/z=492 [M+H]⁺.

### Separation of the Enantiomers:

60 mg of the racemic compound from Example 120 were dissolved in 11 ml of acetonitrile/ethanol and separated into the enantiomers by means of preparative SFC on a chiral phase (see Examples 121 and 122) [column: Phenomenex Amylose II, 5 μm, 250 mm×20 mm; flow rate: 100 ml/min; detection: 210 nm; injection volume: 0.6 ml; temperature: 40° C.; eluent: 70% carbon dioxide/30% ethanol; total run time 11 min].

### Example 121

### (+)-(1RS,2RS,5SR)-2-[4-(Hexyloxy)benzoyl]-5-[(6-methyl-4-oxo-1,2,3-benzotriazin-3(4H)-yl)methyl]cyclopentanecarboxylic acid (enantiomer 1)

Yield: 27 mg; chem. purity=100%; ee=100%
[α]_{D}²⁰=+42.0°, 589 nm, c=0.35 g/100 ml, chloroform

¹H-NMR (400 MHz, DMSO-d₆): δ [ppm]=12.09 (br. s, 1H), 8.12-8.05 (m, 2H), 7.95 (d, 2H), 7.90 (dd, 1H), 7.03 (d, 2H), 4.51 (d, 2H), 4.13-4.02 (m, 3H), 3.23 (t, 1H), 2.93-2.79 (m, 1H), 2.55 (s, 3H), 2.16-2.03 (m, 1H), 1.93-1.82 (m, 1H), 1.78-1.60 (m, 3H), 1.56-1.22 (m, 7H), 0.91-0.83 (m, 3H).

LC/MS (Method 1, ESIpos): Rₜ=1.34 min, m/z=492 [M+H]⁺.

### Example 122

### (−)-(1RS,2RS,5SR)-2-[4-(Hexyloxy)benzoyl]-5-[(6-methyl-4-oxo-1,2,3-benzotriazin-3(4H)-yl)methyl]cyclopentanecarboxylic acid (enantiomer 2)

Yield: 28 mg; chem. purity=100%; ee=100%
[α]_{D}²⁰=−52.6°, 589 nm, c=0.36 g/100 ml, chloroform

¹H-NMR (400 MHz, DMSO-d₆): δ [ppm]=12.09 (br. s, 1H), 8.12-8.04 (m, 2H), 7.95 (d, 2H), 7.90 (dd, 1H), 7.03 (d, 2H), 4.51 (d, 2H), 4.13-4.02 (m, 3H), 3.23 (t, 1H), 2.92-2.80 (m, 1H), 2.55 (s, 3H), 2.16-2.03 (m, 1H), 1.94-1.81 (m, 1H), 1.77-1.60 (m, 3H), 1.56-1.21 (m, 7H), 0.91-0.84 (m, 3H).

LC/MS (Method 1, ESIpos): Rₜ=1.33 min, m/z=492 [M+H]⁺.

### Example 123

### (+/−)-(1RS,2SR,5RS)-2-[(6-Methyl-4-oxo-1,2,3-benzotriazin-3(4H)-yl)methyl]-5-[4-(pentyloxy)benzoyl]cyclopentanecarboxylic acid (racemate)

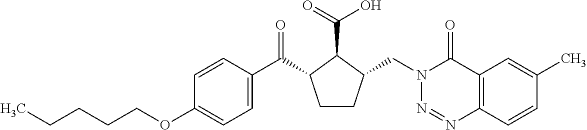 [SMILES: CCCCCOc1ccc(C(=O)[C@H]2CC[C@@H](Cn3nnc4ccc(C)cc4c3=O)[C@@H]2C(=O)O)cc1]

To a solution of 378 mg (0.58 mmol, 88% purity) of the compound from Example 59A in 2.0 ml of dichloromethane was added, at 0° C., 1.0 ml (12.66 mmol) of trifluoroacetic acid. The mixture was stirred at RT for 1 h and then concentrated. The residue was purified by means of preparative HPLC (Method 6). 228 mg (83% of theory, 100% purity) of the title compound were obtained.

¹H-NMR (400 MHz, DMSO-d₆): δ [ppm]=12.12 (s, 1H), 8.11-8.04 (m, 2H), 7.95 (d, 2H), 7.90 (dd, 1H), 7.03 (d, 2H), 4.51 (d, 2H), 4.13-4.02 (m, 3H), 3.23 (t, 1H), 2.93-2.79 (m, 1H), 2.55 (s, 3H), 2.16-2.04 (m, 1H), 1.93-1.82 (m, 1H), 1.78-1.60 (m, 3H), 1.56-1.45 (m, 1H), 1.44-1.27 (m, 4H), 0.89 (t, 3H).

LC/MS (Method 1, ESIpos): Rₜ=1.27 min, m/z=478 [M+H]⁺.

### Separation of the Enantiomers:

220 mg of the racemic compound from Example 123 were dissolved in 11 ml of ethanol while heating and separated into the enantiomers by means of preparative SFC on a chiral phase (see Examples 124 and 125) [column: Phenomenex Amylose II, 5 μm, 250 mm×20 mm; flow rate: 100 ml/min; detection: 210 nm; injection volume: 0.20 ml; temperature: 40° C.; eluent: 70% carbon dioxide/30% ethanol; total run time 12 min].

### Example 124

### (+)-(1RS,2SR,5RS)-2-[(6-Methyl-4-oxo-1,2,3-benzotriazin-3(4H)-yl)methyl]-5-[4-(pentyloxy)benzoyl]cyclopentanecarboxylic acid (enantiomer 1)

Yield: 96 mg; chem. purity=100%; ee=100%
[α]_{D}²⁰=+67.8°, 589 nm, c=0.37 g/100 ml, chloroform

¹H-NMR (400 MHz, DMSO-d₆): δ [ppm]=12.13 (br. s, 1H), 8.11-8.04 (m, 2H), 7.95 (d, 2H), 7.90 (dd, 1H), 7.03 (d, 2H), 4.51 (d, 2H), 4.14-4.01 (m, 4H), 3.22 (t, 1H), 2.93-2.80 (m, 1H), 2.55 (s, 3H), 2.15-2.03 (m, 1H), 1.92-1.82 (m, 1H), 1.78-1.60 (m, 3H), 1.56-1.45 (m, 1H), 1.44-1.28 (m, 4H), 0.92-0.86 (m, 3H).

LC/MS (Method 1, ESIpos): Rₜ=1.27 min, m/z=478 [M+H]⁺.

### Example 125

### (−)-(1RS,2SR,5RS)-2-[(6-Methyl-4-oxo-1,2,3-benzotriazin-3(4H)-yl)methyl]-5-[4-(pentyloxy)benzoyl]cyclopentanecarboxylic acid (enantiomer 2)

Yield: 98 mg; chem. purity=100%; ee=99%
[α]_{D}²⁰=−68.1°, 589 nm, c=0.33 g/100 ml, chloroform

¹H-NMR (400 MHz, DMSO-d₆): δ [ppm]=12.13 (br. s, 1H), 8.11-8.04 (m, 2H), 7.95 (d, 2H), 7.90 (dd, 1H), 7.03 (d, 2H), 4.51 (d, 2H), 4.14-4.01 (m, 4H), 3.22 (t, 1H), 2.93-2.79 (m, 1H), 2.55 (s, 3H), 2.16-2.03 (m, 1H), 1.93-1.82 (m, 1H), 1.78-1.60 (m, 3H), 1.56-1.45 (m, 1H), 1.44-1.27 (m, 4H), 0.89 (t, 3H).

LC/MS (Method 1, ESIpos): Rₜ=1.27 min, m/z=478 [M+H]⁺.

### B. Assessment of Pharmacological Efficacy

The pharmacological activity of the compounds according to the invention can be demonstrated by in vitro and in vivo studies as known to the person skilled in the art. The application examples which follow describe the biological action of the compounds according to the invention, without restricting the invention to these examples.

### ABBREVIATIONS AND ACRONYMS

APMA 4-aminophenylmercuric acetate
Brij®-35 polyoxyethylene lauryl ether
BSA bovine serum albumin
CYP cytochrome P450
Dap (or Dpa) L-2,3-diaminopropionic acid (β-amino-L-alanine)
DMSO dimethyl sulphoxide
Dnp 2,4-dinitrophenyl
EDTA ethylenediaminetetraacetic acid
HEPES 2-[4-(2-hydroxyethyl)piperazin-1-yl]ethanesulphonic acid
HME human macrophage elastase
IC inhibition concentration
Mca (7-methoxycoumarin-4-yl)acetyl
MMP matrix metallopeptidase
MTP microtitre plate
NADP⁺ nicotinamide adenine dinucleotide phosphate (oxidized form)
NADPH nicotinamide adenine dinucleotide phosphate (reduced form)
Nval norvaline
PEG polyethylene glycol
Tris tris(hydroxymethyl)aminomethane
v/v volume to volume ratio (of a solution)
w/w weight to weight ratio (of a solution)

### B-1. In Vitro HME Inhibition Test

The potency of the compounds according to the invention with respect to HME (MMP-12) is determined in an in vitro inhibition test. The HME-mediated amidolytic cleavage of a suitable peptide substrate leads to an increase in fluorescent light therein. The signal intensity of the fluorescent light is directly proportional to the enzyme activity. The active concentration of a test compound at which half the enzyme is inhibited (50% signal intensity of the fluorescent light) is reported as the IC₅₀ value.

### Standard In Vitro HME Inhibition Test:

In a 384 hole microtiter plate, in a total test volume of 41 the test buffer (0.1 M HEPES pH 7.4, 0.15 M NaCl, 0.03 M CaCl₂, 0.004 mM ZnCl₂, 0.02 M EDTA, 0.005% Brij®), the enzyme (0.5 nM HME; from R&D Systems, 917-MP, autocatalytic activation according to the manufacturer's instructions) and the intramolecularly quenched substrate [5 μM Mca-Pro-Leu-Gly-Leu-Glu-Glu-Ala-Dap(Dnp)-NH₂; Bachem, M-2670] are incubated in the absence and presence of the test substance (as a solution in DMSO) at 37° C. for two hours. The fluorescence intensity of the test mixtures is measured (excitation 323 mm, emission 393 nm). The IC₅₀ values are ascertained by plotting the fluorescent light intensity against the active ingredient concentration.

### High-Sensitivity In Vitro HME Inhibition Test:

If sub-nanomolar IC values are found for highly potent test substances in the standard HME inhibition test described above, a modified test is used to determine them more accurately. In this case, an enzyme concentration ten times lower is used (final concentration, for example, 0.05 nM) in order to achieve an elevated test sensitivity. The incubation period chosen for the test is correspondingly longer (for example 16 hours).

### In Vitro HME Inhibition Test in the Presence of Serum Albumin in the Reaction Buffer:

This test corresponds to the standard HME inhibition test described above, except using a modified reaction buffer. This reaction buffer additionally contains bovine serum albumin (BSA, fatty acid-free, A6003, from Sigma-Aldrich) of final concentration 2% (w/w), which corresponds to about half the physiological serum albumin content. The enzyme concentration in this modified test is slightly increased (e.g. 0.75 nM), as is the incubation time (e.g. three hours).

Table 1 below shows, for individual working examples of the invention, the IC₅₀ values from the standard or high-sensitivity HME inhibition test (in some cases as mean values from two or more independent individual determinations and rounded to two significant figures):

TABLE 1
Inhibition of human macrophage
elastase (HME/hMMP-12)

| Example | HME/hMMP-12 |
|---|---|
| No. | IC₅₀ [nM] |
| 1 | 0.052 |
| 2 | 0.51 |
| 3 | 0.055 |
| 4 | 0.049 |
| 5 | 0.070 |
| 6 | 0.17 |
| 7 | 0.26 |
| 8 | 0.21 |
| 9 | 145 |
| 10 | 0.36 |
| 11 | 0.014 |
| 12 | 0.019 |
| 13 | 0.018 |
| 14 | 10 |
| 15 | 0.050 |
| 16 | 0.064 |
| 17 | 48 |
| 18 | 0.11 |
| 19 | 0.027 |
| 20 | 4.6 |
| 21 | 0.045 |
| 22 | 170 |
| 23 | 0.032 |
| 24 | 0.015 |
| 25 | 0.013 |
| 26 | 1.0 |
| 27 | 0.032 |
| 28 | 0.036 |
| 29 | 4.1 |
| 30 | 0.037 |
| 31 | 0.012 |
| 33 | 0.13 |
| 34 | 160 |
| 35 | 0.013 |
| 36 | 0.13 |
| 37 | 0.080 |
| 38 | 350 |
| 39 | 0.044 |
| 40 | 0.048 |
| 41 | 0.010 |
| 42 | 39 |
| 43 | 0.039 |
| 44 | 0.024 |
| 45 | 4.1 |
| 46 | 0.034 |
| 47 | 0.025 |
| 48 | 290 |
| 49 | 0.14 |
| 50 | 0.036 |
| 51 | 30 |
| 52 | 0.017 |
| 53 | 0.027 |
| 54 | 22 |
| 55 | 0.037 |
| 56 | 0.20 |
| 57 | 0.028 |
| 58 | 0.027 |
| 59 | 4.2 |
| 61 | 0.032 |
| 62 | 4.7 |
| 63 | 0.015 |
| 64 | 0.068 |
| 65 | 1.2 |
| 66 | 0.043 |
| 67 | 0.015 |
| 68 | 2.1 |
| 69 | 0.094 |
| 70 | 0.031 |
| 71 | 2.7 |
| 72 | 0.13 |
| 73 | 0.17 |
| 74 | 160 |
| 75 | 0.051 |
| 76 | 0.10 |
| 77 | 0.040 |
| 78 | 8.8 |
| 79 | 0.25 |
| 80 | 0.079 |
| 81 | 25 |
| 82 | 0.12 |
| 83 | 0.11 |
| 84 | 33 |
| 85 | 0.22 |
| 86 | 0.018 |
| 87 | 8.3 |
| 88 | 0.023 |
| 89 | 0.045 |
| 90 | 1.1 |
| 91 | 0.074 |
| 92 | 0.048 |
| 93 | 190 |
| 94 | 0.055 |
| 95 | 0.025 |
| 96 | 120 |
| 97 | 0.40 |
| 98 | 0.10 |
| 99 | 210 |
| 100 | 0.080 |
| 101 | 0.086 |
| 102 | 8.7 |
| 103 | 0.10 |
| 104 | 0.12 |
| 105 | 14 |
| 106 | 0.078 |
| 107 | 0.035 |
| 108 | 470 |
| 109 | 0.14 |
| 110 | 0.087 |
| 111 | 140 |
| 112 | 0.66 |
| 113 | 0.24 |
| 114 | 110 |
| 115 | 0.13 |
| 116 | 0.093 |
| 117 | 0.014 |
| 118 | 150 |
| 119 | 0.048 |
| 120 | 0.10 |
| 121 | 0.078 |
| 122 | 190 |
| 123 | 0.088 |
| 124 | 0.062 |
| 125 | 6.7 |

### B-2. In Vitro MMP Inhibition Tests

The potency of the compounds according to the invention with respect to other MMPs (and hence their selectivity) is likewise determined in in vitro inhibition tests. The MMP-mediated amidolytic cleavage of a suitable peptide substrate leads to an increase in fluorescent light here too. The signal intensity of the fluorescent light is directly proportional to the enzyme activity. The active concentration of a test compound at which half the enzyme is inhibited (50% signal intensity of the fluorescent light) is reported as the IC₅₀ value.

### a) Human MMPs:

### In Vitro MMP-1 Inhibition Test:

Recombinant MMP-1 (from R&D Systems, 901-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 μl. The course of the MMP-1 reaction is measured by measuring the fluorescence intensity (excitation 320 nm, emission 410 nm) over a suitable period of time (for example over 120 min at a temperature of 32° C.).

### In Vitro MMP-2 Inhibition Test:

Recombinant MMP-2 (from R&D Systems, 902-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 μl. The course of the MMP-2 reaction is measured by measuring the fluorescence intensity (excitation 320 nm, emission 410 nm) over a suitable period of time (for example over 120 min at a temperature of 32° C.).

### In Vitro MMP-3 Inhibition Test:

Recombinant MMP-3 (from R&D Systems, 513-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Arg-Pro-Lys-Pro-Val-Glu-Nval-Trp-Arg-Lys(Dnp)-NH₂ substrate (final concentration, for example, 10 μM; R&D Systems, ES-002), so as to result in a total test volume of 50 The course of the MMP-3 reaction is measured by measuring the fluorescence intensity (excitation 320 nm, emission 410 nm) over a suitable period of time (for example over 120 min at a temperature of 32° C.).

### In Vitro MMP-7 Inhibition Test:

Recombinant MMP-7 (from R&D Systems, 907-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 The course of the MMP-7 reaction is measured by measuring the fluorescence intensity (excitation 320 nm, emission 410 nm) over a suitable period of time (for example over 120 min at a temperature of 32° C.).

### In Vitro MMP-8 Inhibition Test:

Recombinant MMP-8 (from R&D Systems, 908-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 μl. The course of the MMP-8 reaction is measured by measuring the fluorescence intensity (excitation 320 nm, emission 410 nm) over a suitable period of time (for example over 120 min at a temperature of 32° C.).

### In Vitro MMP-9 Inhibition Test:

Recombinant MMP-9 (from R&D Systems, 911-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 μl. The course of the MMP-9 reaction is measured by measuring the fluorescence intensity (excitation 320 nm, emission 410 nm) over a suitable period of time (for example over 120 min at a temperature of 32° C.).

### In Vitro MMP-10 Inhibition Test:

Recombinant MMP-10 (from R&D Systems, 910-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Arg-Pro-Lys-Pro-Val-Glu-Nval-Trp-Arg-Lys(Dnp)-NH₂ substrate (final concentration, for example, 10 μM; R&D Systems, ES-002), so as to result in a total test volume of 50 μl. The course of the MMP-10 reaction is measured by measuring the fluorescence intensity (excitation 320 nm, emission 410 nm) over a suitable period of time (for example over 120 min at a temperature of 32° C.).

### In Vitro MMP-13 Inhibition Test:

Recombinant MMP-13 (from R&D Systems, 511-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 The course of the MMP-13 reaction is measured by measuring the fluorescence intensity (excitation 320 nm, emission 410 nm) over a suitable period of time (for example over 120 min at a temperature of 32° C.).

### In Vitro MMP-14 Inhibition Test:

Recombinant MMP-14 (from R&D Systems, 918-MP) is enzymatically activated in accordance with the manufacturer's instructions using recombinant furin (from R&D Systems, 1503-SE). 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration, for example, 5 μM; R&D Systems, ES-010), so as to result in a total test volume of 50 The course of the MMP-14 reaction is measured by measuring the fluorescence intensity (excitation 320 nm, emission 410 nm) over a suitable period of time (for example over 120 min at a temperature of 32° C.).

### In Vitro MMP-16 Inhibition Test:

Recombinant MMP-16 (from R&D Systems, 1785-MP) is enzymatically activated in accordance with the manufacturer's instructions using recombinant furin (from R&D Systems, 1503-SE). 1 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration, for example, 5 μM; R&D Systems, ES-010), so as to result in a total test volume of 50 The course of the MMP-16 reaction is measured by measuring the fluorescence intensity (excitation 320 nm, emission 410 nm) over a suitable period of time (for example over 120 min at a temperature of 32° C.).

Tables 2A and 2B below show, for representative working examples of the invention, the IC₅₀ values from these tests relating to inhibition of human MMPs (in some cases as mean values from two or more independent individual determinations and rounded to two significant figures):

TABLE 2A
Inhibition of human MMPs

| Example | MMP-1 | MMP-2 | MMP-3 | MMP-7 | MMP-8 |
|---|---|---|---|---|---|
| No. | IC₅₀ [nM] | IC₅₀ [nM] | IC₅₀ [nM] | IC₅₀ [nM] | IC₅₀ [nM] |
| 1 | 5200 | 9.3 | 170 | 1100 | 2.5 |
| 2 | >40000 | 290 | 2100 | 5300 | 440 |
| 3 | 16000 | 190 | 1500 | 4900 | 260 |
| 4 | 28000 | 84 | 1100 | 2200 | 14 |
| 5 | >40000 | 220 | 1500 | 2400 | 250 |
| 11 | 3800 | 210 | 100 | 87 | 3.2 |
| 12 | 2500 | 42 | 28 | 160 | 0.67 |
| 13 | 3300 | 80 | 62 | 120 | 0.88 |
| 15 | 8300 | 480 | 220 | 120 | 26 |
| 16 | 7100 | 970 | 350 | 190 | 65 |
| 18 | >40000 | 2000 | 1000 | 910 | 61 |
| 19 | 37000 | 3800 | 3000 | 1600 | 130 |
| 23 | 24000 | 1600 | 540 | 500 | 56 |
| 25 | 2300 | 79 | 63 | 120 | 0.79 |
| 28 | 17000 | 700 | 250 | 300 | 43 |
| 31 | 5400 | 830 | 280 | 79 | 9.1 |
| 35 | 2100 | 450 | 400 | 31 | 2.0 |
| 39 | >40000 | 4600 | 420 | 730 | 290 |
| 40 | 3800 | 440 | 180 | 260 | 6.3 |
| 41 | 3100 | 97 | 41 | 68 | 1.6 |
| 44 | 3600 | 270 | 140 | 140 | 1.1 |
| 52 | 6300 | 1600 | 380 | 340 | 38 |
| 55 | 15000 | 1300 | 440 | 260 | 30 |
| 56 | >40000 | 2800 | 2000 | 1400 | 580 |
| 58 | 8400 | 580 | 300 | 180 | 16 |
| 67 | 5200 | 270 | 140 | 120 | 1.6 |
| 75 | 21000 | 4200 | 170 | 340 | 74 |
| 77 | 2900 | 970 | 100 | 150 | 41 |
| 79 | 15000 | 4000 | 800 | 1300 | 280 |
| 80 | 2500 | 1400 | 150 | 200 | 52 |
| 83 | >40000 | 6700 | 450 | 160 | 260 |
| 86 | 27000 | 4800 | 1300 | 1000 | 150 |
| 88 | 2500 | 190 | 96 | 120 | 6.2 |
| 91 | 5700 | 100 | 190 | 190 | 3.0 |
| 92 | 3100 | 47 | 150 | 110 | 2.4 |
| 97 | >40000 | 2300 | 2000 | 1500 | 310 |
| 98 | 9600 | 440 | 680 | 330 | 64 |
| 100 | >40000 | 540 | 1500 | 1000 | 43 |
| 101 | 14000 | 380 | 880 | 720 | 29 |
| 103 | 9700 | 370 | 340 | 330 | 41 |
| 104 | 3900 | 220 | 260 | 270 | 31 |
| 106 | 12000 | 270 | 450 | 190 | 11 |
| 107 | 11000 | 300 | 730 | 200 | 19 |
| 109 | >40000 | 1100 | 840 | 1700 | 87 |
| 110 | >40000 | 850 | 920 | 1200 | 120 |
| 117 | 4400 | 97 | 110 | 100 | 4.0 |
| 121 | 13000 | 280 | 410 | 350 | 18 |
| 123 | 24000 | 170 | 540 | 390 | 11 |

TABLE 2B
Inhibition of human MMPs

| Example | MMP-9 | MMP-10 | MMP-13 | MMP-14 | MMP-16 |
|---|---|---|---|---|---|
| No. | IC₅₀ [nM] | IC₅₀ [nM] | IC₅₀ [nM] | IC₅₀ [nM] | IC₅₀ [nM] |
| 1 | 4.7 | 33 | 11 | 24 | 130 |
| 2 | 480 | 310 | 400 | 480 | 1000 |
| 3 | 900 | 430 | 340 | 700 | 4000 |
| 4 | 130 | 110 | 58 | 33 | 170 |
| 5 | 960 | 93 | 350 | 620 | 2500 |
| 11 | 18 | 16 | 88 | 150 | 4500 |
| 12 | 5.9 | 15 | 39 | 78 | 86 |
| 13 | 4.4 | 22 | 33 | 150 | 170 |
| 15 | 230 | 26 | 640 | 180 | 520 |
| 16 | 440 | 37 | 1000 | 320 | 1700 |
| 18 | 940 | 200 | 1400 | 1000 | 4000 |
| 19 | 1000 | 270 | 2600 | 3000 | 5400 |
| 23 | 270 | 87 | 1500 | 1200 | 2200 |
| 25 | 3.0 | 19 | 45 | 230 | 290 |
| 28 | 260 | 130 | 620 | 1900 | 2200 |
| 31 | 2100 | 41 | 190 | 400 | 890 |
| 35 | 1400 | 53 | 53 | 170 | 300 |
| 39 | 780 | 170 | 1600 | 6400 | 14000 |
| 40 | 170 | 21 | 380 | 460 | 920 |
| 41 | 51 | 20 | 110 | 110 | 270 |
| 44 | 52 | 28 | 58 | 88 | 100 |
| 52 | 830 | 52 | 1600 | 3600 | 4600 |
| 55 | 940 | 50 | 1700 | 2400 | 5100 |
| 56 | 4900 | 250 | 2400 | >40000 | >40000 |
| 58 | 83 | 29 | 440 | 1300 | 2300 |
| 67 | 61 | 14 | 270 | 96 | 180 |
| 75 | 470 | 57 | 2300 | 1900 | 3600 |
| 77 | 550 | 30 | 770 | 1200 | 3800 |
| 79 | 3200 | 310 | 2700 | 8700 | >40000 |
| 80 | 780 | 53 | 840 | 2600 | 5500 |
| 83 | 2100 | 120 | 2800 | 3900 | >40000 |
| 86 | 1500 | 160 | 6600 | 3900 | 14000 |
| 88 | 55 | 22 | 230 | 440 | 670 |
| 91 | 21 | 21 | 50 | 68 | 1700 |
| 92 | 13 | 15 | 27 | 33 | 110 |
| 97 | 740 | 160 | 1500 | 870 | 4500 |
| 98 | 280 | 55 | 410 | 210 | 670 |
| 100 | 160 | 160 | 360 | 720 | 970 |
| 101 | 100 | 110 | 210 | 380 | 880 |
| 103 | 110 | 35 | 250 | 320 | 14000 |
| 104 | 110 | 50 | 200 | 230 | 540 |
| 106 | 76 | 39 | 120 | 180 | 190 |
| 107 | 89 | 52 | 150 | 290 | 650 |
| 109 | 150 | 100 | 190 | 670 | 3200 |
| 110 | 380 | 130 | 260 | 720 | 2100 |
| 117 | 54 | 26 | 74 | 88 | 260 |
| 121 | 57 | 93 | 300 | 820 | 1900 |
| 123 | 94 | 81 | 230 | 490 | 11000 |

On comparison of the inhibition data shown in Tables 1 and 2A/2B, it is found that the compounds according to the invention in general and the more active stereoisomers thereof in particular have very high inhibitory potency (frequently in the sub-nanomolar range) with respect to HME, and simultaneously high to very high selectivity (generally two to four orders of magnitude or even higher) with respect to related human MMPs.

### b) Rodent MMPs:

### In Vitro Mouse MMP-2 Inhibition Test:

Recombinant mouse MMP-2 (from R&D Systems, 924-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 The course of the MMP-2 reaction is measured by measuring the fluorescence intensity (excitation 320 nm, emission 410 nm) over a suitable period of time (for example over 120 min at a temperature of 32° C.).

### In Vitro Mouse MMP-3 Inhibition Test:

Recombinant mouse MMP-3 (from R&D Systems, 548-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Arg-Pro-Lys-Pro-Val-Glu-Nval-Trp-Arg-Lys(Dnp)-NH₂ substrate (final concentration, for example, 5 μM; R&D Systems, ES-002), so as to result in a total test volume of 50 μl. The course of the MMP-3 reaction is measured by measuring the fluorescence intensity (excitation 320 nm, emission 410 nm) over a suitable period of time (for example over 120 min at a temperature of 32° C.).

### In Vitro Mouse MMP-7 Inhibition Test:

Recombinant mouse MMP-7 (from R&D Systems, 2967-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration, for example, 5 μM; R&D Systems, ES-010), so as to result in a total test volume of 50 μl. The course of the MMP-7 reaction is measured by measuring the fluorescence intensity (excitation 320 nm, emission 410 nm) over a suitable period of time (for example over 120 min at a temperature of 32° C.).

### In Vitro Mouse MMP-8 Inhibition Test:

Recombinant mouse MMP-8 (from R&D Systems, 2904-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration, for example, 5 μM; R&D Systems, ES-010), so as to result in a total test volume of 50 μl. The course of the MMP-8 reaction is measured by measuring the fluorescence intensity (excitation 320 nm, emission 410 nm) over a suitable period of time (for example over 120 min at a temperature of 32° C.).

### In Vitro Mouse MMP-9 Inhibition Test:

Recombinant mouse MMP-9 (from R&D Systems, 909-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 5 μM; R&D Systems, ES-001), so as to result in a total test volume of 50 The course of the MMP-9 reaction is measured by measuring the fluorescence intensity (excitation 320 nm, emission 410 nm) over a suitable period of time (for example over 120 min at a temperature of 32° C.).

### In Vitro Mouse MMP-12 Inhibition Test:

Recombinant mouse MMP-12 (from R&D Systems, 3467-MP) is autocatalytically activated in accordance with the manufacturer's instructions. 1 μl of the test compound to be analysed (as a solution in DMSO, suitable concentrations, for example, 1 nM to 30 μM) is pipetted into 24 μl of activated enzyme (final concentration, for example, 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration, for example, 5 μM; R&D Systems, ES-010), so as to result in a total test volume of 50 The course of the MMP-12 reaction is measured by measuring the fluorescence intensity (excitation 320 nm, emission 410 nm) over a suitable period of time (for example over 120 min at a temperature of 32° C.).

### High-Sensitivity In Vitro Mouse MMP-12 Inhibition Test:

If sub-nanomolar IC values are found for highly potent test substances in the mouse MMP-12 inhibition test described above, a modified test is used to determine them more accurately. In this case, an enzyme concentration ten times lower is used (final concentration, for example, 0.1 nM) in order to achieve an elevated test sensitivity. The incubation period chosen for the test is correspondingly longer (for example 16 hours).

### In Vitro Rat MMP-2 Inhibition Test:

Recombinant rat MMP-2 (from R&D Systems, 924-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 10 μM; R&D Systems, ES-001), so as to result in a total test volume of 50 μl. The course of the MMP-2 reaction is measured by measuring the fluorescence intensity (excitation 320 nm, emission 410 nm) over a suitable period of time (for example over 120 min at a temperature of 32° C.).

### In Vitro Rat MMP-8 Inhibition Test:

Recombinant rat MMP-8 (from R&D Systems, 3245-MP) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration, for example, 5 μM; R&D Systems, ES-010), so as to result in a total test volume of 50 The course of the MMP-8 reaction is measured by measuring the fluorescence intensity (excitation 320 nm, emission 410 nm) over a suitable period of time (for example over 120 min at a temperature of 32° C.).

### In Vitro Rat MMP-9 Inhibition Test:

Recombinant mouse MMP-9 (from R&D Systems, 5427-MM) is chemically activated in accordance with the manufacturer's instructions using APMA. 1 μl of the test compound to be analysed (as a solution in DMSO, suitable concentrations, for example, 1 nM to 30 μM) is pipetted into 24 μl of activated enzyme (final concentration, for example, 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 5 μM; R&D Systems, ES-001), so as to result in a total test volume of 50 The course of the MMP-9 reaction is measured by measuring the fluorescence intensity (excitation 320 nm, emission 410 nm) over a suitable period of time (for example over 120 min at a temperature of 32° C.).

### In Vitro Rat MMP-12 Inhibition Test:

Rat MMP-12 (Uniprot NP_446415.1; construct L96-V277) is expressed with an additional N-terminal His target and a consecutive TEV cleavage sequence by means of a pDEco7 vector in E. coli (BL21). The protein thus expressed in recombinant form forms an intracellular insoluble protein compartment (called an inclusion body). This is solubilized after separation and intensive washing under denaturing conditions. For this purpose, the inclusion body pellet fragment from a 250 ml E. coli culture is taken up in a volume of 120 ml of buffer A (50 mM Tris pH 7.4, 100 mM NaCl, 0.03 mM ZnCl₂, 10 mM CaCl₂, 8 M urea). The soluble protein is renatured by dialysing 60 ml batches of the sample repeatedly at 4-8° C. against buffer B (50 mM Tris pH 7.4, 100 mM NaCl, 0.03 mM ZnCl₂, 10 mM CaCl₂). After the dialysis, the sample is centrifuged (25 000×g). The refolded protein is obtained in the supernatant with a yield of 3.7 mg per 250 ml of E. coli culture. The protein thus obtained is enzymatically active without further purifying operations or protease-mediated cleavage processes.

1 μl of the test compound to be analysed (as a solution in DMSO, suitable concentrations e.g. 1 nM to 30 μM) is pipetted into 24 μl of MMP-12 protein (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration, for example, 5 μmol; R&D Systems, ES-001), so as to result in a total test volume of 50 μl. The course of the MMP-12 reaction is measured by measuring the fluorescence intensity (excitation 320 mm, emission 410 nm) over a suitable period of time (for example over 120 min at a temperature of 32° C.).

Table 3 below shows, for representative working examples of the invention, the IC₅₀ values from the tests relating to inhibition of mouse MMPs (in some cases as mean values from two or more independent individual determinations and rounded to two significant figures):

TABLE 3
Inhibition of mouse MMPs

| Example | MMP-2 | MMP-3 | MMP-7 | MMP-8 | MMP-9 | MMP-12 |
|---|---|---|---|---|---|---|
| No. | IC₅₀ [nM] | IC₅₀ [nM] | IC₅₀ [nM] | IC₅₀ [nM] | IC₅₀ [nM] | IC₅₀ [nM] |
| 1 | 14 | 160 | 46 | 18 | 31 | 0.61 |
| 2 | 400 | 1300 | 140 | 1400 | 1100 | 4.1 |
| 3 | 290 | 1900 | 430 | 810 | 2600 | 7.0 |
| 4 | 100 | 1300 | 160 | 42 | 61 | 1.4 |
| 5 | 270 | 1300 | 160 | 360 | 930 | 1.8 |
| 11 | 250 | 590 | 23 | 18 | 61 | 1.8 |
| 23 | 2400 | 8000 | 69 | 300 | 1700 | 6.5 |
| 28 | 1000 | 8200 | 140 | 120 | 2500 | 7.1 |
| 31 | 990 | 3900 | 44 | 89 | 4300 | 6.4 |
| 39 | 3800 | 9500 | 230 | 640 | >40000 | 11 |
| 40 | 570 | 180 | 220 | 25 | 640 | 1.9 |
| 41 | 180 | 790 | 27 | 16 | 220 | 0.71 |
| 52 | 1800 | 6900 | 170 | 160 | 2800 | 10 |
| 56 | 5000 | >40000 | 990 | 1100 | >40000 | 21 |
| 75 | 4200 | 5900 | 120 | 410 | 2700 | 2.3 |
| 77 | 1400 | 1400 | 54 | 130 | 23000 | 2.0 |
| 79 | 5900 | 9300 | 1100 | 760 | 2100 | 35 |
| 80 | 1500 | 2500 | 130 | 130 | >40000 | 4.3 |
| 83 | 8100 | 5900 | 82 | 1100 | >40000 | 5.7 |
| 91 | 95 | 400 | 22 | 26 | 72 | 0.86 |
| 92 | 57 | 200 | 18 | 16 | 37 | 0.42 |
| 97 | 1900 | 4600 | 160 | 1300 | 1600 | 6.1 |
| 98 | 640 | 730 | 29 | 280 | 460 | 1.2 |
| 100 | 890 | 1800 | 150 | 250 | 1100 | 1.7 |
| 101 | 490 | 1300 | 76 | 120 | 400 | 1.1 |
| 103 | 370 | 1100 | 28 | 270 | 550 | 2.3 |
| 104 | 290 | 490 | 20 | 130 | 330 | 1.1 |
| 109 | 1300 | 1100 | 250 | 450 | 910 | 3.0 |
| 110 | 1100 | 2400 | 220 | 420 | 1100 | 0.72 |
| 117 | 130 | 470 | 16 | 13 | 230 | 0.62 |
| 121 | 420 | 2700 | 100 | 120 | 880 | 4.0 |
| 123 | 370 | 1800 | 100 | 91 | 770 | 4.9 |

On comparison of the inhibition data shown in Table 3, it is found that the compounds according to the invention in general and the more active stereoisomers thereof in particular have very high inhibitory potency (frequently in the nanomolar or even sub-nanomolar range) with respect to mouse MMP-12, and simultaneously high selectivity (generally one to two orders of magnitude or even higher) with respect to related murine MMPs.

### B-3. Animal Model of Pulmonary Emphysema

Elastase-induced pulmonary emphysema in mice, rats and hamsters is a widely used animal model for pulmonary emphysema [The Fas/Fas-ligand pathway does not mediate the apoptosis in elastase-induced emphysema in mice, Sawada et al., Exp. Lung Res. 33, 277-288 (2007)]. The animals receive an orotracheal instillation of porcine pancreas elastase. The treatment of the animals with the test substance starts on the day of the instillation of the porcine pancreas elastase and extends over a period of 3 weeks. At the end of the study, lung compliance is determined and alveolar morphometry is conducted.

### B-4. Animal Model of Silica-Induced Pulmonary Inflammation

Orotracheal administration of silica in mice, rats or hamsters leads to inflammation in the lung [Involvement of leukotrienes in the pathogenesis of silica-induced pulmonary fibrosis in mice, Shimbori et al., Exp. Lung Res. 36, 292-301 (2010)]. The animals are treated with the test substance of the day of instillation of the silica. After 24 hours, a bronchio-alveolar lavage is carried out to determine the cell content and the biomarker.

### B-5. Animal Model of Silica-Induced Pulmonary Fibrosis

Silica-induced pulmonary fibrosis in mice, rats or hamsters is a widely used animal model for pulmonary fibrosis [Involvement of leukotrienes in the pathogenesis of silica-induced pulmonary fibrosis in mice, Shimbori et al., Exp. Lung Res. 36, 292-301 (2010)]. The animals receive an orotracheal instillation of silica. The treatment of the animals with the test substance starts on the day of the instillation of the silica or therapeutically a week later and extends over a period of 6 weeks. At the end of the study, a bronchio-alveolar lavage to determine the cell content and the biomarkers and a histological assessment of pulmonary fibrosis are carried out.

### B-6. Animal Model of ATP-Induced Pulmonary Inflammation

Intratracheal administration of ATP (adenosine triphosphate) in mice leads to inflammation in the lung [Acute lung inflammation and ventilator-induced lung injury caused by ATP via the P2Y receptors: An experimental study, Matsuyama et al., Respir. Res. 9:79 (2008)]. On the day of the instillation of ATP, the animals are treated with the test substance for a duration of 24 h (by gavage, by addition to the feed or drinking water, using an osmotic minipump, by subcutaneous or intraperitoneal injection or by inhalation). At the end of the experiment, a bronchio-alveolar lavage is conducted to determine the cell content and the pro-inflammatory markers.

### B-7. CYP Inhibition Test

The ability of substances to inhibit the CYP enzymes CYP1A2, CYP2C9, CYP2D6 and CYP3A4 in humans is examined using pooled human liver microsomes as enzyme source in the presence of standard substrates (see below) which form CYP-specific metabolites. The inhibition effects are studied at six different concentrations of the test compounds [2.8, 5.6, 8.3, 16.7, 20 (or 25) and 50 μM) and compared with the extent of the CYP-specific metabolite formation of the standard substrates in the absence of the test compounds, and the corresponding IC50 values are calculated. A standard inhibitor that specifically inhibits an individual CYP isoform is always included in the incubation, in order to make results comparable between different series.

The incubation of phenacetin, diclofenac, tolbutamide, dextromethorphan or midazolam with human liver microsomes in the presence of six different concentrations of each test compound (as potential inhibitor) is carried out on a workstation (Tecan, Genesis, Crailsheim, Germany). Standard incubation mixtures contain 1.3 mM NADI)+, 3.3 mM MgCl2×6 H₂O, 3.3 mM glucose 6-phosphate, glucose 6-phosphate dehydrogenase (0.4 U/ml) and 100 mM phosphate buffer (pH 7.4) in a total volume of 200 μl Test compounds are preferably dissolved in acetonitrile. 96-Well plates are incubated for a defined period of time at 37° C. with pooled human liver microsomes. The reactions are stopped by addition of 100 μl of acetonitrile with a suitable internal standard present therein. Precipitated proteins are removed by centrifugation, and the supernatants are combined and analysed by LC-MS/MS.

### B-8. Hepatocyte Assay for Determination of Metabolic Stability

The metabolic stability of test compounds towards hepatocytes is determined by incubating the compounds at low concentrations (preferably below or around 1 μM) and at low cell counts (preferably at 1*10⁶ cells/ml) in order to ensure maximum linearity of kinetic conditions in the experiment. Seven samples from the incubation solution are taken for the LC-MS analysis within a fixed time pattern, in order to determine the half-life (i.e. the degradation) of the particular compound. This half life is used to calculate various “Clearance” parameters (CL) and “Fₘₐₓ” values (see below).

The CL and Fₘₐₓ values are a measure of the phase 1 and phase 2 metabolism of the compounds in the hepatocytes. In order to minimize the influence of the organic solvent on the enzymes in the incubation batches, the concentration thereof is generally limited to 1% (acetonitrile) or 0.1% (DMSO).

For all species and breeds, a hepatocyte cell count in the liver of 1.1*10⁸ cells/g of liver is expected. CL parameters calculated on the basis of half-lives which extend considerably beyond the incubation time (typically 90 minutes) can only be regarded as rough guide values.

The parameters calculated and the meanings thereof are:
Fₘₐₓ well-stirred [%] maximum possible bioavailability after oral administration
Calculation: (1−CL_{blood} well-stirred/QH)*100
CL_{blood} well-stirred [L/(h*kg)] calculated blood clearance (well-stirred model)
Calculation: (QH*CL′_{intrinsic})/(QH+CL′_{intrinsic})
CL′_{intrinsic}[ml/(min*kg)] maximum ability of the liver (of the hepatocytes) to metabolize a compound (assuming that the liver blood flow is not rate-limiting)
Calculation: CL′_{intrinsic, apparent}*species-specific hepatocyte count [1.1*10⁸/g liver]*species-specific liver weight [g/kg]
CL′_{intrinsic, apparent}[ml/(min*mg)] normalizes the elimination constant by dividing it by the hepatocyte cell count x used (x*10⁶/ml)
Calculation: kₑ₁[1/min]/(cell count [x*10⁶]/incubation volume [ml])
(QH=species-specific liver blood flow).

Table 4 below shows, for representative working examples of the invention, the CL and Fₘₐₓ values from this assay after incubation of the compounds with rat hepatocytes (some as mean values from two or more independent individual determinations):

TABLE 4
Calculated blood clearance and bioavailability
after incubation with rat hepatocytes

| Example | CL_{blood} | Fₘₐₓ |
|---|---|---|
| No. | [L/(h*kg)] | [%] |
| 11 | 1.12 | 73.3 |
| 92 | 1.01 | 76.1 |
| 95 | 0.50 | 88.2 |
| 101 | 0.09 | 97.8 |
| 104 | 0.99 | 76.3 |
| 110 | 0.40 | 90.4 |

### B-9. Metabolic Study

To determine the metabolic profile of the compounds according to the invention, they are incubated with liver microsomes or with primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds according to the invention are incubated with a concentration of about 1-10 μM. To this end, stock solutions of the compounds having a concentration of 0.1-1 mM in acetonitrile were prepared, and then pipetted with a 1:100 dilution into the incubation mixture. The liver microsomes are incubated at 37° C. in 50 mM potassium phosphate buffer pH 7.4 with and without NADPH-generating system consisting of 1 mM NADI)⁺, 10 mM glucose-6-phosphate and 1 unit glucose-6-phosphate dehydrogenase. Primary hepatocytes are incubated in suspension in William's E medium, likewise at 37° C. After an incubation time of 0-4 h, the incubation mixtures are stopped with acetonitrile (final concentration about 30%) and the protein was centrifuged off at about 15 000×g. The samples thus stopped are either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable eluent mixtures of acetonitrile and 10 mM aqueous ammonium formate solution or 0.05% aqueous formic acid. The UV chromatograms in conjunction with the mass spectrometry data serve for identification, structural elucidation and quantitative estimation of the metabolites, and for quantitative determination of the metabolic decrease in the compounds according to the invention in the incubation mixtures.

### B-10. Pharmacokinetic Studies In Vivo

The substance to be examined is administered to rats or mice intravenously as a solution (for example in corresponding plasma with a small addition of DMSO or in a PEG/ethanol/water mixture), and peroral administration is effected as a solution (for example in Solutol/ethanol/water or PEG/ethanol/water mixtures) or as a suspension (e.g. in tylose), in each case via a gavage. After administration of the substance, blood is obtained from the animals at fixed time points. It is heparinized, then plasma is obtained from it by centrifugation. The test substance is quantified analytically in the plasma by LC-MS/MS. The plasma concentration/time plots determined in this way are used to calculate, using an internal standard and with the aid of a validated computer program, the pharmacokinetic parameters, such as AUC (area under the concentration/time curve), Cₘₐₓ (maximum plasma concentration), t_{1/2} (half-life), V_{SS} (distribution volume) and CL (clearance), and the absolute and relative bioavailability F and Fᵣₑₗ (i.v./p.o. comparison or comparison of suspension to solution after p.o. administration).

### C. Working Examples of Pharmaceutical Compositions

The compounds according to the invention can be converted to pharmaceutical preparations as follows:

### Tablet:

### Composition:

100 mg of the compound according to the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound according to the invention, lactose and starch is granulated with a 5% solution (w/w) of the PVP in water. The granules are dried and then mixed with the magnesium stearate for 5 minutes. This mixture is compressed using a conventional tabletting press (see above for format of the tablet). The guide value used for the pressing is a pressing force of 15 kN.

### Suspension for Oral Administration:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol; the compound according to the invention is added to the suspension. The water is added while stirring. The mixture is stirred for about 6 h until the swelling of the Rhodigel is complete.

### Solution for Oral Administration:

### Composition:

500 mg of the compound according to the invention, 2.5 g of polysorbate and 97 g of polyethylene glycol 400. 20 g of oral solution correspond to a single dose of 100 mg of the compound according to the invention.

### Production:

The compound according to the invention is suspended in the mixture of polyethylene glycol and polysorbate with stirring. The stirring operation is continued until dissolution of the compound according to the invention is complete.

i.v. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. A compound of the formula (I) 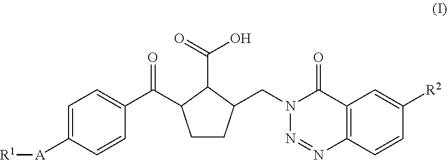
in which
A is —O— or —S—,
R¹ is straight-chain or branched (C₃-C₇)-alkyl or a group of the formula 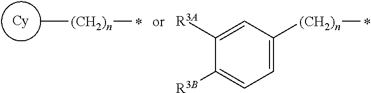
in which
* indicates the linkage to A,
n is the number 1, 2 or 3,
Cy is (C3-C6)-cycloalkyl,
R^{3A} is hydrogen, fluorine, chlorine, cyano, methyl, difluoromethyl, trifluoromethyl, methoxy, trifluoromethoxy, (trifluoromethyl)sulphanyl, methoxycarbonylamino or 2-oxo-1,3-oxazolidin-3-yl,
and
R^{3B} is hydrogen, fluorine, chlorine, methyl or trifluoromethyl,
and
R² is hydrogen, fluoromethyl, methyl, difluoromethyl or trifluoromethyl, or a salt, solvate or solvate of a salt of this compound.

2. The compound according to claim 1 in which
A is —O— or —S—,
R¹ is n-butyl, n-pentyl or n-hexyl or a group of the formula 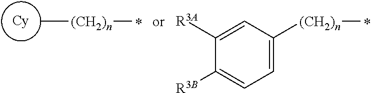
in which
* indicates the linkage to A,
n is the number 1, 2 or 3,
Cy is cyclopentyl or cyclohexyl,
R^{3A} is hydrogen, chlorine, methyl, trifluoromethyl, methoxy, trifluoromethoxy, methoxycarbonylamino or 2-oxo-1,3-oxazolidin-3-yl,
and
R^{3B} is hydrogen, fluorine or chlorine,
and
R² is hydrogen, methyl or trifluoromethyl,
or a salt, solvate or solvate of a salt of this compound.

3. The compound according to claim 1 which
A is —O—,
R¹ is n-pentyl or n-hexyl or a group of the formula 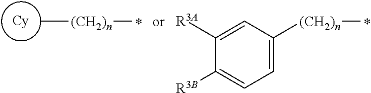
in which
* indicates the linkage to A,
n is the number 1 or 2,
Cy is cyclopentyl or cyclohexyl,
R^{3A} is hydrogen, chlorine, methyl, trifluoromethyl, methoxy or trifluoromethoxy,
and
R^{3B} is hydrogen, fluorine or chlorine,
and
R² is hydrogen, methyl or trifluoromethyl,
or a salt, solvate or solvate of a salt of this compound.

4. The compound according to claim 1 having the formula (I-A) or (I-B) 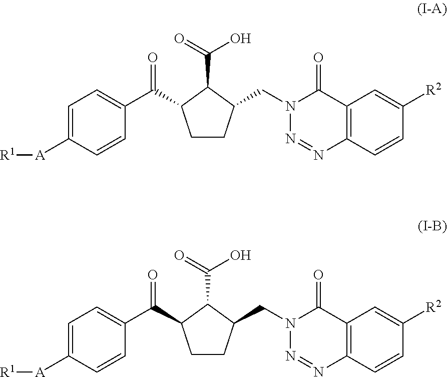
in which R¹ and R² have the definitions defined in claim 1 and the groups bonded to the central cyclopentane ring have a relative trans arrangement, or a mixture of these compounds where A, R¹ and/or R² are each identical in such a mixture of (I-A) and (I-B),
or a salt, solvate or solvate of a salt of these compounds or the mixture thereof.

5. The compound according to claim 1 having the formula (I-A) 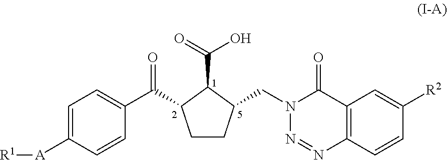
in which R¹ and R² have the definitions defined in claim 1 and the groups bonded to the central cyclopentane ring, in enantiomerically pure form, have a (1S,2S,5R) arrangement relative to one another as shown,
or a salt, solvate or solvate of a salt of this compound.

6. A method of making the compound as defined in claim 1, characterized in that
[A] a compound of the formula (II) 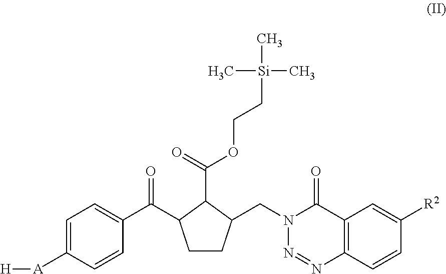
in which A and R¹ have the definitions given in claim 1
is alkylated in the presence of a base with a compound of the formula (III)
R¹—X (III)
in which R¹ has the definition given in claim 1
and
X is a leaving group, for example chlorine, bromine, iodine, mesylate, triflate or tosylate,
to give a compound of the formula (IV) 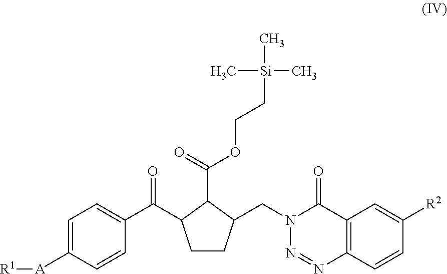
in which A, R¹ and R² have the definitions given in claim 1,
and then the 2-(trimethylsilyl)ethyl ester group is detached with the aid of an acid or a fluoride reagent to give the carboxylic acid of the formula (I) 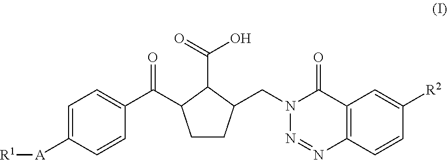
in which A, R¹ and R² have the definitions given in claim 1,
or alternatively, when A in formula (I) is —S—,
[B] a compound of the formula (II-A) 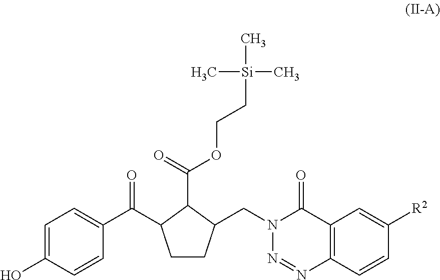
in which R² has the definition given in claim 1
is converted to the corresponding trifluoromethanesulphonate of the formula (V) 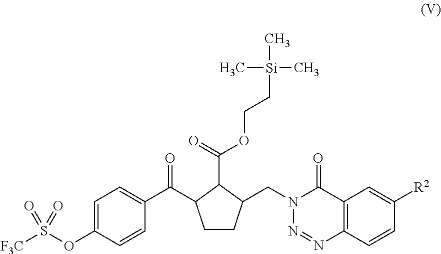
in which R¹ has the definition given in claim 1,
then the latter is reacted in the presence of a suitable palladium catalyst with a thiol of the formula (VI)
R¹—SH (VI)
in which R¹ has the definition given in claim 1
to give a compound of the formula (IV-A) 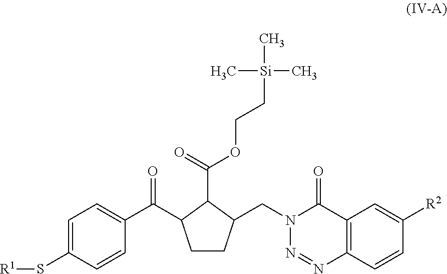
in which R¹ and R² have the definitions given in claim 1,
and subsequently the 2-(trimethylsilyl)ethyl ester group is detached with the aid of an acid or a fluoride reagent to give the carboxylic acid of the formula (I-C) 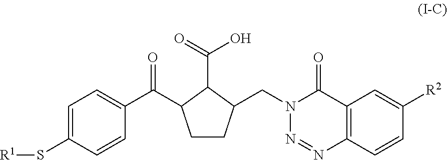
in which R¹ and R² have the definitions given in claims 1 to 5,
and, if appropriate, the compounds of the formula (I) or (I-C) thus obtained are separated into their enantiomers and/or diastereomers and/or converted with the appropriate (i) solvents and/or (ii) bases to their solvates, salts and/or solvates of the salts.

7. (canceled)

8. A method for treatment and/or prevention of at least one of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome comprising administering an effective amount of the compound as defined in claim 1.

9. A method for treatment and/or prevention of at least one of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome comprising administering an effective amount of the compound as defined in claim 1.

10. A medicament comprising the compound as defined in claim 1 in combination with one or more inert, nontoxic, pharmaceutically suitable excipients.

11. A medicament comprising the compound as defined in claim 1 in combination with one or more further active ingredients selected from the group consisting of corticosteroids, beta-adrenergic receptor agonists, antimuscarinic substances, PDE 4 inhibitors, PDE 5 inhibitors, sGC activators, sGC stimulators, HNE inhibitors, prostacyclin analogues, endothelin antagonists, statins, antifibrotic agents, antiinflammatory agents, immunomodulating agents, immunosuppressive agents and cytotoxic agents.

12. A method for treatment and/or prevention of at least one of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome comprising administering an effective amount of the medicament as defined in claim 11.

13. A method for treatment and/or prevention of at least one of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome in humans and animals by administering an effective amount of the medicament as defined in claim 10.
